(19) United States
(12) Patent Application Publication
(10) Pub. No.: US 2024/0317724 A1
(43) Pub. Date: Sep. 26, 2024

(54) 3,4-METHYLENEDIOXYMETHAMPHETAMINE AND RELATED PSYCHEDELICS AND USES THEREOF

(71) Applicant: Terran Biosciences Inc., New York, NY (US)

(72) Inventors: Sam CLARK, Miami, FL (US); Matthew Alexander James DUNCTON, Las Vegas, NV (US)

(21) Appl. No.: 18/576,719

(22) PCT Filed: Jul. 7, 2022
(86) PCT No.: PCT/US2022/036410

§ 371 Date: Jan. 4, 2024

Related U.S. Application Data
(60) Provisional application No. 63/335,108, filed on Apr. 26, 2022.
(60) Provisional application No. 63/289,024, filed on Dec. 13, 2021.
(60) Provisional application No. 63/281,488, filed on Nov. 19, 2021.
(60) Provisional application No. 63/235,539, filed on Aug. 20, 2021.
(60) Provisional application No. 63/219,322, filed on Jul. 7, 2021.

(51) Int. Cl. C07D 405/14; A61K 31/36; A61K 31/4525; A61K 31/4545; C07D 317/58; C07D 405/12; C07D 407/12

(52) U.S. Cl. CPC C07D 405/14; A61K 31/36; A61K 31/4525; A61K 31/4545; C07D 317/58; C07D 405/12; C07D 407/12

(57) ABSTRACT

Described herein are compounds that are derivatives (e.g., prodrugs) of 3,4-Methylenedioxymethamphetamine (MDMA). Also described herein are uses of the compounds provided herein for treating or preventing a disease, disorder, or condition in which an increased level of MDMA is beneficial.

### CROSS REFERENCE TO RELATED APPLICATIONS

This application claims the benefit of U.S. Provisional Patent Application No. 63/219,322, filed Jul. 7, 2021, U.S. Provisional Patent Application No. 63/235,539, filed on Aug. 20, 2021, U.S. Provisional Patent Application No. 63/281,488, filed on Nov. 19, 2021, U.S. Provisional Patent Application No. 63/289,024, filed on Dec. 13, 2021, and U.S. Provisional Patent Application No. 63/335,108, filed Apr. 26, 2022, the content of each of which is incorporated by reference herein in its entirety.

### BACKGROUND

Nearly 1 in 5 adults in the United States suffer from mental illness, and over 50% of Americans will be diagnosed with a psychiatric disorder at some point in their lifetime. 1 in 25 Americans is afflicted with severe mental illness, such as major depression, schizophrenia, or bipolar disorder.

### SUMMARY

In one aspect, provided herein are compounds of Formula (I′), or a stereoisomer, a hydrate, or a pharmaceutically acceptable salt thereof:

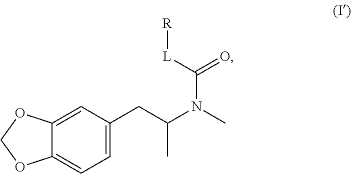

wherein:
L is bond, —O—, or NR′;
R and R are each alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (I′) is a compound of Formula (I), or a stereoisomer, a hydrate, or a pharmaceutically acceptable salt thereof:

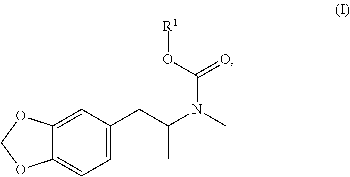

wherein:
R¹ is alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₅ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (I) has a structure of Formula (Ia), or a stereoisomer, a hydrate, or a pharmaceutically acceptable salt thereof:

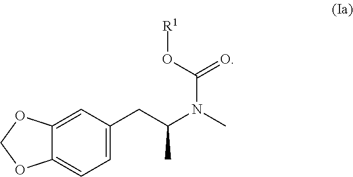

In certain embodiments, the compound of Formula (I) has a structure of Formula (Ib), or a stereoisomer, a hydrate, or a pharmaceutically acceptable salt thereof:

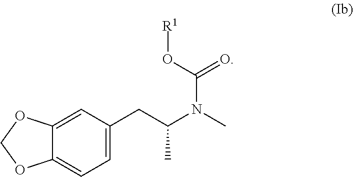

In certain embodiments, R¹ is substituted alkyl.

In certain embodiments, R¹ is alkyl substituted with heteroalkyl, heterocyclylalkyl, or heteroaryl, wherein each of heteroalkyl, heterocyclylalkyl, and heteroaryl is unsubstituted or substituted.

In certain embodiments, R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, R¹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, the compound of Formula (I) has a structure of Formula (I-1), or a stereoisomer, a hydrate, or a pharmaceutically acceptable salt thereof:

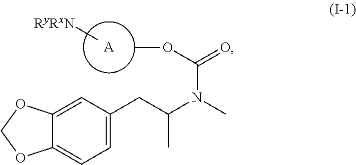

wherein

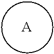

is cycloalkyl or heterocyclylalkyl, and each of R^{x} and R^{y} is alkyl or hydrogen, or R^{x} and R^{y} together with the atom to which they are attached form a heterocyclylalkyl ring.

In certain embodiments, the compound of Formula (I-1) has a structure of Formula (I-1a), or a stereoisomer, a hydrate, or a pharmaceutically acceptable salt thereof:

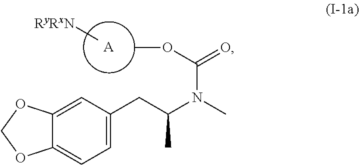

wherein

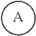

is cycloalkyl or heterocyclylalkyl, and each of R^{x} and R^{y} is alkyl or hydrogen, or R^{x} and R^{y} together with the atom to which they are attached form a heterocyclylalkyl ring.

In certain embodiments, the compound of Formula (I-1) has a structure of Formula (I-1b), or a stereoisomer, a hydrate, or a pharmaceutically acceptable salt thereof:

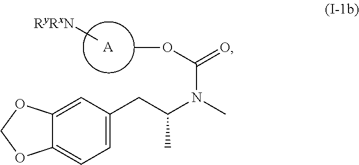

wherein

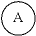

is cycloalkyl or heterocyclylalkyl, and each of R^{x} and R^{y} is alkyl or hydrogen, or R^{x} and R^{y} together with the atom to which they are attached form a heterocyclylalkyl ring.

In certain embodiments, the compound of Formula (I′) has a structure of Formula (II), or a stereoisomer, a hydrate, or a pharmaceutically acceptable salt thereof:

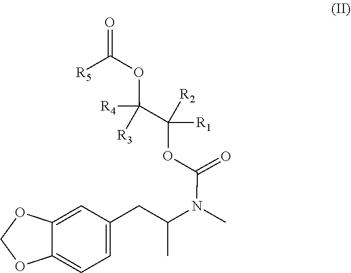

wherein:
each of R¹ and R² is independently hydrogen or alkyl optionally substituted with one or more Q, or R¹ and R² together with the atom to which they are attached form a cycloalkyl ring;
each of R³ and R⁴ is independently hydrogen or alkyl optionally substituted with one or more Q, or R³ and R⁴ together with the atom to which they are attached form a cycloalkyl ring; and
R⁵ is hydrogen, alkyl, cycloalkyl, heteroalkyl, heterocyclylalkyl, aryl, or heteroaryl, each of which is optionally substituted with one or more Q, or R⁵ together with the carbonyl to which R⁵ is attached form an amino acid residue;
   wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
   wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (II) has a structure of Formula (IIa), or a stereoisomer, a hydrate, or a pharmaceutically acceptable salt thereof:

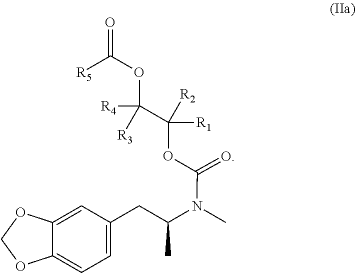

In certain embodiments, the compound of Formula (II) has a structure of Formula (IIb), or a stereoisomer, a hydrate, or a pharmaceutically acceptable salt thereof:

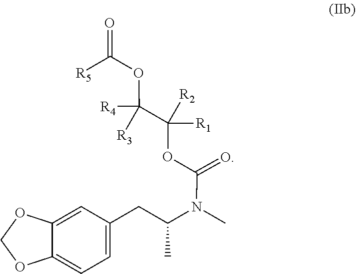

In certain embodiments, R³ and R⁴ are each hydrogen.

In certain embodiments, R³ and R⁴ are each independently alkyl.

In certain embodiments, R³ and R⁴ are each independently methyl.

In certain embodiments, R³ and R⁴ together with the atom to which they are attached form a cycloalkyl or heterocyclyl ring.

In certain embodiments, R¹ and R² are each hydrogen.

In certain embodiments, R³ and R⁴ are each independently alkyl, and R¹ and R² are each hydrogen.

In certain embodiments, each of R¹, R², R³, and R⁴ is hydrogen.

In certain embodiments, R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl, each of which is optionally substituted with one or more Q.

In certain embodiments, R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl.

In certain embodiments, the compound of Formula (I′) is a compound of Formula (III), or a stereoisomer, a hydrate, or a pharmaceutically acceptable salt thereof:

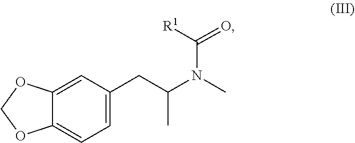

wherein:
R¹ is alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (III) has a structure of Formula (IIIa), or a stereoisomer, a hydrate, or a pharmaceutically acceptable salt thereof:

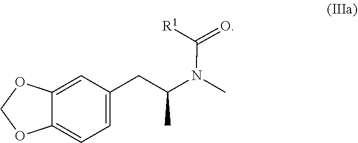

In certain embodiments, the compound of Formula (III) has a structure of Formula (IIIb), or a stereoisomer, a hydrate, or a pharmaceutically acceptable salt thereof:

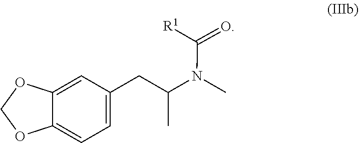

In certain embodiments, R¹ is unsubstituted alkyl.

In certain embodiments, R¹ is substituted alkyl.

In certain embodiments, R¹ is alkyl substituted with heteroalkyl, heterocyclylalkyl, or heteroaryl, wherein each of heteroalkyl, heterocyclylalkyl, and heteroaryl is unsubstituted or substituted.

In certain embodiments, R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, R¹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In certain embodiments, the compound of Formula (I′) has a structure of Formula (IV), or a stereoisomer, a hydrate, or a pharmaceutically acceptable salt thereof:

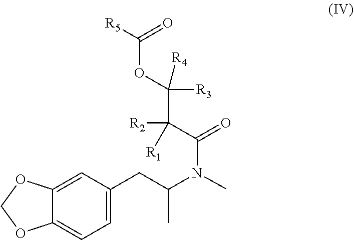

wherein:
each of R¹ and R² is independently hydrogen or alkyl optionally substituted with one or more Q, or R¹ and R² together with the atom to which they are attached form a cycloalkyl or heterocyclyl ring;
each of R³ and R⁴ is independently hydrogen or alkyl optionally substituted with one or more Q, or R³ and R⁴ together with the atom to which they are attached form a cycloalkyl or heterocyclyl ring;
and R⁵ is alkyl, cycloalkyl, heteroalkyl, heterocyclylalkyl, aryl, or heteroaryl, each of which is optionally substituted with one or more Q, or R⁵ together with the carbonyl to which R⁵ is attached form an amino acid residue;
   wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
   wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{c}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (IV) has a structure of Formula (IVa), or a stereoisomer, a hydrate, or a pharmaceutically acceptable salt thereof:

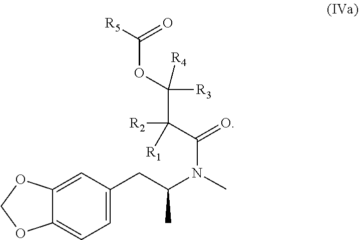

In certain embodiments, the compound of Formula (IV) has a structure of Formula (IVb), or a stereoisomer, a hydrate, or a pharmaceutically acceptable salt thereof:

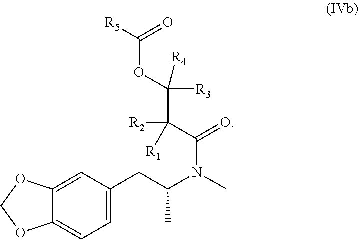

In certain embodiments, the compound of Formula (I′) has a structure of Formula (V), or a stereoisomer, a hydrate, or a pharmaceutically acceptable salt thereof:

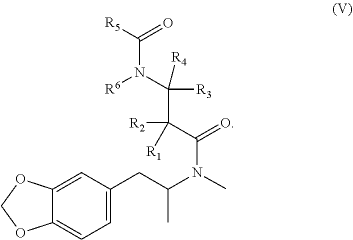

wherein:
each of R¹ and R² is independently hydrogen or alkyl optionally substituted with one or more Q, or R¹ and R² together with the atom to which they are attached form a cycloalkyl or heterocyclyl ring;
each of R³ and R⁴ is independently hydrogen or alkyl optionally substituted with one or more Q, or R³ and R⁴ together with the atom to which they are attached form a cycloalkyl or heterocyclyl ring; and
each of R⁵ and R⁶ is independently hydrogen, alkyl, cycloalkyl, heteroalkyl, heterocyclylalkyl, aryl, or heteroaryl, each of which is optionally substituted with one or more Q;
   wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
   wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (V) has a structure of Formula (Va), or a stereoisomer, a hydrate, or a pharmaceutically acceptable salt thereof:

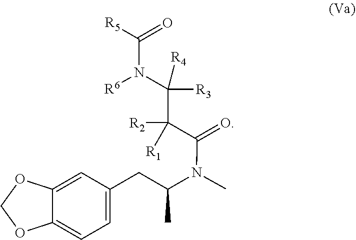

In certain embodiments, the compound of Formula (V) has a structure of Formula (Vb), or a stereoisomer, a hydrate, or a pharmaceutically acceptable salt thereof:

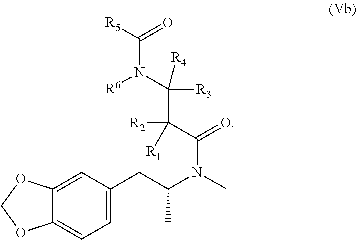

In certain embodiments, R³ and R⁴ are each hydrogen.

In certain embodiments, R³ and R⁴ are each independently alkyl.

In certain embodiments, R³ and R⁴ are each independently methyl.

In certain embodiments, R³ and R⁴ together with the atom to which they are attached form a cycloalkyl or heterocyclyl ring.

In certain embodiments, R¹ and R² are each hydrogen.

In certain embodiments, R³ and R⁴ are each independently alkyl, and R¹ and R² are each hydrogen.

In certain embodiments, each of R¹, R², R³, and R⁴ is hydrogen.

In certain embodiments, R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl, each of which is optionally substituted with one or more Q.

In certain embodiments, R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl.

In certain embodiments, R⁶ is hydrogen or alkyl.

In certain embodiments, R⁶ is hydrogen.

In certain embodiments, R⁶ is alkyl.

In certain embodiments, R⁶ is methyl.

In certain embodiments, the compound of Formula (I′) has a structure of Formula (VI), or a stereoisomer, a hydrate, or a pharmaceutically acceptable salt thereof:

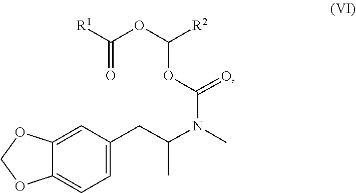

wherein:
R¹ is hydrogen, or R¹ is alkyl, cycloalkyl, heteroalkyl, heterocyclylalkyl, aryl, or heteroaryl, each of which is optionally substituted with one or more Q; or R¹ and the carbonyl to which R¹ is attached form an amino acid residue;
R² is hydrogen or alkyl optionally substituted with one or more Q; and wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
   wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound of Formula (VI) has a structure of Formula (VI-1), or a stereoisomer, a hydrate, or a pharmaceutically acceptable salt thereof:

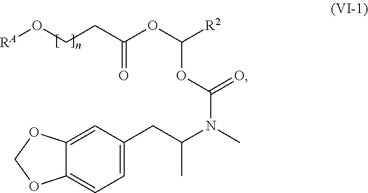

wherein R^{A} is alkyl, heteroalkyl, cycloalkyl, or heterocyclylalkyl, each of which is substituted or unsubstituted; R² is alkyl that is substituted or unsubstituted, or hydrogen; and n is 1, 2, 3, 4, 5, or 6.

In certain embodiments, R^{A} is methyl, ethyl, isopropyl, n-propyl, tert-butyl, n-butyl, n-pentyl, iso-amyl, cyclopropyl, cyclobutyl, cyclopentyl, or cyclohexyl.

In certain embodiments, R^{A} is methyl.

In certain embodiments, the compound of Formula (VI) has a structure of Formula (VI-2), or a stereoisomer, a hydrate, or a pharmaceutically acceptable salt thereof:

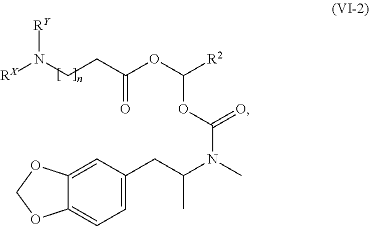

wherein each of R^{X} and R^{Y} is independently hydrogen, alkyl, heteroalkyl, cycloalkyl, or heterocyclylalkyl, wherein alkyl, heteroalkyl, cycloalkyl, or heterocyclylalkyl are substituted or unsubstituted; or R^{X} and R^{Y} together with the atom to which they are attached form a heterocyclylalkyl ring that is substituted or unsubstituted; R² is alkyl that is substituted or unsubstituted, or hydrogen; and n is 1, 2, 3, 4, 5, or 6.

In certain embodiments, each of R^{X} and R^{Y} is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In certain embodiments, the compound of Formula (VI) has a structure of Formula (VI-3), or a stereoisomer, a hydrate, or a pharmaceutically acceptable salt thereof:

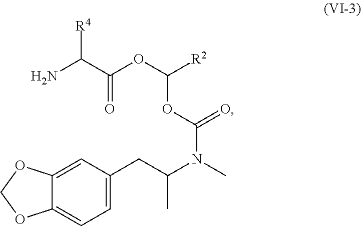

wherein R⁴ is hydrogen, substituted or unsubstituted alkyl, substituted or unsubstituted heteroalkyl, or an amino acid side chain; and R² is alkyl that is substituted or unsubstituted, or hydrogen.

In certain embodiments, the compound of Formula (I′) has a structure of Formula (VIII), or a stereoisomer, a hydrate, or a pharmaceutically acceptable salt thereof:

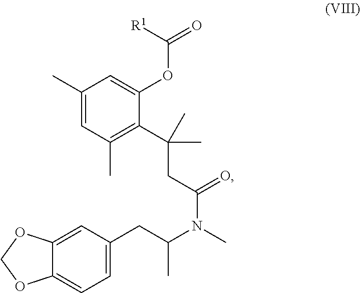

wherein R¹ is hydrogen, or R¹ is alkyl, alkenyl, heteroalkyl, cycloalkyl, heterocyclylalkyl, aryl, or heteroaryl, each of which is optionally substituted with one or more Q, or R¹ and the carbonyl to which R¹ is attached form an amino acid residue.

In certain embodiments, R¹ is hydrogen.

In certain embodiments, R¹ is optionally substituted alkyl or heteroalkyl.

In certain embodiments, R¹ is optionally substituted alkyl.

In certain embodiments, R¹ is unsubstituted alkyl.

In certain embodiments, R¹ is methyl, ethyl, n-propyl, isopropyl, tert-butyl, or n-pentyl.

In certain embodiments, L is bond.

In certain embodiments, L is —O—.

In certain embodiments, L is —NR′—.

In certain embodiments, R is alkyl, cycloalkyl, heteroalkyl, heterocyclylalkyl, aryl, or heteroaryl, each of which is optionally substituted with one or more Q; wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —N^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, R is alkyl or heterocyclylalkyl optionally substituted with one or more Q.

In certain embodiments, R is alkyl substituted with one or more Q.

In certain embodiments, R is heterocyclylalkyl.

In certain embodiments, R is optionally substituted methyl, ethyl, n-propyl, n-butyl, i-butyl, sec-butyl, t-butyl, tetrahydrofuranyl, or tetrahydropyranyl.

In certain embodiments, R is optionally substituted ethyl, n-propyl, n-butyl, i-butyl, sec-butyl, t-butyl, tetrahydrofuranyl, or tetrahydropyranyl, or R is substituted methyl.

In certain embodiments, R is ethyl, n-propyl, i-propyl, i-butyl, sec-butyl, t-butyl, tetrahydrofuranyl, tetrahydropyranyl, —CH₂CH₂—OCH₃, —CH₂CH₂—COOH, —CH₂CH₂CH₂—COOH, —CH₂CH₂CH₂CH₂—COOH, —CH₂—OC(O)C(CH₃)₃, —CH₂CH₂—C(O)OC(CH₃)₃, —CH₂CH₂CH₂—C(O)OC(CH₃)₃, —CH₂CH₂CH₂CH₂—C(O)OC(CH₃)₃, —CH₂N(CH₃)₂, —C(CH₃)NHC(O)OC(CH₃)₃, —C[CH(CH₃)₂]NHC(O)OC(CH₃)₃,

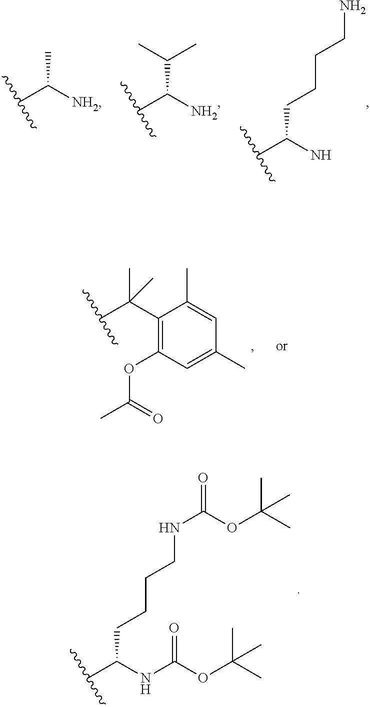

In certain embodiments, R is optionally substituted alkyl, R′ is hydrogen or unsubstituted alkyl.

In certain embodiments, R′ is hydrogen or methyl.

In certain embodiments, the compound provided herein (e.g. a compound of Formula (I′)) is a compound in Table 1.

In certain embodiments, the compound provided herein (e.g. a compound of Formula (I′)) is a compound in Tables 2-7.

In certain embodiments, the compound provided herein (e.g. a compound of Formula (I′)) is selected from the group consisting of:

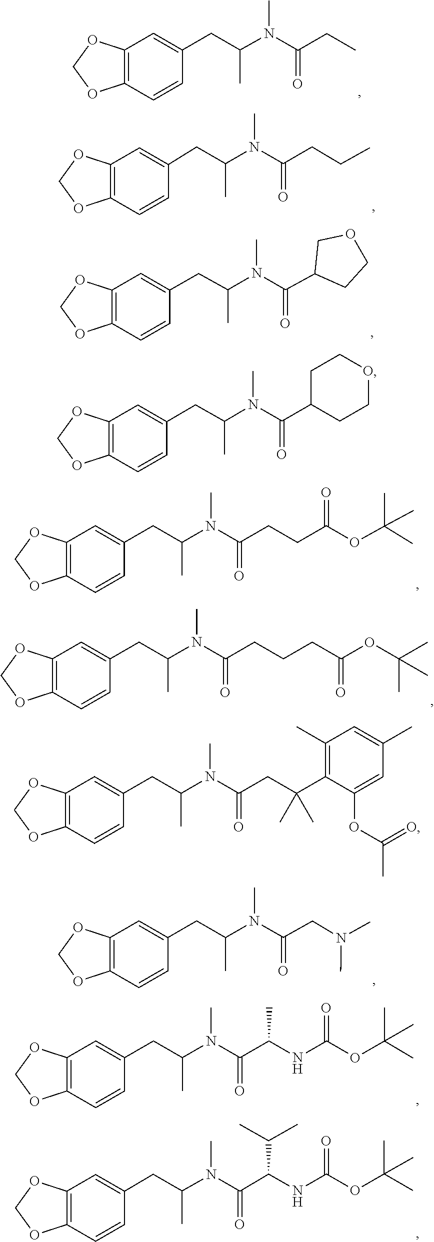

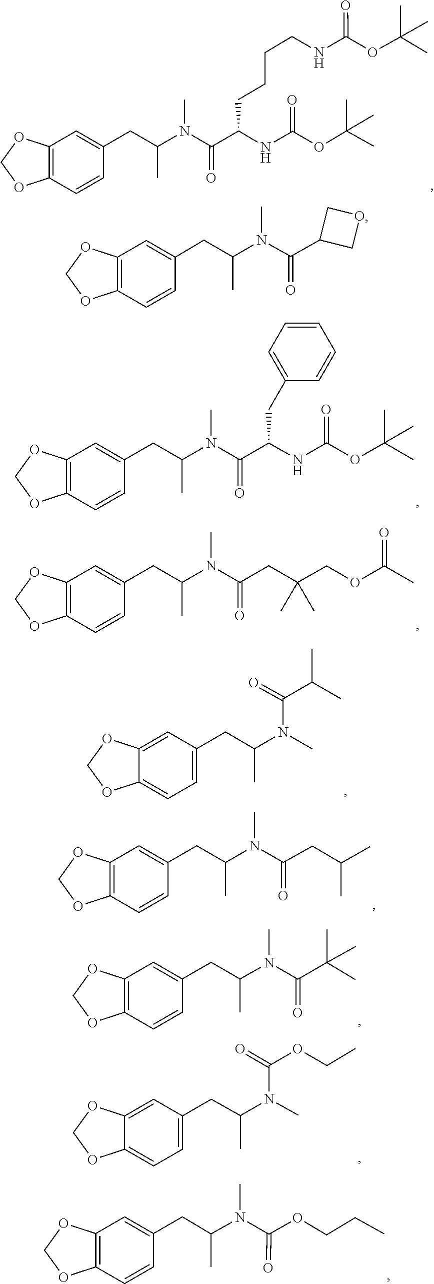

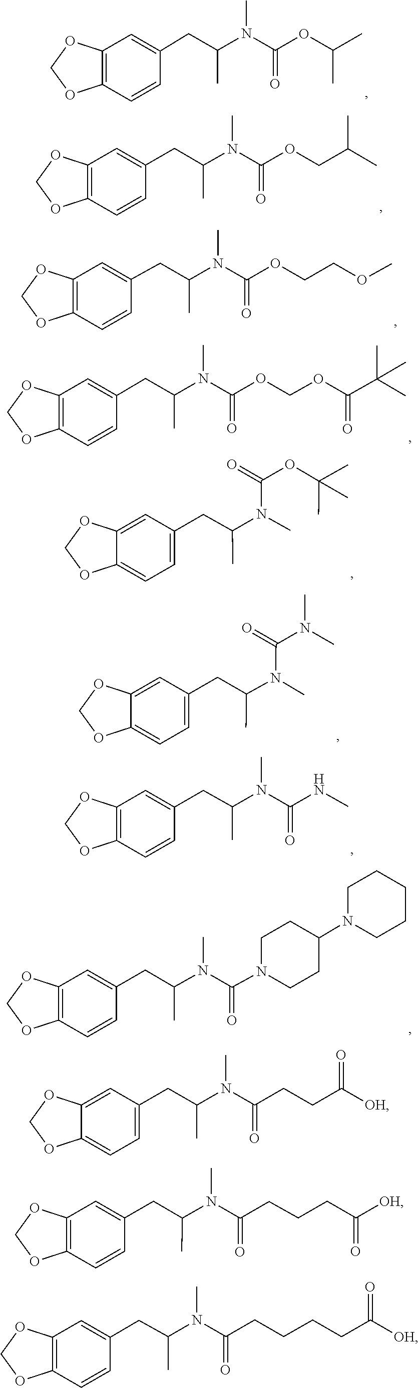

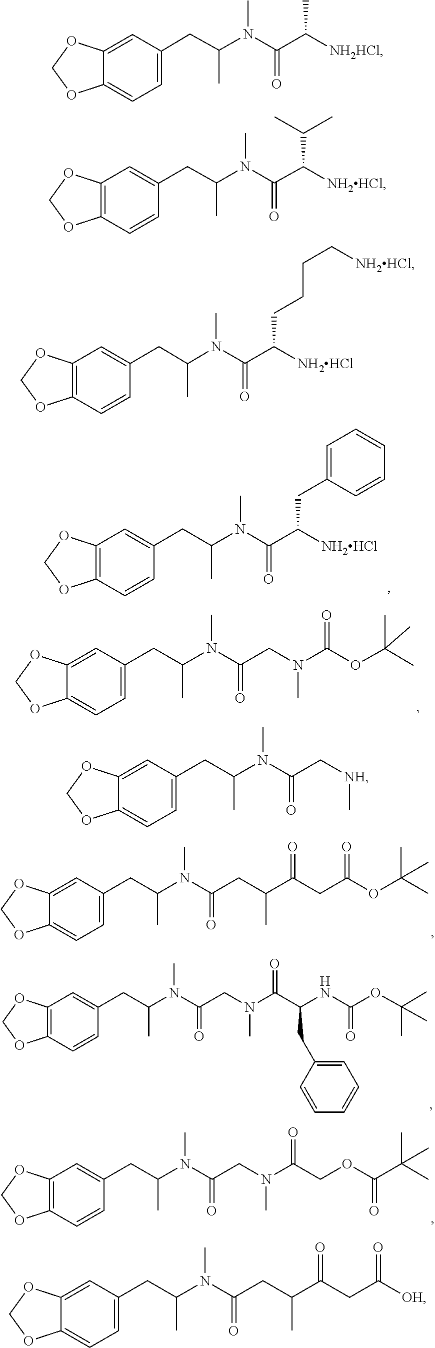

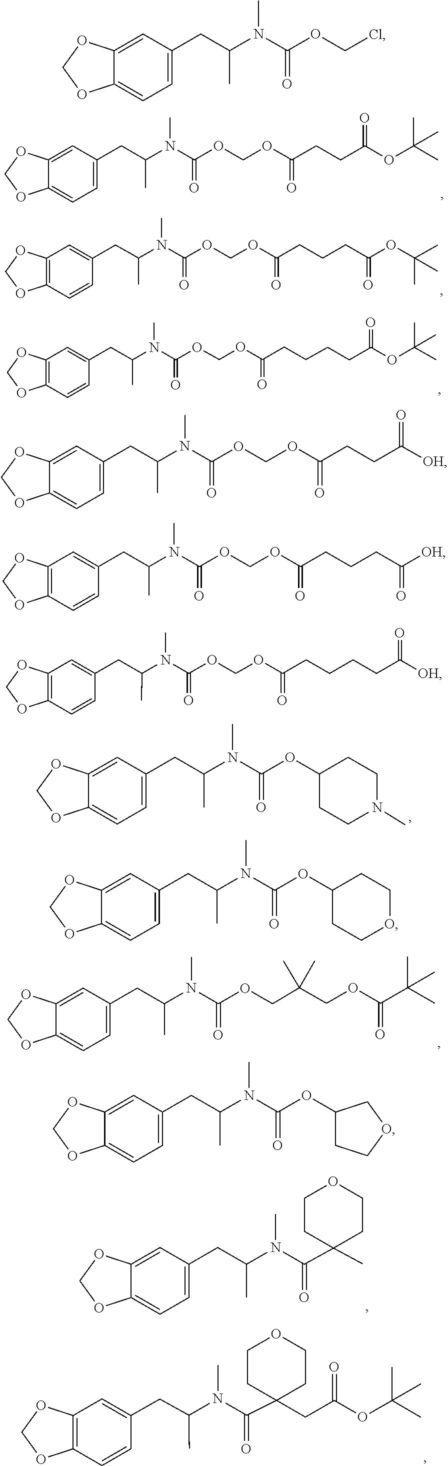

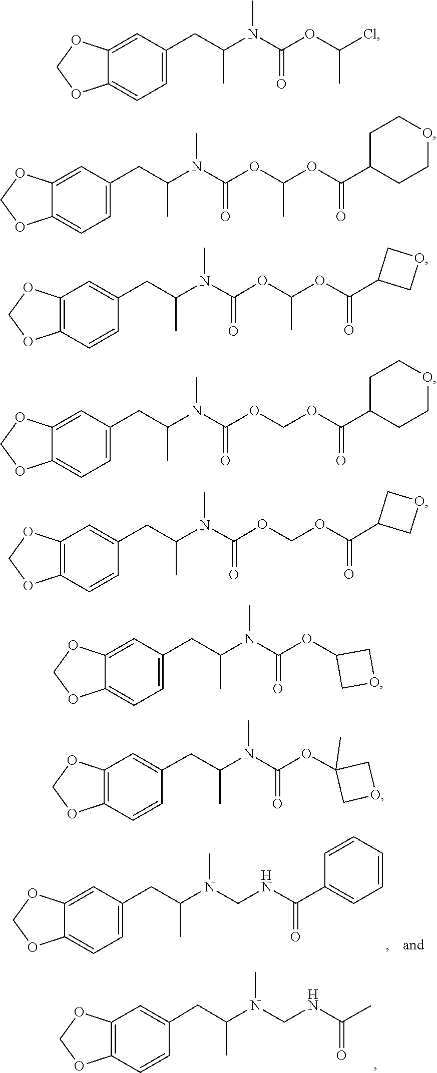

or a stereoisomer, a hydrate, or a pharmaceutically acceptable salt thereof.

In another aspect, provided herein are pharmaceutically compositions comprising the compound provided herein (e.g. a compound of Formula (I′)) or a stereoisomer, hydrate, pharmaceutically acceptable salt thereof, and a pharmaceutically acceptable excipient or carrier.

In yet another aspect, provided herein are methods of treating or preventing a disease, disorder, or condition in which an increased level of 3,4-methylenedioxymethamphetamine (MDMA) is beneficial, comprising administering to a subject in need thereof an effective amount of the compound provided herein (e.g. a compound of Formula (I′)) or a stereoisomer, hydrate, pharmaceutically acceptable salt thereof, or the pharmaceutically composition provided herein (e.g., a pharmaceutical composition comprising a compound of a compound of Formula (I′)).

In certain embodiments, the disease, disorder, or condition comprises post-traumatic stress disorder, major depression, schizophrenia, alzheimer's disease, frontotemporal dementia, Parkinson's disease, Parkinson's dementia, dementia, lewy body dementia, multiple system atrophy, or substance abuse.

In certain embodiments, the disease, disorder, or condition comprises musculoskeletal pain disorder including fibromyalgia, muscle pain, joint stiffness, osteoarthritis, rheumatoid arthritis, muscle cramps.

### BRIEF DESCRIPTION OF THE DRAWINGS

FIG. 1 shows the mean Concentration-Time Profiles of MDMA Following IV & Oral Dosing of MDMA (1 & 10 mg/Kg) to Male SD Rats.

FIG. 2 shows the mean Concentration-Time Profiles of Metabolite MDMA Following Oral Dosing of the N-Methylpiperidin-4-yl carbamate prodrug of MDMA (10 mg/Kg) to Male SD Rats.

FIG. 3 shows the mean Concentration-Time Profiles of Metabolite MDMA Following Oral Dosing of the Pyran-4-yl carbamate prodrug of MDMA (10 mg/Kg) to Male SD Rats.

FIG. 4 shows the mean Concentration-Time Profiles of Metabolite MDMA Following Oral Dosing of the Tert-butyl-glutarate methyleneoxy carbamate prodrug of MDMA (10 mg/Kg) to Male SD Rats.

FIG. 5 shows the Mean Concentration-Time Profiles of Metabolite MDMA Following Oral Dosing of the Pyran-acyloxy-substituted-methylene prodrug of MDMA ((Tetrahydropyran-4-carboxy)-1-ethyleneoxy carbamate) (10 mg/Kg) to Male SD Rats.

FIG. 6 shows the Mean Concentration-Time Profiles of the Lysine prodrug of MDMA and MDMA Following Oral Dosing of the Lysine prodrug of MDMA (10 mg/Kg) to Male SD Rats.

FIG. 7 shows the Mean Concentration-Time Profiles of Metabolite MDMA Following Oral Dosing of the (carbamoyloxy)methyl pivalate prodrug of MDMA (10 mg/Kg) to Male SD Rats.

FIG. 8 shows the Mean Concentration-Time Profiles of Metabolite MDMA Following Oral Dosing of the Glutarate methyleneoxy carbamate prodrug of MDMA (10 mg/Kg) to Male SD Rats.

FIG. 9 shows the Mean Concentration-Time Profiles of Metabolite MDMA Following Oral Dosing of the Trimethyllock prodrug of MDMA (10 mg/Kg) to Male SD Rats.

FIG. 10 shows the Mean Concentration-Time Profiles of Metabolite MDMA Following Oral Dosing of the Methoxyethyl carbamate prodrug of MDMA (10 mg/Kg) to Male SD Rats.

FIG. 11 shows the Mean Concentration-Time Profiles of Metabolite MDMA Following Oral Dosing of the Methyleneoxyadipate carbamate prodrug of MDMA (10 mg/Kg) to Male SD Rats.

FIG. 12 shows the Mean Concentration-Time Profiles of Metabolite MDMA Following Oral Dosing of the Methyleneoxysuccinate carbamate prodrug of MDMA (10 mg/Kg) to Male SD Rats.

FIG. 13 shows the Mean Concentration-Time Profiles of Metabolite MDMA Following Oral Dosing of the Dimethylglycine prodrug of MDMA (10 mg/Kg) to Male SD Rats.

FIG. 14 shows the Mean Concentration-Time Profiles of Metabolite MDMA Following Oral Dosing of the Succinate prodrug of MDMA (10 mg/Kg) to Male SD Rats.

FIG. 15 shows the Mean Concentration-Time Profiles of Metabolite MDMA Following Oral Dosing of the Phenylalanine prodrug of MDMA (10 mg/Kg) to Male SD Rats.

FIG. 16 shows the Mean Concentration-Time Profiles of Metabolite MDMA Following Oral Dosing of the SarcHydroxyacetic pivalate prodrug of MDMA (10 mg/Kg) to Male SD Rats.

FIG. 17 shows the Mean Concentration-Time Profiles of Metabolite MDMA Following Oral Dosing of the Benzamide aminal prodrug of MDMA (10 mg/Kg) to Male SD Rats.

FIG. 18 shows the Mean Concentration-Time Profiles of Metabolite MDMA Following Oral Dosing of the (Tetrahydropyran-4-carboxy)-methyleneoxy carbamate prodrug of MDMA (10 mg/Kg) to Male SD Rats.

FIG. 19 shows the Mean Concentration-Time Profiles of Metabolite MDMA Following Oral Dosing of the Tert-butyl-adipate methyleneoxy carbamate prodrug of MDMA (10 mg/Kg) to Male SD Rats.

FIG. 20 shows the Mean Concentration-Time Profiles of Metabolite MDMA Following Oral Dosing of the Acetamide aminal MDMA prodrug (10 mg/Kg) to Male SD Rats.

FIG. 21 shows the Mean Concentration-Time Profiles of Metabolite MDMA Following Oral Dosing of the Methyleneoxysuccinate (protected) carbamate prodrug of MDMA (10 mg/Kg) to Male SD Rats.

FIG. 22 shows the Mean Concentration-Time Profiles of Metabolite MDMA Following Oral Dosing of the Alanine prodrug of MDMA (10 mg/Kg) to Male SD Rats.

FIG. 23 shows the Mean Concentration-Time Profiles of Metabolite MDMA Following Oral Dosing of the 3-Methyl-oxetan-3-yl carbamate prodrug of MDMA (10 mg/Kg) to Male SD Rats.

FIG. 24 shows the Mean Concentration-Time Profiles of Metabolite MDMA Following Oral Dosing of the (Oxetane-3-carboxy)-1-ethyleneoxy carbamate prodrug of MDMA (10 mg/Kg) to Male SD Rats.

FIG. 25 shows the Mean Concentration-Time Profiles of Metabolite MDMA Following Oral Dosing of the (Oxetane-3-carboxy)-methyleneoxy carbamate prodrug of MDMA (10 mg/Kg) to Male SD Rats.

FIG. 26 shows the Mean Concentration-Time Profiles of Metabolite MDMA Following Oral Dosing of the SarcMal prodrug of MDMA (10 mg/Kg) to Male SD Rats.

### DETAILED DESCRIPTION

Described herein, in certain embodiments, are compositions and methods relating to synthesis of derivatives of 3,4-Methylenedioxymethamphetamine (MDMA). MDMA contains a chiral center and two enantiomers of MDMA are known (R)- and (S)-enantiomers. It is also possible that a prodrug of an individual enantiomer of MDMA may have advantages over the other enantiomer or the racemic mixture.

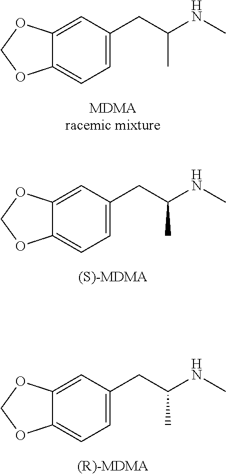

### Compounds of the Disclosure.

In some embodiments, the compounds described herein are prodrugs of 3,4-Methylenedioxymethamphetamine (MDMA). In some embodiments, the compounds described herein are psychedelics with improved pharmacokinetic properties as compared to MDMA (e.g., longer half life, longer tmax, and/or longer tlast, etc.).

There is a need to identify derivatives of MDMA that provide MDMA-like activity upon administration to a subject in need. Although there is a view that amino acid derivatives of MDMA will demonstrate a desired activity (e.g., provide therapeutically-relevant amounts of MDMA upon administration), we demonstrate herein that the data does not support such a position. Instead, we have conducted structure-activity relationship studies based on a carefully constructed experimental design in order to understand which derivatives of MDMA provide MDMA-like activity upon administration to a subject in need.

In one aspect, provided herein are compounds of Formula (I′), or a stereoisomer, a hydrate, or a pharmaceutically acceptable salt thereof:

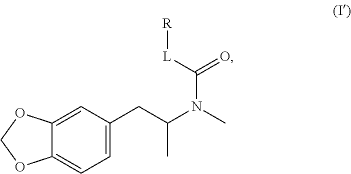

wherein:
L is bond, —O—, or NR′;
R and R are each alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.
   In certain embodiments, L is bond. In certain embodiments, L is —O—. In certain embodiments, L is —NR′—.
   In certain embodiments, R is alkyl, cycloalkyl, heteroalkyl, heterocyclylalkyl, aryl, or heteroaryl, each of which is optionally substituted with one or more Q;
   wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —N^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, R is alkyl or heterocyclylalkyl optionally substituted with one or more Q. In certain embodiments, R is alkyl substituted with one or more Q. In certain embodiments, R is heterocyclylalkyl.

In certain embodiments, R is optionally substituted methyl, ethyl, n-propyl, n-butyl, i-butyl, sec-butyl, t-butyl, tetrahydrofuranyl, or tetrahydropyranyl.

In certain embodiments, R is optionally substituted ethyl, n-propyl, n-butyl, i-butyl, sec-butyl, t-butyl, tetrahydrofuranyl, or tetrahydropyranyl, or R is substituted methyl.

In certain embodiments, R is ethyl, n-propyl, i-propyl, i-butyl, sec-butyl, t-butyl, tetrahydrofuranyl, tetrahydropyranyl, —CH₂CH₂—OCH₃, —CH₂CH₂—COOH, —CH₂CH₂CH₂—COOH, —CH₂CH₂CH₂CH₂—COOH, —CH₂—OC(O)C(CH₃)₃, —CH₂CH₂—C(O)OC(CH₃)₃, —CH₂CH₂CH₂—C(O)OC(CH₃)₃, —CH₂CH₂CH₂CH₂—C(O)OC(CH₃)₃, —CH₂N(CH₃)₂, —C(CH₃)NHC(O)OC(CH₃)₃, —C[CH(CH₃)₂]NHC(O)OC(CH₃)₃,

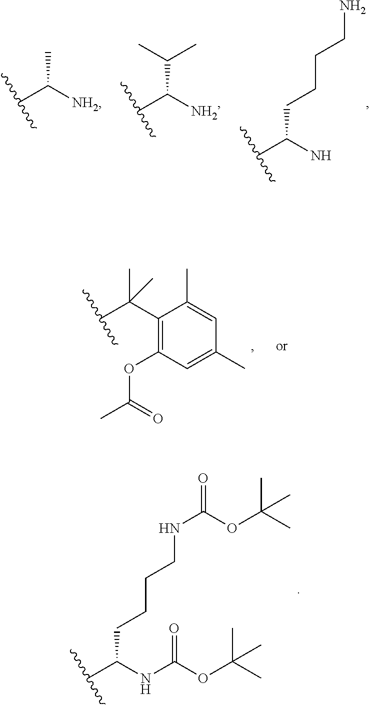

In certain embodiments, R is optionally substituted alkyl, R′ is hydrogen or unsubstituted alkyl.

In certain embodiments, R′ is hydrogen or methyl.

In one aspect, the present disclosure provides a compound of Formula (I), or a pharmaceutically acceptable salt thereof:

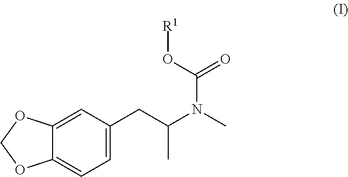

wherein R¹ is alkyl, alkenyl, heteroalkyl, cycloalkyl, heterocyclylalkyl, aryl, or heteroaryl, each of which is substituted or unsubstituted.

In some embodiments is a compound of Formula (I) or a pharmaceutically acceptable salt thereof, wherein R¹ is alkyl that is substituted. In some embodiments is a compound of Formula (I) or a pharmaceutically acceptable salt thereof, wherein R¹ is alkyl substituted with heteroalkyl, heterocyclylalkyl, or heteroaryl, wherein each of heteroalkyl, heterocyclylalkyl, and heteroaryl is unsubstituted or substituted.

In some embodiments is a compound of Formula (I) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or a pharmaceutically acceptable salt thereof, wherein R¹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments, R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (I) or a pharmaceutically acceptable salt thereof, wherein the compound is:

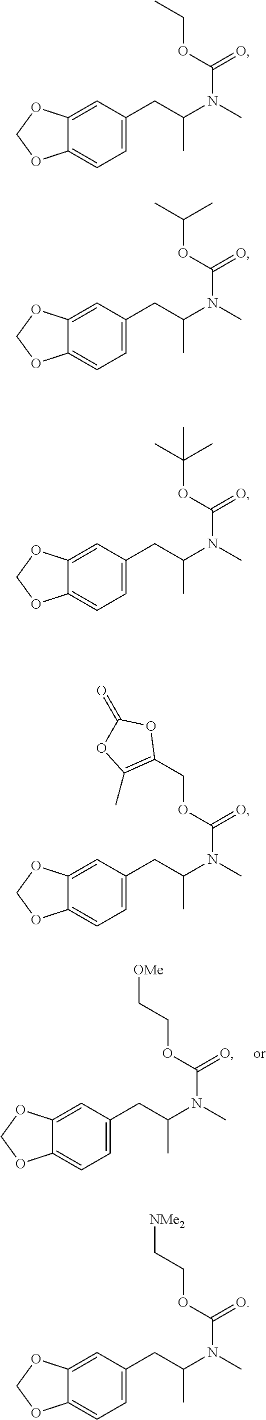 [SMILES: CCOC(=O)N(C)C(C)Cc1ccc2c(c1)OCO2 ; CC(C)OC(=O)N(C)C(C)Cc1ccc2c(c1)OCO2 ; CC(Cc1ccc2c(c1)OCO2)N(C)C(=O)OC(C)(C)C ; Cc1oc(=O)oc1COC(=O)N(C)C(C)Cc1ccc2c(c1)OCO2 ; COCCOC(=O)N(C)C(C)Cc1ccc2c(c1)OCO2 ; CC(Cc1ccc2c(c1)OCO2)N(C)C(=O)OCCN(C)C]

In some embodiments is a compound of Formula (I) or a pharmaceutically acceptable salt thereof, wherein the compound is:

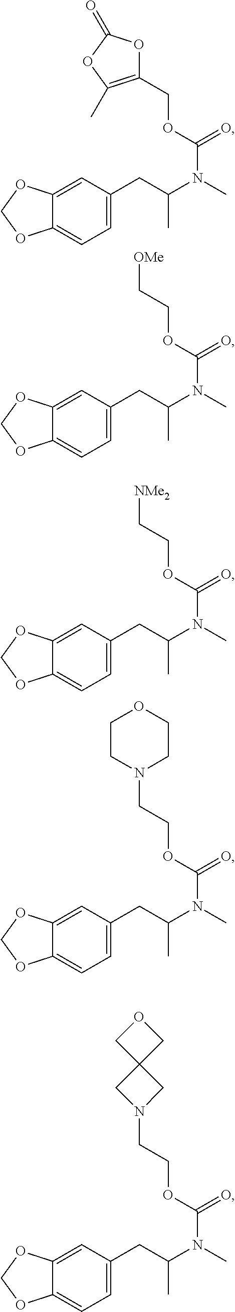

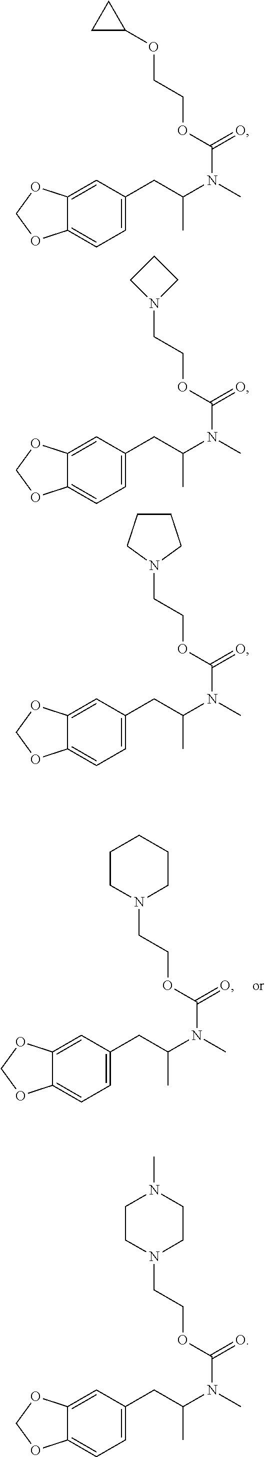

In some embodiments is a compound of Formula (I) or a pharmaceutically acceptable salt thereof, wherein the compound is.

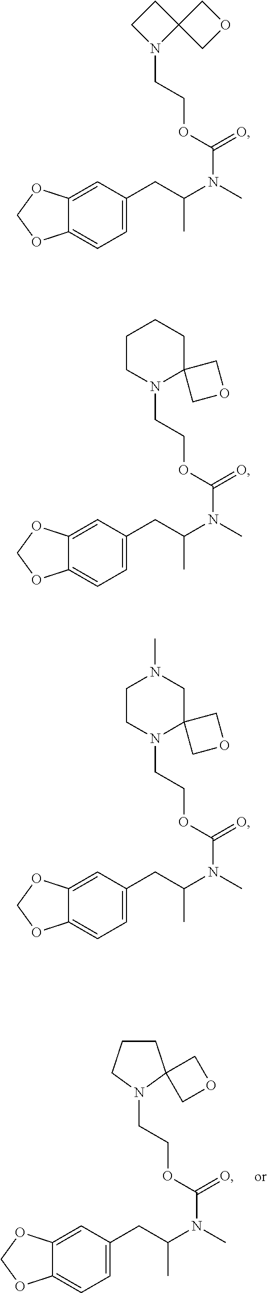 [SMILES: CC(Cc1ccc2c(c1)OCO2)N(C)C(=O)OCCN1CCC12COC2 ; CC(Cc1ccc2c(c1)OCO2)N(C)C(=O)OCCN1CCCCC12COC2 ; CC(Cc1ccc2c(c1)OCO2)N(C)C(=O)OCCN1CCN(C)CC12COC2 ; CC(Cc1ccc2c(c1)OCO2)N(C)C(=O)OCCN1CCCC12COC2]

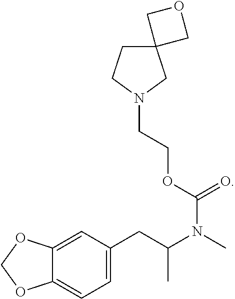 [SMILES: CC(Cc1ccc2c(c1)OCO2)N(C)C(=O)OCCN1CCC2(COC2)C1]

In some embodiments is a compound of Formula (I) or a pharmaceutically acceptable salt thereof, wherein R¹ is cycloalkyl that is substituted or unsubstituted. In some embodiments is a compound of Formula (I) or a pharmaceutically acceptable salt thereof, wherein R¹ is cycloalkyl that is substituted. In some embodiments is a compound of Formula (I) or a pharmaceutically acceptable salt thereof, wherein R¹ is cycloalkyl that is substituted with heteroalkyl, heterocyclylalkyl, or amino. In some embodiments is a compound of Formula (I) or a pharmaceutically acceptable salt thereof, wherein R¹ is cycloalkyl that is substituted with amino, aminoalkyl, or a nitrogen-containing heterocycle.

In some embodiments is a compound of Formula (I) having the structure of Formula (Ia), or a pharmaceutically acceptable salt thereof.

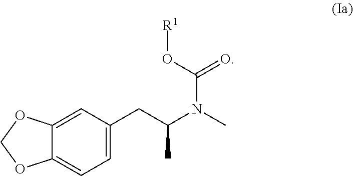

In some embodiments is a compound of Formula (I) or (Ia), or a pharmaceutically acceptable salt thereof, wherein the compound is:

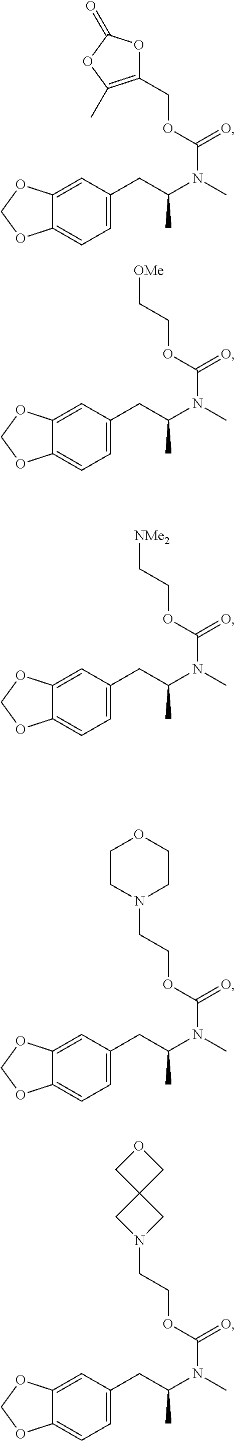 [SMILES: Cc1oc(=O)oc1COC(=O)N(C)[C@@H](C)Cc1ccc2c(c1)OCO2 ; COCCOC(=O)N(C)[C@@H](C)Cc1ccc2c(c1)OCO2 ; C[C@@H](Cc1ccc2c(c1)OCO2)N(C)C(=O)OCCN(C)C ; C[C@@H](Cc1ccc2c(c1)OCO2)N(C)C(=O)OCCN1CCOCC1 ; C[C@@H](Cc1ccc2c(c1)OCO2)N(C)C(=O)OCCN1CC2(COC2)C1]

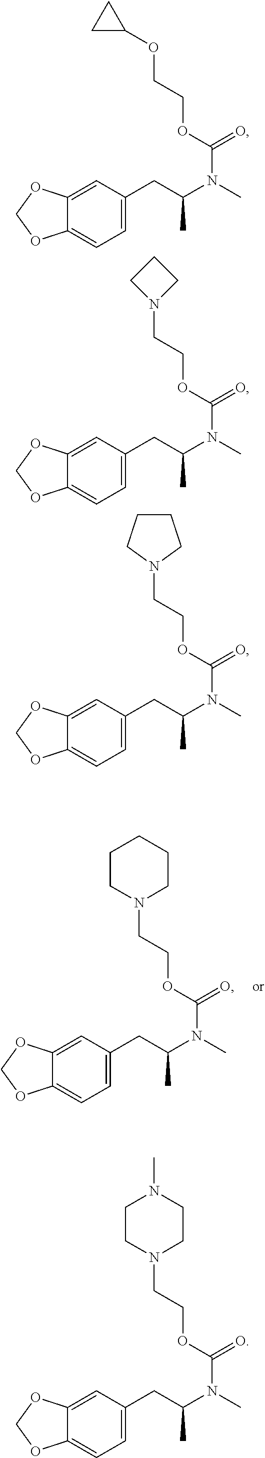

In some embodiments is a compound of Formula (I) or (Ia), or a pharmaceutically acceptable salt thereof, wherein the compound is:

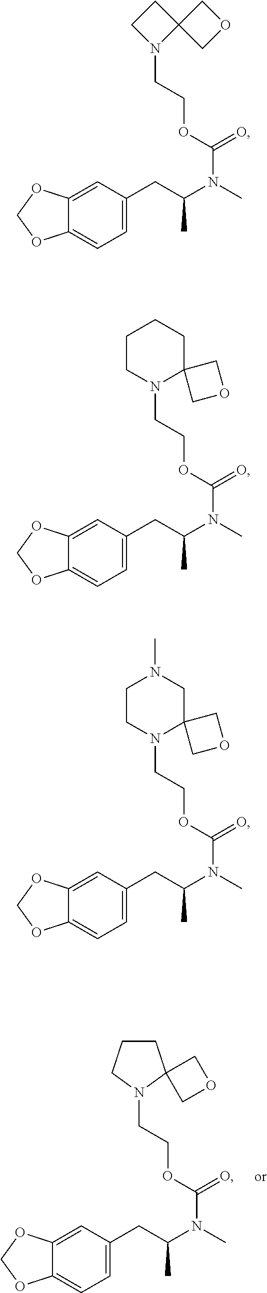 [SMILES: C[C@@H](Cc1ccc2c(c1)OCO2)N(C)C(=O)OCCN1CCC12COC2 ; C[C@@H](Cc1ccc2c(c1)OCO2)N(C)C(=O)OCCN1CCCCC12COC2 ; C[C@@H](Cc1ccc2c(c1)OCO2)N(C)C(=O)OCCN1CCN(C)CC12COC2 ; C[C@@H](Cc1ccc2c(c1)OCO2)N(C)C(=O)OCCN1CCCC12COC2]

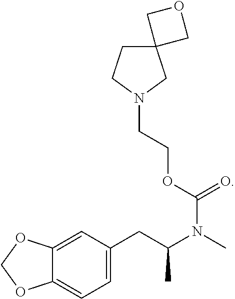 [SMILES: C[C@@H](Cc1ccc2c(c1)OCO2)N(C)C(=O)OCCN1CCC2(COC2)C1]

In some embodiments is a compound of Formula (I) having the structure of Formula (Ib), or a pharmaceutically acceptable salt thereof:

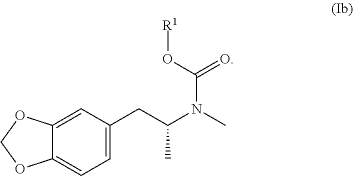

In some embodiments is a compound of Formula (I) or (Ib), or a pharmaceutically acceptable salt thereof, wherein the compound is:

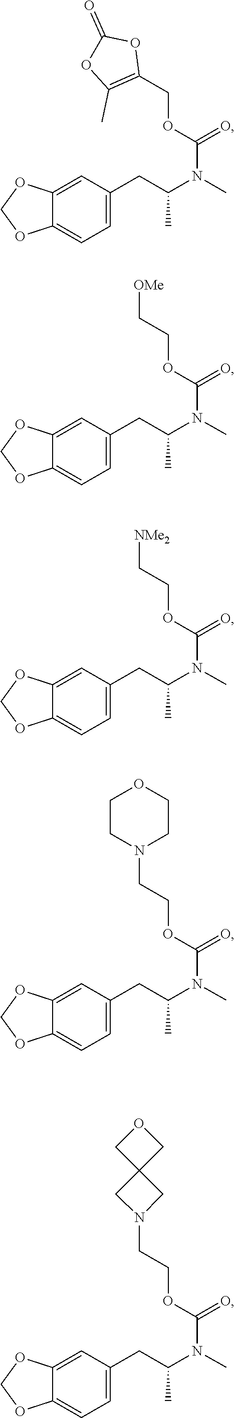 [SMILES: Cc1oc(=O)oc1COC(=O)N(C)[C@H](C)Cc1ccc2c(c1)OCO2 ; COCCOC(=O)N(C)[C@H](C)Cc1ccc2c(c1)OCO2 ; C[C@H](Cc1ccc2c(c1)OCO2)N(C)C(=O)OCCN(C)C ; C[C@H](Cc1ccc2c(c1)OCO2)N(C)C(=O)OCCN1CCOCC1 ; C[C@H](Cc1ccc2c(c1)OCO2)N(C)C(=O)OCCN1CC2(COC2)C1]

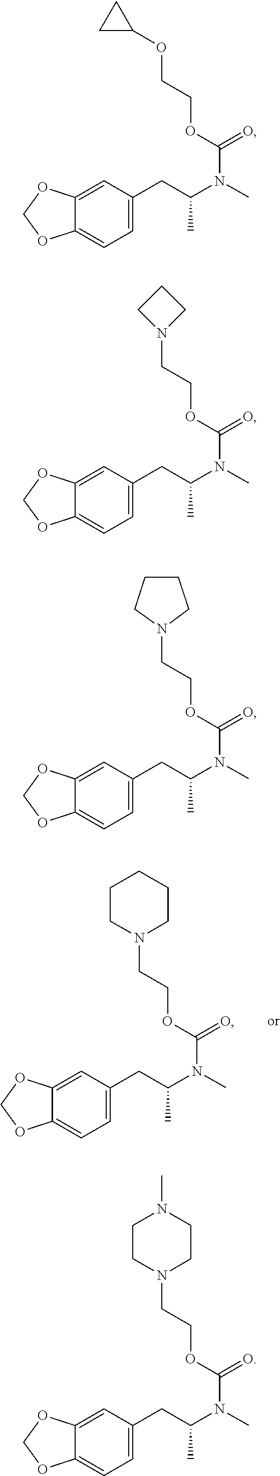 [SMILES: C[C@H](Cc1ccc2c(c1)OCO2)N(C)C(=O)OCCOC1CC1 ; C[C@H](Cc1ccc2c(c1)OCO2)N(C)C(=O)OCCN1CCC1 ; C[C@H](Cc1ccc2c(c1)OCO2)N(C)C(=O)OCCN1CCCC1 ; C[C@H](Cc1ccc2c(c1)OCO2)N(C)C(=O)OCCN1CCCCC1 ; C[C@H](Cc1ccc2c(c1)OCO2)N(C)C(=O)OCCN1CCN(C)CC1]

In some embodiments is a compound of Formula (I) or (Ib), or a pharmaceutically acceptable salt thereof, wherein the compound is:

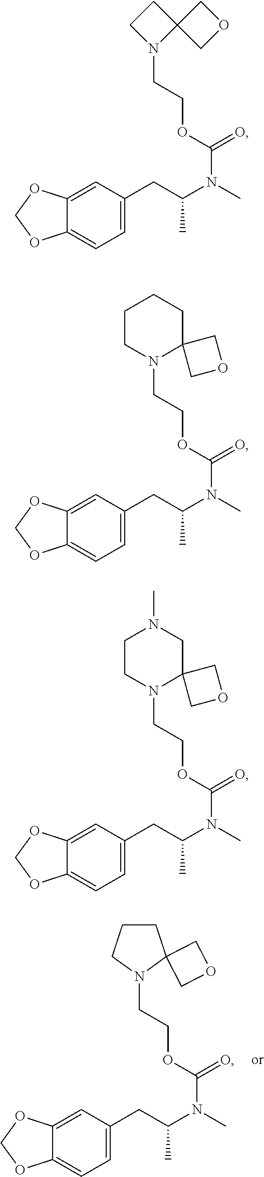

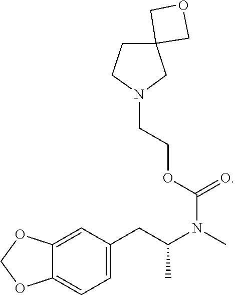 [SMILES: C[C@H](Cc1ccc2c(c1)OCO2)N(C)C(=O)OCCN1CCC2(COC2)C1]

In some embodiments is a compound of Formula (I), (Ia), or (Ib), or a pharmaceutically acceptable salt thereof, wherein if R¹ is unsubstituted alkyl, then R¹ is not tert-butyl.

In some embodiments is a compound of Formula (I) having the structure of Formula (I-1) or a pharmaceutically acceptable salt thereof:

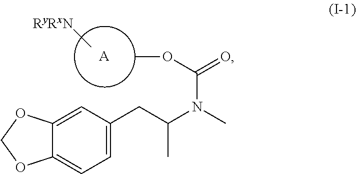

wherein

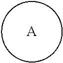

is cycloalkyl or heterocyclylalkyl, and each of R^{x} and R^{y} is alkyl or hydrogen, or R^{x} and R^{y} together with the atom to which they are attached form a heterocyclylalkyl ring.

In some embodiments is a compound of Formula (I) or (I-1) having the structure of Formula (I-1a) or a pharmaceutically acceptable salt thereof:

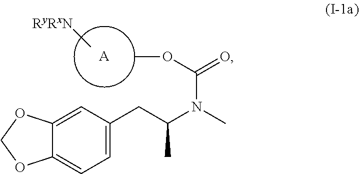

wherein

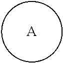

is cycloalkyl or heterocyclylalkyl, and each of R^{x} and R^{y} is alkyl or hydrogen, or R^{x} and R^{y} together with the atom to which they are attached form a heterocyclylalkyl ring.

In some embodiments is a compound of Formula (I) or (I-1) having the structure of Formula (I-1b) or a pharmaceutically acceptable salt thereof:

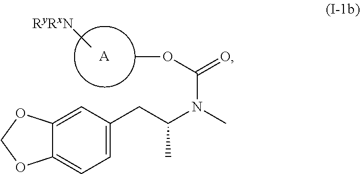

wherein

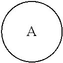

is cycloalkyl or heterocyclylalkyl, and each of R^{x} and R^{y} is alkyl or hydrogen, or R^{x} and R^{y} together with the atom to which they are attached form a heterocyclylalkyl ring.

In some embodiments is a compound of Formula (I) or (I-1) having the structure of Formula (I-1-1) or a pharmaceutically acceptable salt thereof:

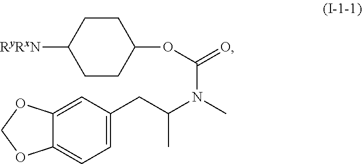

and each of R^{x} and R^{y} is alkyl or hydrogen, or R^{x} and R^{y} together with the atom to which they are attached form a heterocyclylalkyl ring.

In some embodiments is a compound of Formula (I), (I-1), or (I-1-1) having the structure of Formula (I-1-1a) or a pharmaceutically acceptable salt thereof:

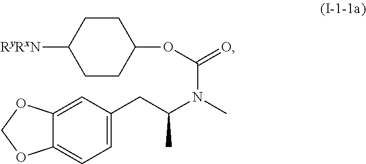

and each of R^{x} and R^{y} is alkyl or hydrogen, or R^{x} and R^{y} together with the atom to which they are attached form a heterocyclylalkyl ring.

In some embodiments is a compound of Formula (I), (I-1), or (I-1-1) having the structure of Formula (I-1-1b) or a pharmaceutically acceptable salt thereof:

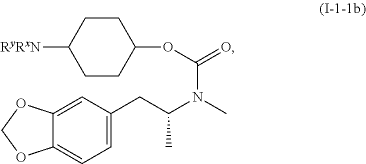

and each of R^{x} and R^{y} is alkyl or hydrogen, or R^{x} and R^{y} together with the atom to which they are attached form a heterocyclylalkyl ring.

In some embodiments is a compound of Formula (I), (I-1), or (I-1-1), or a pharmaceutically acceptable salt thereof, wherein the compound is:

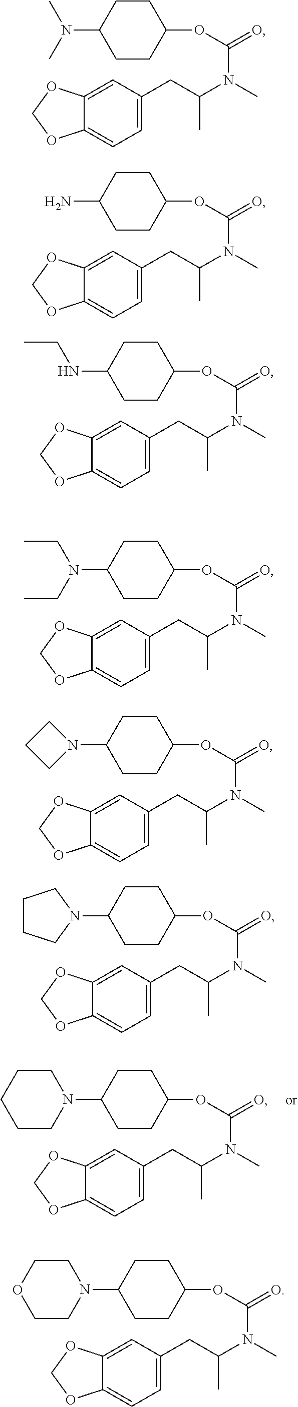

In another aspect, the present disclosure provides a compound of Formula (II), or a pharmaceutically acceptable salt thereof:

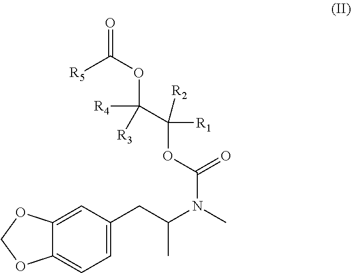

wherein:
each of R¹ and R² is independently alkyl that is substituted or unsubstituted, or hydrogen, or R¹ and R² together with the atom to which they are attached form a cycloalkyl ring;
each of R³ and R⁴ is independently alkyl that is substituted or unsubstituted, or hydrogen, or R³ and R⁴ together with the atom to which they are attached form a cycloalkyl ring;
and R⁵ is alkyl, cycloalkyl, heteroalkyl, heterocyclylalkyl, aryl, or heteroaryl, each of which is substituted or unsubstituted, or R⁵ together with the carbonyl to which R⁵ is attached form an amino acid residue.

In some embodiments is a compound of Formula (II) or a pharmaceutically acceptable salt thereof, R³ and R⁴ are each hydrogen. In some embodiments is a compound of Formula (II) or a pharmaceutically acceptable salt thereof R³ and R⁴ are each independently alkyl. In some embodiments is a compound of Formula (II) or a pharmaceutically acceptable salt thereof R³ and R⁴ are each independently alkyl, and R¹ and R² are each hydrogen. In some embodiments is a compound of Formula (II) or a pharmaceutically acceptable salt thereof, R³ and R⁴ together with the atom to which they are attached form a cycloalkyl ring. In some embodiments is a compound of Formula (II) or a pharmaceutically acceptable salt thereof, R³ and R⁴ together with the atom to which they are attached form a cycloalkyl ring, and R¹ and R² are each hydrogen.

In some embodiments is a compound of Formula (II) or a pharmaceutically acceptable salt thereof, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (II) or a pharmaceutically acceptable salt thereof, wherein each of R¹, R², R³, and R⁴ is hydrogen.

In some embodiments is a compound of Formula (II) or a pharmaceutically acceptable salt thereof, wherein the compound is:

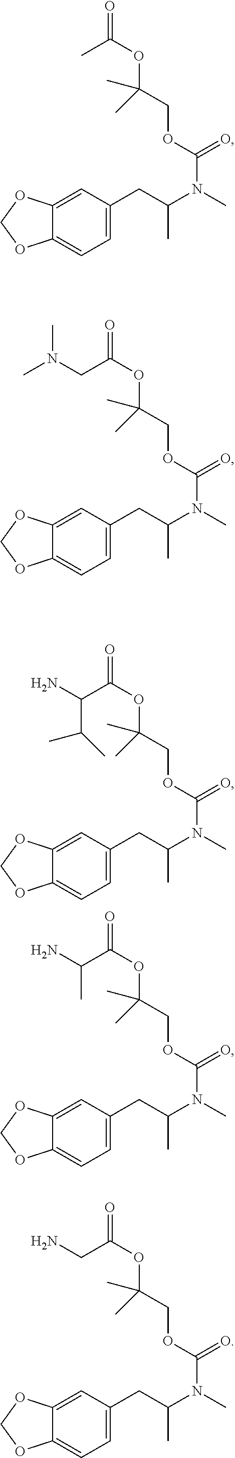

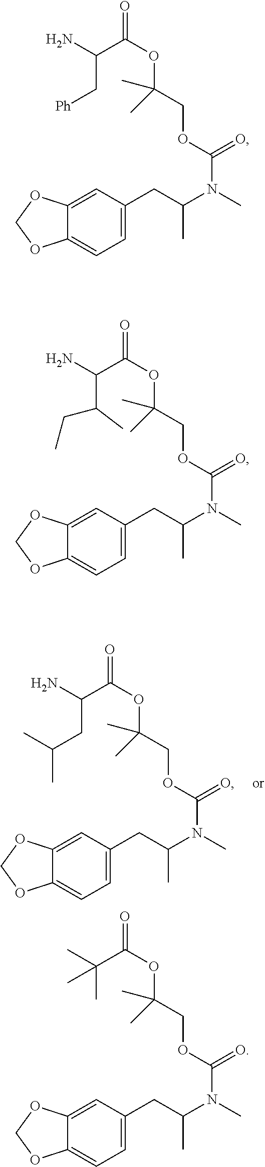

In some embodiments is a compound of Formula (II) or a pharmaceutically acceptable salt thereof, wherein the compound is:

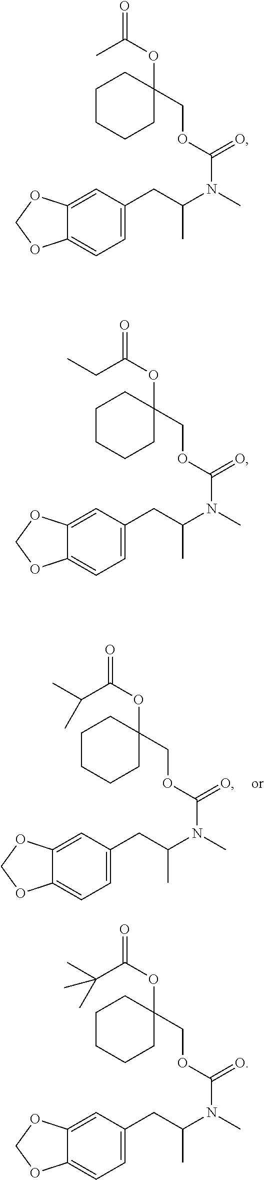 [SMILES: CC(=O)OC1(COC(=O)N(C)C(C)Cc2ccc3c(c2)OCO3)CCCCC1 ; CCC(=O)OC1(COC(=O)N(C)C(C)Cc2ccc3c(c2)OCO3)CCCCC1 ; CC(C)C(=O)OC1(COC(=O)N(C)C(C)Cc2ccc3c(c2)OCO3)CCCCC1 ; CC(Cc1ccc2c(c1)OCO2)N(C)C(=O)OCC1(OC(=O)C(C)(C)C)CCCCC1]

In some embodiments is a compound of Formula (II) or a pharmaceutically acceptable salt thereof, wherein the compound is:

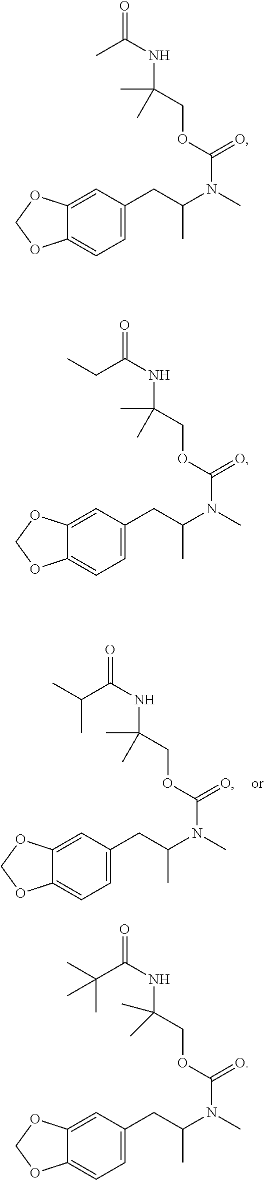 [SMILES: CC(=O)NC(C)(C)COC(=O)N(C)C(C)Cc1ccc2c(c1)OCO2 ; CCC(=O)NC(C)(C)COC(=O)N(C)C(C)Cc1ccc2c(c1)OCO2 ; CC(C)C(=O)NC(C)(C)COC(=O)N(C)C(C)Cc1ccc2c(c1)OCO2 ; CC(Cc1ccc2c(c1)OCO2)N(C)C(=O)OCC(C)(C)NC(=O)C(C)(C)C]

In some embodiments is a compound of Formula (II) having the structure of Formula (IIa), or a pharmaceutically acceptable salt thereof:

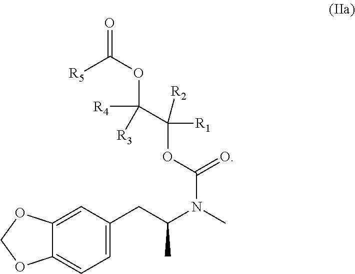

In some embodiments is a compound of Formula (II) having the structure of Formula (IIb), or a pharmaceutically acceptable salt thereof:

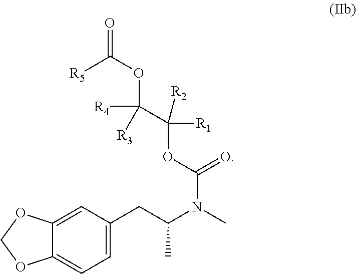

In another aspect, the present disclosure provides a compound of Formula (III), or a pharmaceutically acceptable salt thereof:

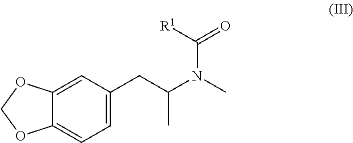

wherein R¹ is alkyl, alkenyl, heteroalkyl, cycloalkyl, heterocyclylalkyl, aryl, or heteroaryl, each of which is substituted or unsubstituted, or R¹ and the carbonyl atom to which R¹ is attached form an amino acid residue.

In some embodiments is a compound of Formula (III) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments, R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (III), or a pharmaceutically acceptable salt thereof, wherein the compound is:

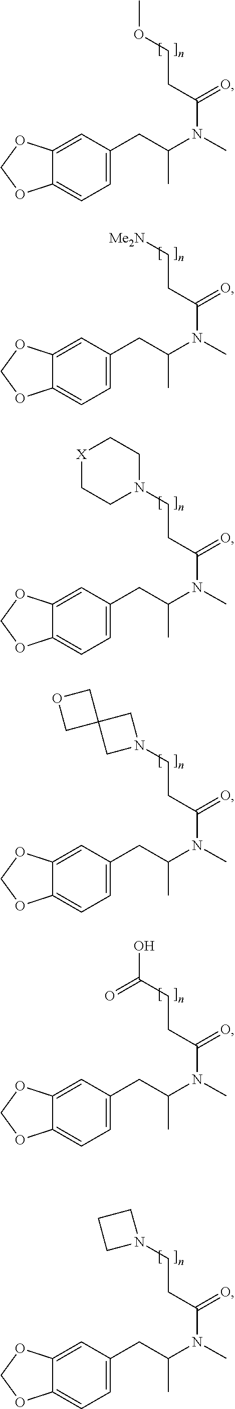

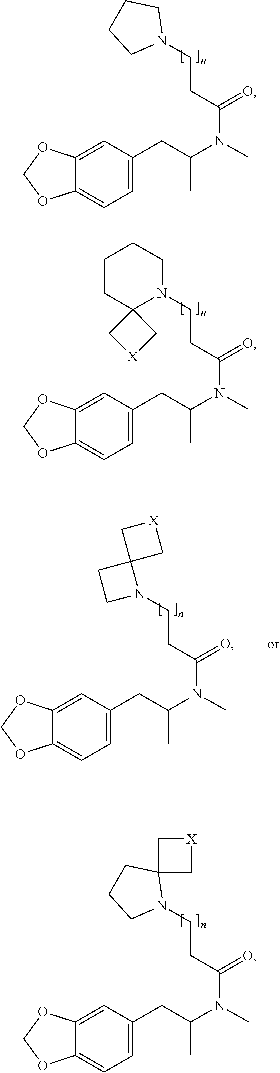

wherein each n is independently 1, 2, 3, 4, 5, or 6; and each X is independently —O—, —S—, —S(O)—, —S(O)₂—, —NH—, or —N(R²)—, wherein each R² is independently alkyl or heteroalkyl, each of which is substituted or unsubstituted.

In another aspect, the present disclosure provides a compound of Formula (III), or a pharmaceutically acceptable salt thereof, wherein the compound is:

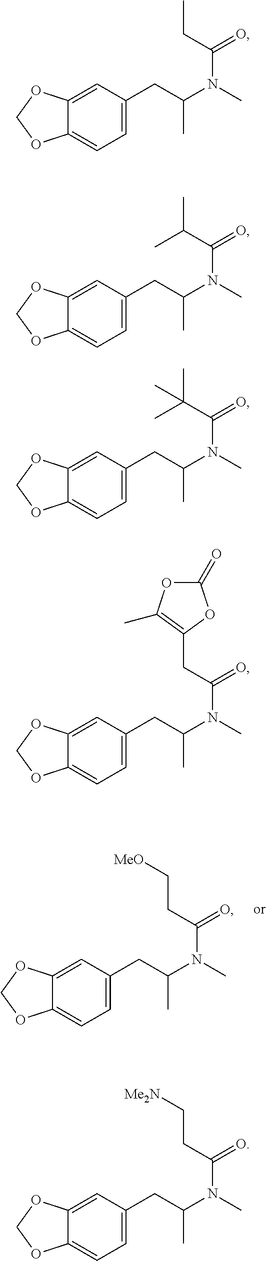

In another aspect, the present disclosure provides a compound of Formula (III), or a pharmaceutically acceptable salt thereof, wherein the compound is:

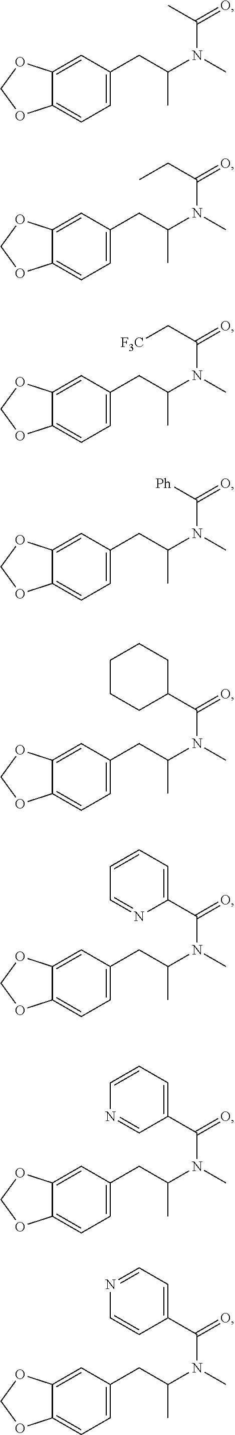

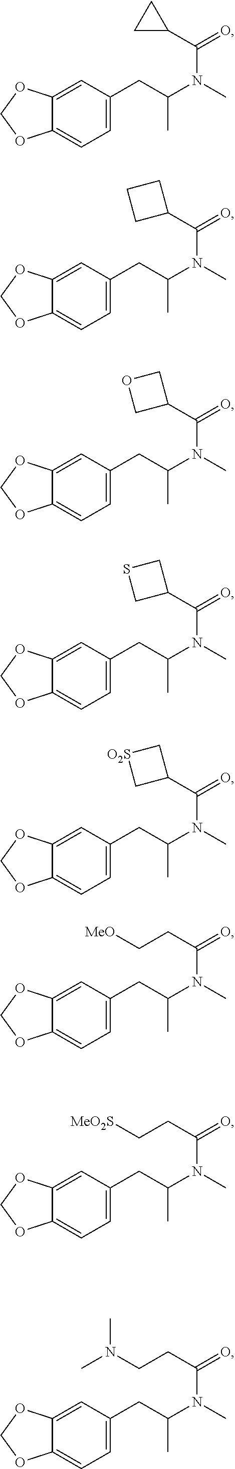

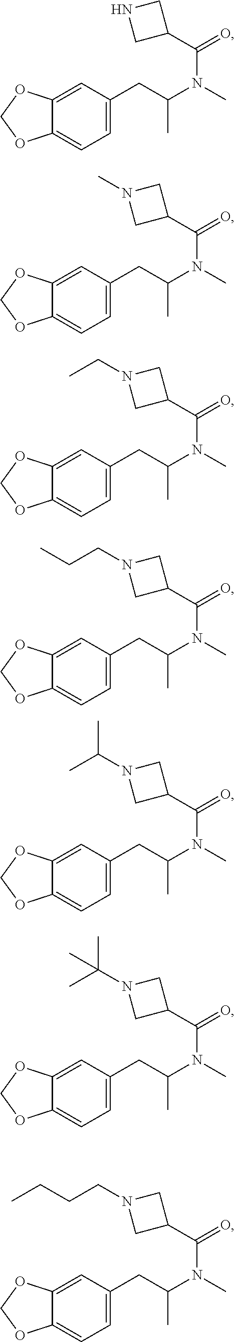

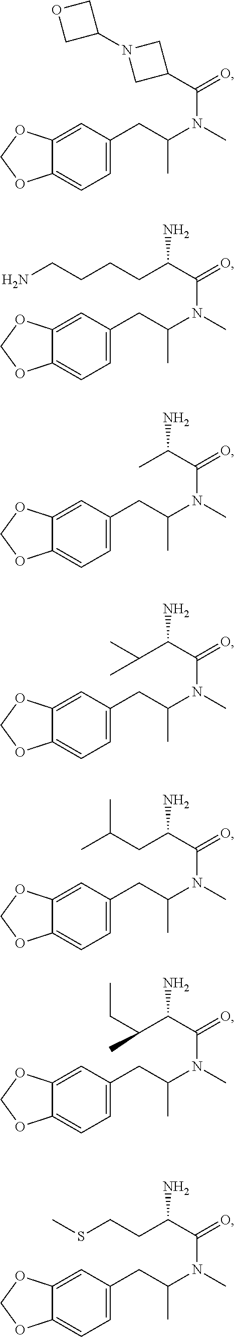

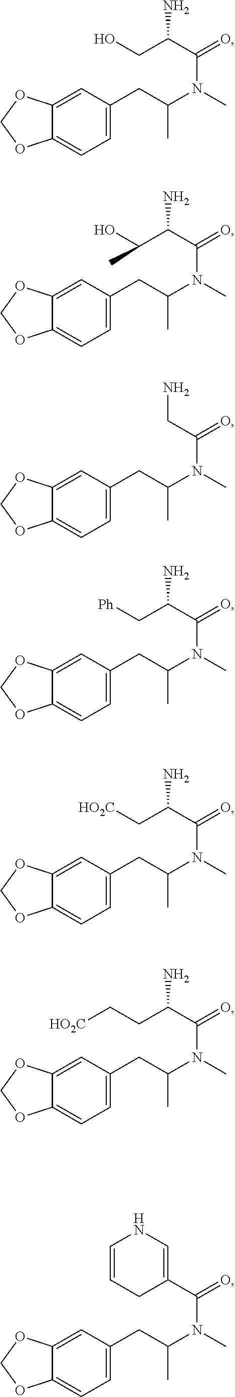

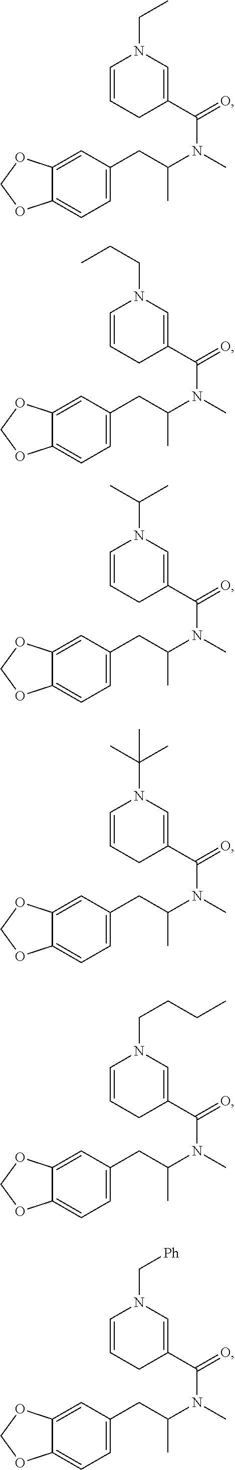

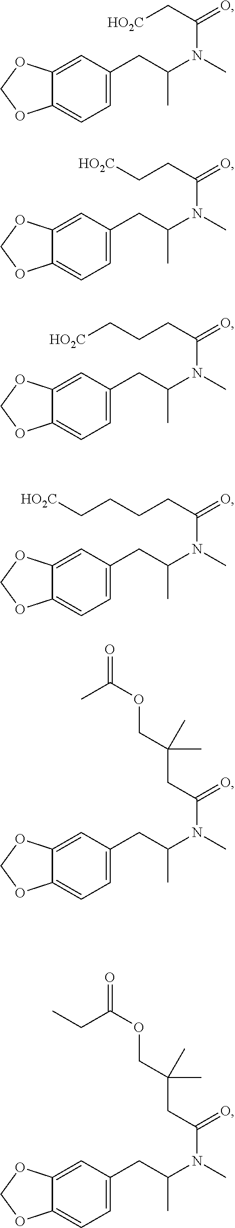

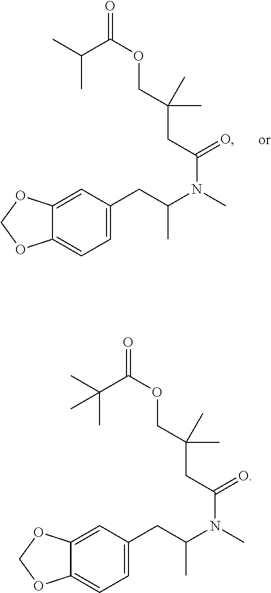 [SMILES: CC(C)C(=O)OCC(C)(C)CC(=O)N(C)C(C)Cc1ccc2c(c1)OCO2 ; "or" ; CC(Cc1ccc2c(c1)OCO2)N(C)C(=O)CC(C)(C)COC(=O)C(C)(C)C]

In some embodiments is a compound of Formula (III) having the structure of Formula (IIIa), or a pharmaceutically acceptable salt thereof:

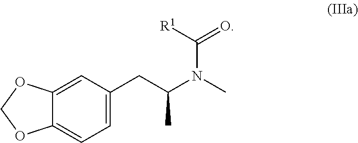

In some embodiments is a compound of Formula (III) or (IIIa), or a pharmaceutically acceptable salt thereof, wherein the compound is:

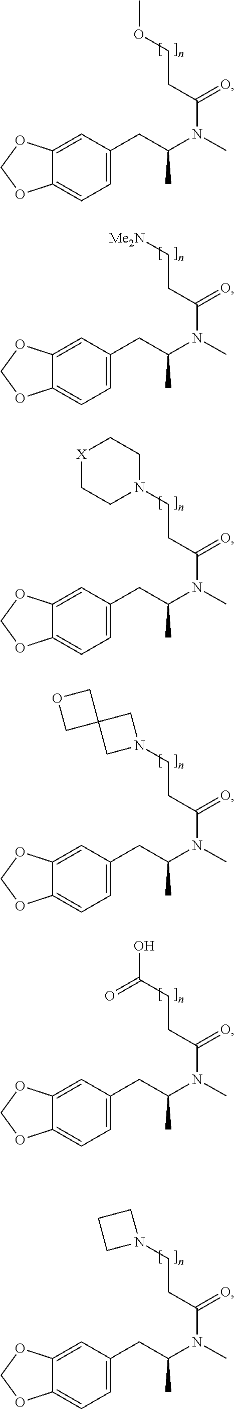

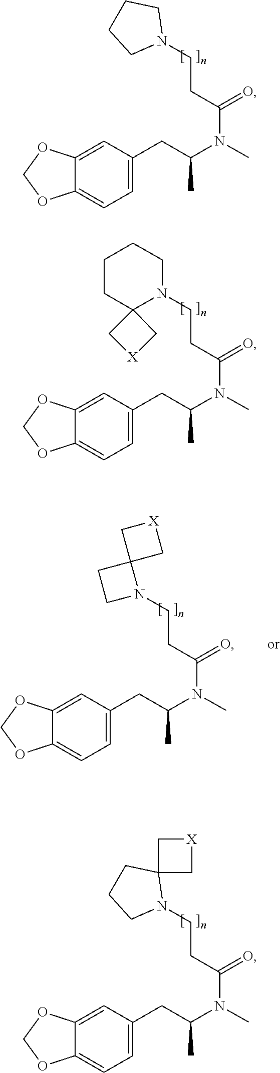

wherein each n is independently 1, 2, 3, 4, 5, or 6; and each X is independently —O—, —S—, —S(O)—, —S(O)₂—, —NH—, or —N(R²)—, wherein each R² is independently alkyl or heteroalkyl, each of which is substituted or unsubstituted.

In some embodiments is a compound of Formula (III) having the structure of Formula (IIIb), or a pharmaceutically acceptable salt thereof:

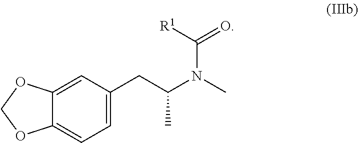

In some embodiments is a compound of Formula (III) or (IIIb), or a pharmaceutically acceptable salt thereof, wherein the compound is:

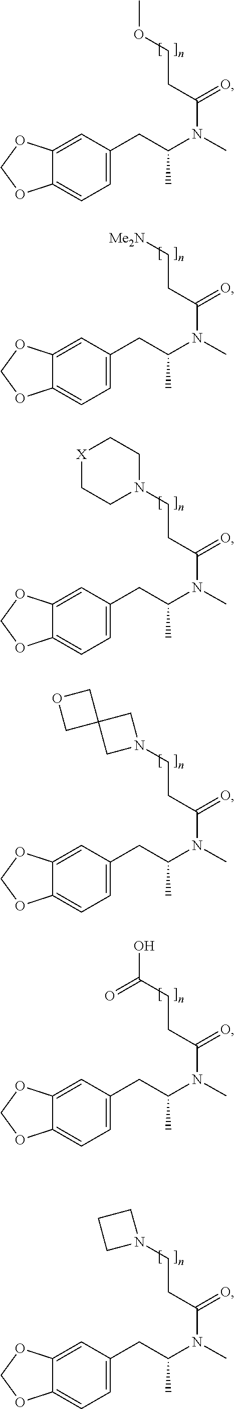

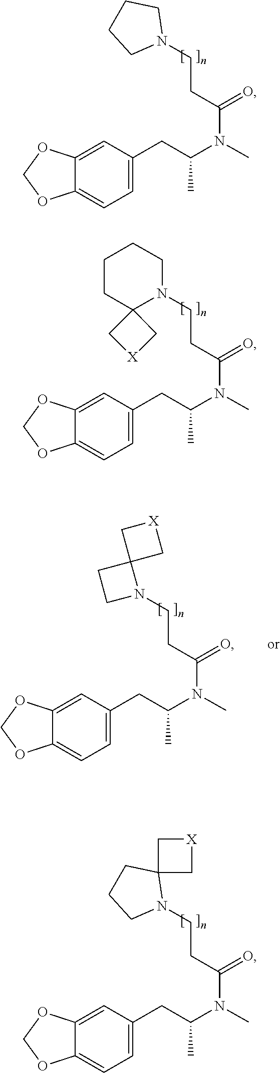

wherein each n is independently 1, 2, 3, 4, 5, or 6; and each X is independently —O—, —S—, —S(O)—, —S(O)₂—, —NH—, or —N(R²)—, wherein each R² is independently alkyl or heteroalkyl, each of which is substituted or unsubstituted.

In another aspect, the present disclosure provides a compound of Formula (IV), or a pharmaceutically acceptable salt thereof:

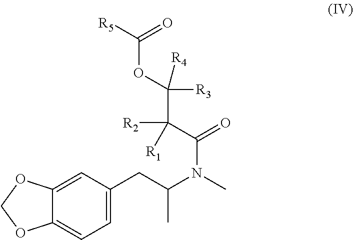

wherein:
each of R¹ and R² is independently alkyl that is substituted or unsubstituted, or hydrogen, or R¹ and R² together with the atom to which they are attached form a cycloalkyl ring;
each of R³ and R⁴ is independently alkyl that is substituted or unsubstituted, or hydrogen, or R³ and R⁴ together with the atom to which they are attached form a cycloalkyl ring;
and R⁵ is alkyl, cycloalkyl, heteroalkyl, heterocyclylalkyl, aryl, or heteroaryl, each of which is substituted or unsubstituted.

In some embodiments is a compound of Formula (IV) or a pharmaceutically acceptable salt thereof, R³ and R⁴ are each hydrogen. In some embodiments is a compound of Formula (IV) or a pharmaceutically acceptable salt thereof R³ and R⁴ are each independently alkyl. In some embodiments is a compound of Formula (IV) or a pharmaceutically acceptable salt thereof R³ and R⁴ are each independently alkyl, and R¹ and R² are each hydrogen. In some embodiments is a compound of Formula (IV) or a pharmaceutically acceptable salt thereof, R³ and R⁴ together with the atom to which they are attached form a cycloalkyl ring. In some embodiments is a compound of Formula (IV) or a pharmaceutically acceptable salt thereof, R³ and R⁴ together with the atom to which they are attached form a cycloalkyl ring, and R¹ and R² are each hydrogen.

In some embodiments is a compound of Formula (IV) or a pharmaceutically acceptable salt thereof, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (IV) or a pharmaceutically acceptable salt thereof, wherein each of R¹, R², R³, and R⁴ is hydrogen.

In some embodiments is a compound of Formula (IV) or a pharmaceutically acceptable salt thereof, wherein the compound is:

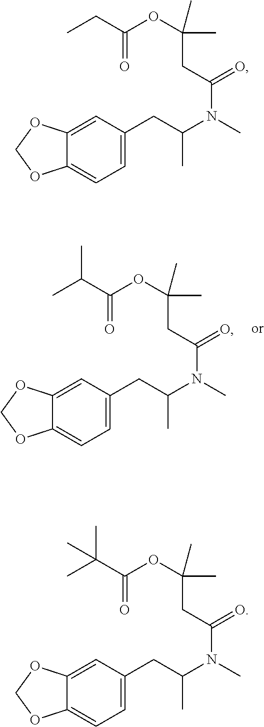 [SMILES: CCC(=O)OC(C)(C)CC(=O)N(C)C(C)Cc1ccc2c(c1)OCO2 ; CC(C)C(=O)OC(C)(C)CC(=O)N(C)C(C)Cc1ccc2c(c1)OCO2 ; CC(Cc1ccc2c(c1)OCO2)N(C)C(=O)CC(C)(C)OC(=O)C(C)(C)C]

In some embodiments is a compound of Formula (IV) having the structure of Formula (IVa), or a pharmaceutically acceptable salt thereof:

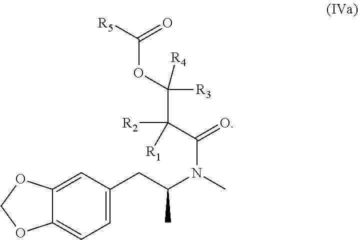

In some embodiments is a compound of Formula (IV) having the structure of Formula (IVb), or a pharmaceutically acceptable salt thereof:

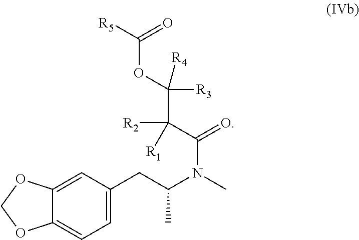

In another aspect, the present disclosure provides a compound of Formula (V), or a pharmaceutically acceptable salt thereof:

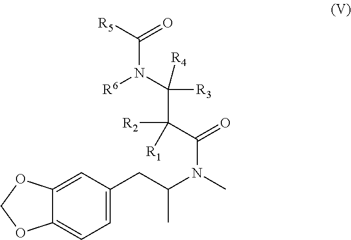

wherein:
each of R¹ and R² is independently alkyl that is substituted or unsubstituted, or hydrogen, or R¹ and R² together with the atom to which they are attached form a cycloalkyl ring;
each of R³ and R⁴ is independently alkyl that is substituted or unsubstituted, or hydrogen, or R³ and R⁴ together with the atom to which they are attached form a cycloalkyl ring;
and each of R⁵ and R⁶ is independently alkyl, cycloalkyl, heteroalkyl, heterocyclylalkyl, aryl, or heteroaryl, each of which is substituted or unsubstituted.

In some embodiments is a compound of Formula (V) or a pharmaceutically acceptable salt thereof, R³ and R⁴ are each hydrogen. In some embodiments is a compound of Formula (V) or a pharmaceutically acceptable salt thereof R³ and R⁴ are each independently alkyl. In some embodiments is a compound of Formula (V) or a pharmaceutically acceptable salt thereof R³ and R⁴ are each independently alkyl, and R¹ and R² are each hydrogen. In some embodiments is a compound of Formula (V) or a pharmaceutically acceptable salt thereof R³ and R⁴ are each independently alkyl, R¹ and R² are each hydrogen, and R⁶ is alkyl or hydrogen. In some embodiments is a compound of Formula (V) or a pharmaceutically acceptable salt thereof, R³ and R⁴ together with the atom to which they are attached form a cycloalkyl ring. In some embodiments is a compound of Formula (V) or a pharmaceutically acceptable salt thereof, R³ and R⁴ together with the atom to which they are attached form a cycloalkyl ring, and R¹ and R² are each hydrogen. In some embodiments is a compound of Formula (V) or a pharmaceutically acceptable salt thereof, R³ and R⁴ together with the atom to which they are attached form a cycloalkyl ring, R¹ and R² are each hydrogen, and R⁶ is alkyl or hydrogen.

In some embodiments is a compound of Formula (V) or a pharmaceutically acceptable salt thereof, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (V) or a pharmaceutically acceptable salt thereof, wherein each of R¹, R², R³, and R⁴ is hydrogen. In some embodiments is a compound of Formula (V) or a pharmaceutically acceptable salt thereof, wherein R⁶ is that is substituted or unsubstituted, hydrogen.

In some embodiments is a compound of Formula (V) having the structure of Formula (Va), or a pharmaceutically acceptable salt thereof:

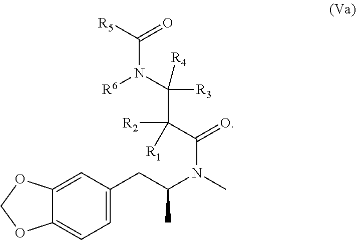

In some embodiments is a compound of Formula (V) having the structure of Formula (Vb), or a pharmaceutically acceptable salt thereof:

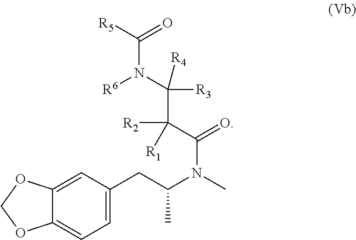

In another aspect, the present disclosure provides a compound of Formula (VI), or a pharmaceutically acceptable salt thereof:

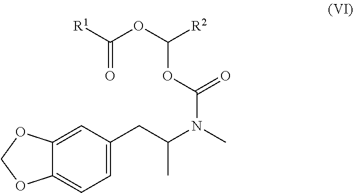

wherein R¹ is alkyl, alkenyl, heteroalkyl, cycloalkyl, heterocyclylalkyl, aryl, or heteroaryl, each of which is substituted or unsubstituted, or R¹ and the carbonyl to which R¹ is attached form an amino acid residue; and R² is alkyl that is substituted or unsubstituted, or hydrogen.

In some embodiments is a compound of Formula (VI) or a pharmaceutically acceptable salt thereof, wherein R¹ together which the carbonyl to which R¹ is attached form an amino acid residue.

In some embodiments is a compound of Formula (VI) or a pharmaceutically acceptable salt thereof, wherein R¹ is alkyl or heteroalkyl that is substituted or unsubstituted. In some embodiments is a compound of Formula (VI) or a pharmaceutically acceptable salt thereof, wherein R¹ is alkyl that is substituted. In some embodiments is a compound of Formula (VI) or a pharmaceutically acceptable salt thereof, wherein R¹ is alkyl that is substituted with heterocyclylalkyl that is substituted or unsubstituted.

In some embodiments is a compound of Formula (VI) or a pharmaceutically acceptable salt thereof, wherein R¹ is heteroalkyl that is substituted. In some embodiments is a compound of Formula (VI) or a pharmaceutically acceptable salt thereof, wherein R¹ is heteroalkyl that is substituted with cycloalkyl or heterocyclylalkyl, wherein cycloalkyl or heterocyclylalkyl are substituted or unsubstituted.

In some embodiments is a compound of Formula (VI) or a pharmaceutically acceptable salt thereof, wherein R¹ is heterocyclylalkyl that is substituted or unsubstituted. In some embodiments is a compound of Formula (VI) or a pharmaceutically acceptable salt thereof, wherein R¹ is heterocyclylalkyl that is substituted with alkyl.

In some embodiments is a compound of Formula (VI) or a pharmaceutically acceptable salt thereof, wherein R¹ is unsubstituted alkyl. In some embodiments is a compound of Formula (VI) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (VI) or a pharmaceutically acceptable salt thereof, wherein R² is methyl, ethyl, n-propyl, isopropyl, tert-butyl, or hydrogen. In some embodiments is a compound of Formula (VI) or a pharmaceutically acceptable salt thereof, wherein R² is methyl or hydrogen. In some embodiments is a compound of Formula (VI) or a pharmaceutically acceptable salt thereof, wherein R² is methyl. In some embodiments is a compound of Formula (VI) or a pharmaceutically acceptable salt thereof, wherein R² is hydrogen.

In some embodiments is a compound of Formula (VI) or a pharmaceutically acceptable salt thereof, wherein the compound is:

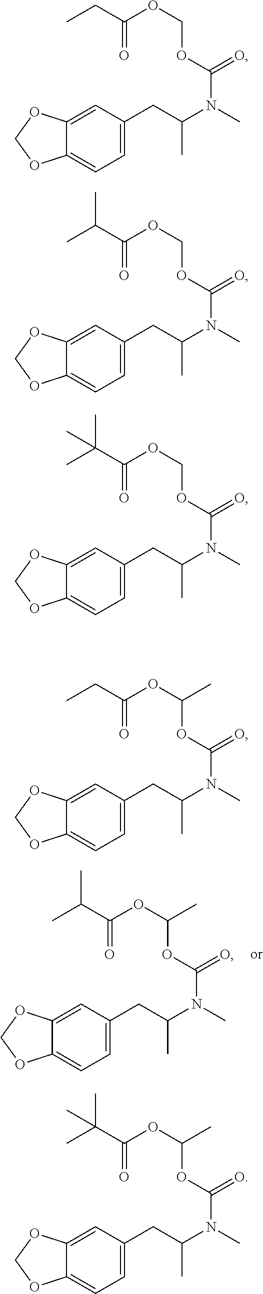

In some embodiments is a compound of Formula (VI) or a pharmaceutically acceptable salt thereof, wherein the compound is:

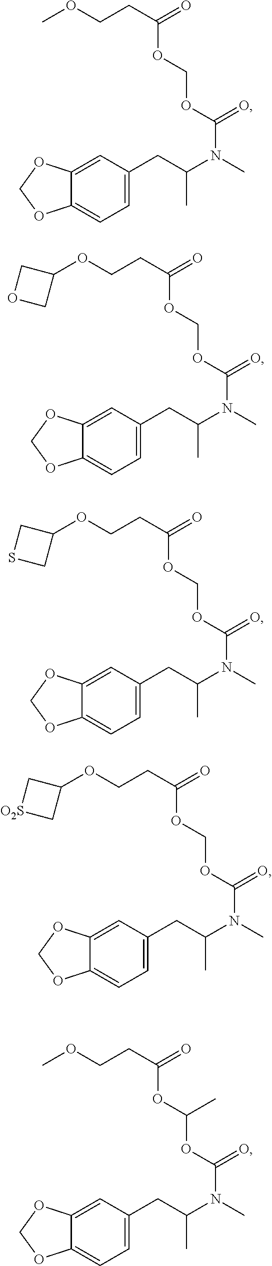

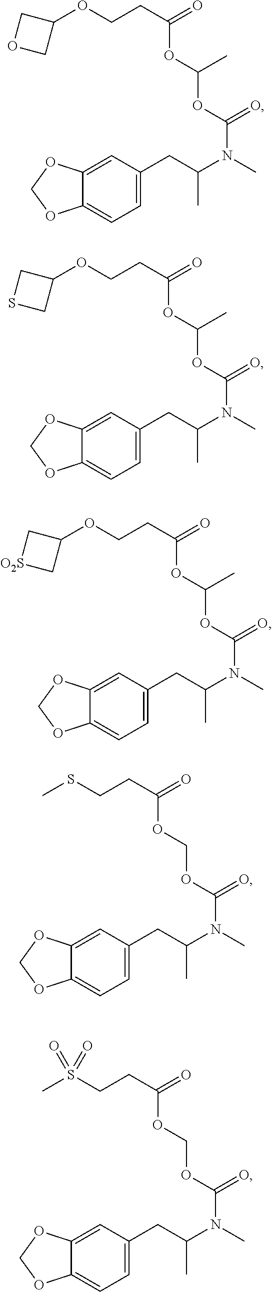

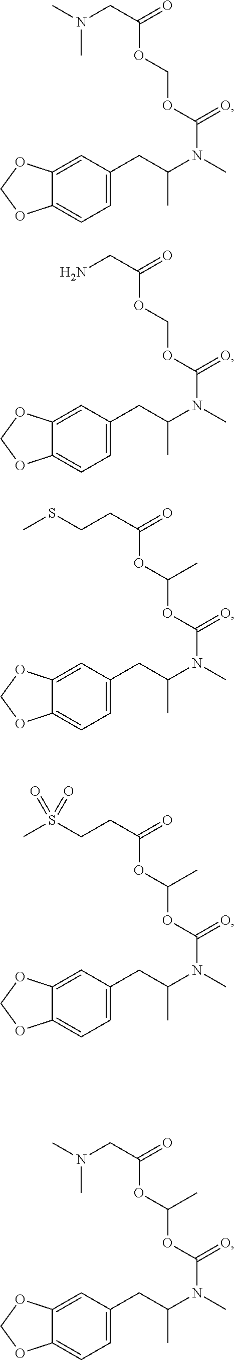

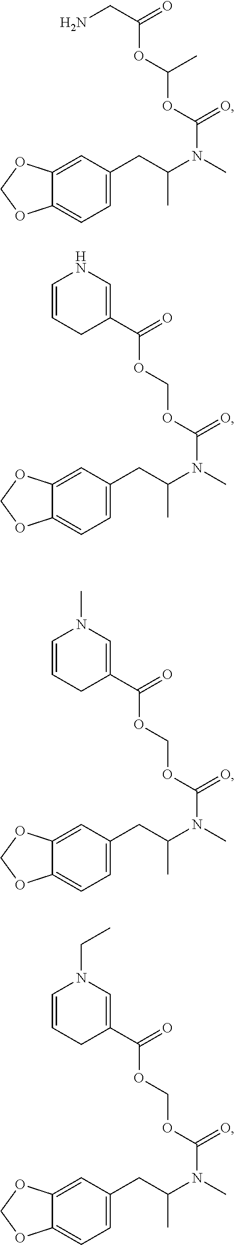

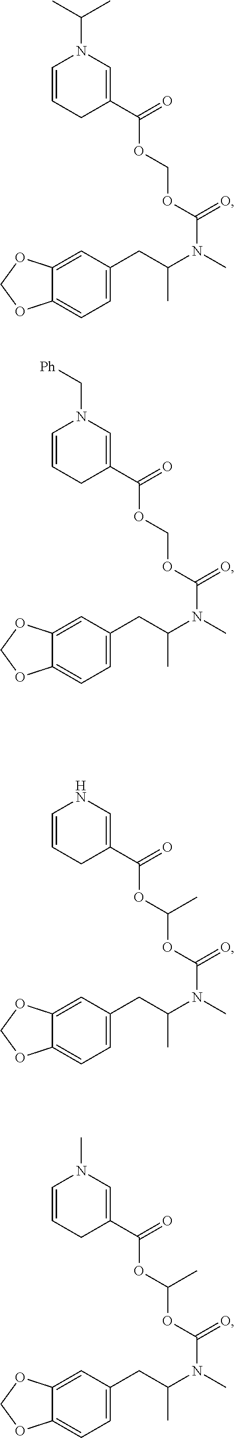 [SMILES: CC(C)N1C=CCC(C(=O)OCOC(=O)N(C)C(C)Cc2ccc3c(c2)OCO3)=C1 ; CC(Cc1ccc2c(c1)OCO2)N(C)C(=O)OCOC(=O)C1=CN(Cc2ccccc2)C=CC1 ; CC(OC(=O)C1=CNC=CC1)OC(=O)N(C)C(C)Cc1ccc2c(c1)OCO2 ; CC(OC(=O)C1=CN(C)C=CC1)OC(=O)N(C)C(C)Cc1ccc2c(c1)OCO2]

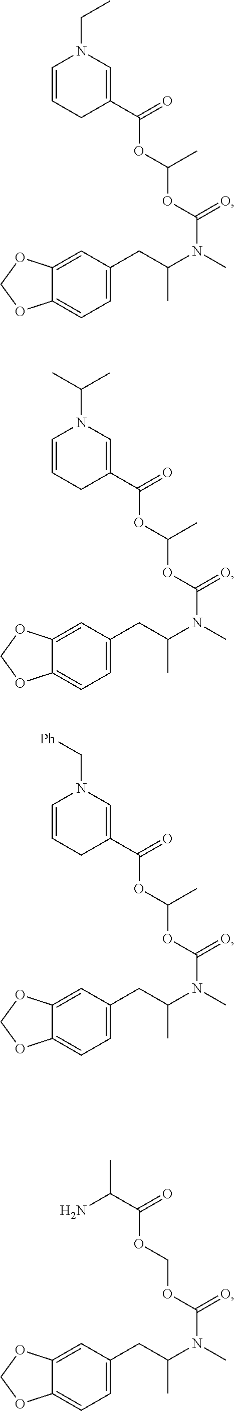 [SMILES: CCN1C=CCC(C(=O)OC(C)OC(=O)N(C)C(C)Cc2ccc3c(c2)OCO3)=C1 ; CC(OC(=O)C1=CN(C(C)C)C=CC1)OC(=O)N(C)C(C)Cc1ccc2c(c1)OCO2 ; CC(OC(=O)C1=CN(Cc2ccccc2)C=CC1)OC(=O)N(C)C(C)Cc1ccc2c(c1)OCO2 ; CC(N)C(=O)OCOC(=O)N(C)C(C)Cc1ccc2c(c1)OCO2]

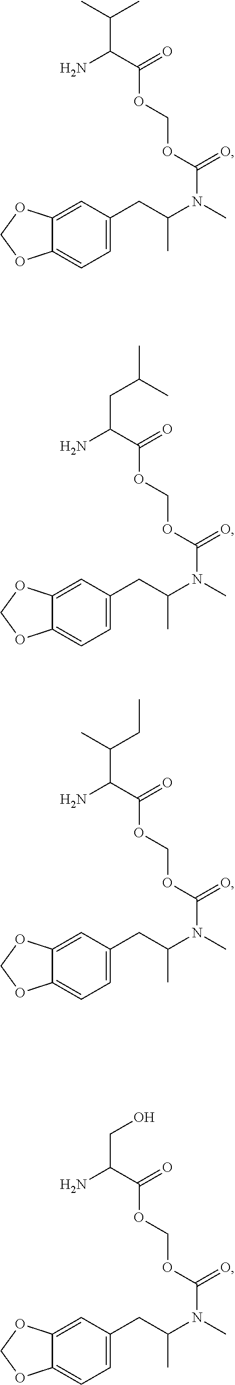 [SMILES: CC(C)C(N)C(=O)OCOC(=O)N(C)C(C)Cc1ccc2c(c1)OCO2 ; CC(C)CC(N)C(=O)OCOC(=O)N(C)C(C)Cc1ccc2c(c1)OCO2 ; CCC(C)C(N)C(=O)OCOC(=O)N(C)C(C)Cc1ccc2c(c1)OCO2 ; CC(Cc1ccc2c(c1)OCO2)N(C)C(=O)OCOC(=O)C(N)CO]

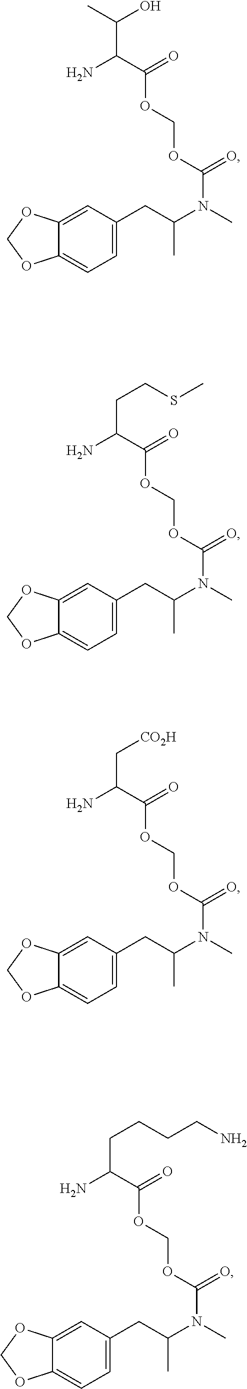 [SMILES: CC(O)C(N)C(=O)OCOC(=O)N(C)C(C)Cc1ccc2c(c1)OCO2 ; CSCCC(N)C(=O)OCOC(=O)N(C)C(C)Cc1ccc2c(c1)OCO2 ; CC(Cc1ccc2c(c1)OCO2)N(C)C(=O)OCOC(=O)C(N)CC(=O)O ; CC(Cc1ccc2c(c1)OCO2)N(C)C(=O)OCOC(=O)C(N)CCCCN]

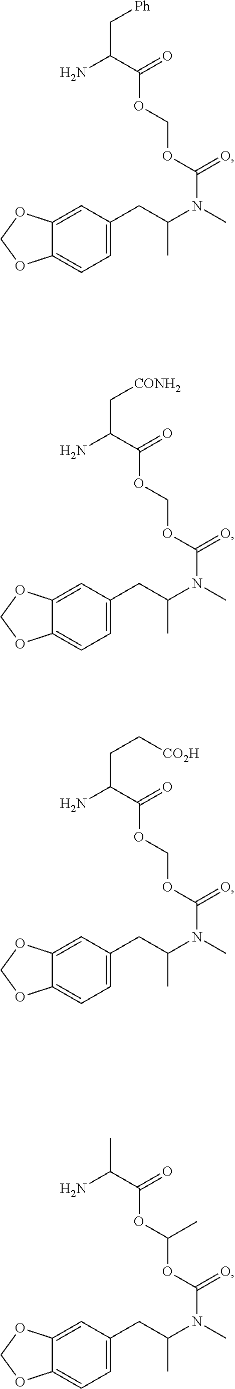 [SMILES: CC(Cc1ccc2c(c1)OCO2)N(C)C(=O)OCOC(=O)C(N)Cc1ccccc1 ; CC(Cc1ccc2c(c1)OCO2)N(C)C(=O)OCOC(=O)C(N)CC(N)=O ; CC(Cc1ccc2c(c1)OCO2)N(C)C(=O)OCOC(=O)C(N)CCC(=O)O ; CC(OC(=O)C(C)N)OC(=O)N(C)C(C)Cc1ccc2c(c1)OCO2]

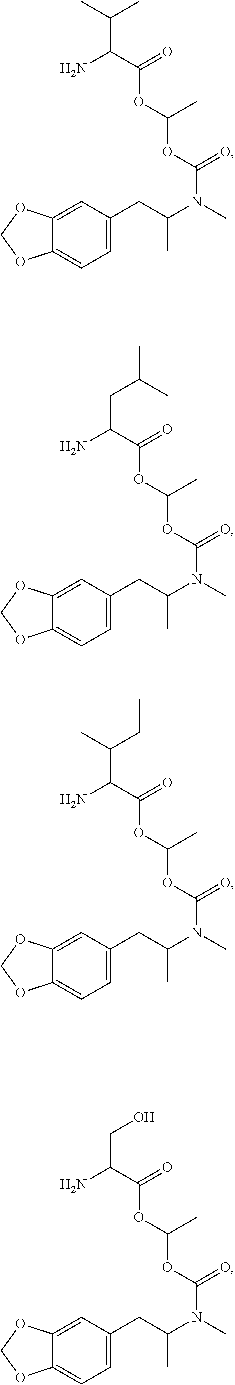 [SMILES: CC(OC(=O)C(N)C(C)C)OC(=O)N(C)C(C)Cc1ccc2c(c1)OCO2 ; CC(C)CC(N)C(=O)OC(C)OC(=O)N(C)C(C)Cc1ccc2c(c1)OCO2 ; CCC(C)C(N)C(=O)OC(C)OC(=O)N(C)C(C)Cc1ccc2c(c1)OCO2 ; CC(OC(=O)C(N)CO)OC(=O)N(C)C(C)Cc1ccc2c(c1)OCO2]

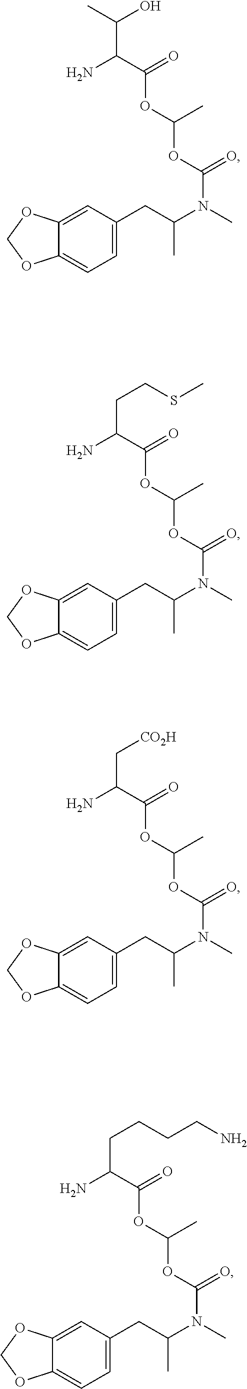 [SMILES: CC(OC(=O)C(N)C(C)O)OC(=O)N(C)C(C)Cc1ccc2c(c1)OCO2 ; CSCCC(N)C(=O)OC(C)OC(=O)N(C)C(C)Cc1ccc2c(c1)OCO2 ; CC(OC(=O)C(N)CC(=O)O)OC(=O)N(C)C(C)Cc1ccc2c(c1)OCO2 ; CC(OC(=O)C(N)CCCCN)OC(=O)N(C)C(C)Cc1ccc2c(c1)OCO2]

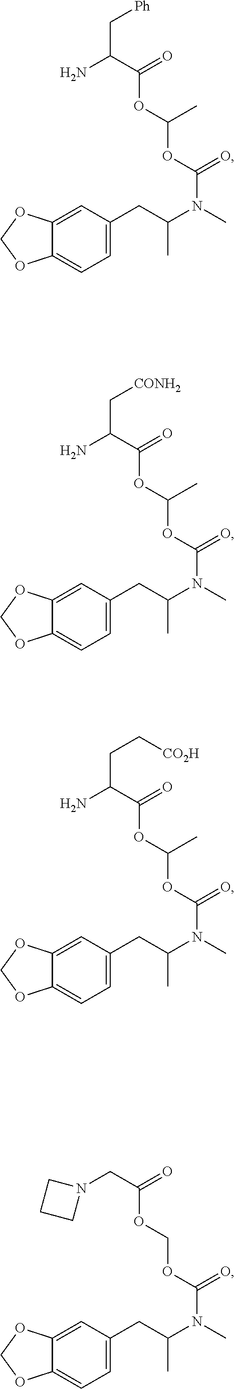 [SMILES: CC(OC(=O)C(N)Cc1ccccc1)OC(=O)N(C)C(C)Cc1ccc2c(c1)OCO2 ; CC(OC(=O)C(N)CC(N)=O)OC(=O)N(C)C(C)Cc1ccc2c(c1)OCO2 ; CC(OC(=O)C(N)CCC(=O)O)OC(=O)N(C)C(C)Cc1ccc2c(c1)OCO2 ; CC(Cc1ccc2c(c1)OCO2)N(C)C(=O)OCOC(=O)CN1CCC1]

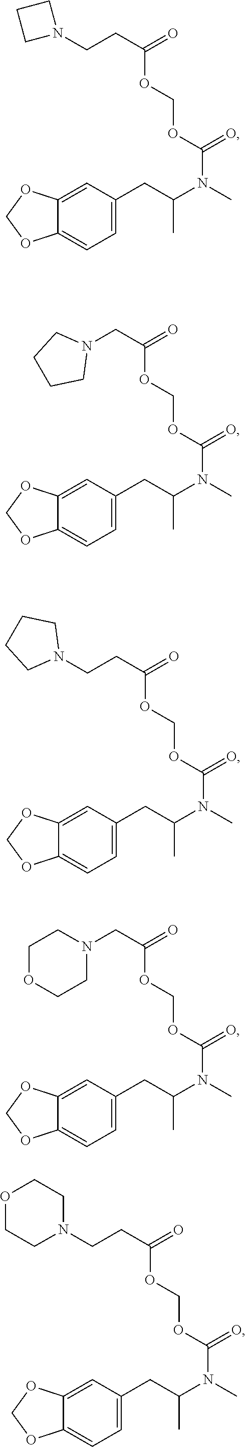

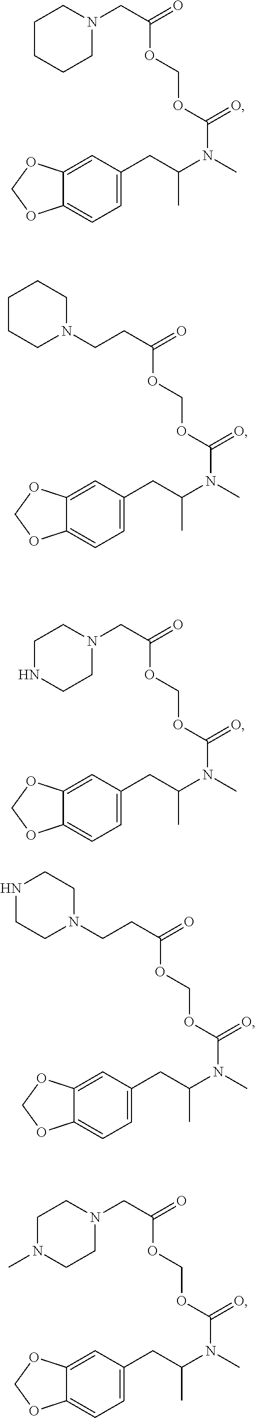

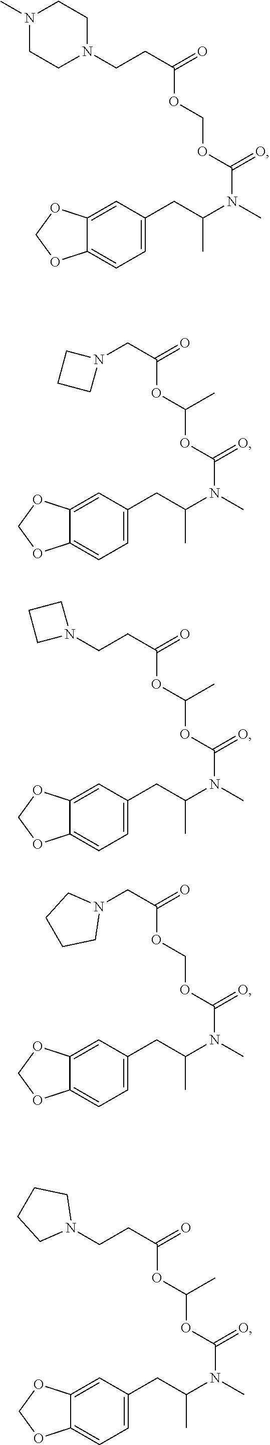

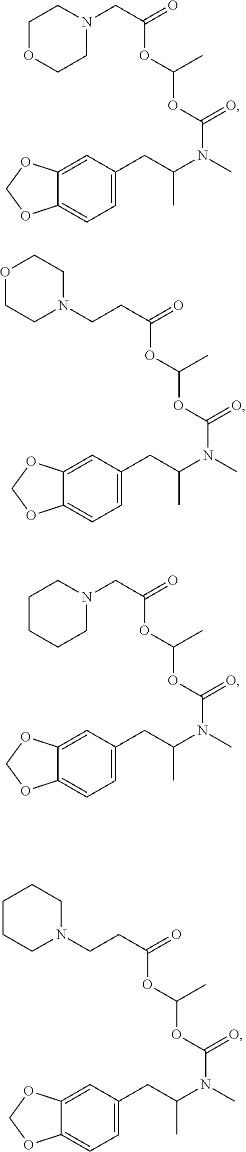

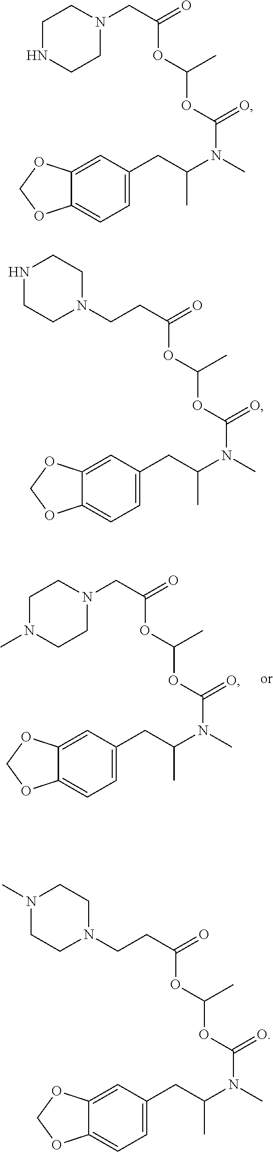

In some embodiments is a compound of Formula (VI) having the structure of Formula (VI-1), or a pharmaceutically acceptable salt thereof:

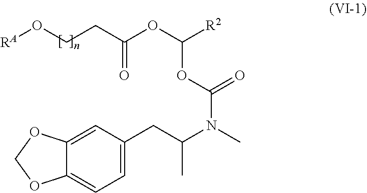

wherein R^{A} is alkyl, heteroalkyl, cycloalkyl, or heterocyclylalkyl, each of which is substituted or unsubstituted; R² is alkyl that is substituted or unsubstituted, or hydrogen; and n is 1, 2, 3, 4, 5, or 6.

In some embodiments is a compound of Formula (VI-1) or a pharmaceutically acceptable salt thereof, wherein R^{A} is methyl, ethyl, isopropyl, n-propyl, tert-butyl, n-butyl, n-pentyl, iso-amyl, cyclopropyl, cyclobutyl, cyclopentyl, or cyclohexyl. In some embodiments is a compound of Formula (VI-1) or a pharmaceutically acceptable salt thereof, wherein R^{A} is methyl.

In some embodiments is a compound of Formula (VI) having the structure of Formula (VI-2), or a pharmaceutically acceptable salt thereof:

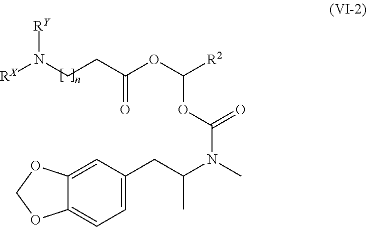

wherein each of R^{X} and R^{Y} is independently hydrogen, alkyl, heteroalkyl, cycloalkyl, or heterocyclylalkyl, wherein alkyl, heteroalkyl, cycloalkyl, or heterocyclylalkyl are substituted or unsubstituted; or R^{X} and R^{Y} together with the atom to which they are attached form a heterocyclylalkyl ring that is substituted or unsubstituted; R² is alkyl that is substituted or unsubstituted, or hydrogen; and n is 1, 2, 3, 4, 5, or 6.

In some embodiments is a compound of Formula (VI-2) or a pharmaceutically acceptable salt thereof, wherein each of R^{X} and R^{Y} is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In some embodiments is a compound of Formula (VI-2) or a pharmaceutically acceptable salt thereof, wherein R^{X} and R^{Y} together with the atom to which they are attached form a piperidine ring, piperazine ring, a morpholine ring, or a pyrrolidine ring, each of which is substituted or unsubstituted.

In some embodiments is a compound of Formula (VI) or (VI-2) having the structure of Formula (VI-2.1), or a pharmaceutically acceptable salt thereof:

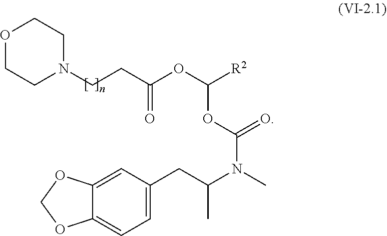

In some embodiments is a compound of Formula (VI) or (VI-2) having the structure of Formula (VI-2.2), or a pharmaceutically acceptable salt thereof:

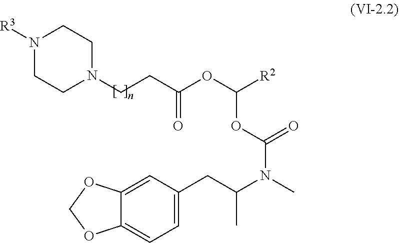

wherein R³ is hydrogen, alkyl, heteroalkyl, or cycloalkyl, wherein alkyl, heteroalkyl, or cycloalkyl is substituted or unsubstituted; R² is alkyl that is substituted or unsubstituted, or hydrogen; and n is 1, 2, 3, 4, 5, or 6.

In some embodiments is a compound of Formula (VI-2.2) or a pharmaceutically acceptable salt thereof, wherein R³ is methyl, ethyl, n-propyl, isopropyl, or —CH(Et)₂. In some embodiments is a compound of Formula (VI-2.2) or a pharmaceutically acceptable salt thereof, wherein R³ is hydrogen.

In some embodiments is a compound of Formula (VI) having the structure of Formula (VI-3), or a pharmaceutically acceptable salt thereof:

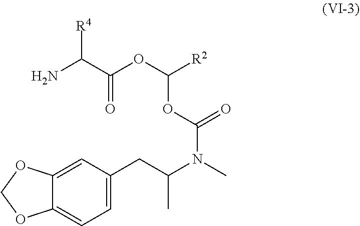

wherein R⁴ is hydrogen, substituted or unsubstituted alkyl, substituted or unsubstituted heteroalkyl, or an amino acid side chain; and R² is alkyl that is substituted or unsubstituted, or hydrogen.

In some embodiments is a compound of Formula (VI-3) or a pharmaceutically acceptable salt thereof, wherein R⁴ is the amino acid side chain. In some embodiments is a compound of Formula (VI-3) or a pharmaceutically acceptable salt thereof, wherein R⁴ is hydrogen. In some embodiments is a compound of Formula (VI-3) or a pharmaceutically acceptable salt thereof, wherein R⁴ is methyl, isopropyl, —CH(Me)Et, —CH₂CH(Me)₂, or —CH₂Ph.

In some embodiments is a compound of Formula (VI) or (VI-3), or a pharmaceutically acceptable salt thereof, wherein the compound is:

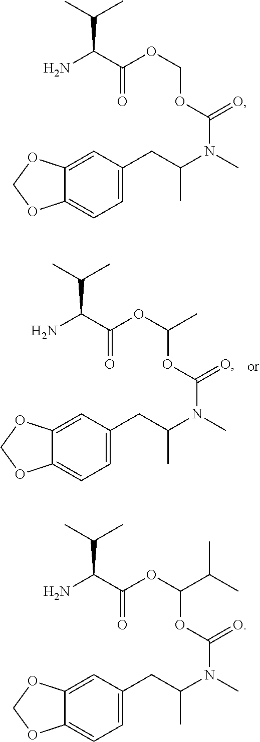 [SMILES: CC(C)[C@H](N)C(=O)OCOC(=O)N(C)C(C)Cc1ccc2c(c1)OCO2 ; CC(OC(=O)[C@@H](N)C(C)C)OC(=O)N(C)C(C)Cc1ccc2c(c1)OCO2 ; CC(C)C(OC(=O)[C@@H](N)C(C)C)OC(=O)N(C)C(C)Cc1ccc2c(c1)OCO2]

In some embodiments is a compound of Formula (VI) having the structure of Formula (VIa), or a pharmaceutically acceptable salt thereof:

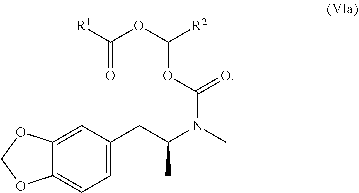

In some embodiments is a compound of Formula (VI) or (VIa) having the structure of Formula (VI-1a), or a pharmaceutically acceptable salt thereof:

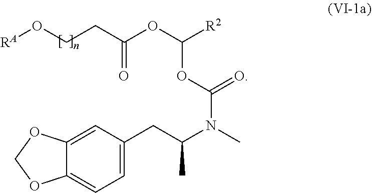

In some embodiments is a compound of Formula (VI), (VIa), or (VI-2), having the structure of Formula (VI-2a), or a pharmaceutically acceptable salt thereof:

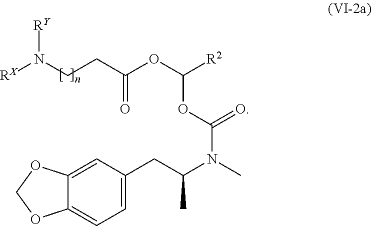

In some embodiments is a compound of Formula (VI), (VIa), (VI-2), or (VI-2a) having the structure of Formula (VI-2.1a), or a pharmaceutically acceptable salt thereof:

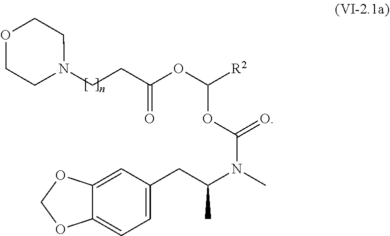

In some embodiments is a compound of Formula (VI), (VIa), (VI-2), or (VI-2a) having the structure of Formula (VI-2.2a), or a pharmaceutically acceptable salt thereof:

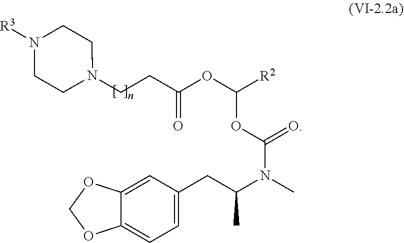

In some embodiments is a compound of Formula (VI), (VIa), or (VI-3) having the structure of Formula (VI-3a), or a pharmaceutically acceptable salt thereof:

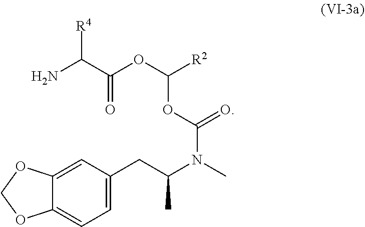

In some embodiments is a compound of Formula (VI) having the structure of Formula (VIb), or a pharmaceutically acceptable salt thereof:

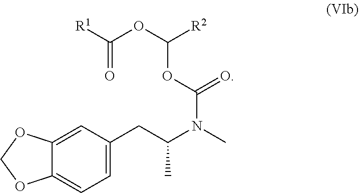

In some embodiments is a compound of Formula (VI) or (VIb) having the structure of Formula (VI-1b), or a pharmaceutically acceptable salt thereof:

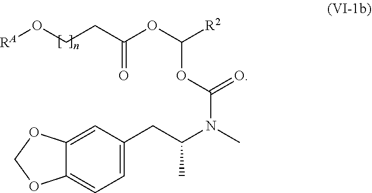

In some embodiments is a compound of Formula (VI), (VIb), or (VI-2), having the structure of Formula (VI-2b), or a pharmaceutically acceptable salt thereof:

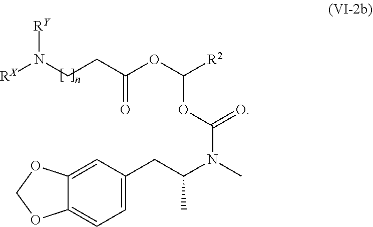

In some embodiments is a compound of Formula (VI), (VIb), (VI-2), or (VI-2b) having the structure of Formula (VI-2.1b), or a pharmaceutically acceptable salt thereof:

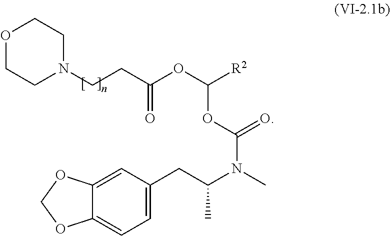

In some embodiments is a compound of Formula (VI), (VIb), (VI-2), or (VI-2b) having the structure of Formula (VI-2.2b), or a pharmaceutically acceptable salt thereof:

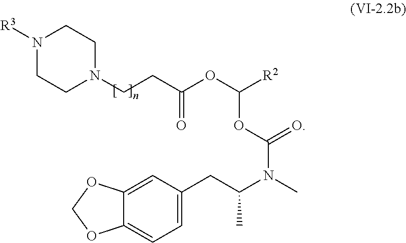

In some embodiments is a compound of Formula (VI), (VIb), or (VI-3) having the structure of Formula (VI-3b), or a pharmaceutically acceptable salt thereof:

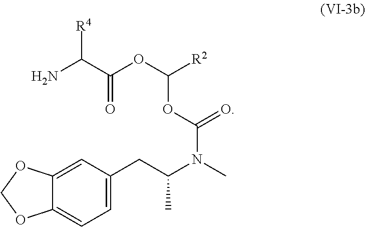

In some embodiments is a compound of Formula (VI), (VIa), (VIb), (VI-1), (VI-1a), (VI-1b), (VI-2), (VI-2a), (VI-2b), (VI-2.1), (VI-2.1a), (VI-2.1b), (VI-2.2), (VI-2.2a), (VI-2.2b) (VI-3), (VI-3a), or (VI-3b), or a pharmaceutically acceptable salt thereof, wherein R² is hydrogen. In some embodiments is a compound of Formula (VI), (VIa), (VIb), (VI-1), (VI-1a), (VI-1b), (VI-2), (VI-2a), (VI-2b), (VI-2.1), (VI-2.1a), (VI-2.1b), (VI-2.2), (VI-2.2a), (VI-2.2b) (VI-3), (VI-3a), or (VI-3b), or a pharmaceutically acceptable salt thereof, wherein R² is methyl, ethyl, n-propyl, isopropyl, or —CH(Et)₂.

In some embodiments is a compound of Formula (VI-1), (VI-1a), (VI-1b), (VI-2), (VI-2a), (VI-2b), (VI-2.1), (VI-2.1a), (VI-2.1b), (VI-2.2), (VI-2.2a), or (VI-2.2b), or a pharmaceutically acceptable salt thereof, wherein n is 1.

In another aspect, the present disclosure provides a compound of Formula (VII), or a pharmaceutically acceptable salt thereof:

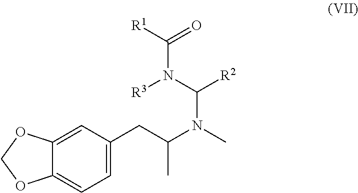

wherein R¹ is alkyl, alkenyl, heteroalkyl, cycloalkyl, heterocyclylalkyl, aryl, or heteroaryl, each of which is substituted or unsubstituted, or R¹ and the carbonyl to which R¹ is attached form an amino acid residue; and each of R² and R³ is alkyl that is substituted or unsubstituted, or hydrogen.

In some embodiments is a compound of Formula (VII) or a pharmaceutically acceptable salt thereof, wherein R¹ together which the carbonyl to which R¹ is attached form an amino acid residue.

In some embodiments is a compound of Formula (VII) or a pharmaceutically acceptable salt thereof, wherein R¹ is alkyl or heteroalkyl that is substituted or unsubstituted. In some embodiments is a compound of Formula (VII) or a pharmaceutically acceptable salt thereof, wherein R¹ is alkyl that is substituted. In some embodiments is a compound of Formula (VI) or a pharmaceutically acceptable salt thereof, wherein R¹ is alkyl that is substituted with heterocyclylalkyl that is substituted or unsubstituted.

In some embodiments is a compound of Formula (VII) or a pharmaceutically acceptable salt thereof, wherein R¹ is heteroalkyl that is substituted. In some embodiments is a compound of Formula (VII) or a pharmaceutically acceptable salt thereof, wherein R¹ is heteroalkyl that is substituted with cycloalkyl or heterocyclylalkyl, wherein cycloalkyl or heterocyclylalkyl are substituted or unsubstituted.

In some embodiments is a compound of Formula (VII) or a pharmaceutically acceptable salt thereof, wherein R¹ is heterocyclylalkyl that is substituted or unsubstituted. In some embodiments is a compound of Formula (VI) or a pharmaceutically acceptable salt thereof, wherein R¹ is heterocyclylalkyl that is substituted with alkyl.

In some embodiments is a compound of Formula (VII) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (VII) or a pharmaceutically acceptable salt thereof, wherein each of R² and R³ is methyl, ethyl, n-propyl, isopropyl, tert-butyl, or hydrogen. In some embodiments is a compound of Formula (VII) or a pharmaceutically acceptable salt thereof, wherein R² is methyl or hydrogen. In some embodiments is a compound of Formula (VII) or a pharmaceutically acceptable salt thereof, wherein R³ is methyl or hydrogen. In some embodiments is a compound of Formula (VII) or a pharmaceutically acceptable salt thereof, wherein R² is methyl. In some embodiments is a compound of Formula (VII) or a pharmaceutically acceptable salt thereof, wherein R² is hydrogen. In some embodiments is a compound of Formula (VII) or a pharmaceutically acceptable salt thereof, wherein R³ is methyl. In some embodiments is a compound of Formula (VII) or a pharmaceutically acceptable salt thereof, wherein R³ is hydrogen.

In some embodiments is a compound of Formula (VII) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (VII) or a pharmaceutically acceptable salt thereof, wherein the compound is:

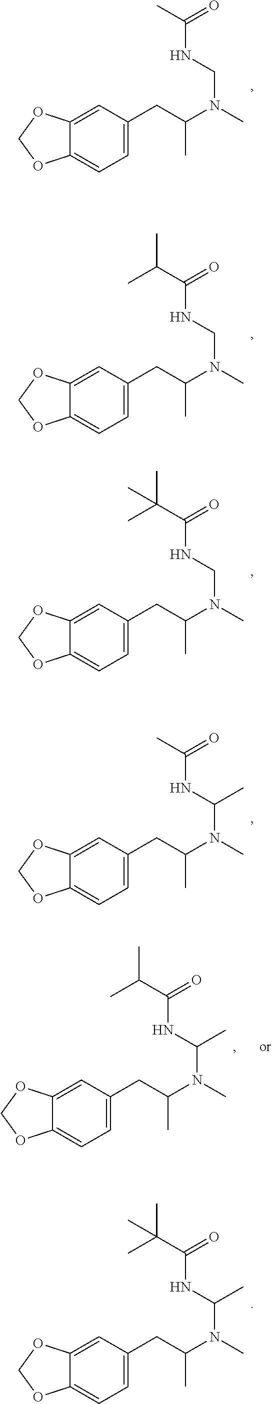 [SMILES: CC(=O)NCN(C)C(C)Cc1ccc2c(c1)OCO2 ; CC(C)C(=O)NCN(C)C(C)Cc1ccc2c(c1)OCO2 ; CC(Cc1ccc2c(c1)OCO2)N(C)CNC(=O)C(C)(C)C ; CC(=O)NC(C)N(C)C(C)Cc1ccc2c(c1)OCO2 ; CC(C)C(=O)NC(C)N(C)C(C)Cc1ccc2c(c1)OCO2 ; CC(Cc1ccc2c(c1)OCO2)N(C)C(C)NC(=O)C(C)(C)C]

In some embodiments is a compound of Formula (VII) or a pharmaceutically acceptable salt thereof, wherein the compound is:

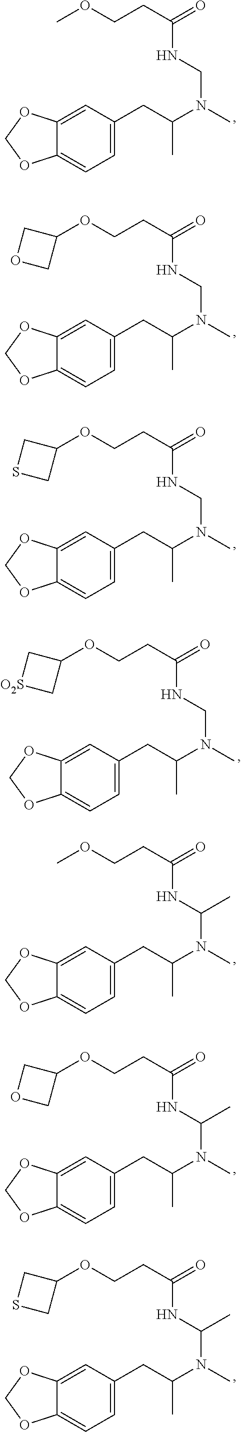

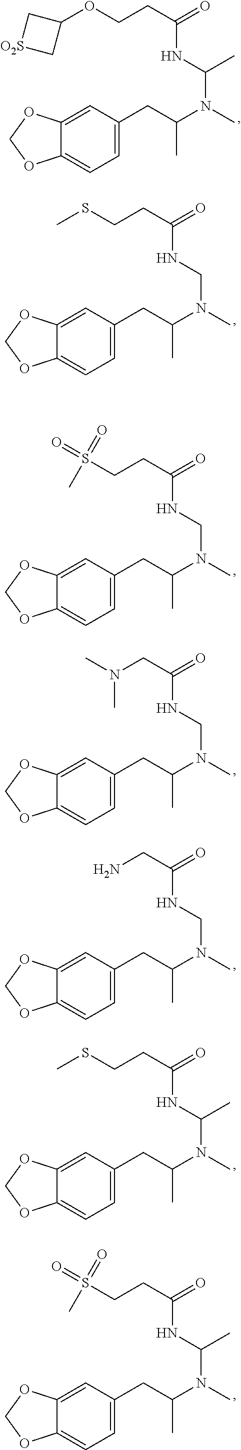

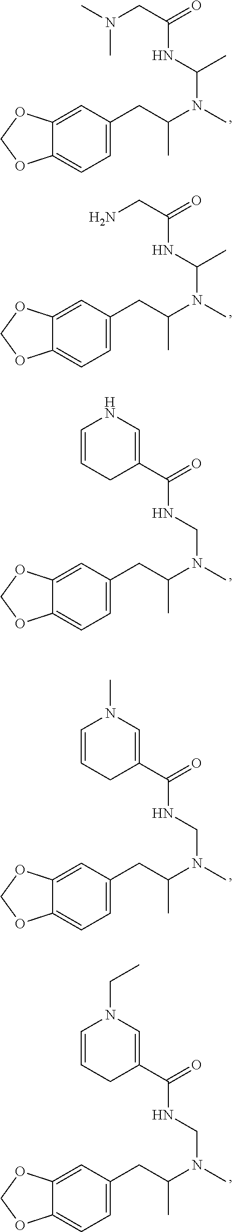 [SMILES: CC(Cc1ccc2c(c1)OCO2)N(C)C(C)NC(=O)CN(C)C ; CC(Cc1ccc2c(c1)OCO2)N(C)C(C)NC(=O)CN ; CC(Cc1ccc2c(c1)OCO2)N(C)CNC(=O)C1=CNC=CC1 ; CC(Cc1ccc2c(c1)OCO2)N(C)CNC(=O)C1=CN(C)C=CC1 ; CCN1C=CCC(C(=O)NCN(C)C(C)Cc2ccc3c(c2)OCO3)=C1]

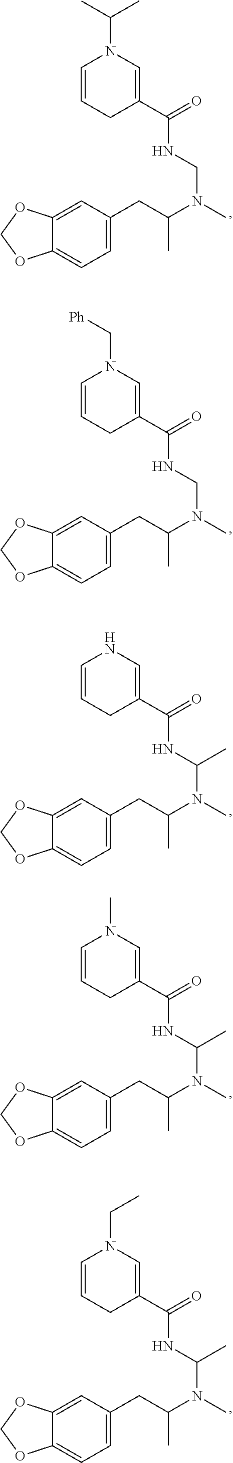

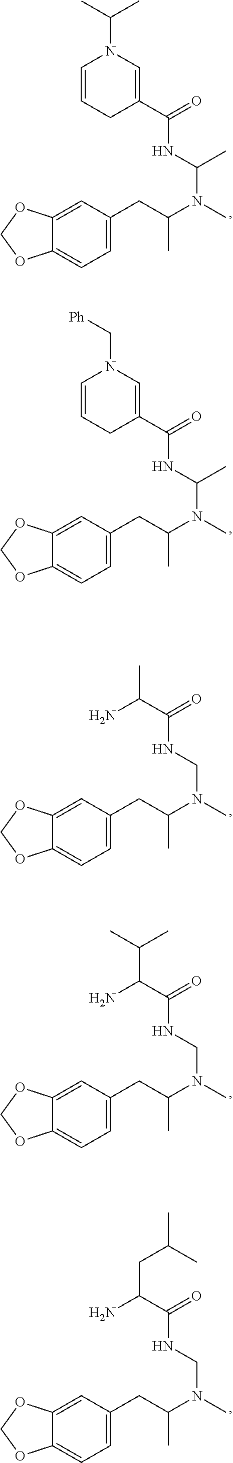 [SMILES: CC(C)N1C=CCC(C(=O)NC(C)N(C)C(C)Cc2ccc3c(c2)OCO3)=C1 ; CC(Cc1ccc2c(c1)OCO2)N(C)C(C)NC(=O)C1=CN(Cc2ccccc2)C=CC1 ; CC(N)C(=O)NCN(C)C(C)Cc1ccc2c(c1)OCO2 ; CC(C)C(N)C(=O)NCN(C)C(C)Cc1ccc2c(c1)OCO2 ; CC(C)CC(N)C(=O)NCN(C)C(C)Cc1ccc2c(c1)OCO2]

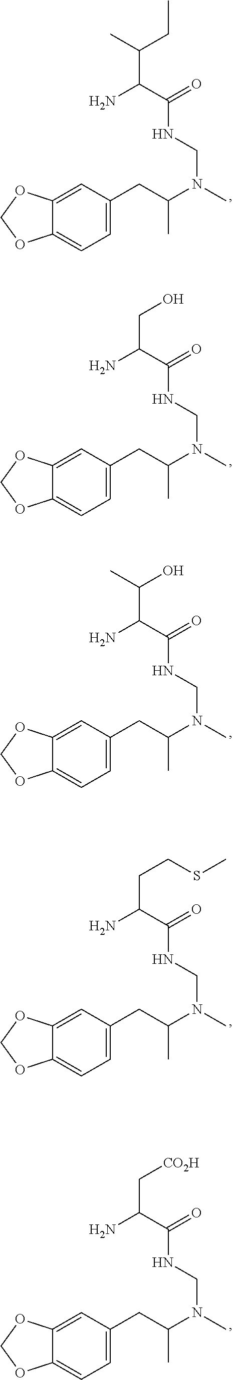 [SMILES: CCC(C)C(N)C(=O)NCN(C)C(C)Cc1ccc2c(c1)OCO2 ; CC(Cc1ccc2c(c1)OCO2)N(C)CNC(=O)C(N)CO ; CC(O)C(N)C(=O)NCN(C)C(C)Cc1ccc2c(c1)OCO2 ; CSCCC(N)C(=O)NCN(C)C(C)Cc1ccc2c(c1)OCO2 ; CC(Cc1ccc2c(c1)OCO2)N(C)CNC(=O)C(N)CC(=O)O]

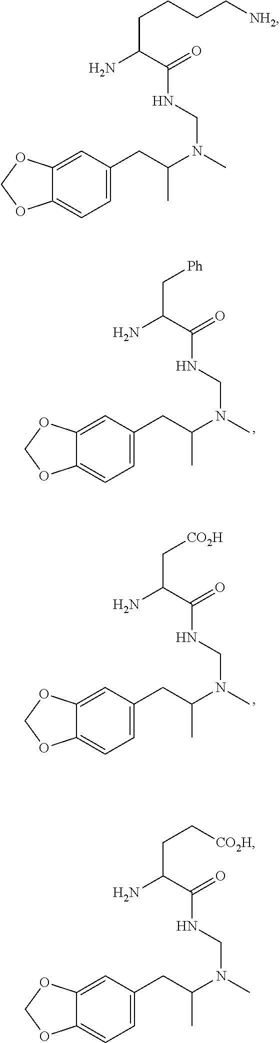 [SMILES: CC(Cc1ccc2c(c1)OCO2)N(C)CNC(=O)C(N)CCCCN ; CC(Cc1ccc2c(c1)OCO2)N(C)CNC(=O)C(N)Cc1ccccc1 ; CC(Cc1ccc2c(c1)OCO2)N(C)CNC(=O)C(N)CC(=O)O ; CC(Cc1ccc2c(c1)OCO2)N(C)CNC(=O)C(N)CCC(=O)O]

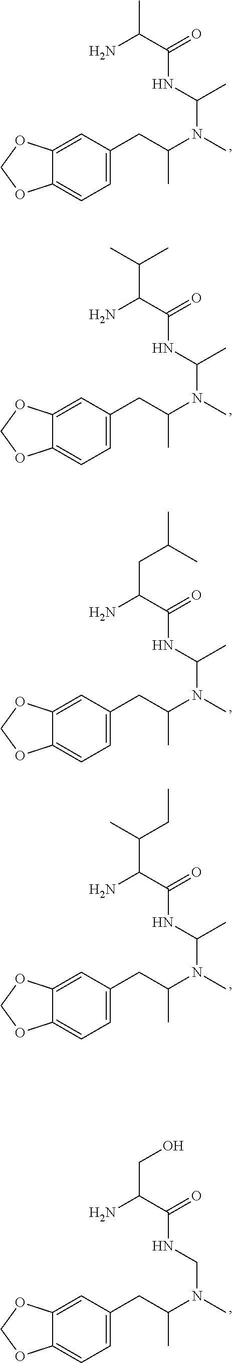

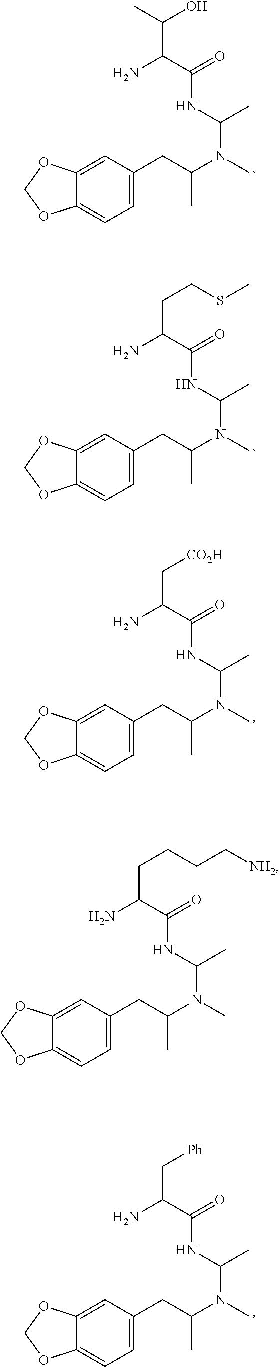 [SMILES: CC(O)C(N)C(=O)NC(C)N(C)C(C)Cc1ccc2c(c1)OCO2 ; CSCCC(N)C(=O)NC(C)N(C)C(C)Cc1ccc2c(c1)OCO2 ; CC(Cc1ccc2c(c1)OCO2)N(C)C(C)NC(=O)C(N)CC(=O)O ; CC(Cc1ccc2c(c1)OCO2)N(C)C(C)NC(=O)C(N)CCCCN ; CC(Cc1ccc2c(c1)OCO2)N(C)C(C)NC(=O)C(N)Cc1ccccc1]

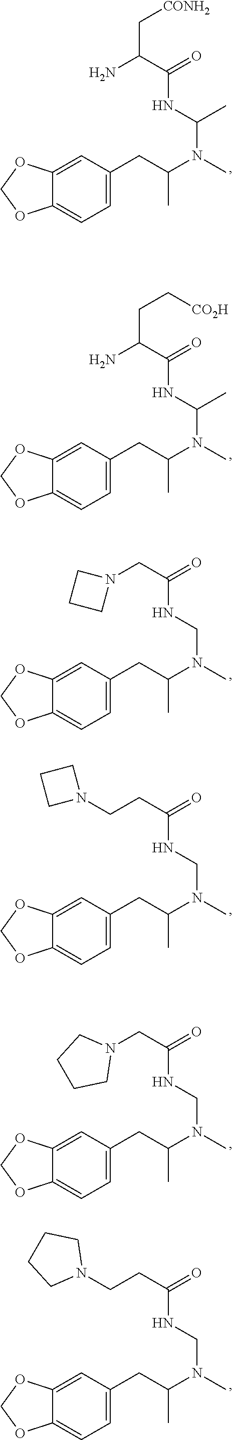

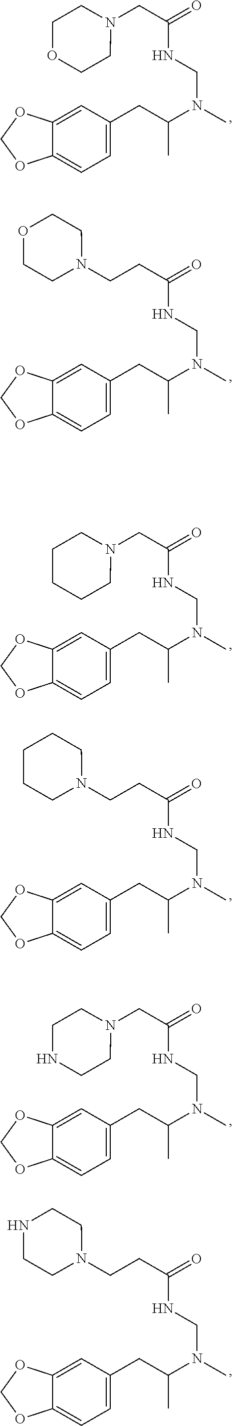

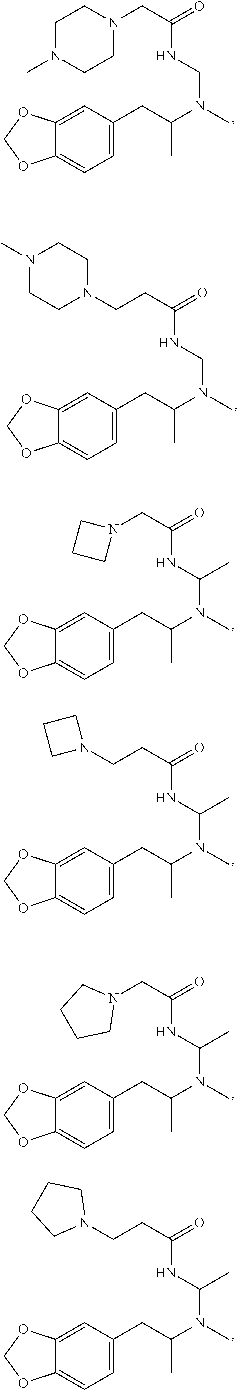

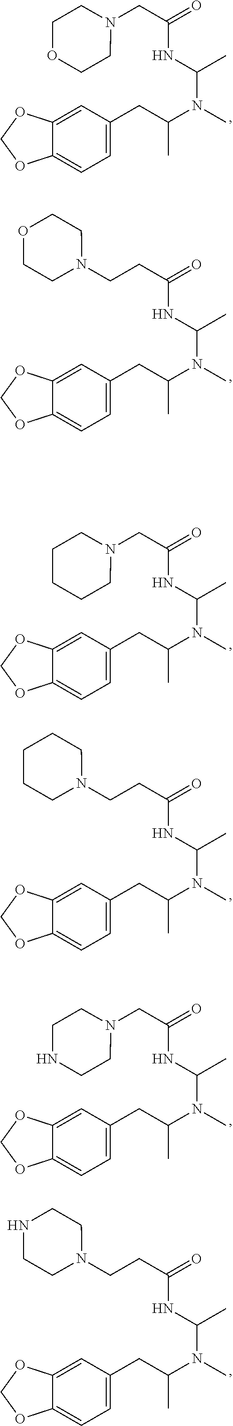

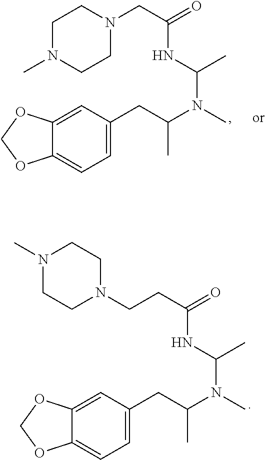

In some embodiments is a compound of Formula (VII) having the structure of Formula (VII-1), or a pharmaceutically acceptable salt thereof:

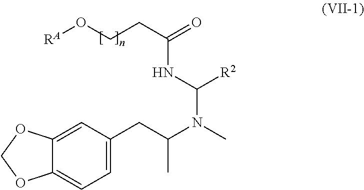

wherein R^{A} is alkyl, heteroalkyl, cycloalkyl, or heterocyclylalkyl, each of which is substituted or unsubstituted; R² is hydrogen or alkyl that is substituted or unsubstituted; and n is 1, 2, 3, 4, 5, or 6.

In some embodiments is a compound of Formula (VII-1) or a pharmaceutically acceptable salt thereof, wherein R^{A} is methyl, ethyl, isopropyl, n-propyl, tert-butyl, n-butyl, n-pentyl, iso-amyl, cyclopropyl, cyclobutyl, cyclopentyl, or cyclohexyl. In some embodiments is a compound of Formula (VII-1) or a pharmaceutically acceptable salt thereof, wherein R^{A} is methyl.

In some embodiments is a compound of Formula (VII) having the structure of Formula (VII-2), or a pharmaceutically acceptable salt thereof:

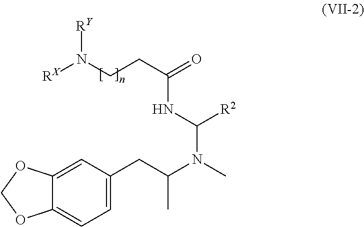

wherein each of R^{X} and R^{Y} is independently hydrogen, alkyl, heteroalkyl, cycloalkyl, or heterocyclylalkyl, wherein alkyl, heteroalkyl, cycloalkyl, or heterocyclylalkyl are substituted or unsubstituted; or R^{X} and R^{Y} together with the atom to which they are attached form a heterocyclylalkyl ring that is substituted or unsubstituted; R² is alkyl that is substituted or unsubstituted, or hydrogen; and n is 1, 2, 3, 4, 5, or 6.

In some embodiments is a compound of Formula (VII-2) or a pharmaceutically acceptable salt thereof, wherein each of R^{X} and R^{Y} is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In some embodiments is a compound of Formula (VII-2) or a pharmaceutically acceptable salt thereof, wherein R^{X} and R^{Y} together with the atom to which they are attached form a piperidine ring, piperazine ring, a morpholine ring, or a pyrrolidine ring, each of which is substituted or unsubstituted.

In some embodiments is a compound of Formula (VII) or (VII-2) having the structure of Formula (VII-2.1), or a pharmaceutically acceptable salt thereof:

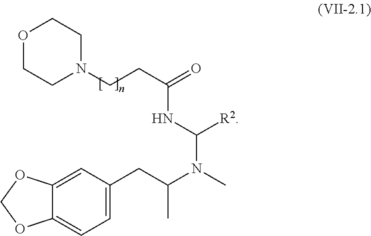

In some embodiments is a compound of Formula (VII) or (VII-2) having the structure of Formula (VII-2.2), or a pharmaceutically acceptable salt thereof:

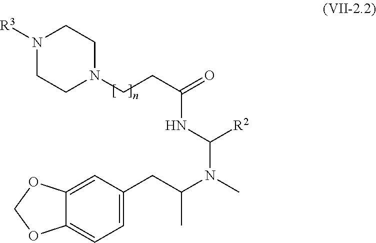

wherein R³ is hydrogen, alkyl, heteroalkyl, or cycloalkyl, wherein alkyl, heteroalkyl, and cycloalkyl are substituted or unsubstituted; R² is alkyl that is substituted or unsubstituted, or hydrogen; and n is 1, 2, 3, 4, 5, or 6.

In some embodiments is a compound of Formula (VII-2.2) or a pharmaceutically acceptable salt thereof, wherein R³ is methyl, ethyl, n-propyl, isopropyl, or —CH(Et)₂. In some embodiments is a compound of Formula (VII-2.2) or a pharmaceutically acceptable salt thereof, wherein R³ is hydrogen.

In some embodiments is a compound of Formula (VII) having the structure of Formula (VII-3), or a pharmaceutically acceptable salt thereof:

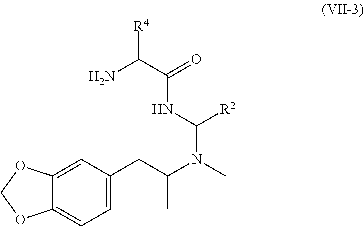

wherein R⁴ is hydrogen, substituted or unsubstituted alkyl, substituted or unsubstituted heteroalkyl, or an amino acid side chain; and R² is alkyl that is substituted or unsubstituted, or hydrogen.

In some embodiments is a compound of Formula (VII-3) or a pharmaceutically acceptable salt thereof, wherein R⁴ is an amino acid side chain. In some embodiments is a compound of Formula (VII-3) or a pharmaceutically acceptable salt thereof, wherein R⁴ is hydrogen. In some embodiments is a compound of Formula (VII-3) or a pharmaceutically acceptable salt thereof, wherein R⁴ is methyl, isopropyl, —CH(Me)Et, —CH₂CH(Me)₂, or —CH₂Ph.

In some embodiments is a compound of Formula (VII) or (VII-3), or a pharmaceutically acceptable salt thereof, wherein the compound is:

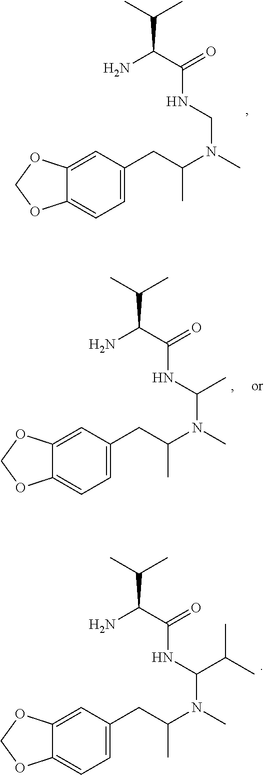 [SMILES: CC(C)[C@H](N)C(=O)NCN(C)C(C)Cc1ccc2c(c1)OCO2 ; CC(C)[C@H](N)C(=O)NC(C)N(C)C(C)Cc1ccc2c(c1)OCO2 ; CC(C)C(NC(=O)[C@@H](N)C(C)C)N(C)C(C)Cc1ccc2c(c1)OCO2]

In some embodiments is a compound of Formula (VII) having the structure of Formula (VIIa), or a pharmaceutically acceptable salt thereof:

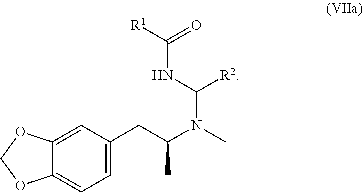

In some embodiments is a compound of Formula (VII) or (VIIa) having the structure of Formula (VII-1a), or a pharmaceutically acceptable salt thereof:

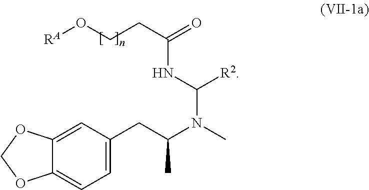

In some embodiments is a compound of Formula (VII), (VIIa), or (VII-2), having the structure of Formula (VII-2a), or a pharmaceutically acceptable salt thereof:

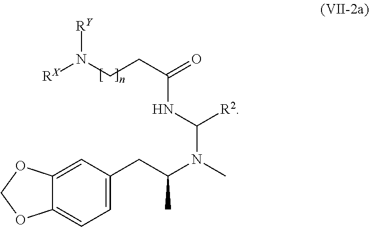

In some embodiments is a compound of Formula (VII), (VIIa), (VII-2), or (VII-2a) having the structure of Formula (VII-2.1a), or a pharmaceutically acceptable salt thereof:

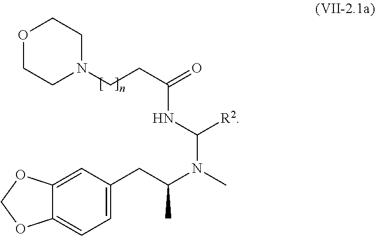

In some embodiments is a compound of Formula (VII), (VIIa), (VII-2), or (VII-2a) having the structure of Formula (VII-2.2a), or a pharmaceutically acceptable salt thereof:

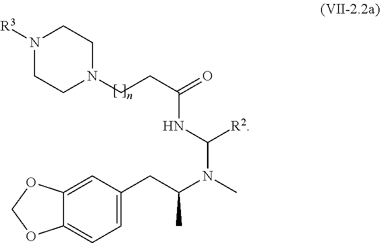

In some embodiments is a compound of Formula (VII), (VIIa), or (VII-3) having the structure of Formula (VII-3a), or a pharmaceutically acceptable salt thereof:

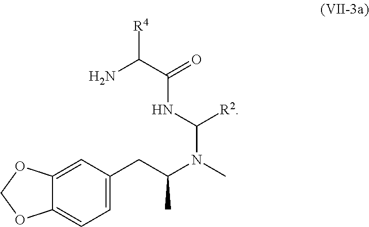

In some embodiments is a compound of Formula (VII) having the structure of Formula (VIIb), or a pharmaceutically acceptable salt thereof:

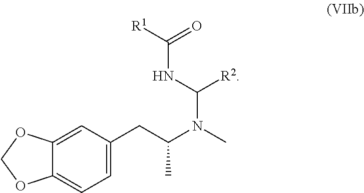

In some embodiments is a compound of Formula (VII) or (VIIb) having the structure of Formula (VII-1b), or a pharmaceutically acceptable salt thereof:

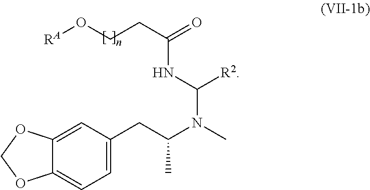

In some embodiments is a compound of Formula (VII), (VIIb), or (VII-2), having the structure of Formula (VII-2b), or a pharmaceutically acceptable salt thereof:

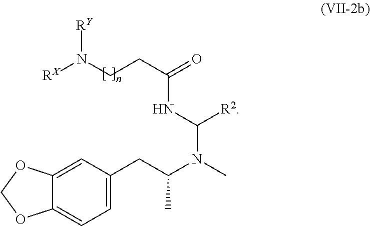

In some embodiments is a compound of Formula (VII), (VIIb), (VII-2), or (VII-2b) having the structure of Formula (VII-2.1b), or a pharmaceutically acceptable salt thereof:

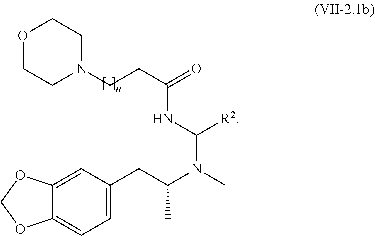

In some embodiments is a compound of Formula (VII), (VIIb), (VII-2), or (VII-2b) having the structure of Formula (VII-2.2b), or a pharmaceutically acceptable salt thereof:

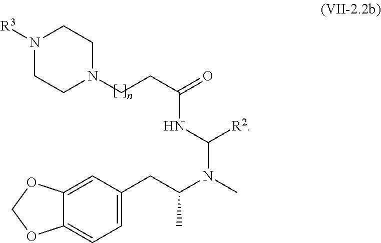

In some embodiments is a compound of Formula (VII), (VIIb), or (VII-3) having the structure of Formula (VII-3b), or a pharmaceutically acceptable salt thereof:

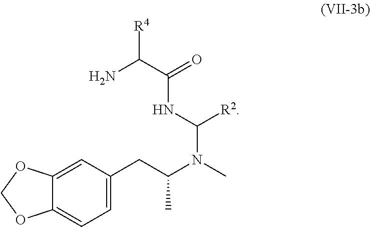

In some embodiments is a compound of Formula (VII), (VIIa), (VIIb), (VII-1), (VII-1a), (VII-1b), (VII-2), (VII-2a), (VII-2b), (VII-2.1), (VII-2.1a), (VII-2.1b), (VII-2.2), (VII-2.2a), (VII-2.2b) (VII-3), (VII-3a), or (VII-3b), or a pharmaceutically acceptable salt thereof, wherein R² is hydrogen. In some embodiments is a compound of Formula (VII), (VIIa), (VIIb), (VII-1), (VII-1a), (VII-1b), (VII-2), (VII-2a), (VII-2b), (VII-2.1), (VII-2.1a), (VII-2.1b), (VII-2.2), (VII-2.2a), (VII-2.2b) (VII-3), (VII-3a), or (VII-3b), or a pharmaceutically acceptable salt thereof, wherein R² is methyl, ethyl, n-propyl, isopropyl, or —CH(Et)₂.

In some embodiments is a compound of Formula (VII-1), (VII-1a), (VII-1b), (VII-2), (VII-2a), (VII-2b), (VII-2.1), (VII-2.1a), (VII-2.1b), (VII-2.2), (VII-2.2a), or (VII-2.2b), or a pharmaceutically acceptable salt thereof, wherein n is 1.

In another aspect, the present disclosure provides a compound of Formula (VIII), or a pharmaceutically acceptable salt thereof:

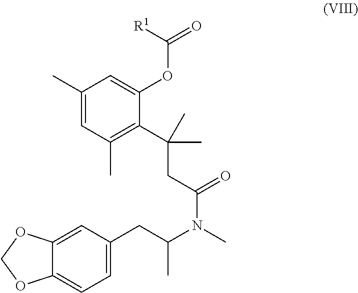

wherein R¹ is alkyl, alkenyl, heteroalkyl, cycloalkyl, heterocyclylalkyl, aryl, or heteroaryl, each of which is substituted or unsubstituted, or R¹ and the carbonyl to which R¹ is attached form an amino acid residue.

In some embodiments is a compound of Formula (VIII) or a pharmaceutically acceptable salt thereof, wherein R¹ is alkyl or heteroalkyl that is substituted or unsubstituted. In some embodiments is a compound of Formula (VII) or a pharmaceutically acceptable salt thereof, wherein R¹ is alkyl that is substituted. In some embodiments is a compound of Formula (VI) or a pharmaceutically acceptable salt thereof, wherein R¹ is alkyl that is substituted with heterocyclylalkyl that is substituted or unsubstituted.

In some embodiments is a compound of Formula (VIII) or a pharmaceutically acceptable salt thereof, wherein R¹ is heteroalkyl that is substituted. In some embodiments is a compound of Formula (VII) or a pharmaceutically acceptable salt thereof, wherein R¹ is heteroalkyl that is substituted with cycloalkyl or heterocyclylalkyl, wherein cycloalkyl or heterocyclylalkyl are substituted or unsubstituted.

In some embodiments is a compound of Formula (VIII) or a pharmaceutically acceptable salt thereof, wherein R¹ is heterocyclylalkyl that is substituted or unsubstituted. In some embodiments is a compound of Formula (VIII) or a pharmaceutically acceptable salt thereof, wherein R¹ is heterocyclylalkyl that is substituted with alkyl.

In some embodiments is a compound of Formula (VIII) or a pharmaceutically acceptable salt thereof, wherein the compound is:

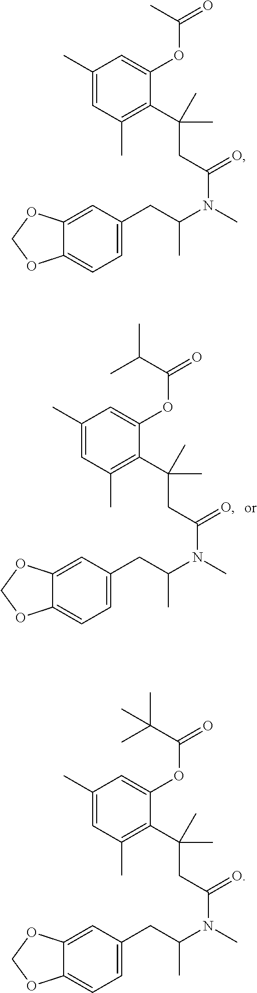 [SMILES: CC(=O)Oc1cc(C)cc(C)c1C(C)(C)CC(=O)N(C)C(C)Cc1ccc2c(c1)OCO2 ; Cc1cc(C)c(C(C)(C)CC(=O)N(C)C(C)Cc2ccc3c(c2)OCO3)c(OC(=O)C(C)C)c1 ; Cc1cc(C)c(C(C)(C)CC(=O)N(C)C(C)Cc2ccc3c(c2)OCO3)c(OC(=O)C(C)(C)C)c1]

In some embodiments is a compound of Formula (VIII) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, —CH₂CH₂OMe, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (VIII) having the structure of Formula (VIIIa), or a pharmaceutically acceptable salt thereof:

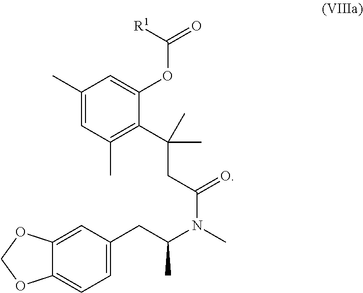

In some embodiments is a compound of Formula (VIII) having the structure of Formula (VIIIb), or a pharmaceutically acceptable salt thereof:

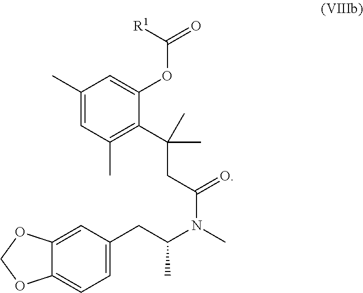

In another aspect, the present disclosure provides a compound of Formula (IX), or a pharmaceutically acceptable salt thereof:

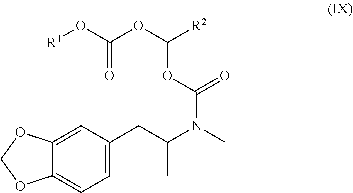

wherein R¹ is alkyl, alkenyl, heteroalkyl, cycloalkyl, heterocyclylalkyl, aryl, or heteroaryl, each of which is substituted or unsubstituted; and R² is alkyl that is substituted or unsubstituted, or hydrogen.

In some embodiments is a compound of Formula (IX) or a pharmaceutically acceptable salt thereof, wherein R¹ is alkyl or heteroalkyl that is substituted or unsubstituted. In some embodiments is a compound of Formula (IX) or a pharmaceutically acceptable salt thereof, wherein R¹ is alkyl that is substituted. In some embodiments is a compound of Formula (VI) or a pharmaceutically acceptable salt thereof, wherein R¹ is alkyl that is substituted with heterocyclylalkyl that is substituted or unsubstituted.

In some embodiments is a compound of Formula (IX) or a pharmaceutically acceptable salt thereof, wherein R¹ is heteroalkyl that is substituted. In some embodiments is a compound of Formula (VII) or a pharmaceutically acceptable salt thereof, wherein R¹ is heteroalkyl that is substituted with cycloalkyl or heterocyclylalkyl, wherein cycloalkyl or heterocyclylalkyl are substituted or unsubstituted.

In some embodiments is a compound of Formula (IX) or a pharmaceutically acceptable salt thereof, wherein R¹ is heterocyclylalkyl that is substituted or unsubstituted. In some embodiments is a compound of Formula (IX) or a pharmaceutically acceptable salt thereof, wherein R¹ is heterocyclylalkyl that is substituted with alkyl.

In some embodiments is a compound of Formula (VII) or a pharmaceutically acceptable salt thereof, wherein R² is unsubstituted alkyl. In some embodiments is a compound of Formula (VII) or a pharmaceutically acceptable salt thereof, wherein R² is methyl, ethyl, n-propyl, isopropyl, tert-butyl, or hydrogen. In some embodiments is a compound of Formula (VII) or a pharmaceutically acceptable salt thereof, wherein R² is methyl or hydrogen. In some embodiments is a compound of Formula (VII) or a pharmaceutically acceptable salt thereof, wherein R² is methyl. In some embodiments is a compound of Formula (VII) or a pharmaceutically acceptable salt thereof, wherein R² is hydrogen.

In some embodiments is a compound of Formula (IX) or a pharmaceutically acceptable salt thereof, wherein the compound is:

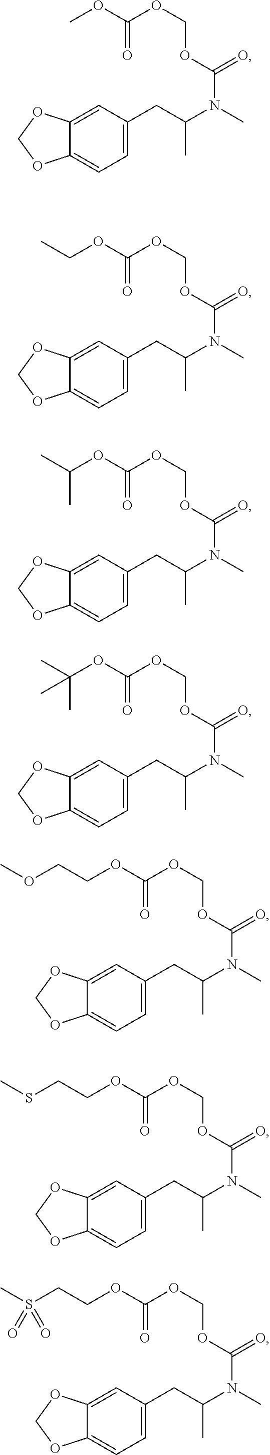

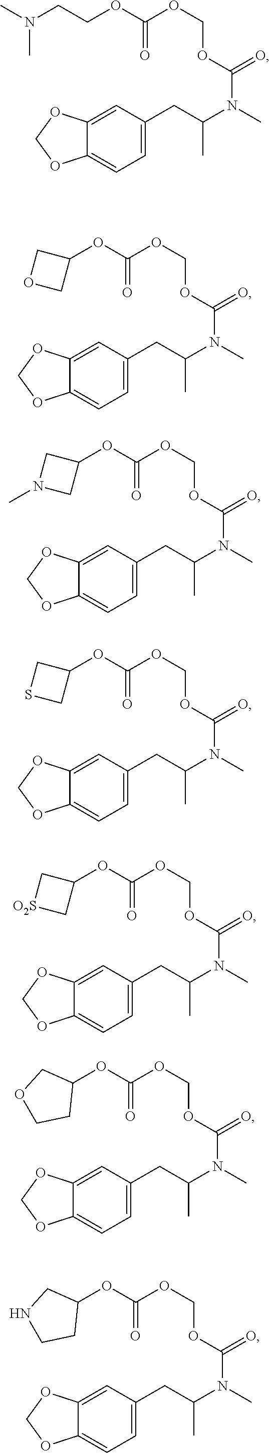

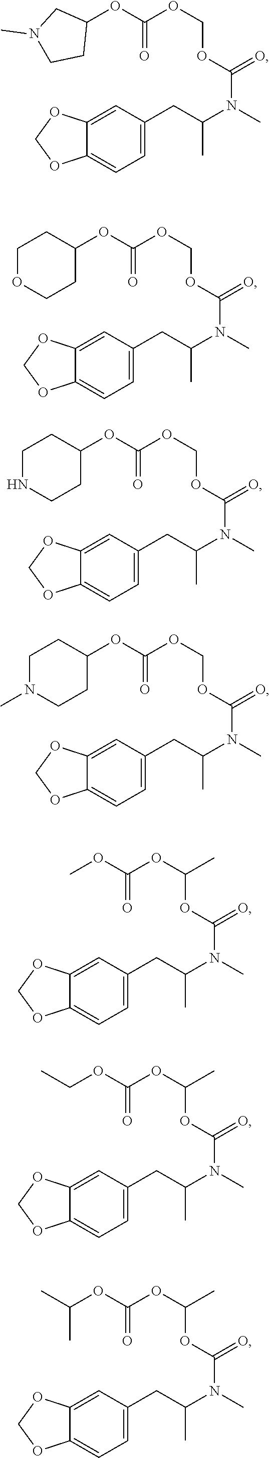

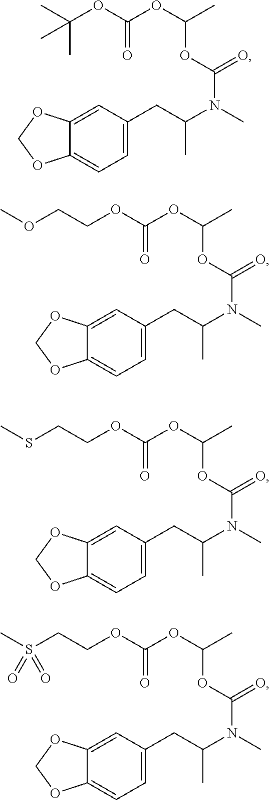

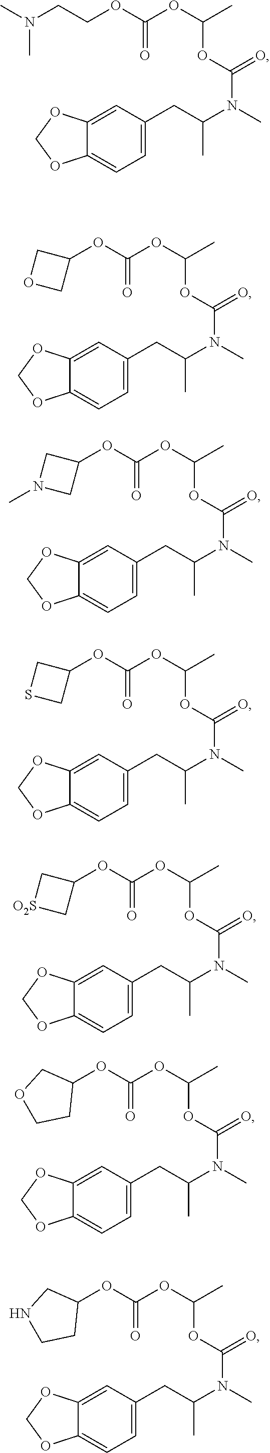

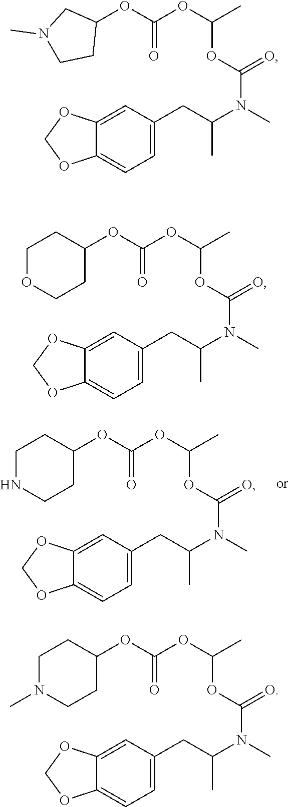

In some embodiments is a compound of Formula (IX) or a pharmaceutically acceptable salt thereof, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, —CH₂CH₂OMe, vinyl, phenyl, 2-pyridyl, 3-pyridyl, 4-pyridyl, 2-pyrimidyl, 4-pyrimidyl, 5-pyrimidyl, or 6-pyrimidyl.

In some embodiments is a compound of Formula (IX), or a pharmaceutically acceptable salt thereof, wherein R² is hydrogen. In some embodiments is a compound of Formula (IX), or a pharmaceutically acceptable salt thereof, wherein R² is methyl, ethyl, n-propyl, isopropyl, or —CH(Et)₂.

In some embodiments is a compound of Formula (IX) having the structure of Formula (IXa), or a pharmaceutically acceptable salt thereof:

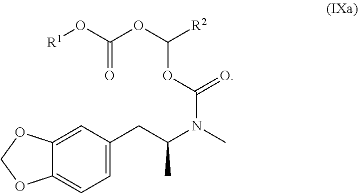

In some embodiments is a compound of Formula (IX) having the structure of Formula (IXb), or a pharmaceutically acceptable salt thereof:

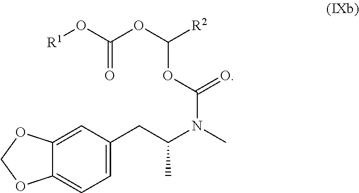

In certain embodiments, the compound of Formula (I′) is a compound in Table 1.

In certain embodiments, the compound of Formula (I′) is a compound in Tables 2-7.

In certain embodiments, the compound of Formula (I′) is selected from the group consisting of:

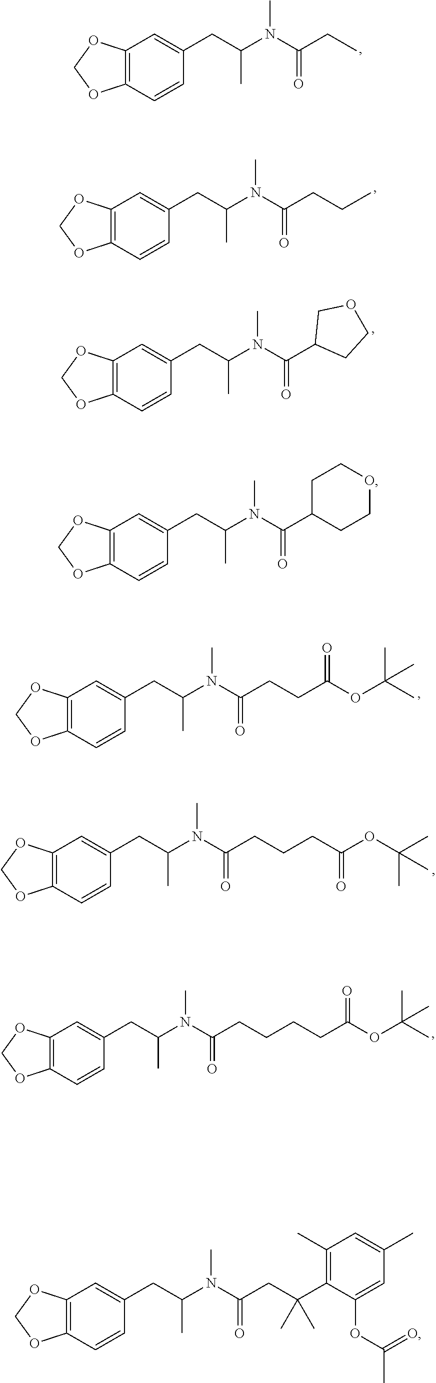

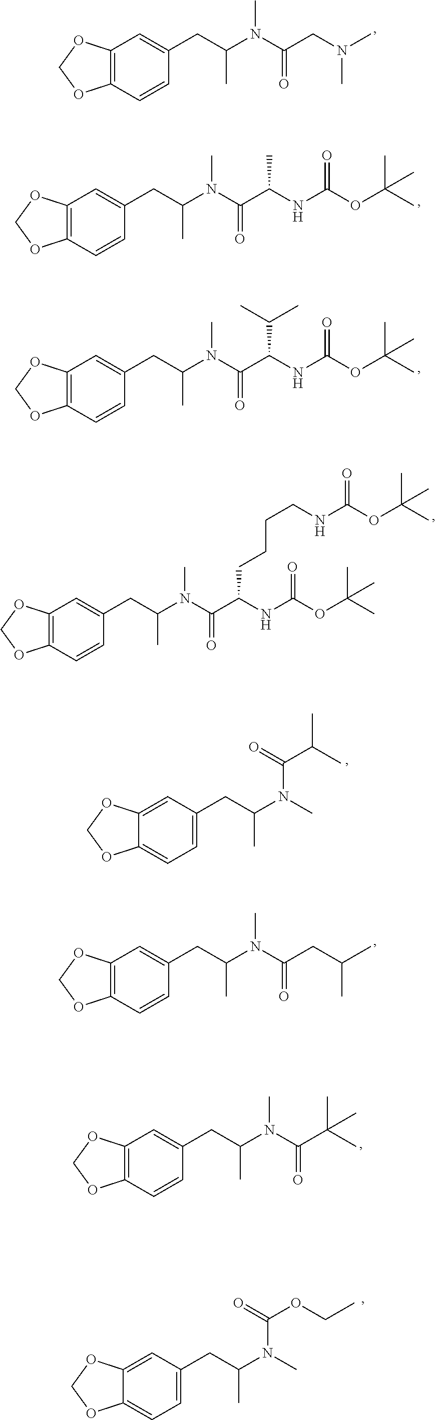

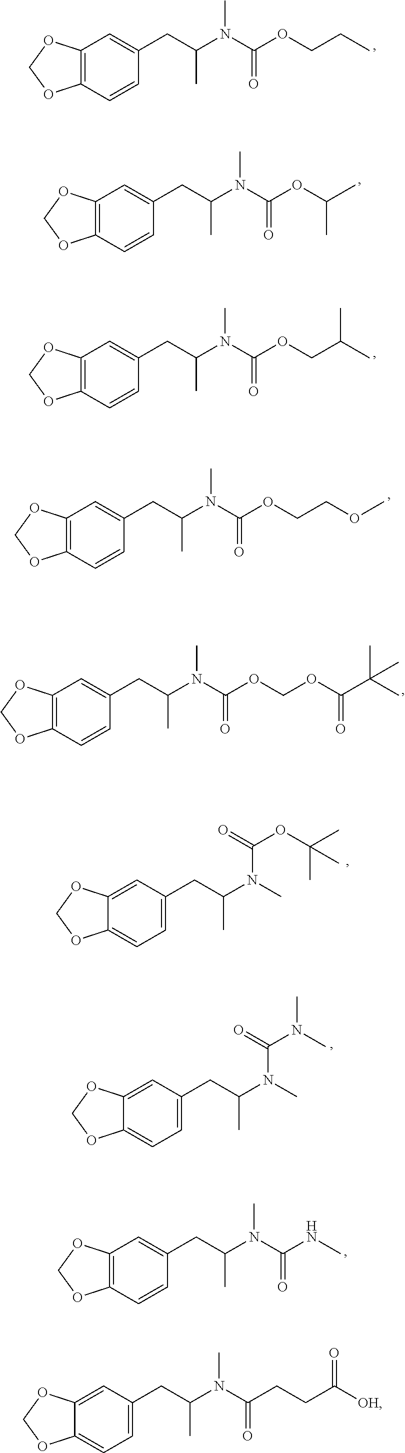

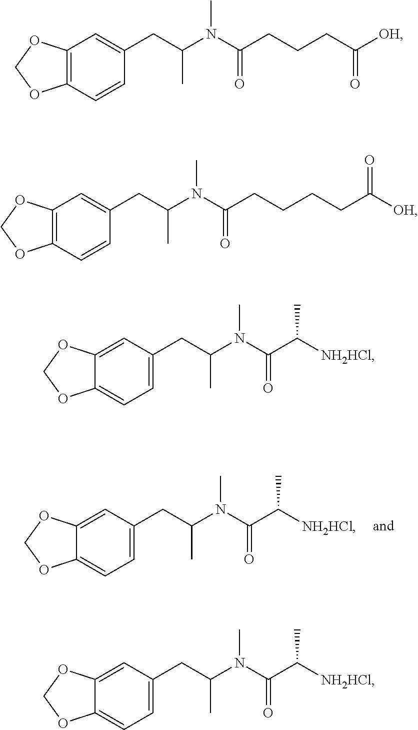

or a stereoisomer, a hydrate, or a pharmaceutically acceptable salt thereof.

Compounds provided herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclylalkyl groups, heteroaryl groups, cycloalkyl groups, acyl groups, acyloxy groups, carbamate groups, amide groups, ureido groups, epoxy groups, and ester groups.

Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Alkyl groups can include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cycloalkyl groups include cyclopropyl, cyclobutyl, cyclopentyl, cyclohexyl, cycloheptlyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl groups include straight, branched, and cyclic alkynyl groups. The triple bond of an alkylnyl group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —OR^{a} where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocyclylalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like.

In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocyclylalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocyclylalkyl radical may be a monocyclic, bicyclic, tricyclic, or tetracyclic ring system, which may include fused (when fused with an aryl or a heteroaryl ring, the heterocyclylalkyl is bonded through a non-aromatic ring atom) or bridged ring systems; and the nitrogen, carbon, or sulfur atoms in the heterocyclylalkyl radical may be optionally oxidized; the nitrogen atom may be optionally quaternized.

Representative heterocyclylalkyls include, but are not limited to, heterocyclylalkyls having from two to fifteen carbon atoms (C₂-C₁₅ heterocyclylalkyl), from two to ten carbon atoms (C₂-C₁₀ heterocyclylalkyl), from two to eight carbon atoms (C₂-C₈ heterocyclylalkyl), from two to six carbon atoms (C₂-C₆ heterocyclylalkyl), from two to five carbon atoms (C₂-C₅ heterocyclylalkyl), or two to four carbon atoms (C₂-C₄ heterocyclylalkyl). In some embodiments, the heterocyclylalkyl is a 3- to 6-membered heterocyclylalkyl. In some embodiments, the cycloalkyl is a 5- to 6-membered heterocyclylalkyl. Examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). Unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocyclylalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

In some embodiments, the present disclosure provides a deuterated analogue of any compound disclosed herein. A deuterated analogue can include a compound herein where one or more ¹H atoms is replaced with a deuterium atom. A deuterated analogue of Compound 1:

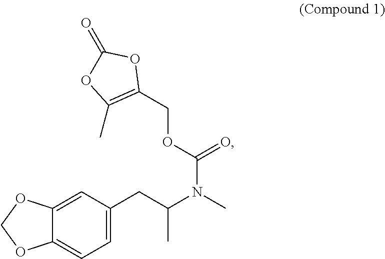 [SMILES: Cc1oc(=O)oc1COC(=O)N(C)C(C)Cc1ccc2c(c1)OCO2]

can be, for example

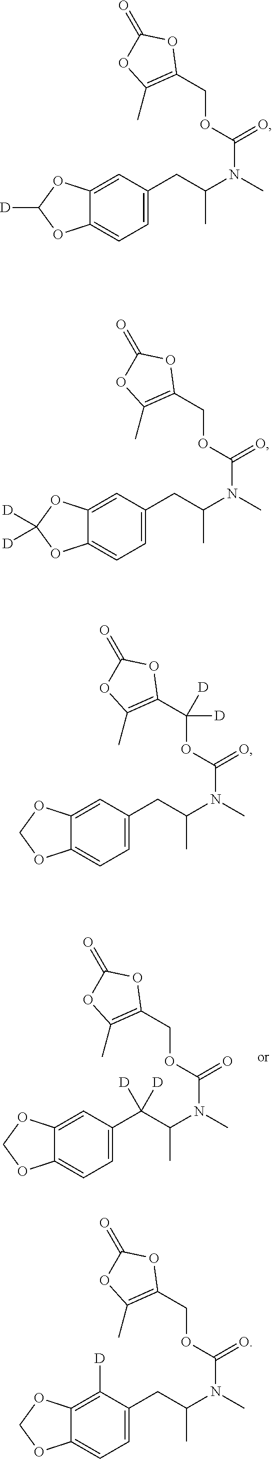 [SMILES: [2H]C1Oc2ccc(CC(C)N(C)C(=O)OCc3oc(=O)oc3C)cc2O1 ; [2H]C1([2H])Oc2ccc(CC(C)N(C)C(=O)OCc3oc(=O)oc3C)cc2O1 ; [2H]C([2H])(OC(=O)N(C)C(C)Cc1ccc2c(c1)OCO2)c1oc(=O)oc1C ; [2H]C([2H])(c1ccc2c(c1)OCO2)C(C)N(C)C(=O)OCc1oc(=O)oc1C ; [2H]c1c(CC(C)N(C)C(=O)OCc2oc(=O)oc2C)ccc2c1OCO2]

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Pharmaceutical Compositions.

According to another embodiment, the present disclosure provides a composition comprising a compound of the present disclosure and a pharmaceutically acceptable carrier, adjuvant, or vehicle. The amount of compound in the composition is an amount effective to treat the relevant disease, disorder, or condition in a patient in need thereof (an “effective amount”). In some embodiments, a composition of the present disclosure is formulated for oral administration to a patient.

The term “pharmaceutically acceptable carrier, adjuvant, or vehicle” refers to a nontoxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, enterally, intracistemally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the composition is administered orally, intraperitoneally, or intravenously. In some embodiments, the composition is a transmucosal formulation. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

To aid in delivery of the composition, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, the pharmaceutically acceptable composition is formulated for oral administration. Such formulations may be administered with or without food. In some embodiments, the pharmaceutically acceptable composition is administered without food. In other embodiments, the pharmaceutically acceptable composition is administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Therapeutic agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Selected compounds of the disclosure with corresponding simplified molecular-input line-entry system (SMILES) strings are provided in Table 1.

TABLE 1

| | Structure |
|---|---|
| Cpd | SMILES |
| 1 | 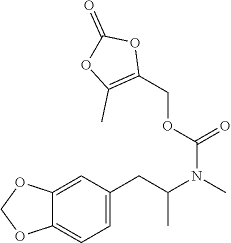 [SMILES: Cc1oc(=O)oc1COC(=O)N(C)C(C)Cc1ccc2c(c1)OCO2] |
| | CC(CC1═CC═C2OCOC2═C1)N |
| | (C(OCC(O3)═C(C)OC3═O)═O)C |
| 2 | 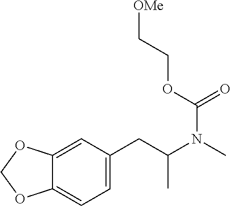 [SMILES: COCCOC(=O)N(C)C(C)Cc1ccc2c(c1)OCO2] |
| | CC(CC1═CC═C2OCOC2═C1)N |
| | (C(OCCOC)═O)C |
| 3 | 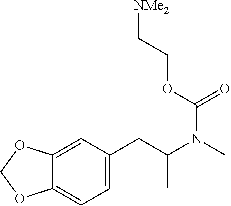 [SMILES: CC(Cc1ccc2c(c1)OCO2)N(C)C(=O)OCCN(C)C] |
| | CC(CC1═CC═C2OCOC2═C1)N |
| | (C(OCCN(C)C)═O)C |
| 4 | 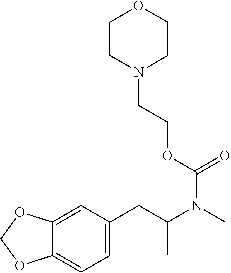 [SMILES: CC(Cc1ccc2c(c1)OCO2)N(C)C(=O)OCCN1CCOCC1] |
| | CC(CC1═CC═C2OCOC2═C1)N |
| | (C(OCCN3CCOCC3)═O)C |
| 5 | 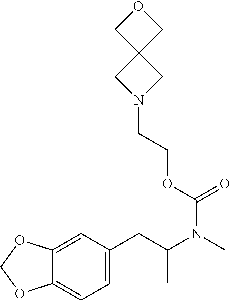 [SMILES: CC(Cc1ccc2c(c1)OCO2)N(C)C(=O)OCCN1CC2(COC2)C1] |
| | CC(CC1═CC═C2OCOC2═C1)N |
| | (C(OCCN3CC4(COC4)C3)═O)C |
| 6 | 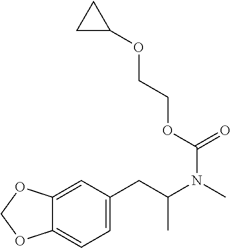 [SMILES: CC(Cc1ccc2c(c1)OCO2)N(C)C(=O)OCCOC1CC1] |
| | CC(CC1═CC═C2OCOC2═C1) |
| | N(C(OCCOC3CC3)═O)C |
| 7 | 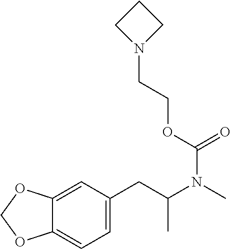 [SMILES: CC(Cc1ccc2c(c1)OCO2)N(C)C(=O)OCCN1CCC1] |
| | CC(CC1═CC═C2OCOC2═C1) |
| | N(C(OCCN3CCC3)═O)C |
| 8 | 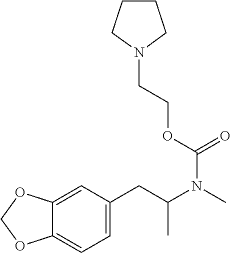 [SMILES: CC(Cc1ccc2c(c1)OCO2)N(C)C(=O)OCCN1CCCC1] |
| | CC(CC1═CC═C2OCOC2═C1) |
| | N(C(OCCN3CCCC3)═O)C |
| 9 | 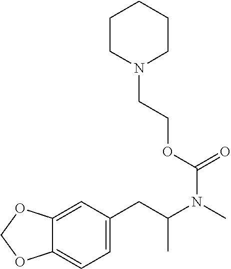 [SMILES: CC(Cc1ccc2c(c1)OCO2)N(C)C(=O)OCCN1CCCCC1] |
| | CC(CC1═CC═C2OCOC2═C1) |
| | N(C(OCCN3CCCCC3)═O)C |
| 10 | 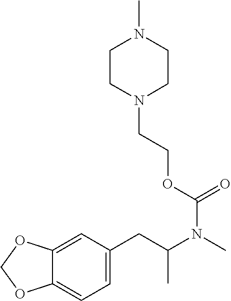 [SMILES: CC(Cc1ccc2c(c1)OCO2)N(C)C(=O)OCCN1CCN(C)CC1] |
| | CC(CC1═CC═C2OCOC2═C1) |
| | N(C(OCCN3CCN(C)CC3)═O)C |
| 11 | 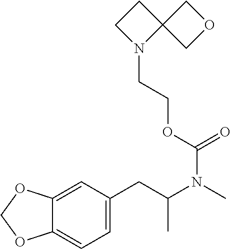 [SMILES: CC(Cc1ccc2c(c1)OCO2)N(C)C(=O)OCCN1CCC12COC2] |
| | CC(CC1═CC═C2OCOC2═C1) |
| | N(C(OCCN3C4(COC4)CC3)═O)C |
| 12 | 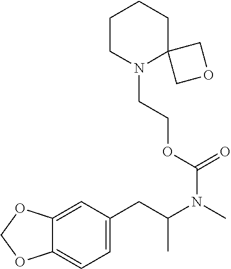 [SMILES: CC(Cc1ccc2c(c1)OCO2)N(C)C(=O)OCCN1CCCCC12COC2] |
| | CC(CC1═CC═C2OCOC2═C1) |
| | N(C(OCCN3C4(COC4)CCCC3)═O) |
| 13 | 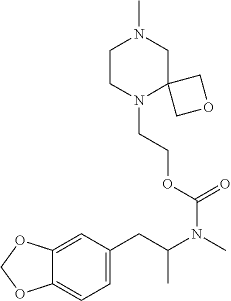 [SMILES: CC(Cc1ccc2c(c1)OCO2)N(C)C(=O)OCCN1CCN(C)CC12COC2] |
| | CC(CC1═CC═C2OCOC2═C1) |
| | N(C(OCCN3C4(COC4)CN(C)CC3) |
| 14 | 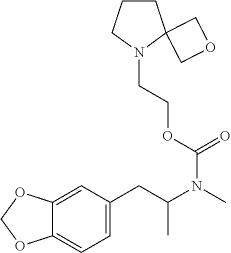 [SMILES: CC(Cc1ccc2c(c1)OCO2)N(C)C(=O)OCCN1CCCC12COC2] |
| | CC(CC1═CC═C2OCOC2═C1) |
| | N(C(OCCN3C4(COC4)CCC3)═O)C |
| 15 | 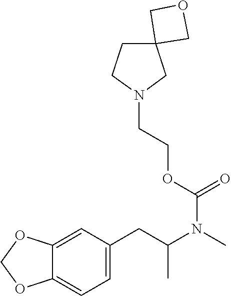 [SMILES: CC(Cc1ccc2c(c1)OCO2)N(C)C(=O)OCCN1CCC2(COC2)C1] |
| | CC(CC1═CC═C2OCOC2═C1) |
| | N(C(OCCN3CC4(COC4)CC3)═O)C |
| 16 | 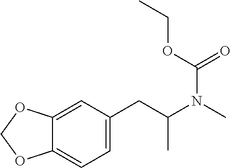 [SMILES: CCOC(=O)N(C)C(C)Cc1ccc2c(c1)OCO2] |
| | CC(CC1═CC═C2OCOC2═C1) |
| | N(C(OCC)═O)C |
| 17 | 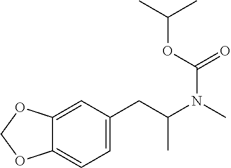 [SMILES: CC(C)OC(=O)N(C)C(C)Cc1ccc2c(c1)OCO2] |
| | CC(CC1═CC═C2OCOC2═C1) |
| | N(C(OC(C)C)═O)C |
| 18 | 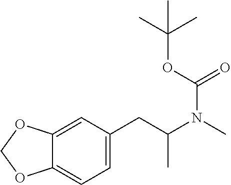 [SMILES: CC(Cc1ccc2c(c1)OCO2)N(C)C(=O)OC(C)(C)C] |
| | CC(CC1═CC═C2OCOC2═C1) |
| | N(C(OC(C)(C)C)═O)C |
| 19 | 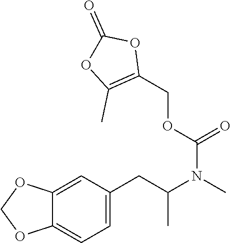 [SMILES: Cc1oc(=O)oc1COC(=O)N(C)C(C)Cc1ccc2c(c1)OCO2] |
| | CC(CC1═CC═C2OCOC2═C1) |
| | N(C(OCC(O3)═C(C)OC3═O)═O)C |
| 20 | 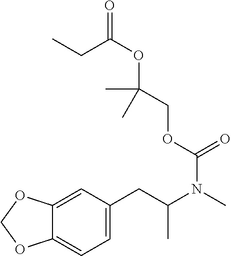 [SMILES: CCC(=O)OC(C)(C)COC(=O)N(C)C(C)Cc1ccc2c(c1)OCO2] |
| | CC(CC1═CC═C2OCOC2═C1) |
| | N(C(OCC(C)(C)OC(CC)═O)═O)C |
| 21 | 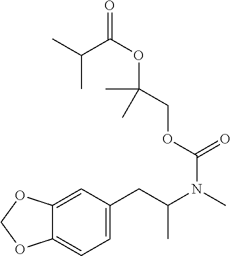 [SMILES: CC(C)C(=O)OC(C)(C)COC(=O)N(C)C(C)Cc1ccc2c(c1)OCO2] |
| | CC(CC1═CC═C2OCOC2═C1) |
| | N(C(OCC(C)(C)OC(C(C)C)═O)═O) |
| 22 | 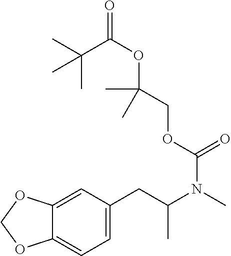 [SMILES: CC(Cc1ccc2c(c1)OCO2)N(C)C(=O)OCC(C)(C)OC(=O)C(C)(C)C] |
| | CC(CC1═CC═C2OCOC2═C1) |
| | N(C(OCC(C)(C)OC(C(C)(C)C)═ |
| | O)═O)C |
| 23 | 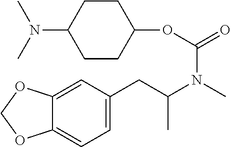 [SMILES: CC(Cc1ccc2c(c1)OCO2)N(C)C(=O)OC1CCC(N(C)C)CC1] |
| | CN(C(OC1CCC(N(C)C)CC1)═O) |
| | C(C)CC2═CC═C3OCOC3═C2 |
| 24 | 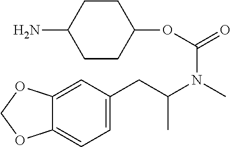 [SMILES: CC(Cc1ccc2c(c1)OCO2)N(C)C(=O)OC1CCC(N)CC1] |
| | CN(C(OC1CCC(N)CC1)═O)C(C) |
| | CC2═CC═C3OCOC3═C2 |
| 25 | 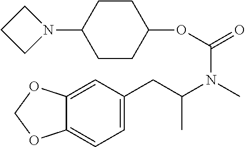 [SMILES: CC(Cc1ccc2c(c1)OCO2)N(C)C(=O)OC1CCC(N2CCC2)CC1] |
| | CN(C(OC1CCC(N2CCC2)CC1)═ |
| | O)C(C)CC3═CC═C4OCOC4═C3 |
| 26 | 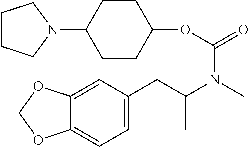 [SMILES: CC(Cc1ccc2c(c1)OCO2)N(C)C(=O)OC1CCC(N2CCCC2)CC1] |
| | CN(C(OC1CCC(N2CCCC2) |
| | CC1)═O)C(C)CC3═CC═C4OCOC4═C3 |
| 27 | 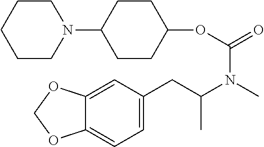 [SMILES: CC(Cc1ccc2c(c1)OCO2)N(C)C(=O)OC1CCC(N2CCCCC2)CC1] |
| | CN(C(OC1CCC(N2CCCCC2) |
| | CC1)═O)C(C)CC3═CC═C4OCOC4═C3 |
| 28 | 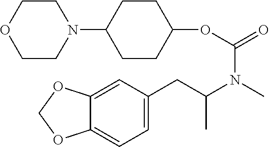 [SMILES: CC(Cc1ccc2c(c1)OCO2)N(C)C(=O)OC1CCC(N2CCOCC2)CC1] |
| | CN(C(OC1CCC(N2CCOCC2)CC |
| | 1)═O)C(C)CC3═CC═C4OCOC4═C3 |
| 29 | 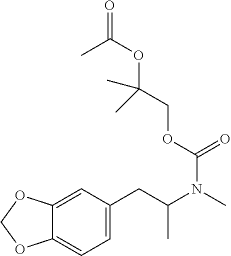 [SMILES: CC(=O)OC(C)(C)COC(=O)N(C)C(C)Cc1ccc2c(c1)OCO2] |
| | CN(C(OCC(C)(C)OC(C)═O)═O) |
| | C(C)CC1═CC═C2OCOC2═C1 |
| 30 | 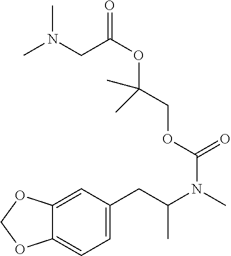 [SMILES: CC(Cc1ccc2c(c1)OCO2)N(C)C(=O)OCC(C)(C)OC(=O)CN(C)C] |
| | CN(C(OCC(C)(C)OC(CN(C)C)═ |
| | O)═O)C(C)CC1═CC═ |
| | C2OCOC2═C1 |
| 31 | 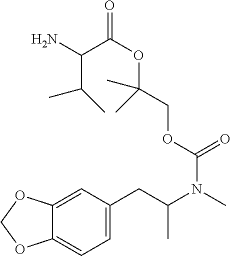 [SMILES: CC(C)C(N)C(=O)OC(C)(C)COC(=O)N(C)C(C)Cc1ccc2c(c1)OCO2] |
| | CN(C(OCC(C)(C)OC(C(C(C)C) |
| | N)═O)═O)C(C)CC1═CC═C2OCO |
| | C2═C1 |
| 32 | 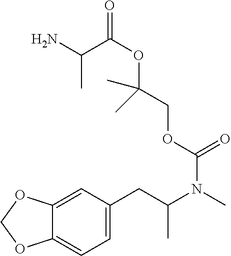 [SMILES: CC(N)C(=O)OC(C)(C)COC(=O)N(C)C(C)Cc1ccc2c(c1)OCO2] |
| | CN(C(OCC(C)(C)OC(C(C)N)═ |
| | O)═O)C(C)CC1═CC═C2OCOC2═C |
| | 1 |
| 33 | 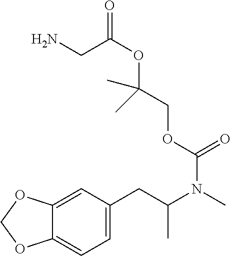 [SMILES: CC(Cc1ccc2c(c1)OCO2)N(C)C(=O)OCC(C)(C)OC(=O)CN] |
| | CN(C(OCC(C)(C)OC(CN)═O)═ |
| | O)C(C)CC1═CC═C2OCOC2═C1 |
| 34 | 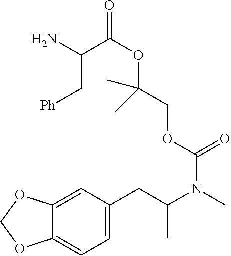 [SMILES: CC(Cc1ccc2c(c1)OCO2)N(C)C(=O)OCC(C)(C)OC(=O)C(N)Cc1ccccc1] |
| | CN(C(OCC(C)(C)OC(C(CC1═C |
| | C═CC═C1)N)═O)═O)C(C)CC2═ |
| | CC═C3OCOC3═C2 |
| 35 | 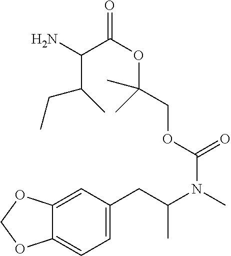 [SMILES: CCC(C)C(N)C(=O)OC(C)(C)COC(=O)N(C)C(C)Cc1ccc2c(c1)OCO2] |
| | CN(C(OCC(C)(C)OC(C(C(C)CC) |
| | N)═O)═O)C(C)CC1═CC═C2OCO |
| | C2═C1 |
| 36 | 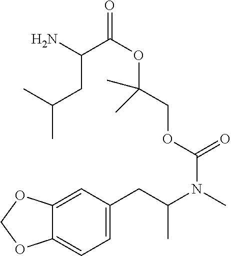 [SMILES: CC(C)CC(N)C(=O)OC(C)(C)COC(=O)N(C)C(C)Cc1ccc2c(c1)OCO2] |
| | CN(C(OCC(C)(C)OC(C(CC(C)C) |
| | N)═O)═O)C(C)CC1═CC═C2OCO |
| | C2═C1 |
| 37 | 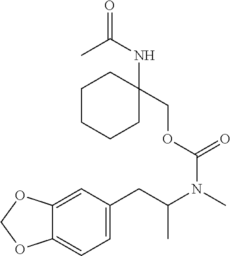 [SMILES: CC(=O)NC1(COC(=O)N(C)C(C)Cc2ccc3c(c2)OCO3)CCCCC1] |
| | CN(C(OCC1(CCCCC1)OC(C)═O)═ |
| | O)C(C)CC2═CC═C3OCOC3═C2 |
| 38 | 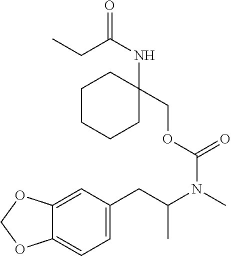 [SMILES: CCC(=O)NC1(COC(=O)N(C)C(C)Cc2ccc3c(c2)OCO3)CCCCC1] |
| | CN(C(OCC[19](CCCCC@19)OC |
| | (CC)═O)═O)C(C)CC[4]═CC═C[7] |
| | OCOC(@8)═C@5 |
| 39 | 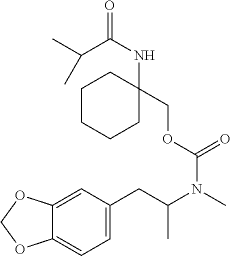 [SMILES: CC(C)C(=O)NC1(COC(=O)N(C)C(C)Cc2ccc3c(c2)OCO3)CCCCC1] |
| | CN(C(OCC1(CCCCC1)OC(C(C) |
| | C)═O)═O)C(C)CC2═CC═C3OCO |
| | C3═C2 |
| 40 | 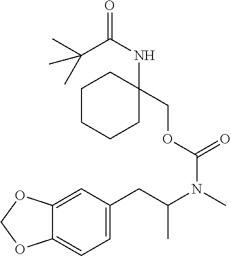 [SMILES: CC(Cc1ccc2c(c1)OCO2)N(C)C(=O)OCC1(NC(=O)C(C)(C)C)CCCCC1] |
| | CN(C(OCC1(CCCCC1)OC(C(C) |
| | (C)C)═O)═O)C(C)CC2═CC═C3O |
| | COC3═C2 |
| 41 | 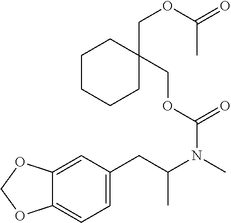 [SMILES: CC(=O)OCC1(COC(=O)N(C)C(C)Cc2ccc3c(c2)OCO3)CCCCC1] |
| | CN(C(OCC1(CCCCC1)COC(C)═ |
| | O)═O)C(C)CC2═CC═ |
| | C3OCOC3═C2 |
| 42 | 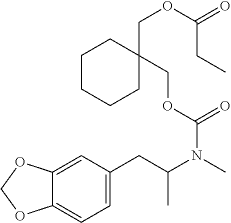 [SMILES: CCC(=O)OCC1(COC(=O)N(C)C(C)Cc2ccc3c(c2)OCO3)CCCCC1] |
| | CN(C(OCC1(CCCCC1)COC |
| | (CC)═O)═O)C(C)CC2═CC═ |
| | C3OCOC3═C2 |
| 43 | 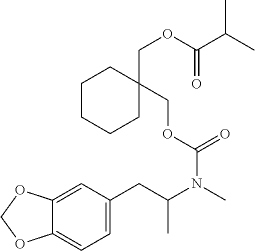 [SMILES: CC(C)C(=O)OCC1(COC(=O)N(C)C(C)Cc2ccc3c(c2)OCO3)CCCCC1] |
| | CN(C(OCCI(CCCCC1)COC(C |
| | (C)C)═O)═O)C(C)CC2═CC═C3OC |
| | OC3═C2 |
| 44 | 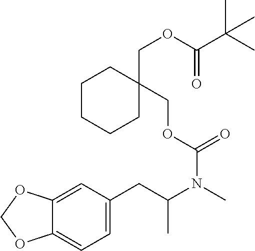 [SMILES: CC(Cc1ccc2c(c1)OCO2)N(C)C(=O)OCC1(COC(=O)C(C)(C)C)CCCCC1] |
| | CN(C(OCC1(CCCCC1)COC(C |
| | (C)(C)C)═O)═O)C(C)CC2═CC═C3 |
| | OCOC3═C2 |
| 45 | 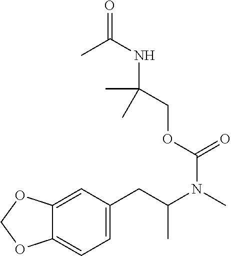 [SMILES: CC(=O)NC(C)(C)COC(=O)N(C)C(C)Cc1ccc2c(c1)OCO2] |
| | CN(C(OCC(C)(C)NC(C)═O)═O) |
| | C(C)CC1═CC═C2OCOC2═C1 |
| 46 | 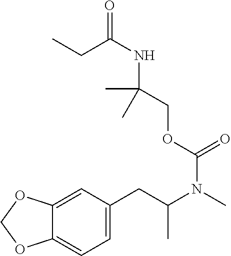 [SMILES: CCC(=O)NC(C)(C)COC(=O)N(C)C(C)Cc1ccc2c(c1)OCO2] |
| | CN(C(OCC(C)(C)NC(CC)═O)═ |
| | O)C(C)CC1═CC═C2OCOC2═C1 |
| 47 | 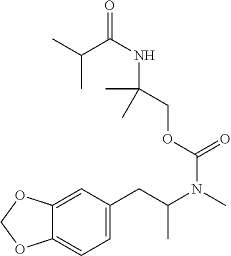 [SMILES: CC(C)C(=O)NC(C)(C)COC(=O)N(C)C(C)Cc1ccc2c(c1)OCO2] |
| | CN(C(OCC(C)(C)NC(C(C)(C) |
| | C)═O)═O)C(C)CC1═CC═C2OCOC |
| | 2═C1 |
| 48 | 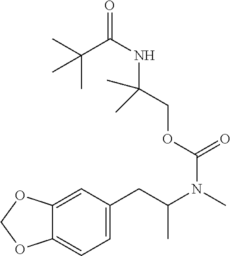 [SMILES: CC(Cc1ccc2c(c1)OCO2)N(C)C(=O)OCC(C)(C)NC(=O)C(C)(C)C] |
| | CN(C(OCC(C)(C)NC(C(C)(C) |
| | C)═O)═O)C(C)CC1═CC═C2OCOC |
| | 2═C1 |
| 49 | 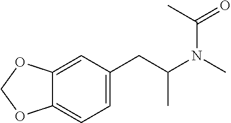 [SMILES: CC(=O)N(C)C(C)Cc1ccc2c(c1)OCO2] |
| | CC(CC1═CC═C2OCOC2═C1) |
| | N(C(C)═O)C |
| 50 | 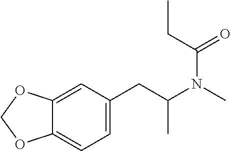 [SMILES: CCC(=O)N(C)C(C)Cc1ccc2c(c1)OCO2] |
| | CC(CC1═CC═C2OCOC2═C1) |
| | N(C(CC)═O)C |
| 51 | 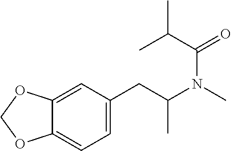 [SMILES: CC(C)C(=O)N(C)C(C)Cc1ccc2c(c1)OCO2] |
| | CC(CC1═CC═C2OCOC2═C1) |
| | N(C(C(C)C)═O)C |
| 52 | 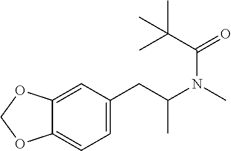 [SMILES: CC(Cc1ccc2c(c1)OCO2)N(C)C(=O)C(C)(C)C] |
| | CC(CC1═CC═C2OCOC2═C1) |
| | N(C(C(C)(C)C)═O)C |
| 53 | 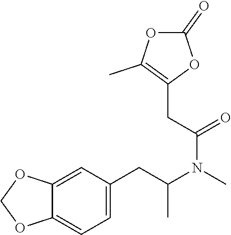 [SMILES: Cc1oc(=O)oc1CC(=O)N(C)C(C)Cc1ccc2c(c1)OCO2] |
| | CC(CC1═CC═C2OCOC2═C1) |
| | N(C(CC(O3)═C(C)OC3═O)═O)C |
| 54 | 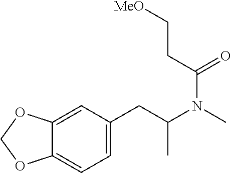 [SMILES: COCCC(=O)N(C)C(C)Cc1ccc2c(c1)OCO2] |
| | CC(CC1═CC═C2OCOC2═C1) |
| | N(C(CCOC)═O)C |
| 55 | 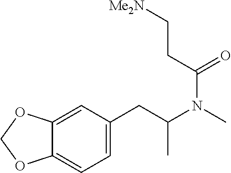 [SMILES: CC(Cc1ccc2c(c1)OCO2)N(C)C(=O)CCN(C)C] |
| | CC(CC1═CC═C2OCOC2═C1) |
| | N(C(CCN(C)C)═O)C |
| 56 | 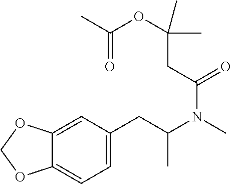 [SMILES: CC(=O)OC(C)(C)CC(=O)N(C)C(C)Cc1ccc2c(c1)OCO2] |
| | CC(CC1═CC═C2OCOC2═C1) |
| | N(C(CC(C)(C)OC(C)═O)═O)C |
| 57 | 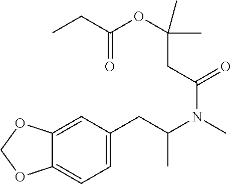 [SMILES: CCC(=O)OC(C)(C)CC(=O)N(C)C(C)Cc1ccc2c(c1)OCO2] |
| | CC(CC1═CC═C2OCOC2═C1) |
| | N(C(CC(C)(C)OC(CC)═O)═O)C |
| 58 | 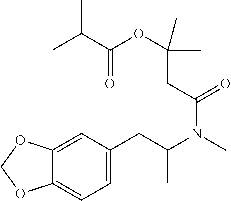 [SMILES: CC(C)C(=O)OC(C)(C)CC(=O)N(C)C(C)Cc1ccc2c(c1)OCO2] |
| | CC(CC1═CC═C2OCOC2═C1) |
| | N(C(CC(C)(C)OC(C(C)C)═O)═O)C |
| 59 | 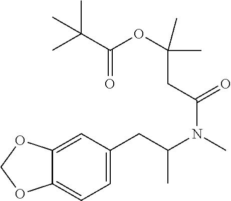 [SMILES: CC(Cc1ccc2c(c1)OCO2)N(C)C(=O)CC(C)(C)OC(=O)C(C)(C)C] |
| | CC(CC1═CC═C2OCOC2═C1) |
| | N(C(CC(C)(C)OC(C(C)(C)C)═O)═ |
| | O)C |
| 60 | 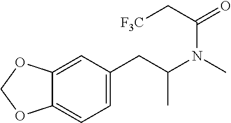 [SMILES: CC(Cc1ccc2c(c1)OCO2)N(C)C(=O)CC(F)(F)F] |
| | CN(C(CC(F)(F)F)═O)C(C)CC1═ |
| | CC═C2OCOC2═C1 |
| 61 | 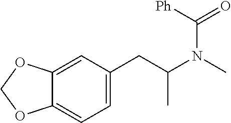 [SMILES: CC(Cc1ccc2c(c1)OCO2)N(C)C(=O)c1ccccc1] |
| | CN(C(C1═CC═CC═C1)═O)C(C) |
| | CC2═CC═C3OCOC3═C2 |
| 62 | 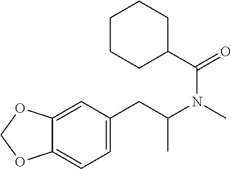 [SMILES: CC(Cc1ccc2c(c1)OCO2)N(C)C(=O)C1CCCCC1] |
| | CN(C(C1CCCCC1)═O)C(C) |
| | CC2═CC═C3OCOC3═C2 |
| 63 | 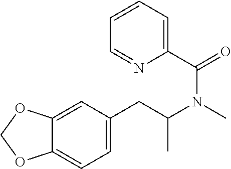 [SMILES: CC(Cc1ccc2c(c1)OCO2)N(C)C(=O)c1ccccn1] |
| | CN(C(C1═CC═CC═N1)═O)C(C) |
| | CC2═CC═C3OCOC3═C2 |
| 64 | 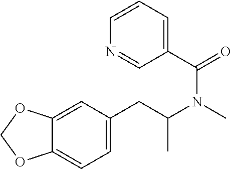 [SMILES: CC(Cc1ccc2c(c1)OCO2)N(C)C(=O)c1cccnc1] |
| | CN(C(C1═CC═CN═C1)═O)C(C) |
| | CC2═CC═C3OCOC3═C2 |
| 65 | 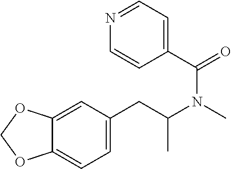 [SMILES: CC(Cc1ccc2c(c1)OCO2)N(C)C(=O)c1ccncc1] |
| | CN(C(C1═CC═NC═C1)═O)C(C) |
| | CC2═CC═C3OCOC3═C2 |
| 66 | 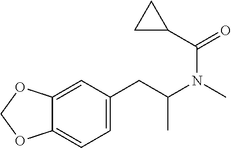 [SMILES: CC(Cc1ccc2c(c1)OCO2)N(C)C(=O)C1CC1] |
| | CN(C(C1CC1)═O)C(C)CC2═ |
| | CC═C3OCOC3═C2 |
| 67 | 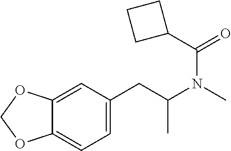 [SMILES: CC(Cc1ccc2c(c1)OCO2)N(C)C(=O)C1CCC1] |
| | CN(C(C1CCC1)═O)C(C)CC2═C |
| | C═C3OCOC3═C2 |
| 68 | 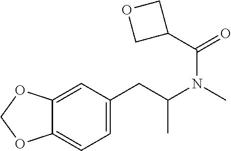 [SMILES: CC(Cc1ccc2c(c1)OCO2)N(C)C(=O)C1COC1] |
| | CN(C(C1COC1)═O)C(C)CC2═C |
| | C═C3OCOC3═C2 |
| 69 | 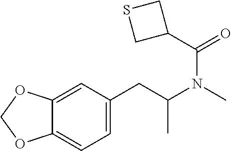 [SMILES: CC(Cc1ccc2c(c1)OCO2)N(C)C(=O)C1CSC1] |
| | CN(C(C1CSC1)═O)C(C)CC2═C |
| | C═C3OCOC3═C2 |
| 70 | 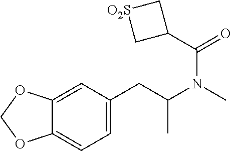 [SMILES: CC(Cc1ccc2c(c1)OCO2)N(C)C(=O)C1CS(=O)(=O)C1] |
| | CN(C(C1CS(C1)(═O)═O)═O)C |
| | (C)CC2═CC═C3OCOC3═C2 |
| 71 | 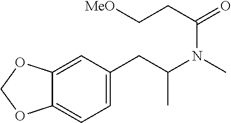 [SMILES: COCCC(=O)N(C)C(C)Cc1ccc2c(c1)OCO2] |
| | CN(C(CCOC)═O)C(C)CC1═CC═ |
| | C2OCOC2═C1 |
| 72 | 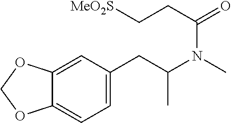 [SMILES: CC(Cc1ccc2c(c1)OCO2)N(C)C(=O)CCS(C)(=O)=O] |
| | CN(C(CCS(═O)(C)═O)═O)C(C)C |
| | C1═CC═C2OCOC2═C1 |
| 73 | 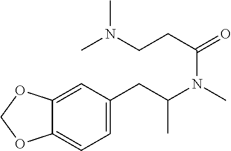 [SMILES: CC(Cc1ccc2c(c1)OCO2)N(C)C(=O)CCN(C)C] |
| | CN(C(CCN(C)C)═O)C(C)CC1═C |
| | C═C2OCOC2═C1 |
| 74 | 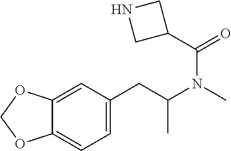 [SMILES: CC(Cc1ccc2c(c1)OCO2)N(C)C(=O)C1CNC1] |
| | CN(C(C1CNC1)═O)C(C)CC2═C |
| | C═C3OCOC3═C2 |
| 75 | 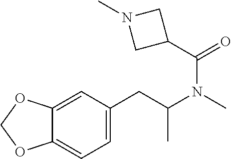 [SMILES: CC(Cc1ccc2c(c1)OCO2)N(C)C(=O)C1CN(C)C1] |
| | CN(C(C1CN(C)C1)═O)C(C) |
| | CC2═CC═C3OCOC3═C2 |
| 76 | 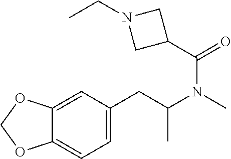 [SMILES: CCN1CC(C(=O)N(C)C(C)Cc2ccc3c(c2)OCO3)C1] |
| | CN(C(C1CN(CC)C1)═O)C(C)CC |
| | 2═CC═C3OCOC3═C2 |
| 77 | 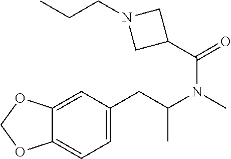 [SMILES: CCCN1CC(C(=O)N(C)C(C)Cc2ccc3c(c2)OCO3)C1] |
| | CN(C(C1CN(CCC)C1)═O)C(C)C |
| | C2═CC═C3OCOC3═C2 |
| 78 | 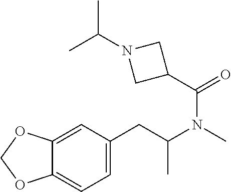 [SMILES: CC(C)N1CC(C(=O)N(C)C(C)Cc2ccc3c(c2)OCO3)C1] |
| | CN(C(C1CN(C(C)C)C1)═O)C(C) |
| | CC2═CC═C3OCOC3═C2 |
| 79 | 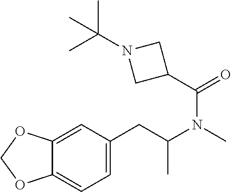 [SMILES: CC(Cc1ccc2c(c1)OCO2)N(C)C(=O)C1CN(C(C)(C)C)C1] |
| | CN(C(C1CN(C(C)(C)C)C1)═O)C |
| | (C)CC2═CC═C3OCOC3═C2 |
| 80 | 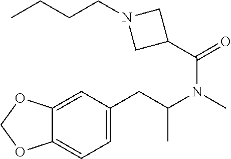 [SMILES: CCCCN1CC(C(=O)N(C)C(C)Cc2ccc3c(c2)OCO3)C1] |
| | CN(C(C1CN(CCCC)C1)═O)C(C) |
| | CC2═CC═C3OCOC3═C2 |
| 81 | 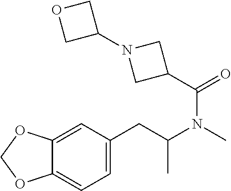 [SMILES: CC(Cc1ccc2c(c1)OCO2)N(C)C(=O)C1CN(C2COC2)C1] |
| | CN(C(C1CN(C2COC2)C1)═O) |
| | C(C)CC3═CC═C4OCOC4═C3 |
| 82 | 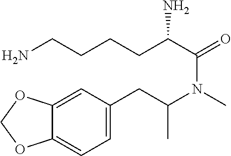 [SMILES: CC(Cc1ccc2c(c1)OCO2)N(C)C(=O)[C@@H](N)CCCCN] |
| | CN(C([C@@H](N)CCCCN)═O) |
| | C(C)CC1═CC═C2OCOC2═C1 |
| 83 | 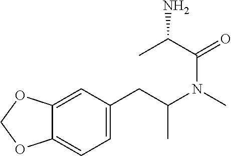 [SMILES: CC(Cc1ccc2c(c1)OCO2)N(C)C(=O)[C@H](C)N] |
| | CN(C([C@@H](N)C)═O)C(C)C |
| | C1═CC═C2OCOC2═C1 |
| 84 | 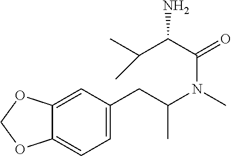 [SMILES: CC(C)[C@H](N)C(=O)N(C)C(C)Cc1ccc2c(c1)OCO2] |
| | CN(C([C@@H](N)C(C)C)═O) |
| | C(C)CC1═CC═C2OCOC2═C1 |
| 85 | 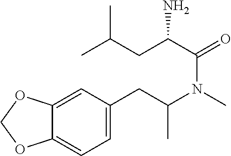 [SMILES: CC(C)C[C@H](N)C(=O)N(C)C(C)Cc1ccc2c(c1)OCO2] |
| | CN(C([C@@H](N)CC(C)C)═O) |
| | C(C)CC1═CC═C2OCOC2═C1 |
| 86 | 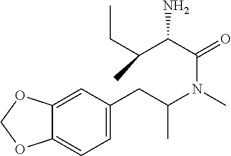 [SMILES: CC[C@H](C)[C@H](N)C(=O)N(C)C(C)Cc1ccc2c(c1)OCO2] |
| | CN(C([C@@H](N)[C@@H](C) |
| | CC)═O)C(C)CC1═CC═C2OCOC |
| | 2═C1 |
| 87 | 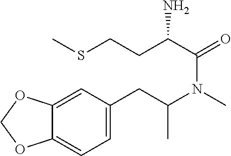 [SMILES: CSCC[C@H](N)C(=O)N(C)C(C)Cc1ccc2c(c1)OCO2] |
| | CN(C([C@@H](N)CCSC)═O) |
| | C(C)CC1═CC═C2OCOC2═C1 |
| 88 | 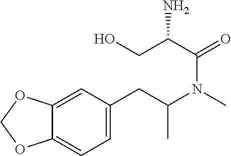 [SMILES: CC(Cc1ccc2c(c1)OCO2)N(C)C(=O)[C@@H](N)CO] |
| | CN(C([C@@H](N)CO)═O)C(C) |
| | CC1═CC═C2OCOC2═C1 |
| 89 | 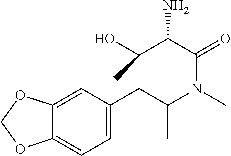 [SMILES: CC(Cc1ccc2c(c1)OCO2)N(C)C(=O)[C@@H](N)[C@@H](C)O] |
| | CN(C([C@@H](N)[C@@H](C) |
| | O)═O)C(C)CC1═CC═ |
| | C2OCOC2═C1 |
| 90 | 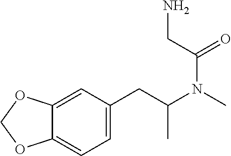 [SMILES: CC(Cc1ccc2c(c1)OCO2)N(C)C(=O)CN] |
| | CN(C(CN)═O)C(C)CC1═CC═C2 |
| | OCOC2═C1 |
| 91 | 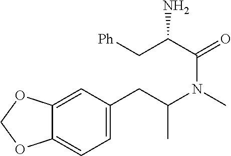 [SMILES: CC(Cc1ccc2c(c1)OCO2)N(C)C(=O)[C@@H](N)Cc1ccccc1] |
| | CN(C([C@@H](N)CC1═CC═ |
| | CC═C1)═O)C(C)CC2═CC═C3OCO |
| | C3═C2 |
| 92 | 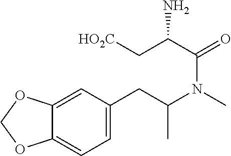 [SMILES: CC(Cc1ccc2c(c1)OCO2)N(C)C(=O)[C@@H](N)CC(=O)O] |
| | CN(C([C@@H](N)CC(O)═O)═ |
| | O)C(C)CC1═CC═C2OCOC2═C1 |
| 93 | 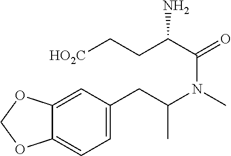 [SMILES: CC(Cc1ccc2c(c1)OCO2)N(C)C(=O)[C@@H](N)CCC(=O)O] |
| | CN(C([C@H](N)CCC(O)═O)═O) |
| | C(C)CC1═CC═C2OCOC2═C1 |
| 94 | 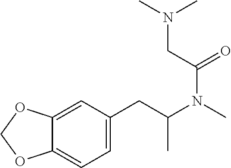 [SMILES: CC(Cc1ccc2c(c1)OCO2)N(C)C(=O)CN(C)C] |
| | CN(C(CN(C)C)═O)C(C)CC1═ |
| | CC═C2OCOC2═C1 |
| 95 | 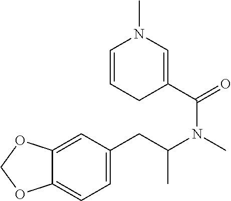 [SMILES: CC(Cc1ccc2c(c1)OCO2)N(C)C(=O)C1=CN(C)C=CC1] |
| | CN(C(C1═CN(C)C═CC1)═O)C |
| | (C)CC2═CC═C3OCOC3═C2 |
| 96 | 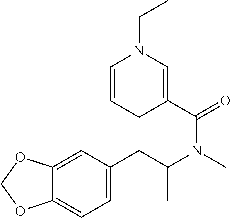 [SMILES: CCN1C=CCC(C(=O)N(C)C(C)Cc2ccc3c(c2)OCO3)=C1] |
| | CN(C(C1═CN(CC)C═CC1)═O) |
| | C(C)CC2═CC═C3OCOC3═C2 |
| 97 | 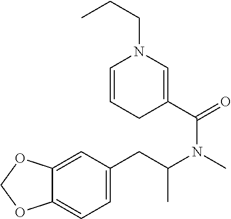 [SMILES: CCCN1C=CCC(C(=O)N(C)C(C)Cc2ccc3c(c2)OCO3)=C1] |
| | CN(C(C1═CN(CCC)C═CC1)═O) |
| | C(C)CC2═CC═C3OCOC3═C2 |
| 98 | 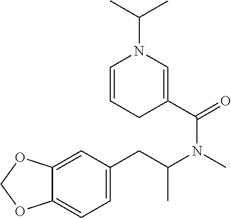 [SMILES: CC(C)N1C=CCC(C(=O)N(C)C(C)Cc2ccc3c(c2)OCO3)=C1] |
| | CN(C(C1═CN(C(C)C)C═CC1)═ |
| | O)C(C)CC2═CC═C3OCOC3═C2 |
| 99 | 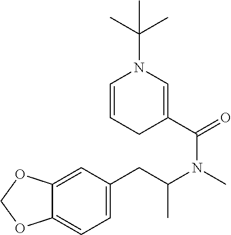 [SMILES: CC(Cc1ccc2c(c1)OCO2)N(C)C(=O)C1=CN(C(C)(C)C)C=CC1] |
| | CN(C(C1═CN(C(C)(C)C)C═ |
| | CC1)═O)C(C)CC2═CC═C3OCOC3═C2 |
| 100 | 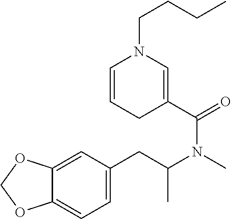 [SMILES: CCCCN1C=CCC(C(=O)N(C)C(C)Cc2ccc3c(c2)OCO3)=C1] |
| | CN(C(C1═CN(CCCC)C═CC1)═ |
| | O)C(C)CC2═CC═C3OCOC3═C2 |
| 101 | 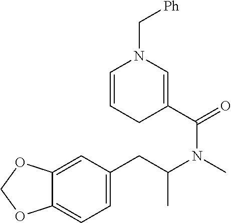 [SMILES: CC(Cc1ccc2c(c1)OCO2)N(C)C(=O)C1=CN(Cc2ccccc2)C=CC1] |
| | CN(C(C1═CN(CC2═CC═CC═C2) |
| | C═CC1)═O)C(C)CC3═CC═C4OC |
| | OC4═C3 |
| 102 | 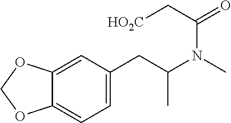 [SMILES: CC(Cc1ccc2c(c1)OCO2)N(C)C(=O)CC(=O)O] |
| | CN(C(CC(O)═O)═O)C(C)CC1═C |
| | C═C2OCOC2═C1 |
| 103 | 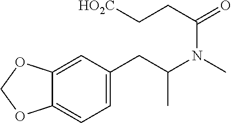 [SMILES: CC(Cc1ccc2c(c1)OCO2)N(C)C(=O)CCC(=O)O] |
| | CN(C(CCC(O)═O)═O)C(C)CC1═ |
| | CC═C2OCOC2═C1 |
| 104 | 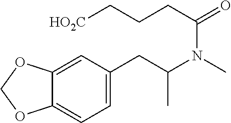 [SMILES: CC(Cc1ccc2c(c1)OCO2)N(C)C(=O)CCCC(=O)O] |
| | CN(C(CCCC(O)═O)═O)C(C) |
| | CC1═CC═C2OCOC2═C1 |
| 105 | 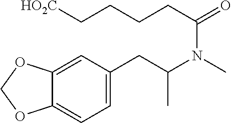 [SMILES: CC(Cc1ccc2c(c1)OCO2)N(C)C(=O)CCCCC(=O)O] |
| | CN(C(CCCCC(O)═O)═O)C(C)C |
| | C1═CC═C2OCOC2═C1 |
| 106 | 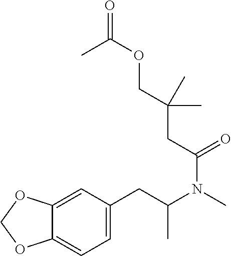 [SMILES: CC(=O)OCC(C)(C)CC(=O)N(C)C(C)Cc1ccc2c(c1)OCO2] |
| | CN(C(CC(C)(C)COC(C)═O)═O) |
| | C(C)CC1═CC═C2OCOC2═C1 |
| 107 | 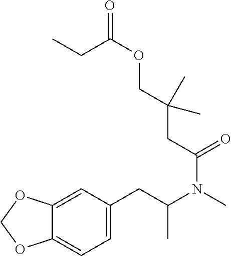 [SMILES: CCC(=O)OCC(C)(C)CC(=O)N(C)C(C)Cc1ccc2c(c1)OCO2] |
| | CN(C(CC(C)(C)COC(CC)═O)═ |
| | O)C(C)CC1═CC═C2OCOC2═C1 |
| 108 | 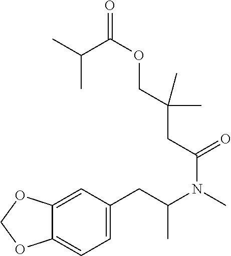 [SMILES: CC(C)C(=O)OCC(C)(C)CC(=O)N(C)C(C)Cc1ccc2c(c1)OCO2] |
| | CN(C(CC(C)(C)COC(C(C)C)═ |
| | O)═O)C(C)CC1═CC═C2OCOC2═C1 |
| 109 | 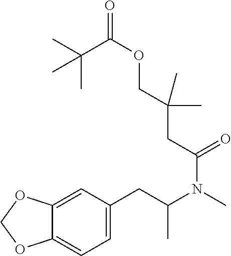 [SMILES: CC(Cc1ccc2c(c1)OCO2)N(C)C(=O)CC(C)(C)COC(=O)C(C)(C)C] |
| | CN(C(CC(C)(C)COC(C(C)(C) |
| | C)═O)═O)C(C)CC1═CC═ |
| | C2OCOC2═C1 |
| 110 | 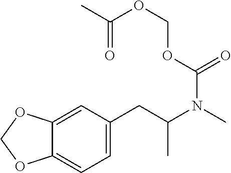 [SMILES: CC(=O)OCOC(=O)N(C)C(C)Cc1ccc2c(c1)OCO2] |
| | CC(CC1═CC═C2OCOC2═C1) |
| | N(C(OCOC(C)═O)═O)C |
| 111 | 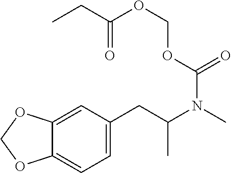 [SMILES: CCC(=O)OCOC(=O)N(C)C(C)Cc1ccc2c(c1)OCO2] |
| | CC(CC1═CC═C2OCOC2═C1) |
| | N(C(OCOC(CC)═O)═O)C |
| 112 | 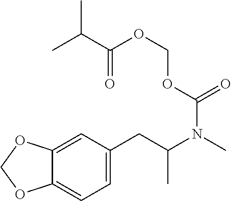 [SMILES: CC(C)C(=O)OCOC(=O)N(C)C(C)Cc1ccc2c(c1)OCO2] |
| | CC(CC1═CC═C2OCOC2═C1) |
| | N(C(OCOC(C(C)C)═O)═O)C |
| 113 | 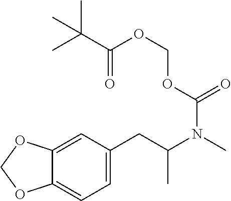 [SMILES: CC(Cc1ccc2c(c1)OCO2)N(C)C(=O)OCOC(=O)C(C)(C)C] |
| | CC(CC1═CC═C2OCOC2═C1) |
| | N(C(OCOC(C(C)(C)C)═O)═O)C |
| 114 | 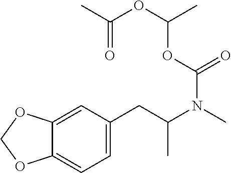 [SMILES: CC(=O)OC(C)OC(=O)N(C)C(C)Cc1ccc2c(c1)OCO2] |
| | CC(CC1═CC═C2OCOC2═C1) |
| | N(C(OC(C)OC(C)═O)═O)C |
| 115 | 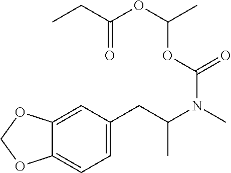 [SMILES: CCC(=O)OC(C)OC(=O)N(C)C(C)Cc1ccc2c(c1)OCO2] |
| | CC(CC1═CC═C2OCOC2═C1) |
| | N(C(OC(C)OC(CC)═O)═O)C |
| 116 | 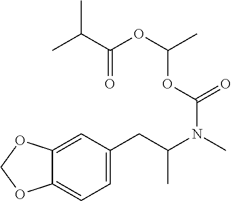 [SMILES: CC(OC(=O)C(C)C)OC(=O)N(C)C(C)Cc1ccc2c(c1)OCO2] |
| | CC(CC1═CC═C2OCOC2═C1) |
| | N(C(OC(C)OC(C(C)C)═O)═O)C |
| 117 | 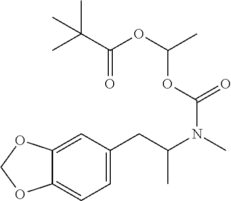 [SMILES: CC(OC(=O)N(C)C(C)Cc1ccc2c(c1)OCO2)OC(=O)C(C)(C)C] |
| | CC(CC1═CC═C2OCOC2═C1) |
| | N(C(OC(C)OC(C(C)(C)C)═O)═O)C |
| 118 | 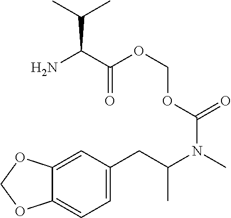 [SMILES: CC(C)[C@H](N)C(=O)OCOC(=O)N(C)C(C)Cc1ccc2c(c1)OCO2] |
| | CC(CC1═CC═C2OCOC2═C1) |
| | N(C(OCOC([C@@H](N)C(C)C)═O)═O)C |
| 119 | 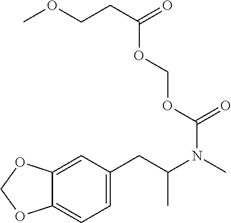 [SMILES: COCCC(=O)OCOC(=O)N(C)C(C)Cc1ccc2c(c1)OCO2] |
| | CN(C(OCOC(CCOC)═O)═O)C |
| | (C)CC1═CC═C2OCOC2═C1 |
| 120 | 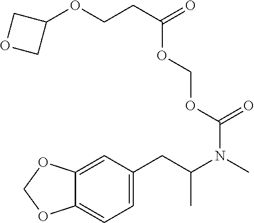 [SMILES: CC(Cc1ccc2c(c1)OCO2)N(C)C(=O)OCOC(=O)CCOC1COC1] |
| | CN(C(OCOC(CCOC1COC1)═ |
| | O)═O)C(C)CC2═CC═C3OCOC3═C |
| | 2 |
| 121 | 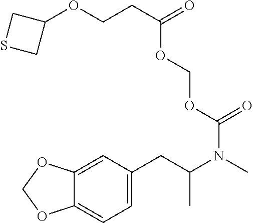 [SMILES: CC(Cc1ccc2c(c1)OCO2)N(C)C(=O)OCOC(=O)CCOC1CSC1] |
| | CN(C(OCOC(CCOC1CSC1)═ |
| | O)═O)C(C)CC2═CC═C3OCOC3═C |
| | 2 |
| 122 | 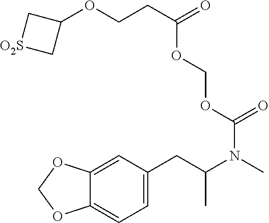 [SMILES: CC(Cc1ccc2c(c1)OCO2)N(C)C(=O)OCOC(=O)CCOC1CS(=O)(=O)C1] |
| | CN(C(OCOC(CCOC1CS(C1)(═ |
| | O)═O)═O)═O)C(C)CC2═CC═C3O |
| | COC3═C2 |
| 123 | 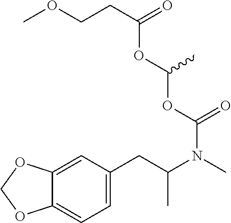 [SMILES: COCCC(=O)OC(C)OC(=O)N(C)C(C)Cc1ccc2c(c1)OCO2] |
| | CN(C(OC(C)OC(CCOC)═O)═O) |
| | C(C)CC1═CC═C2OCOC2═C1 |
| 124 | 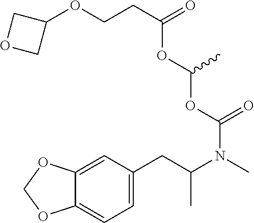 [SMILES: CC(OC(=O)CCOC1COC1)OC(=O)N(C)C(C)Cc1ccc2c(c1)OCO2] |
| | CN(C(OC(C)OC(CCOC1COC1)═ |
| | O)═O)C(C)CC2═CC═ |
| | C3OCOC3═C2 |
| 125 | 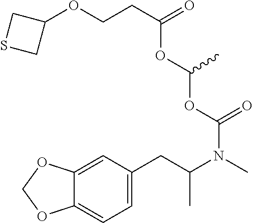 [SMILES: CC(OC(=O)CCOC1CSC1)OC(=O)N(C)C(C)Cc1ccc2c(c1)OCO2] |
| | CN(C(OC(C)OC(CCOC1CSC1)═ |
| | O)═O)C(C)CC2═CC═ |
| | C3OCOC3═C2 |
| 126 | 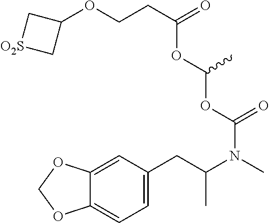 [SMILES: CC(OC(=O)CCOC1CS(=O)(=O)C1)OC(=O)N(C)C(C)Cc1ccc2c(c1)OCO2] |
| | CN(C(OC(C)OC(CCOCICS(C1) |
| | (═O)═O)═O)═O)C(C)CC2═CC═C |
| | 3OCOC3═C2 |
| 127 | 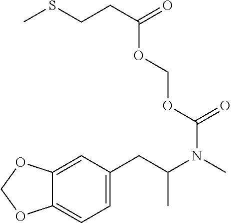 [SMILES: CSCCC(=O)OCOC(=O)N(C)C(C)Cc1ccc2c(c1)OCO2] |
| | CN(C(OCOC(CCSC)═O)═O)C |
| | (C)CC1═CC═C2OCOC2═C1 |
| 128 | 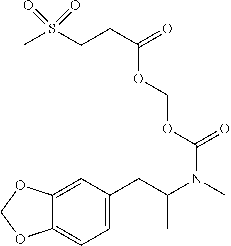 [SMILES: CC(Cc1ccc2c(c1)OCO2)N(C)C(=O)OCOC(=O)CCS(C)(=O)=O] |
| | CN(C(OCOC(CCS(C)(═O)═O)═ |
| | O)═O)C(C)CC1═CC═ |
| | C2OCOC2═C1 |
| 129 | 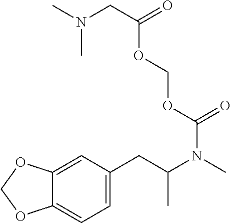 [SMILES: CC(Cc1ccc2c(c1)OCO2)N(C)C(=O)OCOC(=O)CN(C)C] |
| | CN(C(OCOC(CN(C)C)═O)═O) |
| | C(C)CC1═CC═C2OCOC2═C1 |
| 130 | 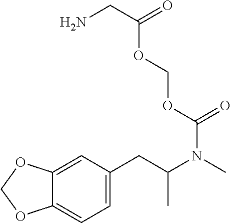 [SMILES: CC(Cc1ccc2c(c1)OCO2)N(C)C(=O)OCOC(=O)CN] |
| | CN(C(OCOC(CN)═O)═O)C(C)C |
| | C1═CC═C2OCOC2═C1 |
| 131 | 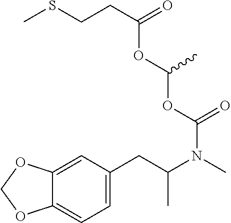 [SMILES: CSCCC(=O)OC(C)OC(=O)N(C)C(C)Cc1ccc2c(c1)OCO2] |
| | CN(C(OC(C)OC(CCSC)═O)═O) |
| | C(C)CC1═CC═C2OCOC2═C1 |
| 132 | 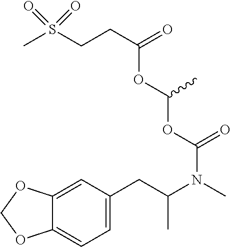 [SMILES: CC(OC(=O)CCS(C)(=O)=O)OC(=O)N(C)C(C)Cc1ccc2c(c1)OCO2] |
| | CN(C(OC(C)OC(CCS(C)(═O)═ |
| | O)═O)═O)C(C)CC1═CC═C2OCOC |
| | 2═C1 |
| 133 | 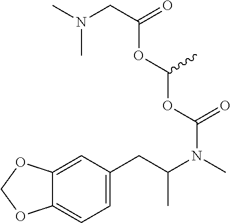 [SMILES: CC(OC(=O)CN(C)C)OC(=O)N(C)C(C)Cc1ccc2c(c1)OCO2] |
| | CN(C(OC(C)OC(CN(C)C)═O)═ |
| | O)C(C)CC1═CC═C2OCOC2═C1 |
| 134 | 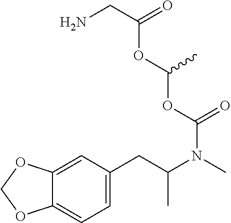 [SMILES: CC(OC(=O)CN)OC(=O)N(C)C(C)Cc1ccc2c(c1)OCO2] |
| | CN(C(OC(C)OC(CN)═O)═O)C |
| | (C)CC1═CC═C2OCOC2═C1 |
| 135 | 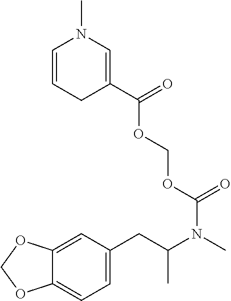 [SMILES: CC(Cc1ccc2c(c1)OCO2)N(C)C(=O)OCOC(=O)C1=CN(C)C=CC1] |
| | CN(C(OCOC(C1═CN(C)C═ |
| | CC1)═O)═O)C(C)CC2═CC═C3OCOC |
| | 3═C2 |
| 136 | 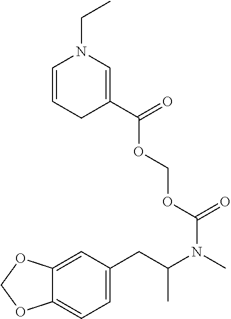 [SMILES: CCN1C=CCC(C(=O)OCOC(=O)N(C)C(C)Cc2ccc3c(c2)OCO3)=C1] |
| | CN(C(OCOC(C1═CN(CC)C═ |
| | CC1)═O)═O)C(C)CC2═CC═C3OCO |
| | C3═C2 |
| 137 | 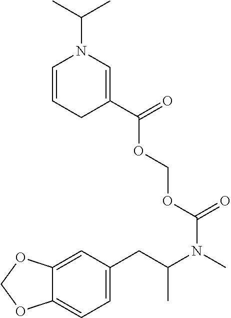 [SMILES: CC(C)N1C=CCC(C(=O)OCOC(=O)N(C)C(C)Cc2ccc3c(c2)OCO3)=C1] |
| | CN(C(OCOC(C1═CN(C(C)C)C═ |
| | CC1)═O)═O)C(C)CC2═CC═C3O |
| | COC3═C2 |
| 138 | 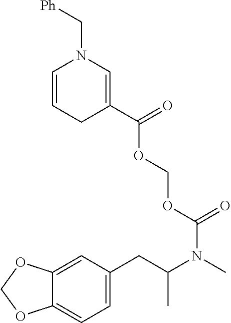 [SMILES: CC(Cc1ccc2c(c1)OCO2)N(C)C(=O)OCOC(=O)C1=CN(Cc2ccccc2)C=CC1] |
| | CN(C(OCOC(C1═CN(CC2═CC═ |
| | CC═C2)C═CC1)═O)═O)C(C) |
| | CC3═CC═C4OCOC4═C3 |
| 139 | 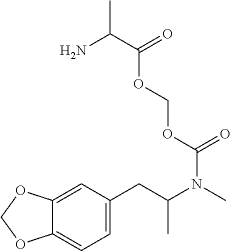 [SMILES: CC(N)C(=O)OCOC(=O)N(C)C(C)Cc1ccc2c(c1)OCO2] |
| | CN(C(OCOC(C(C)N)═O)═O)C |
| | (C)CC1═CC═C2OCOC2═C1 |
| 140 | 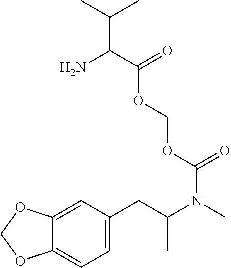 [SMILES: CC(C)C(N)C(=O)OCOC(=O)N(C)C(C)Cc1ccc2c(c1)OCO2] |
| | CN(C(OCOC(C(C(C)C)N)═O)═ |
| | O)C(C)CC1═CC═C2OCOC2═C1 |
| 141 | 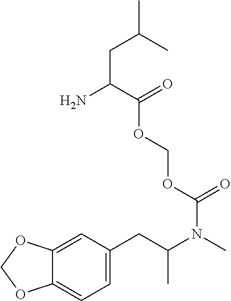 [SMILES: CC(C)CC(N)C(=O)OCOC(=O)N(C)C(C)Cc1ccc2c(c1)OCO2] |
| | CN(C(OCOC(C(CC(C)C)N)═O)═ |
| | O)C(C)CC1═CC═C2OCOC2═C1 |
| 142 | 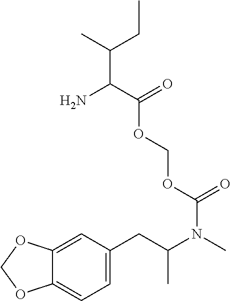 [SMILES: CCC(C)C(N)C(=O)OCOC(=O)N(C)C(C)Cc1ccc2c(c1)OCO2] |
| | CN(C(OCOC(C(C(C)CC)N)═O)═ |
| | O)C(C)CC1═CC═C2OCOC2═C1 |
| 143 | 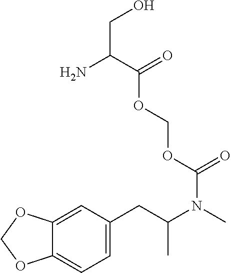 [SMILES: CC(Cc1ccc2c(c1)OCO2)N(C)C(=O)OCOC(=O)C(N)CO] |
| | CN(C(OCOC(C(CO)N)═O)═O)C |
| | (C)CC1═CC═C2OCOC2═C1 |
| 144 | 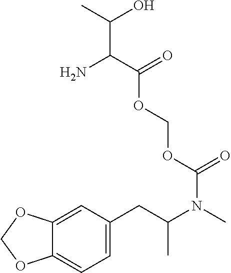 [SMILES: CC(O)C(N)C(=O)OCOC(=O)N(C)C(C)Cc1ccc2c(c1)OCO2] |
| | CN(C(OCOC(C(C(C)O)N)═O)═ |
| | O)C(C)CC1═CC═C2OCOC2═C1 |
| 145 | 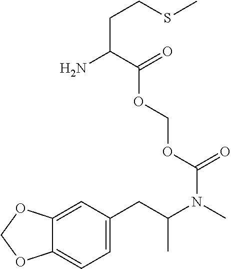 [SMILES: CSCCC(N)C(=O)OCOC(=O)N(C)C(C)Cc1ccc2c(c1)OCO2] |
| | CN(C(OCOC(C(CCSC)N)═O)═ |
| | O)C(C)CC1═CC═C2OCOC2═C1 |
| 146 | 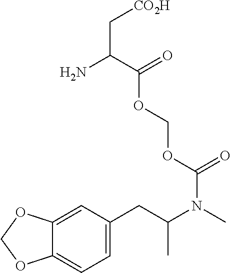 [SMILES: CC(Cc1ccc2c(c1)OCO2)N(C)C(=O)OCOC(=O)C(N)CC(=O)O] |
| | CN(C(OCOC(C(CC(O)═O)N)═ |
| | O)═O)C(C)CC1═CC═C2OCOC2═ |
| | C1 |
| 147 | 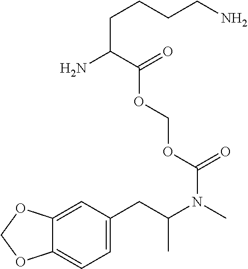 [SMILES: CC(Cc1ccc2c(c1)OCO2)N(C)C(=O)OCOC(=O)C(N)CCCCN] |
| | CN(C(OCOC(C(CCCCN)N)═O)═ |
| | O)C(C)CC1═CC═C2OCOC2═C1 |
| 148 | 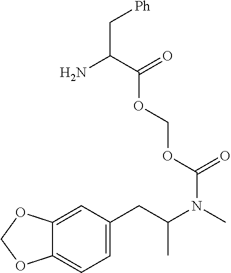 [SMILES: CC(Cc1ccc2c(c1)OCO2)N(C)C(=O)OCOC(=O)C(N)Cc1ccccc1] |
| | CN(C(OCOC(C(CC1═CC═CC═C |
| | 1)N)═O)═O)C(C)CC2═CC═C3O |
| | COC3═C2 |
| 149 | 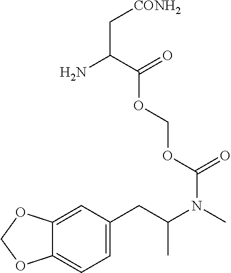 [SMILES: CC(Cc1ccc2c(c1)OCO2)N(C)C(=O)OCOC(=O)C(N)CC(N)=O] |
| | CN(C(OCOC(C(CC(N)═O)N)═ |
| | O)═O)C(C)CC1═CC═C2OCOC2═C |
| | 1 |
| 150 | 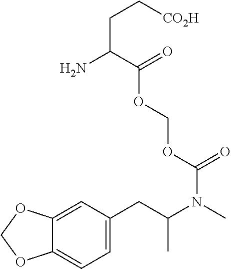 [SMILES: CC(Cc1ccc2c(c1)OCO2)N(C)C(=O)OCOC(=O)C(N)CCC(=O)O] |
| | CN(C(OCOC(C(CCC(O)═O)N)═ |
| | O)═O)C(C)CC1═CC═ |
| | C2OCOC2═C1 |
| 151 | 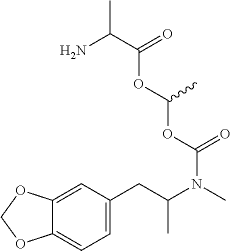 [SMILES: CC(OC(=O)C(C)N)OC(=O)N(C)C(C)Cc1ccc2c(c1)OCO2] |
| | CN(C(OC(C)OC(C(C)N)═O)═O) |
| | C(C)CC1═CC═C2OCOC2═C1 |
| 152 | 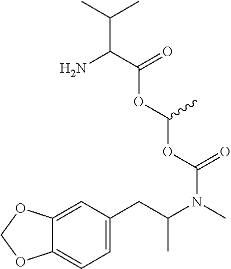 [SMILES: CC(OC(=O)C(N)C(C)C)OC(=O)N(C)C(C)Cc1ccc2c(c1)OCO2] |
| | CN(C(OC(C)OC(C(C(C)C)N)═ |
| | O)═O)C(C)CC1═CC═C2OCOC2═C |
| | 1 |
| 153 | 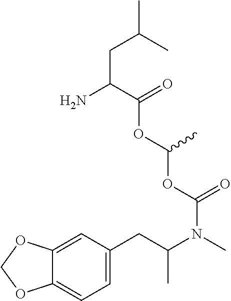 [SMILES: CC(C)CC(N)C(=O)OC(C)OC(=O)N(C)C(C)Cc1ccc2c(c1)OCO2] |
| | CN(C(OC(C)OC(C(CC(C)C)N)═ |
| | O)═O)C(C)CC1═CC═ |
| | C2OCOC2═C1 |
| 154 | 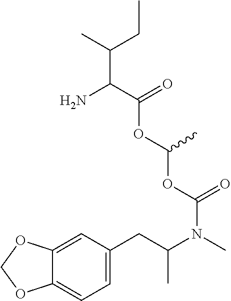 [SMILES: CCC(C)C(N)C(=O)OC(C)OC(=O)N(C)C(C)Cc1ccc2c(c1)OCO2] |
| | CN(C(OC(C)OC(C(C(C)CC)N)═ |
| | O)═O)C(C)CC1═CC═ |
| | C2OCOC2═C1 |
| 155 | 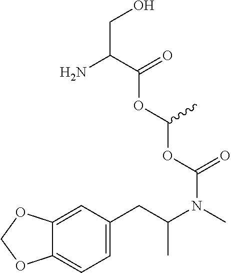 [SMILES: CC(OC(=O)C(N)CO)OC(=O)N(C)C(C)Cc1ccc2c(c1)OCO2] |
| | CN(C(OC(C)OC(C(CO)N)═O)═ |
| | O)C(C)CC1═CC═C2OCOC2═C1 |
| 156 | 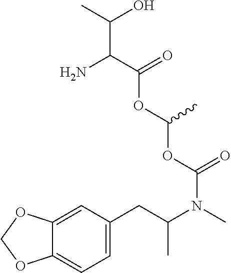 [SMILES: CC(OC(=O)C(N)C(C)O)OC(=O)N(C)C(C)Cc1ccc2c(c1)OCO2] |
| | CN(C(OC(C)OC(C(C(C)O)N)═ |
| | O)═O)C(C)CC1═CC═C2OCOC2═ |
| | C1 |
| 157 | 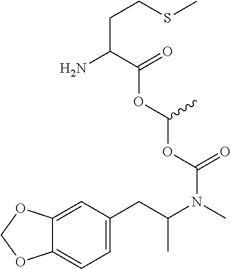 [SMILES: CSCCC(N)C(=O)OC(C)OC(=O)N(C)C(C)Cc1ccc2c(c1)OCO2] |
| | CN(C(OC(C)OC(C(CCSC)N)═ |
| | O)═O)C(C)CC1═CC═C2OCOC2═C |
| | 1 |
| 158 | 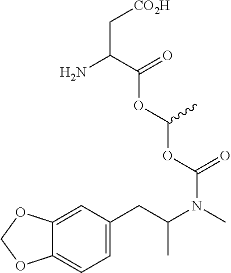 [SMILES: CC(OC(=O)C(N)CC(=O)O)OC(=O)N(C)C(C)Cc1ccc2c(c1)OCO2] |
| | CN(C(OC(C)OC(C(CC(O)═O) |
| | N)═O)═O)C(C)CC1═CC═C2OCOC |
| | 2═C1 |
| 159 | 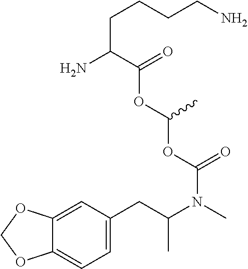 [SMILES: CC(OC(=O)C(N)CCCCN)OC(=O)N(C)C(C)Cc1ccc2c(c1)OCO2] |
| | CN(C(OC(C)OC(C(CCCCN)N)═ |
| | O)═O)C(C)CC1═CC═ |
| | C2OCOC2═C1 |
| 160 | 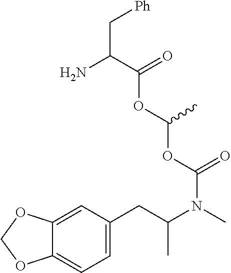 [SMILES: CC(OC(=O)C(N)Cc1ccccc1)OC(=O)N(C)C(C)Cc1ccc2c(c1)OCO2] |
| | CN(C(OC(C)OC(C(CC1═CC═ |
| | CC═C1)N)═O)═O)C(C)CC2═CC═C3 |
| | OCOC3═C2 |
| 161 | 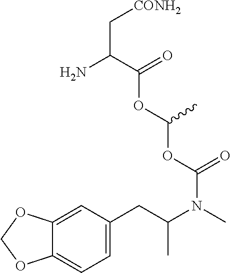 [SMILES: CC(OC(=O)C(N)CC(N)=O)OC(=O)N(C)C(C)Cc1ccc2c(c1)OCO2] |
| | CN(C(OC(C)OC(C(CC(N)═O) |
| | N)═O)═O)C(C)CC1═CC═C2OCOC |
| | 2═C1 |
| 162 | 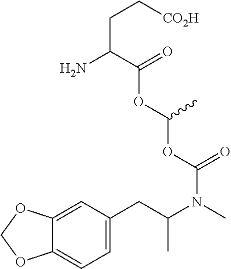 [SMILES: CC(OC(=O)C(N)CCC(=O)O)OC(=O)N(C)C(C)Cc1ccc2c(c1)OCO2] |
| | CN(C(OC(C)OC(C(CCC(O)═O) |
| | N)═O)═O)C(C)CC1═CC═C2OCO |
| | C2═C1 |
| 163 | 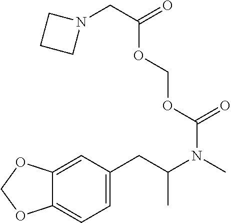 [SMILES: CC(Cc1ccc2c(c1)OCO2)N(C)C(=O)OCOC(=O)CN1CCC1] |
| | CN(C(OCOC(CN1CCC1)═O)═O) |
| | C(C)CC2═CC═C3OCOC3═C2 |
| 164 | 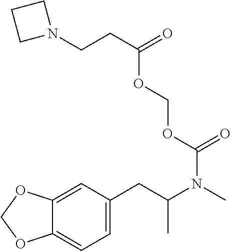 [SMILES: CC(Cc1ccc2c(c1)OCO2)N(C)C(=O)OCOC(=O)CCN1CCC1] |
| | CN(C(OCOC(CCN1CCC1)═O)═O) |
| | C(C)CC2═CC═C3OCOC3═C2 |
| 165 | 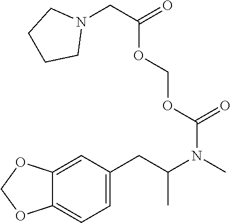 [SMILES: CC(Cc1ccc2c(c1)OCO2)N(C)C(=O)OCOC(=O)CN1CCCC1] |
| | CN(C(OCOC(CN1CCCC1)═O)═ |
| | O)C(C)CC2═CC═C3OCOC3═C2 |
| 166 | 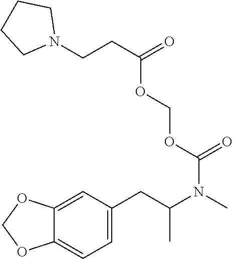 [SMILES: CC(Cc1ccc2c(c1)OCO2)N(C)C(=O)OCOC(=O)CCN1CCCC1] |
| | CN(C(OCOC(CCN1CCCC1)═ |
| | O)═O)C(C)CC2═CC═C3OCOC3═ |
| | C2 |
| 167 | 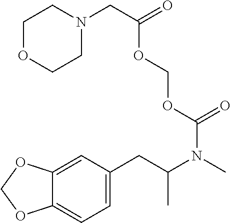 [SMILES: CC(Cc1ccc2c(c1)OCO2)N(C)C(=O)OCOC(=O)CN1CCOCC1] |
| | CN(C(OCOC(CNICCOCC1)═ |
| | O)═O)C(C)CC2═CC═C3OCOC3═ |
| | C2 |
| 168 | 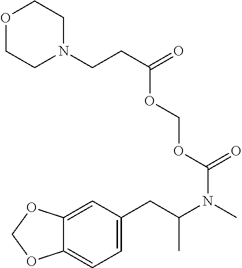 [SMILES: CC(Cc1ccc2c(c1)OCO2)N(C)C(=O)OCOC(=O)CCN1CCOCC1] |
| | CN(C(OCOC(CCNICCOCC1)═ |
| | O)═O)C(C)CC2═CC═ |
| | C3OCOC3═C2 |
| 169 | 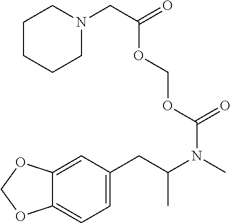 [SMILES: CC(Cc1ccc2c(c1)OCO2)N(C)C(=O)OCOC(=O)CN1CCCCC1] |
| | CN(C(OCOC(CN1CCCCC1)═ |
| | O)═O)C(C)CC2═CC═C3OCOC3═ |
| | C2 |
| 170 | 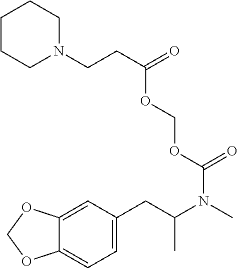 [SMILES: CC(Cc1ccc2c(c1)OCO2)N(C)C(=O)OCOC(=O)CCN1CCCCC1] |
| | CN(C(OCOC(CCNICCCCC1)═ |
| | O)═O)C(C)CC2═CC═C3OCOC3═ |
| | C2 |
| 171 | 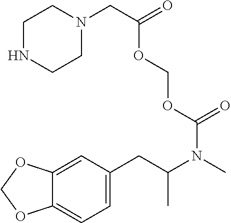 [SMILES: CC(Cc1ccc2c(c1)OCO2)N(C)C(=O)OCOC(=O)CN1CCNCC1] |
| | CN(C(OCOC(CN1CCNCC1)═ |
| | O)═O)C(C)CC2═CC═C3OCOC3═ |
| | C2 |
| 172 | 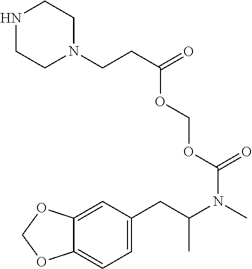 [SMILES: CC(Cc1ccc2c(c1)OCO2)N(C)C(=O)OCOC(=O)CCN1CCNCC1] |
| | CN(C(OCOC(CCN1CCNCC1)═ |
| | O)═O)C(C)CC2═CC═C3OCOC3═ |
| | C2 |
| 173 | 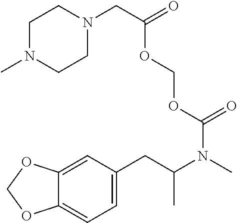 [SMILES: CC(Cc1ccc2c(c1)OCO2)N(C)C(=O)OCOC(=O)CN1CCN(C)CC1] |
| | CN(C(OCOC(CN1CCN(C)CC1)═ |
| | O)═O)C(C)CC2═CC═C3OCOC3═ |
| | C2 |
| 174 | 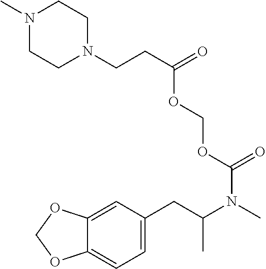 [SMILES: CC(Cc1ccc2c(c1)OCO2)N(C)C(=O)OCOC(=O)CCN1CCN(C)CC1] |
| | CN(C(OCOC(CCNICCN(C) |
| | CC1)═O)═O)C(C)CC2═CC═ |
| | C3OCOC3═C2 |
| 175 | 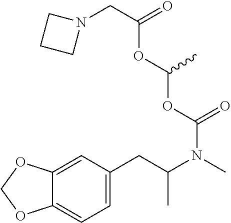 [SMILES: CC(OC(=O)CN1CCC1)OC(=O)N(C)C(C)Cc1ccc2c(c1)OCO2] |
| | CN(C(OC(C)OC(CN1CCC1)═ |
| | O)═O)C(C)CC2═CC═C3OCOC3═C |
| | 2 |
| 176 | 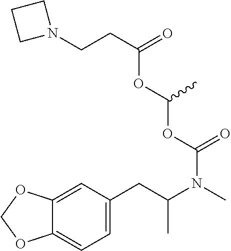 [SMILES: CC(OC(=O)CCN1CCC1)OC(=O)N(C)C(C)Cc1ccc2c(c1)OCO2] |
| | CN(C(OC(C)OC(CCN1CCC1)═ |
| | O)═O)C(C)CC2═CC═C3OCOC3═ |
| | C2 |
| 177 | 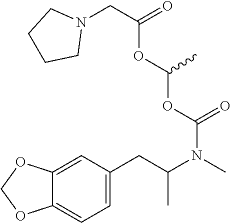 [SMILES: CC(OC(=O)CN1CCCC1)OC(=O)N(C)C(C)Cc1ccc2c(c1)OCO2] |
| | CN(C(OC(C)OC(CN1CCCC1)═ |
| | O)═O)C(C)CC2═CC═C3OCOC3═ |
| | C2 |
| 178 | 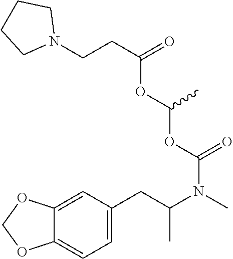 [SMILES: CC(OC(=O)CCN1CCCC1)OC(=O)N(C)C(C)Cc1ccc2c(c1)OCO2] |
| | CN(C(OC(C)OC(CCN1CCCC1)═ |
| | O)═O)C(C)CC2═CC═C3OCOC3═ |
| | C2 |
| 179 | 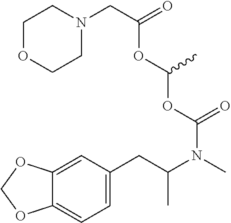 [SMILES: CC(OC(=O)CN1CCOCC1)OC(=O)N(C)C(C)Cc1ccc2c(c1)OCO2] |
| | CN(C(OC(C)OC(CN1CCOCC1)═ |
| | O)═O)C(C)CC2═CC═C3OCOC3═ |
| | C2 |
| 180 | 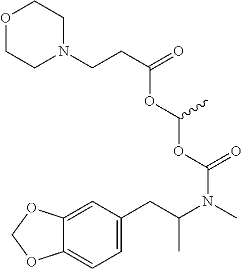 [SMILES: CC(OC(=O)CCN1CCOCC1)OC(=O)N(C)C(C)Cc1ccc2c(c1)OCO2] |
| | CN(C(OC(C)OC(CCN1CCOCC1)═ |
| | O)═O)C(C)CC2═CC═C3OCOC3═ |
| | C2 |
| 181 | 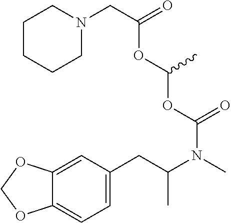 [SMILES: CC(OC(=O)CN1CCCCC1)OC(=O)N(C)C(C)Cc1ccc2c(c1)OCO2] |
| | CN(C(OC(C)OC(CN1CCCCC1)═ |
| | O)═O)C(C)CC2═CC═C3OCOC3═ |
| | C2 |
| 182 | 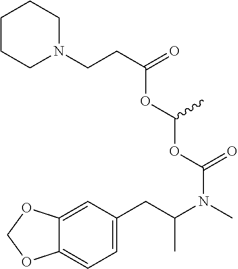 [SMILES: CC(OC(=O)CCN1CCCCC1)OC(=O)N(C)C(C)Cc1ccc2c(c1)OCO2] |
| | CN(C(OC(C)OC(CCN1CCCCC1)═ |
| | O)═O)C(C)CC2═CC═C3OCOC |
| | 3═C2 |
| 183 | 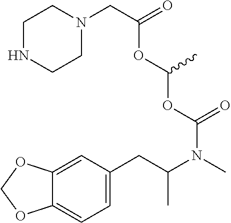 [SMILES: CC(OC(=O)CN1CCNCC1)OC(=O)N(C)C(C)Cc1ccc2c(c1)OCO2] |
| | CN(C(OC(C)OC(CN1CCNCC1)═ |
| | O)═O)C(C)CC2═CC═C3OCOC3═C2 |
| 184 | 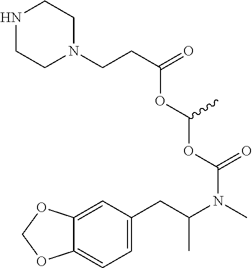 [SMILES: CC(OC(=O)CCN1CCNCC1)OC(=O)N(C)C(C)Cc1ccc2c(c1)OCO2] |
| | CN(C(OC(C)OC(CCN1CCNCC1)═ |
| | O)═O)C(C)CC2═CC═C3OCOC3═ |
| | C2 |
| 185 | 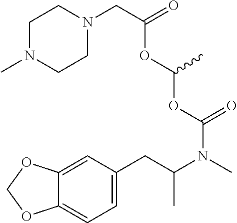 [SMILES: CC(OC(=O)CN1CCN(C)CC1)OC(=O)N(C)C(C)Cc1ccc2c(c1)OCO2] |
| | CN(C(OC(C)OC(CN1CCN(C) |
| | CC1)═O)═O)C(C)CC2═CC═C3OCO |
| | C3═C2 |
| 186 | 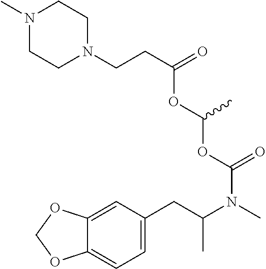 [SMILES: CC(OC(=O)CCN1CCN(C)CC1)OC(=O)N(C)C(C)Cc1ccc2c(c1)OCO2] |
| | CN(C(OC(C)OC(CCN1CCN(C)CC1)═ |
| | O)═O)C(C)CC2═CC=C3OCOC3═C2 |
| 187 | 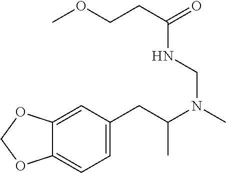 [SMILES: COCCC(=O)NCN(C)C(C)Cc1ccc2c(c1)OCO2] |
| | CN(CNC(CCOC)═O)C(C)CC1═C |
| | C═C2OCOC2═C1 |
| 188 | 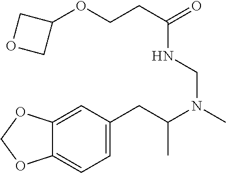 [SMILES: CC(Cc1ccc2c(c1)OCO2)N(C)CNC(=O)CCOC1COC1] |
| | CN(CNC(CCOC1COC1)═O)C(C) |
| | CC2═CC═C3OCOC3═C2 |
| 189 | 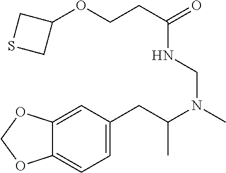 [SMILES: CC(Cc1ccc2c(c1)OCO2)N(C)CNC(=O)CCOC1CSC1] |
| | CN(CNC(CCOC1CSC1)═O)C(C) |
| | CC2═CC═C3OCOC3═C2 |
| 190 | 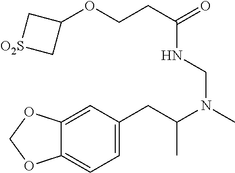 [SMILES: CC(Cc1ccc2c(c1)OCO2)N(C)CNC(=O)CCOC1CS(=O)(=O)C1] |
| | CN(CNC(CCOCICS(C1)(═O)═ |
| | O)═O)C(C)CC2═CC═C3OCOC3═ |
| | C2 |
| 191 | 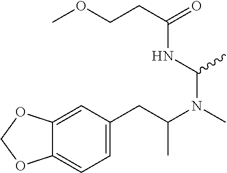 [SMILES: COCCC(=O)NC(C)N(C)C(C)Cc1ccc2c(c1)OCO2] |
| | CN(C(C)NC(CCOC)═O)C(C)CC |
| | 1═CC═C2OCOC2═C1 |
| 192 | 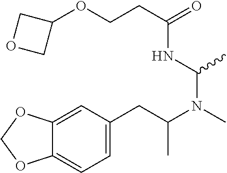 [SMILES: CC(Cc1ccc2c(c1)OCO2)N(C)C(C)NC(=O)CCOC1COC1] |
| | CN(C(C)NC(CCOC1COC1)═O)C |
| | (C)CC2═CC═C3OCOC3═C2 |
| 193 | 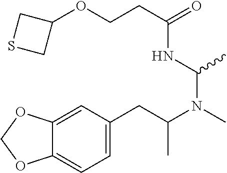 [SMILES: CC(Cc1ccc2c(c1)OCO2)N(C)C(C)NC(=O)CCOC1CSC1] |
| | CN(C(C)NC(CCOC1CSC1)═O)C |
| | (C)CC2═CC═C3OCOC3═C2 |
| 194 | 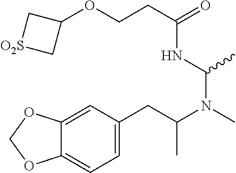 [SMILES: CC(Cc1ccc2c(c1)OCO2)N(C)C(C)NC(=O)CCOC1CS(=O)(=O)C1] |
| | CN(C(C)NC(CCOC1CS(C1)(═ |
| | O)═O)═O)C(C)CC2═CC═C3OCOC |
| | 3═C2 |
| 195 | 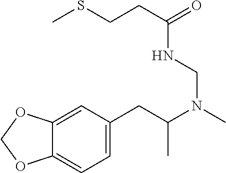 [SMILES: CSCCC(=O)NCN(C)C(C)Cc1ccc2c(c1)OCO2] |
| | CN(CNC(CCSC)═O)C(C)CC1═C |
| | C═C2OCOC2═C1 |
| 196 | 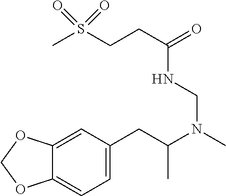 [SMILES: CC(Cc1ccc2c(c1)OCO2)N(C)CNC(=O)CCS(C)(=O)=O] |
| | CN(CNC(CCS(C)(═O)═O)═O) |
| | C(C)CC1═CC═C2OCOC2═C1 |
| 197 | 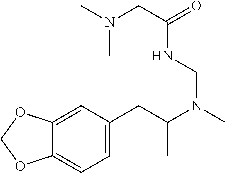 [SMILES: CC(Cc1ccc2c(c1)OCO2)N(C)CNC(=O)CN(C)C] |
| | CN(CNC(CN(C)C)═O)C(C)CC1═ |
| | CC═C2OCOC2═C1 |
| 198 | 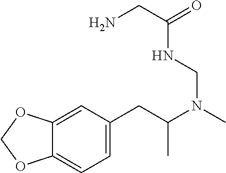 [SMILES: CC(Cc1ccc2c(c1)OCO2)N(C)CNC(=O)CN] |
| | CN(CNC(CN)═O)C(C)CC1═CC═ |
| | C2OCOC2═C1 |
| 199 | 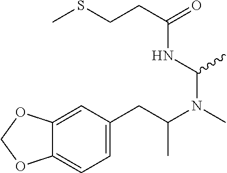 [SMILES: CSCCC(=O)NC(C)N(C)C(C)Cc1ccc2c(c1)OCO2] |
| | CN(C(C)NC(CCSC)═O)C(C) |
| | CC1═CC═C2OCOC2═C1 |
| 200 | 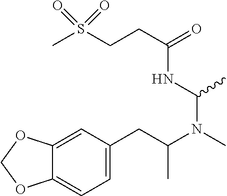 [SMILES: CC(Cc1ccc2c(c1)OCO2)N(C)C(C)NC(=O)CCS(C)(=O)=O] |
| | CN(C(C)NC(CCS(C)(═O)═O)═O) |
| | C(C)CC1═CC═C2OCOC2═C1 |
| 201 | 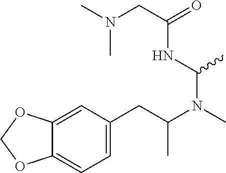 [SMILES: CC(Cc1ccc2c(c1)OCO2)N(C)C(C)NC(=O)CN(C)C] |
| | CN(C(C)NC(CN(C)C)═O)C(C)C |
| | C1═CC═C2OCOC2═C1 |
| 202 | 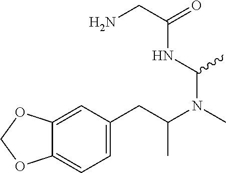 [SMILES: CC(Cc1ccc2c(c1)OCO2)N(C)C(C)NC(=O)CN] |
| | CN(C(C)NC(CN)═O)C(C)CC1═C |
| | C═C2OCOC2═C1 |
| 203 | 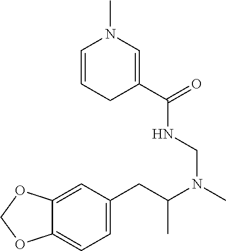 [SMILES: CC(Cc1ccc2c(c1)OCO2)N(C)CNC(=O)C1=CN(C)C=CC1] |
| | CC(CC1═CC═C2OCOC2═C1) |
| | N(C)CNC(C3═CN(C)C═CC3)═O |
| 204 | 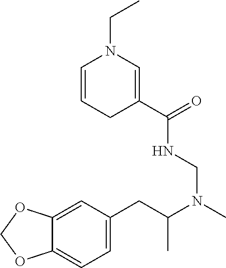 [SMILES: CCN1C=CCC(C(=O)NCN(C)C(C)Cc2ccc3c(c2)OCO3)=C1] |
| | CC(CC1═CC═C2OCOC2═C1) |
| | N(C)CNC(C3═CN(CC)C═CC3)═O |
| 205 | 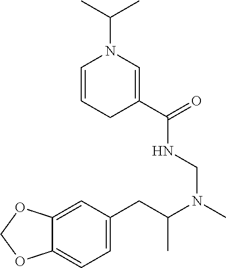 [SMILES: CC(C)N1C=CCC(C(=O)NCN(C)C(C)Cc2ccc3c(c2)OCO3)=C1] |
| | CC(CC1═CC═C2OCOC2═C1) |
| | N(C)CNC(C3═CN(C(C)C)C═CC3)═O |
| 206 | 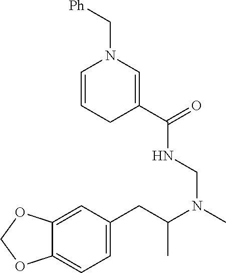 [SMILES: CC(Cc1ccc2c(c1)OCO2)N(C)CNC(=O)C1=CN(Cc2ccccc2)C=CC1] |
| | CC(CC1═CC═C2OCOC2═C1) |
| | N(C)CNC(C3═CN(CC4═CC═CC═C |
| | 4)C═CC3)═O |
| 207 | 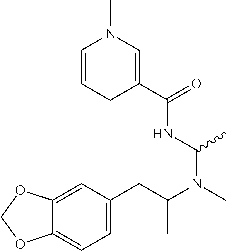 [SMILES: CC(Cc1ccc2c(c1)OCO2)N(C)C(C)NC(=O)C1=CN(C)C=CC1] |
| | CC(CC1═CC═C2OCOC2═C1) |
| | N(C)C(C)NC(C3═CN(C)C═CC3)═O |
| 208 | 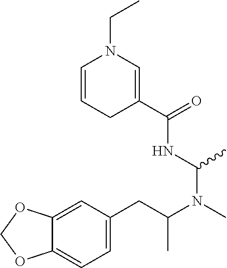 [SMILES: CCN1C=CCC(C(=O)NC(C)N(C)C(C)Cc2ccc3c(c2)OCO3)=C1] |
| | CC(CC1═CC═C2OCOC2═C1) |
| | N(C)C(C)NC(C3═CN(CC)C═CC3)═ |
| | O |
| 209 | 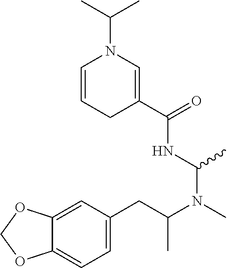 [SMILES: CC(C)N1C=CCC(C(=O)NC(C)N(C)C(C)Cc2ccc3c(c2)OCO3)=C1] |
| | CC(CC1═CC═C2OCOC2═C1) |
| | N(C)C(C)NC(C3═CN(C(C)C)C═ |
| | CC3)═O |
| 210 | 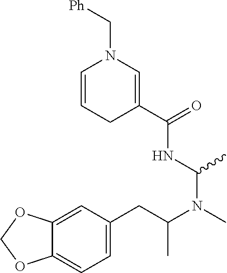 [SMILES: CC(Cc1ccc2c(c1)OCO2)N(C)C(C)NC(=O)C1=CN(Cc2ccccc2)C=CC1] |
| | CC(CC1═CC═C2OCOC2═C1)N |
| | (C)C(C)NC(C3═CN(CC4═CC═ |
| | CC═C4)C═CC3)═O |
| 211 | 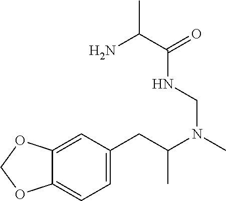 [SMILES: CC(N)C(=O)NCN(C)C(C)Cc1ccc2c(c1)OCO2] |
| | CC(CC1═CC═C2OCOC2═C1)N |
| | (C)CNC(C(N)C)═O |
| 212 | 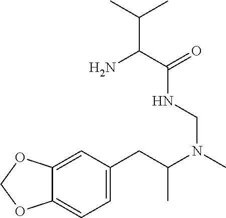 [SMILES: CC(C)C(N)C(=O)NCN(C)C(C)Cc1ccc2c(c1)OCO2] |
| | CC(CC1═CC═C2OCOC2═C1)N |
| | (C)CNC(C(N)C(C)C)═O |
| 213 | 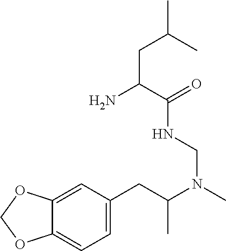 [SMILES: CC(C)CC(N)C(=O)NCN(C)C(C)Cc1ccc2c(c1)OCO2] |
| | CC(CC1═CC═C2OCOC2═C1)N |
| | (C)CNC(C(N)CC(C)C)═O |
| 214 | 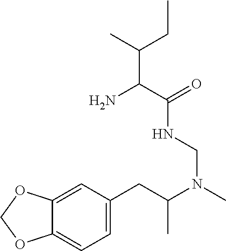 [SMILES: CCC(C)C(N)C(=O)NCN(C)C(C)Cc1ccc2c(c1)OCO2] |
| | CC(CC1═CC═C2OCOC2═C1)N |
| | (C)CNC(C(N)C(C)CC)═O |
| 215 | 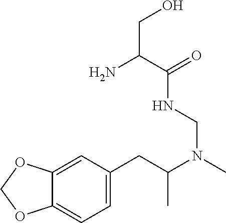 [SMILES: CC(Cc1ccc2c(c1)OCO2)N(C)CNC(=O)C(N)CO] |
| | CC(CC1═CC═C2OCOC2═C1)N |
| | (C)CNC(C(N)CO)═O |
| 216 | 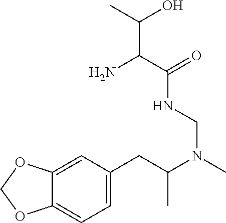 [SMILES: CC(O)C(N)C(=O)NCN(C)C(C)Cc1ccc2c(c1)OCO2] |
| | CC(CC1═CC═C2OCOC2═C1)N |
| | (C)CNC(C(N)C(C)O)═O |
| 217 | 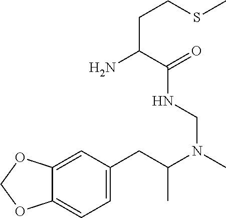 [SMILES: CSCCC(N)C(=O)NCN(C)C(C)Cc1ccc2c(c1)OCO2] |
| | CC(CC1═CC═C2OCOC2═C1)N |
| | (C)CNC(C(N)CCSC)═O |
| 218 | 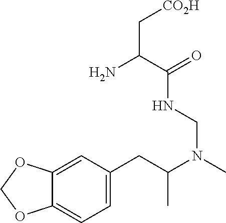 [SMILES: CC(Cc1ccc2c(c1)OCO2)N(C)CNC(=O)C(N)CC(=O)O] |
| | CC(CC1═CC═C2OCOC2═C1)N |
| | (C)CNC(C(N)CC(O)═O)═O |
| 219 | 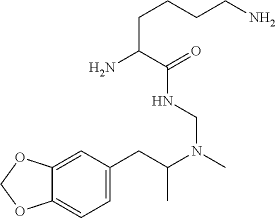 [SMILES: CC(Cc1ccc2c(c1)OCO2)N(C)CNC(=O)C(N)CCCCN] |
| | CC(CC1═CC═C2OCOC2═C1)N |
| | (C)CNC(C(N)CCCCN)═O |
| 220 | 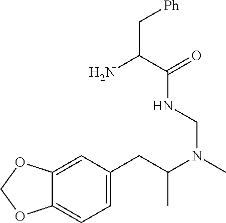 [SMILES: CC(Cc1ccc2c(c1)OCO2)N(C)CNC(=O)C(N)Cc1ccccc1] |
| | CC(CC1═CC═C2OCOC2═C1)N |
| | (C)CNC(C(N)CC3═CC═CC═C3)═ |
| | O |
| 221 | 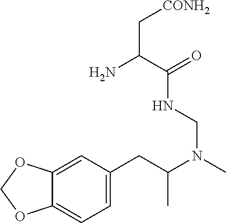 [SMILES: CC(Cc1ccc2c(c1)OCO2)N(C)CNC(=O)C(N)CC(N)=O] |
| | CC(CC1═CC═C2OCOC2═C1)N |
| | (C)CNC(C(N)CC(N)═O)═O |
| 222 | 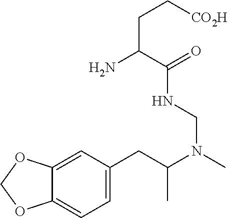 [SMILES: CC(Cc1ccc2c(c1)OCO2)N(C)CNC(=O)C(N)CCC(=O)O] |
| | CC(CC1═CC═C2OCOC2═C1)N |
| | (C)CNC(C(N)CCC(O)═O)═O |
| 223 | 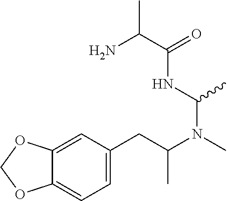 [SMILES: CC(N)C(=O)NC(C)N(C)C(C)Cc1ccc2c(c1)OCO2] |
| | CC(CC1═CC═C2OCOC2═C1)N |
| | (C)C(C)NC(C(N)C)═O |
| 224 | 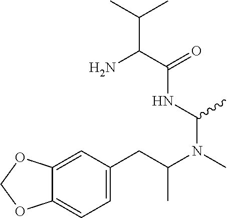 [SMILES: CC(C)C(N)C(=O)NC(C)N(C)C(C)Cc1ccc2c(c1)OCO2] |
| | CC(CC1═CC═C2OCOC2═C1) |
| | N(C)C(C)NC(C(N)C(C)C)═O |
| 226 | 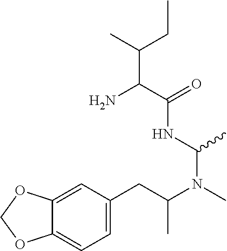 [SMILES: CCC(C)C(N)C(=O)NC(C)N(C)C(C)Cc1ccc2c(c1)OCO2] |
| | CC(CC1═CC═C2OCOC2═C1)N |
| | (C)C(C)NC(C(N)C(C)CC)═O |
| 227 | 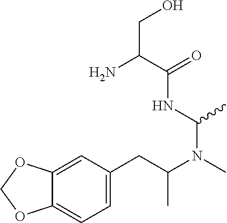 [SMILES: CC(Cc1ccc2c(c1)OCO2)N(C)C(C)NC(=O)C(N)CO] |
| | CC(CC1═CC═C2OCOC2═C1)N |
| | (C)C(C)NC(C(N)CO)═O |
| 228 | 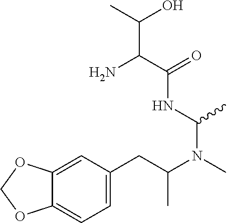 [SMILES: CC(O)C(N)C(=O)NC(C)N(C)C(C)Cc1ccc2c(c1)OCO2] |
| | CC(CC1═CC═C2OCOC2═C1)N |
| | (C)C(C)NC(C(N)C(C)O)═O |
| 229 | 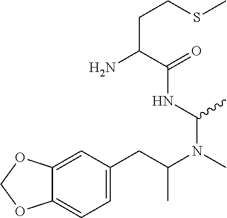 [SMILES: CSCCC(N)C(=O)NC(C)N(C)C(C)Cc1ccc2c(c1)OCO2] |
| | CC(CC1═CC═C2OCOC2═C1)N |
| | (C)C(C)NC(C(N)CCSC)═O |
| 230 | 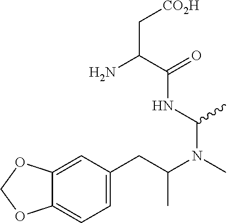 [SMILES: CC(Cc1ccc2c(c1)OCO2)N(C)C(C)NC(=O)C(N)CC(=O)O] |
| | CC(CC1═CC═C2OCOC2═C1)N |
| | (C)C(C)NC(C(N)CC(O)═O)═O |
| 231 | 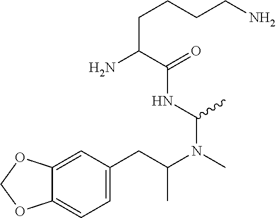 [SMILES: CC(Cc1ccc2c(c1)OCO2)N(C)C(C)NC(=O)C(N)CCCCN] |
| | CC(CC1═CC═C2OCOC2═C1)N |
| | (C)C(C)NC(C(N)CCCCN)═O |
| 232 | 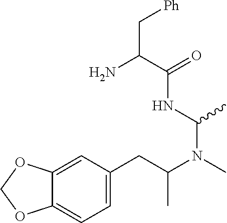 [SMILES: CC(Cc1ccc2c(c1)OCO2)N(C)C(C)NC(=O)C(N)Cc1ccccc1] |
| | CC(CC1═CC═C2OCOC2═C1)N |
| | (C)C(C)NC(C(N)CC3═CC═CC═ |
| | C3)═O |
| 233 | 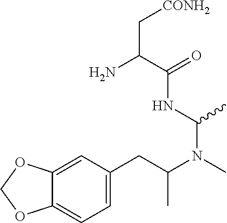 [SMILES: CC(Cc1ccc2c(c1)OCO2)N(C)C(C)NC(=O)C(N)CC(N)=O] |
| | CC(CC1═CC═C2OCOC2═C1)N |
| | (C)C(C)NC(C(N)CC(N)═O)═O |
| 234 | 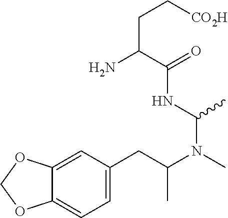 [SMILES: CC(Cc1ccc2c(c1)OCO2)N(C)C(C)NC(=O)C(N)CCC(=O)O] |
| | CC(CC1═CC═C2OCOC2═C1)N |
| | (C)C(C)NC(C(N)CCC(O)═O)═O |
| 235 | 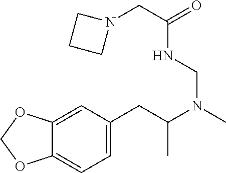 [SMILES: CC(Cc1ccc2c(c1)OCO2)N(C)CNC(=O)CN1CCC1] |
| | CC(CC1═CC═C2OCOC2═C1)N |
| | (C)CNC(CN3CCC3)═O |
| 236 | 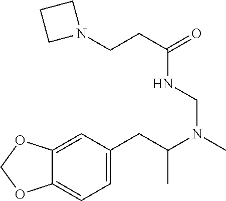 [SMILES: CC(Cc1ccc2c(c1)OCO2)N(C)CNC(=O)CCN1CCC1] |
| | CC(CC1═CC═C2OCOC2═C1)N |
| | (C)CNC(CCN3CCC3)═O |
| 237 | 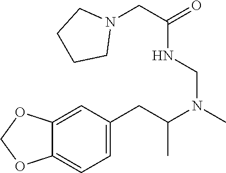 [SMILES: CC(Cc1ccc2c(c1)OCO2)N(C)CNC(=O)CN1CCCC1] |
| | CC(CC1═CC═C2OCOC2═C1)N |
| | (C)CNC(CN3CCCC3)═O |
| 238 | 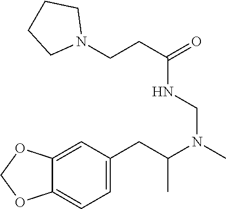 [SMILES: CC(Cc1ccc2c(c1)OCO2)N(C)CNC(=O)CCN1CCCC1] |
| | CC(CC1═CC═C2OCOC2═C1)N |
| | (C)CNC(CCN3CCCC3)═O |
| 239 | 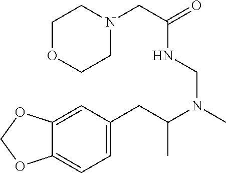 [SMILES: CC(Cc1ccc2c(c1)OCO2)N(C)CNC(=O)CN1CCOCC1] |
| | CC(CC1═CC═C2OCOC2═C1)N |
| | (C)CNC(CN3CCOCC3)═O |
| 240 | 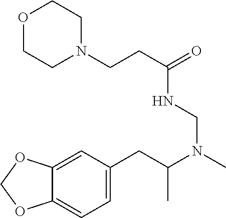 [SMILES: CC(Cc1ccc2c(c1)OCO2)N(C)CNC(=O)CCN1CCOCC1] |
| | CC(CC1═CC═C2OCOC2═C1)N |
| | (C)CNC(CCN3CCOCC3)═O |
| 241 | 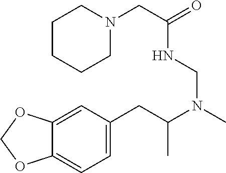 [SMILES: CC(Cc1ccc2c(c1)OCO2)N(C)CNC(=O)CN1CCCCC1] |
| | CC(CC1═CC═C2OCOC2═C1)N |
| | (C)CNC(CN3CCCCC3)═O |
| 242 | 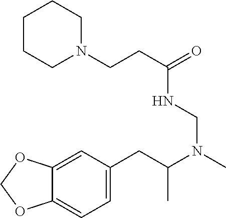 [SMILES: CC(Cc1ccc2c(c1)OCO2)N(C)CNC(=O)CCN1CCCCC1] |
| | CC(CC1═CC═C2OCOC2═C1)N |
| | (C)CNC(CCN3CCCCC3)═O |
| 243 | 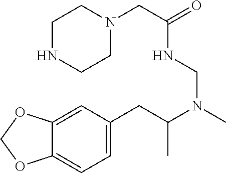 [SMILES: CC(Cc1ccc2c(c1)OCO2)N(C)CNC(=O)CN1CCNCC1] |
| | CC(CC1═CC═C2OCOC2═C1)N |
| | (C)CNC(CN3CCNCC3)═O |
| 244 | 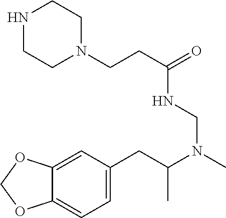 [SMILES: CC(Cc1ccc2c(c1)OCO2)N(C)CNC(=O)CCN1CCNCC1] |
| | CC(CC1═CC═C2OCOC2═C1)N |
| | (C)CNC(CCN3CCNCC3)═O |
| 245 | 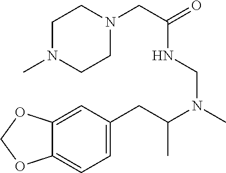 [SMILES: CC(Cc1ccc2c(c1)OCO2)N(C)CNC(=O)CN1CCN(C)CC1] |
| | CC(CC1═CC═C2OCOC2═C1)N |
| | (C)CNC(CN3CCN(C)CC3)═O |
| 246 | 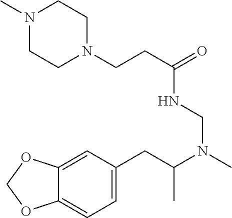 |
| | CC(CC1═CC═C2OCOC2═C1)N |
| | (C)CNC(CCN3CCN(C)CC3)═O |
| 247 | 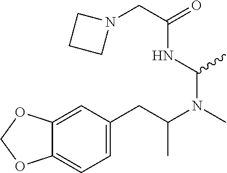 [SMILES: CC(Cc1ccc2c(c1)OCO2)N(C)C(C)NC(=O)CN1CCC1] |
| | CC(CC1═CC═C2OCOC2═C1)N |
| | (C)C(C)NC(CN3CCC3)═O |
| 248 | 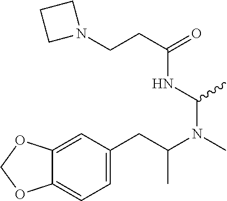 [SMILES: CC(Cc1ccc2c(c1)OCO2)N(C)C(C)NC(=O)CCN1CCC1] |
| | CC(CC1═CC═C2OCOC2═C1)N |
| | (C)C(C)NC(CCN3CCC3)═O |
| 249 | 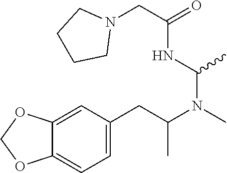 [SMILES: CC(Cc1ccc2c(c1)OCO2)N(C)C(C)NC(=O)CN1CCCC1] |
| | CC(CC1═CC═C2OCOC2═C1)N |
| | (C)C(C)NC(CN3CCCC3)═O |
| 250 | 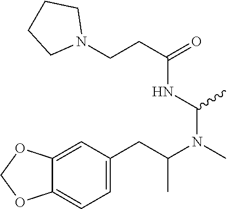 [SMILES: CC(Cc1ccc2c(c1)OCO2)N(C)C(C)NC(=O)CCN1CCCC1] |
| | CC(CC1═CC═C2OCOC2═C1)N |
| | (C)C(C)NC(CCN3CCCC3)═O |
| 251 | 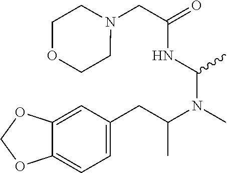 [SMILES: CC(Cc1ccc2c(c1)OCO2)N(C)C(C)NC(=O)CN1CCOCC1] |
| | CC(CC1═CC═C2OCOC2═C1)N |
| | (C)C(C)NC(CN3CCOCC3)═O |
| 252 | 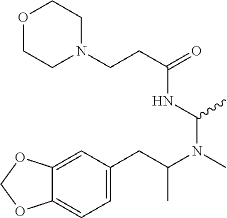 [SMILES: CC(Cc1ccc2c(c1)OCO2)N(C)C(C)NC(=O)CCN1CCOCC1] |
| | CC(CC1═CC═C2OCOC2═C1)N |
| | (C)C(C)NC(CCN3CCOCC3)═O |
| 253 | 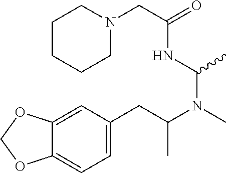 [SMILES: CC(Cc1ccc2c(c1)OCO2)N(C)C(C)NC(=O)CN1CCCCC1] |
| | CC(CC1═CC═C2OCOC2═C1)N |
| | (C)C(C)NC(CN3CCCCC3)═O |
| 254 | 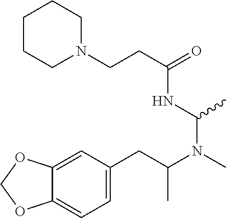 [SMILES: CC(Cc1ccc2c(c1)OCO2)N(C)C(C)NC(=O)CCN1CCCCC1] |
| | CC(CC1═CC═C2OCOC2═C1)N |
| | (C)C(C)NC(CCN3CCCCC3)═O |
| 255 | 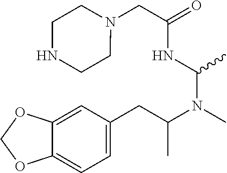 [SMILES: CC(Cc1ccc2c(c1)OCO2)N(C)C(C)NC(=O)CN1CCNCC1] |
| | CC(CC1═CC═C2OCOC2═C1)N |
| | (C)C(C)NC(CN3CCNCC3)═O |
| 256 | 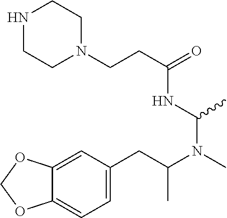 [SMILES: CC(Cc1ccc2c(c1)OCO2)N(C)C(C)NC(=O)CCN1CCNCC1] |
| | CC(CC1═CC═C2OCOC2═C1)N |
| | (C)C(C)NC(CCN3CCNCC3)═O |
| 257 | 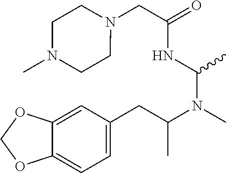 [SMILES: CC(Cc1ccc2c(c1)OCO2)N(C)C(C)NC(=O)CN1CCN(C)CC1] |
| | CC(CC1═CC═C2OCOC2═C1)N |
| | (C)C(C)NC(CN3CCN(C)CC3)═O |
| 258 | 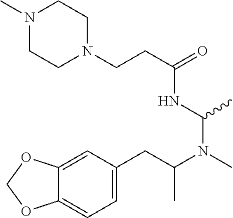 [SMILES: CC(Cc1ccc2c(c1)OCO2)N(C)C(C)NC(=O)CCN1CCN(C)CC1] |
| | CC(CC1═CC═C2OCOC2═C1)N |
| | (C)C(C)NC(CCN3CCN(C)CC3)═O |
| 259 | 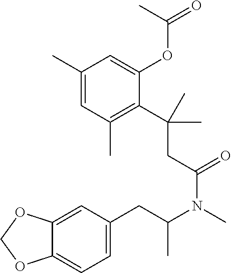 [SMILES: CC(=O)Oc1cc(C)cc(C)c1C(C)(C)CC(=O)N(C)C(C)Cc1ccc2c(c1)OCO2] |
| | CC(CC1═CC═C2OCOC2═C1)N |
| | (C(CC(C)(C)C(C(OC(C)═O)═CC |
| | (C)═C3)═C3C)═O)C |
| 260 | 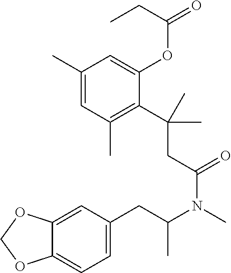 [SMILES: CCC(=O)Oc1cc(C)cc(C)c1C(C)(C)CC(=O)N(C)C(C)Cc1ccc2c(c1)OCO2] |
| | CC(CC1═CC═C2OCOC2═C1)N |
| | (C(CC(C)(C)C(C(OC(CC)═O)═C |
| | C(C)═C3)═C3C)═O)C |
| 261 | 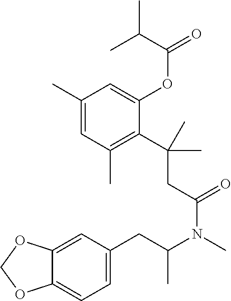 [SMILES: Cc1cc(C)c(C(C)(C)CC(=O)N(C)C(C)Cc2ccc3c(c2)OCO3)c(OC(=O)C(C)C)c1] |
| | CC(CC1═CC═C2OCOC2═C1)N |
| | (C(CC(C)(C)C(C(OC(C(C)C)═ |
| | O)═CC(C)═C3)═C3C)═O)C |
| 262 | 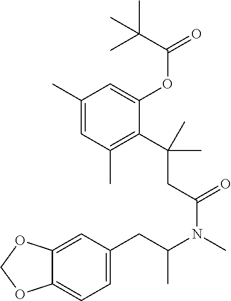 [SMILES: Cc1cc(C)c(C(C)(C)CC(=O)N(C)C(C)Cc2ccc3c(c2)OCO3)c(OC(=O)C(C)(C)C)c1] |
| | CC(CC1═CC═C2OCOC2═C1)N |
| | (C(CC(C)(C)C(C(OC(C(C)(C)C)═ |
| | O)═CC(C)═C3)═C3C)═O)C |
| 263 | 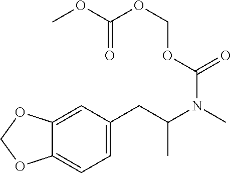 [SMILES: COC(=O)OCOC(=O)N(C)C(C)Cc1ccc2c(c1)OCO2] |
| | CC(CC1═CC═C2OCOC2═C1)N |
| | (C(OCOC(OC)═O)═O)C |
| 264 | 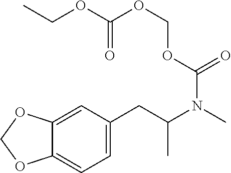 [SMILES: CCOC(=O)OCOC(=O)N(C)C(C)Cc1ccc2c(c1)OCO2] |
| | CC(CC1═CC═C2OCOC2═C1)N |
| | (C(OCOC(OCC)═O)═O)C |
| 266 | 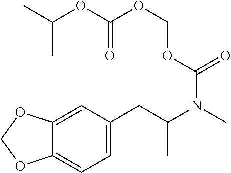 [SMILES: CC(C)OC(=O)OCOC(=O)N(C)C(C)Cc1ccc2c(c1)OCO2] |
| | CC(CC1═CC═C2OCOC2═C1)N |
| | (C(OCOC(OC(C)C)═O)═O)C |
| 267 | 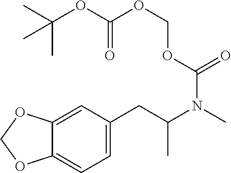 [SMILES: CC(Cc1ccc2c(c1)OCO2)N(C)C(=O)OCOC(=O)OC(C)(C)C] |
| | CC(CC1═CC═C2OCOC2═C1)N |
| | (C(OCOC(OC(C)(C)C)═O)═O)C |
| 268 | 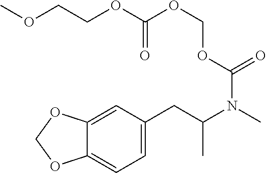 [SMILES: COCCOC(=O)OCOC(=O)N(C)C(C)Cc1ccc2c(c1)OCO2] |
| | CC(CC1═CC═C2OCOC2═C1)N |
| | (C(OCOC(OCCOC)═O)═O)C |
| 269 | 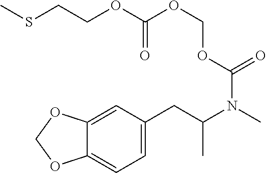 [SMILES: CSCCOC(=O)OCOC(=O)N(C)C(C)Cc1ccc2c(c1)OCO2] |
| | CC(CC1═CC═C2OCOC2═C1)N |
| | (C(OCOC(OCCSC)═O)═O)C |
| 270 | 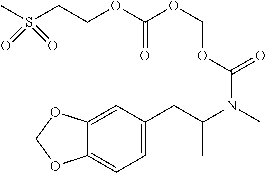 [SMILES: CC(Cc1ccc2c(c1)OCO2)N(C)C(=O)OCOC(=O)OCCS(C)(=O)=O] |
| | CC(CC1═CC═C2OCOC2═C1)N |
| | (C(OCOC(OCCS(C)(═O)═O)═O)═ |
| | O)C |
| 271 | 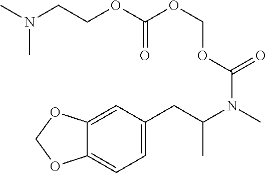 [SMILES: CC(Cc1ccc2c(c1)OCO2)N(C)C(=O)OCOC(=O)OCCN(C)C] |
| | CC(CC1═CC═C2OCOC2═C1)N |
| | (C(OCOC(OCCN(C)C)═O)═O)C |
| 272 | 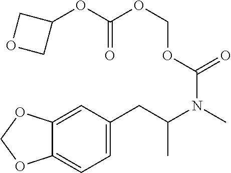 [SMILES: CC(Cc1ccc2c(c1)OCO2)N(C)C(=O)OCOC(=O)OC1COC1] |
| | CC(CC1═CC═C2OCOC2═C1)N |
| | (C(OCOC(OC3COC3)═O)═O)C |
| 273 | 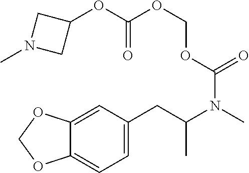 [SMILES: CC(Cc1ccc2c(c1)OCO2)N(C)C(=O)OCOC(=O)OC1CN(C)C1] |
| | CC(CC1═CC═C2OCOC2═C1)N |
| | (C(OCOC(OC3CN(C)C3)═O)═O) |
| | C |
| 274 | 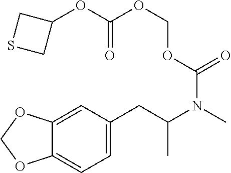 [SMILES: CC(Cc1ccc2c(c1)OCO2)N(C)C(=O)OCOC(=O)OC1CSC1] |
| | CC(CC1═CC═C2OCOC2═C1)N |
| | (C(OCOC(OC3CSC3)═O)═O)C |
| 275 | 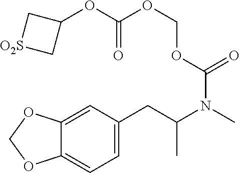 [SMILES: CC(Cc1ccc2c(c1)OCO2)N(C)C(=O)OCOC(=O)OC1CS(=O)(=O)C1] |
| | CC(CC1═CC═C2OCOC2═C1)N |
| | (C(OCOC(OC3CS(C3)(═O)═O)═ |
| | O)═O)C |
| 276 | 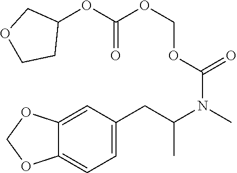 [SMILES: CC(Cc1ccc2c(c1)OCO2)N(C)C(=O)OCOC(=O)OC1CCOC1] |
| | CC(CC1═CC═C2OCOC2═C1)N |
| | (C(OCOC(OC3COCC3)═O)═O)C |
| 277 | 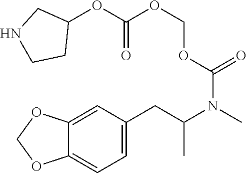 [SMILES: CC(Cc1ccc2c(c1)OCO2)N(C)C(=O)OCOC(=O)OC1CCNC1] |
| | CC(CC1═CC═C2OCOC2═C1)N |
| | (C(OCOC(OC3CNCC3)═O)═O)C1 |
| 278 | 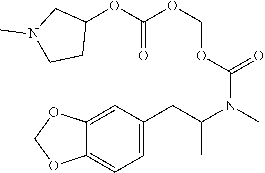 [SMILES: CC(Cc1ccc2c(c1)OCO2)N(C)C(=O)OCOC(=O)OC1CCN(C)C1] |
| | CC(CC1═CC═C2OCOC2═C1)N |
| | (C(OCOC(OC3CN(C)CC3)═O)═O)C |
| 279 | 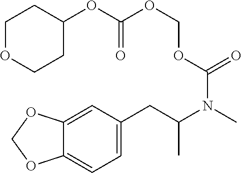 [SMILES: CC(Cc1ccc2c(c1)OCO2)N(C)C(=O)OCOC(=O)OC1CCOCC1] |
| | CC(CC1═CC═C2OCOC2═C1)N |
| | (C(OCOC(OC3CCOCC3)═O)═O) |
| | C |
| 280 | 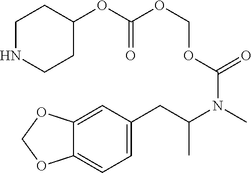 [SMILES: CC(Cc1ccc2c(c1)OCO2)N(C)C(=O)OCOC(=O)OC1CCNCC1] |
| | CC(CC1═CC═C2OCOC2═C1)N |
| | (C(OCOC(OC3CCNCC3)═O)═O) |
| | C |
| 281 | 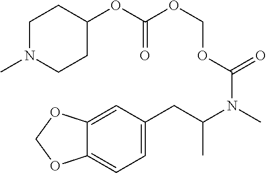 [SMILES: CC(Cc1ccc2c(c1)OCO2)N(C)C(=O)OCOC(=O)OC1CCN(C)CC1] |
| | CC(CC1═CC═C2OCOC2═C1)N |
| | (C(OCOC(OC3CCN(C)CC3)═O)═ |
| | O)C |
| 282 | 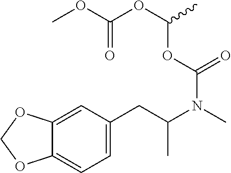 [SMILES: COC(=O)OC(C)OC(=O)N(C)C(C)Cc1ccc2c(c1)OCO2] |
| | CC(CC1═CC═C2OCOC2═C1)N |
| | (C(OC(C)OC(OC)═O)═O)C |
| 283 | 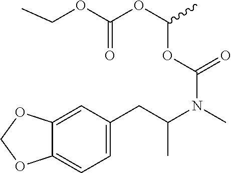 [SMILES: CCOC(=O)OC(C)OC(=O)N(C)C(C)Cc1ccc2c(c1)OCO2] |
| | CC(CC1═CC═C2OCOC2═C1)N |
| | (C(OC(C)OC(OCC)═O)═O)C |
| 284 | 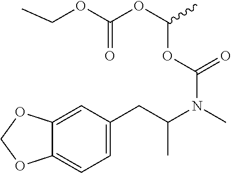 [SMILES: CCOC(=O)OC(C)OC(=O)N(C)C(C)Cc1ccc2c(c1)OCO2] |
| | CC(CC1═CC═C2OCOC2═C1)N |
| | (C(OC(C)OC(OC(C)C)═O)═O)C |
| 285 | 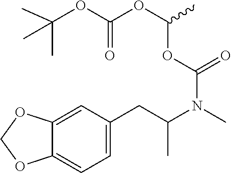 [SMILES: CC(OC(=O)OC(C)(C)C)OC(=O)N(C)C(C)Cc1ccc2c(c1)OCO2] |
| | CC(CC1═CC═C2OCOC2═C1)N |
| | (C(OC(C)OC(OC(C)(C)C)═O)═O) |
| | C |
| 286 | 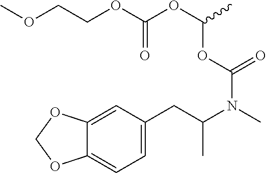 [SMILES: COCCOC(=O)OC(C)OC(=O)N(C)C(C)Cc1ccc2c(c1)OCO2] |
| | CC(CC1═CC═C2OCOC2═C1)N |
| | (C(OC(C)OC(OCCOC)═O)═O)C |
| 287 | 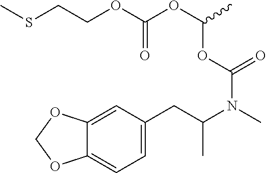 [SMILES: CSCCOC(=O)OC(C)OC(=O)N(C)C(C)Cc1ccc2c(c1)OCO2] |
| | CC(CC1═CC═C2OCOC2═C1)N |
| | (C(OC(C)OC(OCCSC)═O)═O)C |
| 288 | 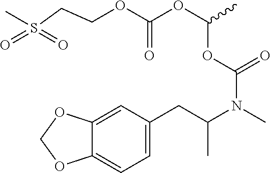 [SMILES: CC(OC(=O)OCCS(C)(=O)=O)OC(=O)N(C)C(C)Cc1ccc2c(c1)OCO2] |
| | CC(CC1═CC═C2OCOC2═C1)N |
| | (C(OC(C)OC(OCCS(C)(═O)═O)═ |
| | O)═O)C |
| 289 | 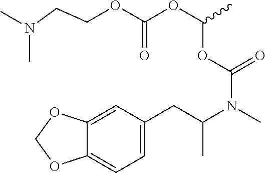 [SMILES: CC(OC(=O)OCCN(C)C)OC(=O)N(C)C(C)Cc1ccc2c(c1)OCO2] |
| | CC(CC1═CC═C2OCOC2═C1)N |
| | (C(OC(C)OC(OCCN(C)C)═O)═O) |
| | C |
| 290 | 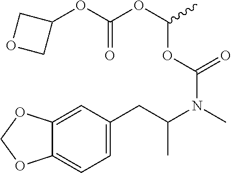 [SMILES: CC(OC(=O)OC1COC1)OC(=O)N(C)C(C)Cc1ccc2c(c1)OCO2] |
| | CC(CC1═CC═C2OCOC2═C1) |
| | N(C(OC(C)OC(OC3COC3)═O)═O) |
| | C |
| 291 | 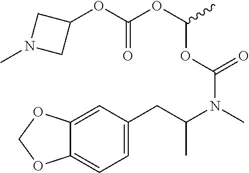 [SMILES: CC(OC(=O)OC1CN(C)C1)OC(=O)N(C)C(C)Cc1ccc2c(c1)OCO2] |
| | CC(CC1═CC═C2OCOC2═C1)N |
| | (C(OC(C)OC(OC3CN(C)C3)═ |
| | O)═O)C |
| 292 | 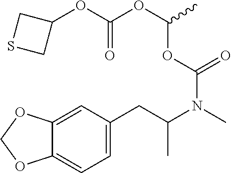 [SMILES: CC(OC(=O)OC1CSC1)OC(=O)N(C)C(C)Cc1ccc2c(c1)OCO2] |
| | CC(CC1═CC═C2OCOC2═C1) |
| | N(C(OC(C)OC(OC3CSC3)═O)═O)C |
| 293 | 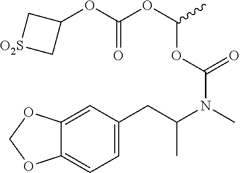 [SMILES: CC(OC(=O)OC1CS(=O)(=O)C1)OC(=O)N(C)C(C)Cc1ccc2c(c1)OCO2] |
| | CC(CC1═CC═C2OCOC2═C1)N |
| | (C(OC(C)OC(OC3CS(C3)(═O)═ |
| | O)═O)═O)C |
| 294 | 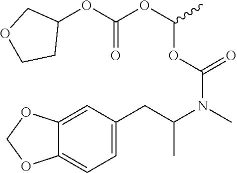 [SMILES: CC(OC(=O)OC1CCOC1)OC(=O)N(C)C(C)Cc1ccc2c(c1)OCO2] |
| | CC(CC1═CC═C2OCOC2═C1)N |
| | (C(OC(C)OC(OC3COCC3)═O)═O)C |
| 295 | 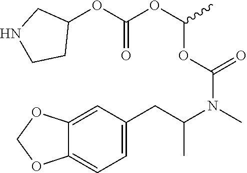 [SMILES: CC(OC(=O)OC1CCNC1)OC(=O)N(C)C(C)Cc1ccc2c(c1)OCO2] |
| | CC(CC1═CC═C2OCOC2═C1)N |
| | (C(OC(C)OC(OC3CNCC3)═O)═O)C |
| 296 | 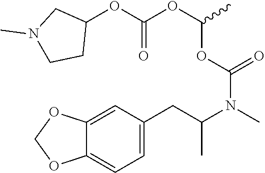 [SMILES: CC(OC(=O)OC1CCN(C)C1)OC(=O)N(C)C(C)Cc1ccc2c(c1)OCO2] |
| | CC(CC1═CC═C2OCOC2═C1)N |
| | (C(OC(C)OC(OC3CN(C)CC3)═ |
| | O)═O)C |
| 297 | 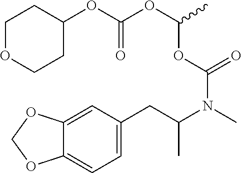 [SMILES: CC(OC(=O)OC1CCOCC1)OC(=O)N(C)C(C)Cc1ccc2c(c1)OCO2] |
| | CC(CC1═CC═C2OCOC2═C1)N |
| | (C(OC(C)OC(OC3CCOCC3)═O)═ |
| | O)C |
| 298 | 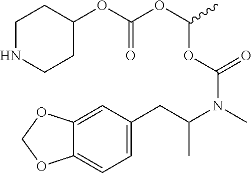 [SMILES: CC(OC(=O)OC1CCNCC1)OC(=O)N(C)C(C)Cc1ccc2c(c1)OCO2] |
| | CC(CC1═CC═C2OCOC2═C1)N |
| | (C(OC(C)OC(OC3CCNCC3)═O)═ |
| | O)C |
| 299 | 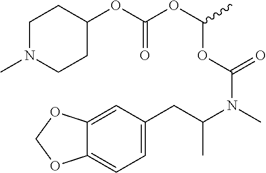 [SMILES: CC(OC(=O)OC1CCN(C)CC1)OC(=O)N(C)C(C)Cc1ccc2c(c1)OCO2] |
| | CC(CC1═CC═C2OCOC2═C1)N |
| | (C(OC(C)OC(OC3CCN(C)CC3)═ |
| | O)═O)C |
| 300 | 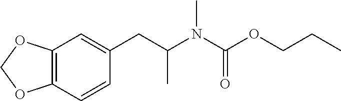 [SMILES: CCCOC(=O)N(C)C(C)Cc1ccc2c(c1)OCO2] |
| 301 | 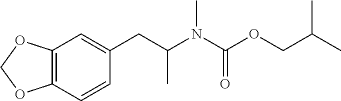 [SMILES: CC(C)COC(=O)N(C)C(C)Cc1ccc2c(c1)OCO2] |
| 302 | 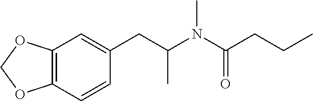 [SMILES: CCCC(=O)N(C)C(C)Cc1ccc2c(c1)OCO2] |
| 303 | 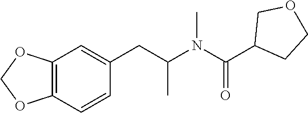 [SMILES: CC(Cc1ccc2c(c1)OCO2)N(C)C(=O)C1CCOC1] |
| 304 | 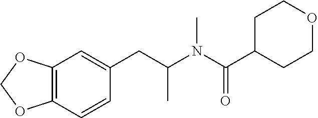 [SMILES: CC(Cc1ccc2c(c1)OCO2)N(C)C(=O)C1CCOCC1] |
| 305 | 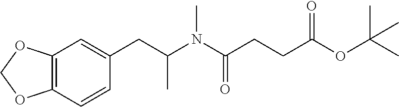 [SMILES: CC(Cc1ccc2c(c1)OCO2)N(C)C(=O)CCC(=O)OC(C)(C)C] |
| 306 | 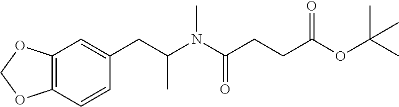 [SMILES: CC(Cc1ccc2c(c1)OCO2)N(C)C(=O)CCC(=O)OC(C)(C)C] |
| 307 | 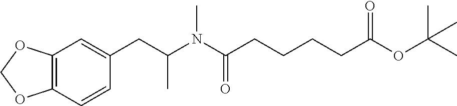 [SMILES: CC(Cc1ccc2c(c1)OCO2)N(C)C(=O)CCCCC(=O)OC(C)(C)C] |
| 308 | 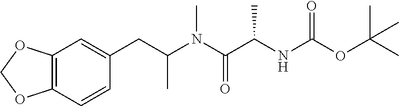 [SMILES: CC(Cc1ccc2c(c1)OCO2)N(C)C(=O)[C@H](C)NC(=O)OC(C)(C)C] |
| 309 | 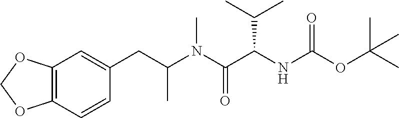 [SMILES: CC(C)[C@H](NC(=O)OC(C)(C)C)C(=O)N(C)C(C)Cc1ccc2c(c1)OCO2] |
| 310 | 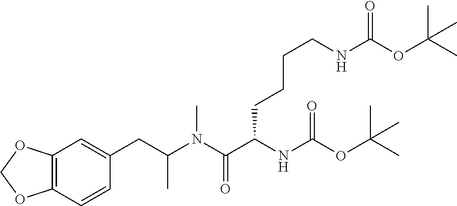 [SMILES: CC(Cc1ccc2c(c1)OCO2)N(C)C(=O)[C@H](CCCCNC(=O)OC(C)(C)C)NC(=O)OC(C)(C)C] |
| 311 | 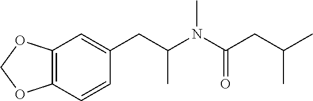 [SMILES: CC(C)CC(=O)N(C)C(C)Cc1ccc2c(c1)OCO2] |
| 312 | 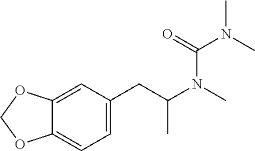 [SMILES: CC(Cc1ccc2c(c1)OCO2)N(C)C(=O)N(C)C] |
| 313 | 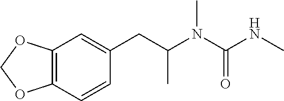 [SMILES: CNC(=O)N(C)C(C)Cc1ccc2c(c1)OCO2] |
| 314 | 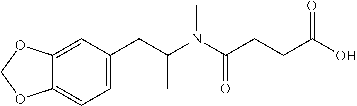 [SMILES: CC(Cc1ccc2c(c1)OCO2)N(C)C(=O)CCC(=O)O] |
| 315 | 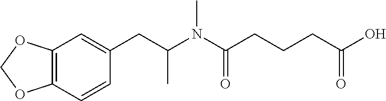 [SMILES: CC(Cc1ccc2c(c1)OCO2)N(C)C(=O)CCCC(=O)O] |
| 316 | 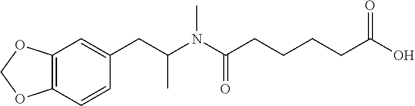 [SMILES: CC(Cc1ccc2c(c1)OCO2)N(C)C(=O)CCCCC(=O)O] |
| 317 | 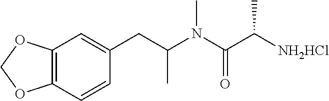 |
| 318 | 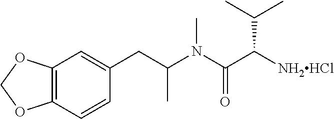 |
| 319 | 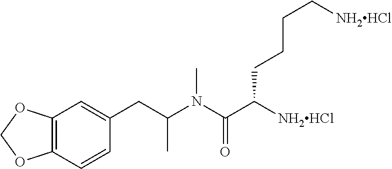 |
| 320 | 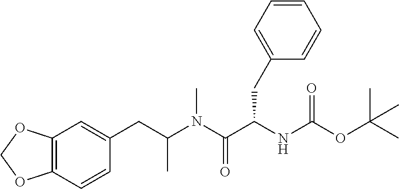 [SMILES: CC(Cc1ccc2c(c1)OCO2)N(C)C(=O)[C@H](Cc1ccccc1)NC(=O)OC(C)(C)C] |
| 321 | 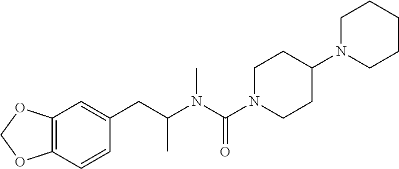 [SMILES: CC(Cc1ccc2c(c1)OCO2)N(C)C(=O)N1CCC(N2CCCCC2)CC1] |
| 322 | 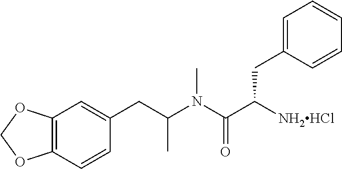 |
| 323 | 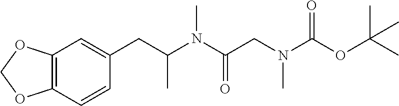 [SMILES: CC(Cc1ccc2c(c1)OCO2)N(C)C(=O)CN(C)C(=O)OC(C)(C)C] |
| 324 | 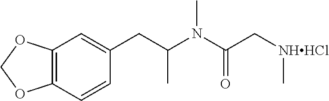 |
| 325 | 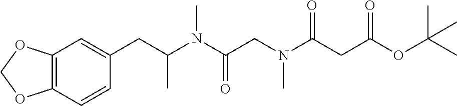 [SMILES: CC(Cc1ccc2c(c1)OCO2)N(C)C(=O)CN(C)C(=O)CC(=O)OC(C)(C)C] |
| 326 | 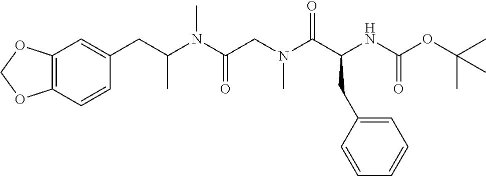 [SMILES: CC(Cc1ccc2c(c1)OCO2)N(C)C(=O)CN(C)C(=O)[C@H](Cc1ccccc1)NC(=O)OC(C)(C)C] |
| 327 | 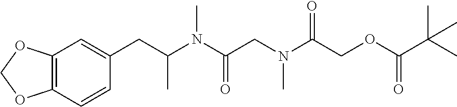 [SMILES: CC(Cc1ccc2c(c1)OCO2)N(C)C(=O)CN(C)C(=O)COC(=O)C(C)(C)C] |
| 328 | 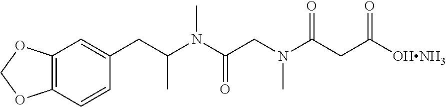 |
| 328A | 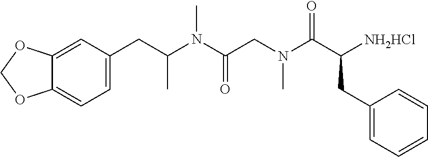 |
| 329 | 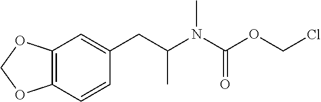 [SMILES: CC(Cc1ccc2c(c1)OCO2)N(C)C(=O)OCCl] |
| 330 | 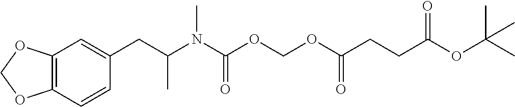 [SMILES: CC(Cc1ccc2c(c1)OCO2)N(C)C(=O)OCOC(=O)CCC(=O)OC(C)(C)C] |
| 331 | 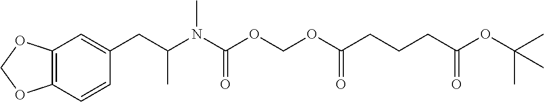 [SMILES: CC(Cc1ccc2c(c1)OCO2)N(C)C(=O)OCOC(=O)CCCC(=O)OC(C)(C)C] |
| 332 | 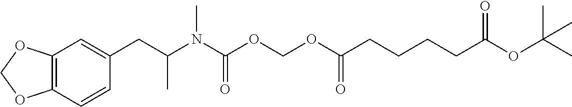 [SMILES: CC(Cc1ccc2c(c1)OCO2)N(C)C(=O)OCOC(=O)CCCCC(=O)OC(C)(C)C] |
| 333 | 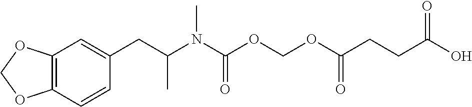 [SMILES: CC(Cc1ccc2c(c1)OCO2)N(C)C(=O)OCOC(=O)CCC(=O)O] |
| 334 | 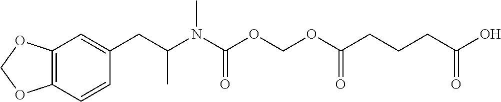 [SMILES: CC(Cc1ccc2c(c1)OCO2)N(C)C(=O)OCOC(=O)CCCC(=O)O] |
| 335 | 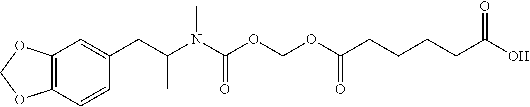 [SMILES: CC(Cc1ccc2c(c1)OCO2)N(C)C(=O)OCOC(=O)CCCCC(=O)O] |
| 336 | 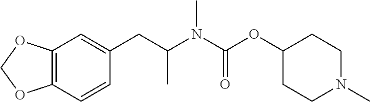 [SMILES: CC(Cc1ccc2c(c1)OCO2)N(C)C(=O)OC1CCN(C)CC1] |
| 337 | 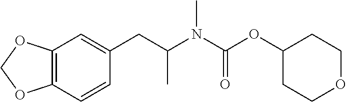 [SMILES: CC(Cc1ccc2c(c1)OCO2)N(C)C(=O)OC1CCOCC1] |
| 338 | 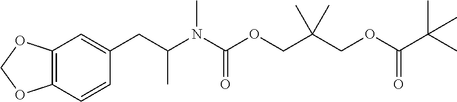 [SMILES: CC(Cc1ccc2c(c1)OCO2)N(C)C(=O)OCC(C)(C)COC(=O)C(C)(C)C] |
| 339 | 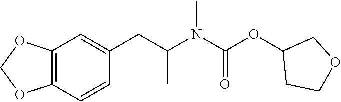 [SMILES: CC(Cc1ccc2c(c1)OCO2)N(C)C(=O)OC1CCOC1] |
| 340 | 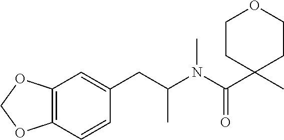 [SMILES: CC(Cc1ccc2c(c1)OCO2)N(C)C(=O)C1(C)CCOCC1] |
| 341 | 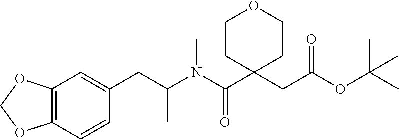 [SMILES: CC(Cc1ccc2c(c1)OCO2)N(C)C(=O)C1(CC(=O)OC(C)(C)C)CCOCC1] |
| 342 | 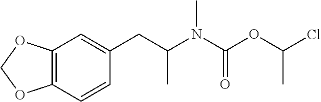 [SMILES: CC(Cl)OC(=O)N(C)C(C)Cc1ccc2c(c1)OCO2] |
| 343 | 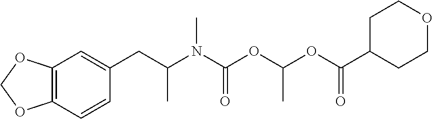 [SMILES: CC(OC(=O)C1CCOCC1)OC(=O)N(C)C(C)Cc1ccc2c(c1)OCO2] |
| 344 | 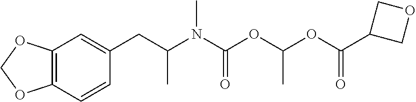 [SMILES: CC(OC(=O)C1COC1)OC(=O)N(C)C(C)Cc1ccc2c(c1)OCO2] |
| 345 | 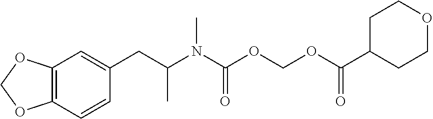 [SMILES: CC(Cc1ccc2c(c1)OCO2)N(C)C(=O)OCOC(=O)C1CCOCC1] |
| 346 | 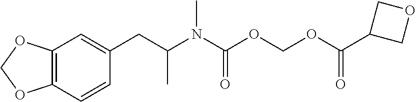 [SMILES: CC(Cc1ccc2c(c1)OCO2)N(C)C(=O)OCOC(=O)C1COC1] |
| 347 | 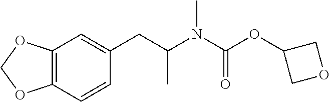 [SMILES: CC(Cc1ccc2c(c1)OCO2)N(C)C(=O)OC1COC1] |
| 348 | 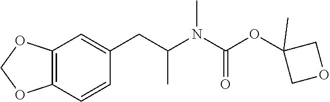 [SMILES: CC(Cc1ccc2c(c1)OCO2)N(C)C(=O)OC1(C)COC1] |
| 349 | 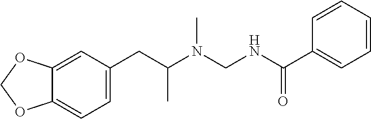 [SMILES: CC(Cc1ccc2c(c1)OCO2)N(C)CNC(=O)c1ccccc1] |
| 350 | 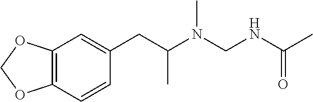 [SMILES: CC(=O)NCN(C)C(C)Cc1ccc2c(c1)OCO2] |

In some embodiments, the compound described herein is a compound selected from Table 1.

In some embodiments, the compound described herein a compound selected from Table 1A below.

| Cpd | Structure | Name |
|---|---|---|
| 50 | 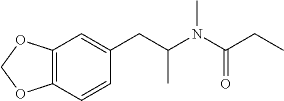 [SMILES: CCC(=O)N(C)C(C)Cc1ccc2c(c1)OCO2] | N-[2-(2H-1,3-Benzodioxol-5-y1)-1- methyl-ethyl]-N-methylpropionamide |
| 302 | 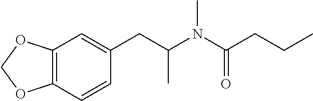 [SMILES: CCCC(=O)N(C)C(C)Cc1ccc2c(c1)OCO2] | N-[2-(2H-1,3-Benzodioxol-5-y1)-1- methyl-ethyl]-N-methylbutyramide |
| 303 | 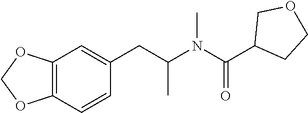 [SMILES: CC(Cc1ccc2c(c1)OCO2)N(C)C(=O)C1CCOC1] | N-[2-(2H-1,3-Benzodioxol-5-y1)-1- methyl-ethyl]-N-methyl-tetrahydro-3- furamide |
| 304 | 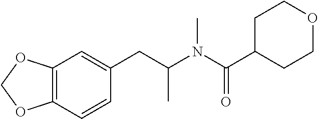 [SMILES: CC(Cc1ccc2c(c1)OCO2)N(C)C(=O)C1CCOCC1] | N-[2-(2H-1,3-Benzodioxol-5-y1)-1- methyl-ethyl]-N-methyl-tetrahydro-2H- pyran-4-carboxamide |
| 305 | 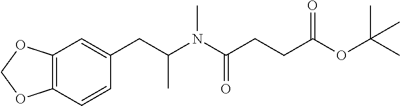 [SMILES: CC(Cc1ccc2c(c1)OCO2)N(C)C(=O)CCC(=O)OC(C)(C)C] | tert-Butyl 3-{[2-(2H-1,3-benzodioxol-5- y1)-1-methyl-ethyl]-N- methylcarbamoyl } propionate |
| 306 | 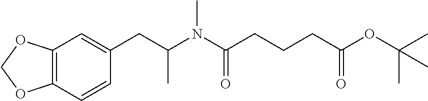 [SMILES: CC(Cc1ccc2c(c1)OCO2)N(C)C(=O)CCCC(=O)OC(C)(C)C] | tert-Butyl 3-{[2-(2H-1,3-benzodioxol-5- y1)-1-methyl-ethyl]-N- methylcarbamoyl } propionate |
| 307 | 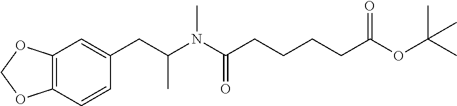 [SMILES: CC(Cc1ccc2c(c1)OCO2)N(C)C(=O)CCCCC(=O)OC(C)(C)C] | tert-Butyl 5-{[2-(2//-1,3-benzodioxol-5- yl)-1-methyl-ethyl]-N- methylcarbamoyl }valerate |
| 259 | 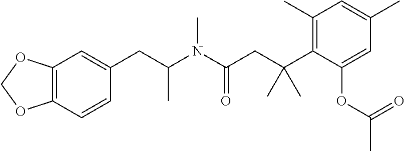 [SMILES: CC(=O)Oc1cc(C)cc(C)c1C(C)(C)CC(=O)N(C)C(C)Cc1ccc2c(c1)OCO2] | 2-(2-{[2-(2H-1,3-Benzodioxol-5-y1)-1- methylethyl]-N-methylcarbamoyl}-1,1- dimethylethyl)-3,5-xylyl acetate |
| 94 | 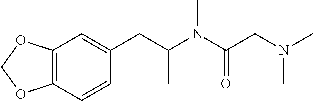 [SMILES: CC(Cc1ccc2c(c1)OCO2)N(C)C(=O)CN(C)C] | N-[2-(2H-1,3-Benzodioxol-5-y1)-1- methyl-ethyl]-N- methyl(dimethylamino)acetamide |
| 308 | 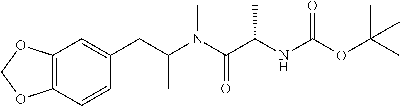 [SMILES: CC(Cc1ccc2c(c1)OCO2)N(C)C(=O)[C@H](C)NC(=O)OC(C)(C)C] | (S)-1-{[2-(2H-1,3-Benzodioxol-5-y1)-1- methyl-ethy1]-N- methylcarbamoyl }ethylamino-tert- butylformylate |
| 309 | 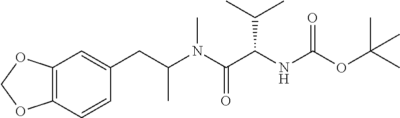 [SMILES: CC(C)[C@H](NC(=O)OC(C)(C)C)C(=O)N(C)C(C)Cc1ccc2c(c1)OCO2] | (S)-1-{[2-(2H-1,3-Benzodioxol-5-y1)-1- methyl-ethyl]-N-methylcarbamoyl}-2- methylpropylamino-tert-butylformylate |
| 310 | 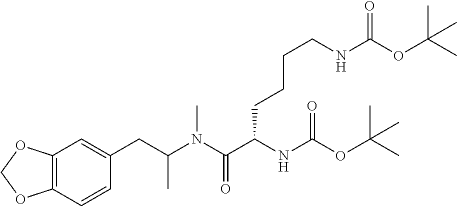 [SMILES: CC(Cc1ccc2c(c1)OCO2)N(C)C(=O)[C@H](CCCCNC(=O)OC(C)(C)C)NC(=O)OC(C)(C)C] | (S)-1-{[2-(2H-1,3-Benzodioxol-5-y1)-1- methyl-ethyl]-N-methylcarbamoy1}-5- (tert-butoxycarbonylamino)pentylamino- tert-butylformylate |
| 68 | 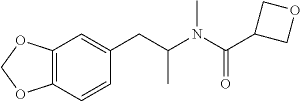 [SMILES: CC(Cc1ccc2c(c1)OCO2)N(C)C(=O)C1COC1] | N-[2-(2H-1,3-Benzodioxol-5-y1)-1- methyl-ethyl]-N-methyl-3- oxetanecarboxamide |
| 320 | 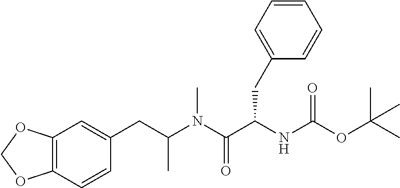 [SMILES: CC(Cc1ccc2c(c1)OCO2)N(C)C(=O)[C@H](Cc1ccccc1)NC(=O)OC(C)(C)C] | (S)-1-{[2-(2H-1,3-Benzodioxol-5-y1)-1- methyl-ethyl]-N-methylcarbamoy1}-2- phenylethylamino-tert-butylformylate |
| 106 | 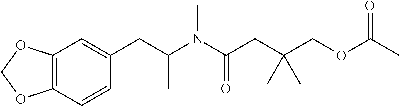 [SMILES: CC(=O)OCC(C)(C)CC(=O)N(C)C(C)Cc1ccc2c(c1)OCO2] | (3-{[2-(2H-1,3-Benzodioxol-5-yl)-1- methylethyl]-N-methylcarbamoy1}-2,2- dimethylpropyl acetate |
| 51 | 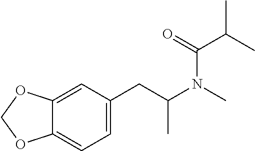 [SMILES: CC(C)C(=O)N(C)C(C)Cc1ccc2c(c1)OCO2] | N-[2-(1,3-Benzodioxol-5-y1)-1-methyl- ethyl]-N,2-dimethyl-propanamide |
| 311 | 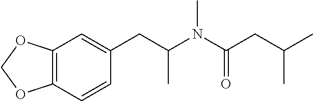 [SMILES: CC(C)CC(=O)N(C)C(C)Cc1ccc2c(c1)OCO2] | N-[2-(2H-1,3-Benzodioxol-5-y1)-1- methyl-ethyl]-N-methyl-3- methylbutyramide |
| 52 | 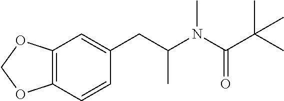 [SMILES: CC(Cc1ccc2c(c1)OCO2)N(C)C(=O)C(C)(C)C] | N-[2-(2H-1,3-Benzodioxol-5-y1)-1- methyl-ethyl]-N-methyl-2,2- dimethylpropionamide |
| 16 | 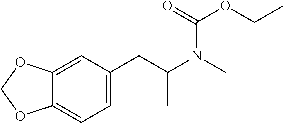 [SMILES: CCOC(=O)N(C)C(C)Cc1ccc2c(c1)OCO2] | Ethyl N-[2-(1,3-benzodioxol-5-yl)-1- methyl-ethyl]-N-methyl-carbamate |
| 300 | 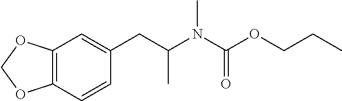 [SMILES: CCCOC(=O)N(C)C(C)Cc1ccc2c(c1)OCO2] | propyl N-[2-(1,3-benzodioxol-5-yl)-1- methyl-ethyl]-N-methyl-carbamate |
| 17 | 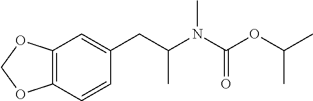 [SMILES: CC(C)OC(=O)N(C)C(C)Cc1ccc2c(c1)OCO2] | isopropyl N-[2-(1,3-benzodioxol-5-yl)-1- methyl-ethyl]-N-methyl-carbamate |
| 301 | 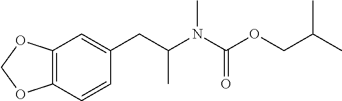 [SMILES: CC(C)COC(=O)N(C)C(C)Cc1ccc2c(c1)OCO2] | isobutyl N-[2-(1,3-benzodioxol-5-y1)-1- methyl-ethyl]-N-methyl-carbamate |
| 2 | 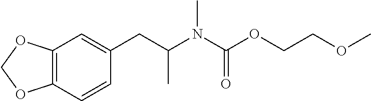 [SMILES: COCCOC(=O)N(C)C(C)Cc1ccc2c(c1)OCO2] | 2-methoxyethyl N-[2-(1,3-benzodioxol-5- y1)-1-methyl-ethyl]-N-methyl-carbamate |
| 113 | 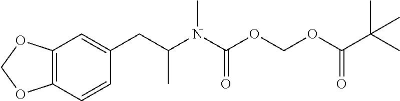 [SMILES: CC(Cc1ccc2c(c1)OCO2)N(C)C(=O)OCOC(=O)C(C)(C)C] | [[2-(1,3-benzodioxol-5-y1)-1-methyl- ethyl]-methyl-carbamoyl]oxymethyl-2,2- dimethylpropanoate |
| 18 | 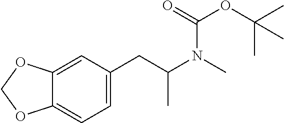 [SMILES: CC(Cc1ccc2c(c1)OCO2)N(C)C(=O)OC(C)(C)C] | tert-Butyl N-[2-(1,3-benzodioxol-5-y1)-1- methyl-ethyl]-N-methyl-carbamate |
| 312 | 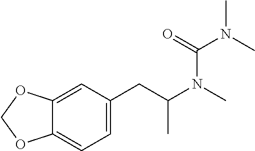 [SMILES: CC(Cc1ccc2c(c1)OCO2)N(C)C(=O)N(C)C] | 1-[2-(2H-1,3-Benzodioxol-5-y1)-1- methyl-ethyl]-1,3,3-trimethylurea |
| 313 | 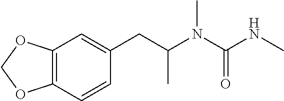 [SMILES: CNC(=O)N(C)C(C)Cc1ccc2c(c1)OCO2] | 1-[2-(2H-1,3-Benzodioxol-5-y1)-1- methyl-ethyl]-1,3-dimethylurea |
| 321 | 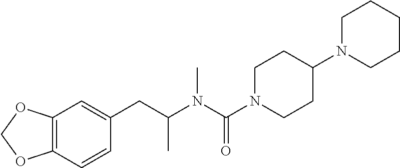 [SMILES: CC(Cc1ccc2c(c1)OCO2)N(C)C(=O)N1CCC(N2CCCCC2)CC1] | N-[2-(1,3-Benzodioxol-5-yl)-1-methyl- ethyl]-N-methyl-4-(1- piperidyl)piperidine-1-carboxamide |
| 314 | 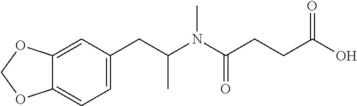 [SMILES: CC(Cc1ccc2c(c1)OCO2)N(C)C(=O)CCC(=O)O] | 3-{[2-(2H-1,3-Benzodioxol-5-y1)-1- methyl-ethyl]-N- methylcarbamoyl} propionic acid |
| 315 | 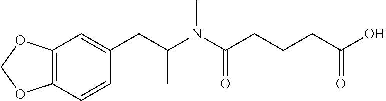 [SMILES: CC(Cc1ccc2c(c1)OCO2)N(C)C(=O)CCCC(=O)O] | 4-{[2-(2H-1,3-Benzodioxol-5-y1)-1- methyl-ethyl]-N-methylcarbamoyl}butyric acid |
| 316 | 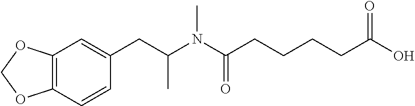 [SMILES: CC(Cc1ccc2c(c1)OCO2)N(C)C(=O)CCCCC(=O)O] | 5-{[2-(2H-1,3-Benzodioxol-5-yl)-1- methyl-ethyl]-N-methylcarbamoyl }valeric acid |
| 317 | 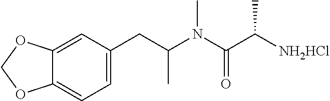 | 5-{[2-(2/-1,3-Benzodioxol-5-y1)-1- methyl-ethyl]-N-methylcarbamoyl}valeric acid |
| 318 | 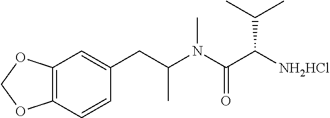 | (2S)-2-amino-N-[2-(1,3-benzodioxol-5- yl)-1-methyl-ethyl]-N,3-dimethyl- butanamide hydrochloride |
| 319 | 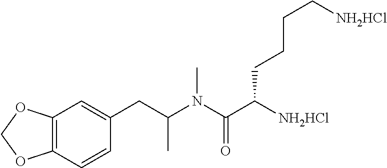 | (2S)-2,6-diamino-N-[2-(1,3-benzodioxol- 5-y1)-1-methyl-ethyl]-N-methyl- hexanamide dihydrochloride |
| 322 | 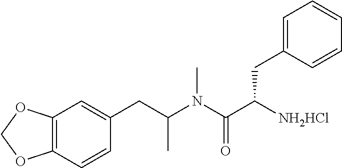 | (2S)-2-Amino-N-[2-(1,3-benzodioxol-5- yl)-1-methyl-ethyl]-N-methyl-3-phenyl- propanamide hydrochloride |
| 323 | 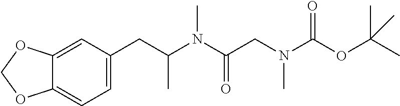 [SMILES: CC(Cc1ccc2c(c1)OCO2)N(C)C(=O)CN(C)C(=O)OC(C)(C)C] | tert-Butyl N-[2-[[2-(1,3-benzodioxol-5- yl)-1-methyl-ethyl]-methyl-amino]-2-oxo- ethyl]-N-methyl-carbamate |
| 324 | 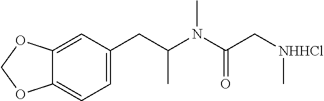 | N-[2-(1,3-Benzodioxol-5-y1)-1-methyl- ethyl]-N-methyl-2- (methylamino)acetamide hydrochloride |
| 325 | 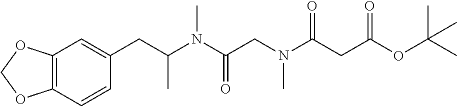 [SMILES: CC(Cc1ccc2c(c1)OCO2)N(C)C(=O)CN(C)C(=O)CC(=O)OC(C)(C)C] | tert-Butyl [({[2-(2H-1,3-benzodioxol-5- y1)-1-methyl-ethyl]-N- methylcarbamoyl }methyl)-N- methylcarbamoyl]acetate |
| 326 | 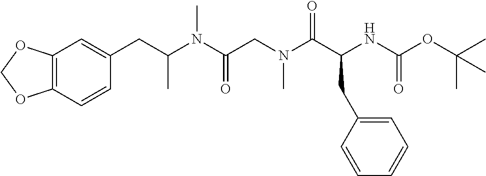 [SMILES: CC(Cc1ccc2c(c1)OCO2)N(C)C(=O)CN(C)C(=O)[C@H](Cc1ccccc1)NC(=O)OC(C)(C)C] | (S)-1-[{{[2-(2H-1,3-Benzodioxol-5-y1)-1- methylethyl]-N- methylcarbamoyl }methyl)-N- methylcarbamoyl]-2-phenylethylamino- tert-butylformylate |
| 327 | 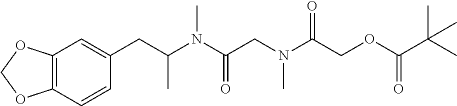 [SMILES: CC(Cc1ccc2c(c1)OCO2)N(C)C(=O)CN(C)C(=O)COC(=O)C(C)(C)C] | [{{[2-(2H-1,3-Benzodioxol-5-y1)-1- methylethyl]-N- methylcarbamoyl }methyl)-N- methylcarbamoyl]methyl 2,2- dimethylpropionate |
| 328 | 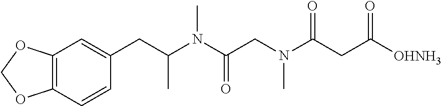 | Ammonium 3-[2-[[2-(1,3-benzodioxol-5- yl)-1-methyl-ethyl]-methyl-amino]-2-oxo- ethyl]-methyl-amino]-3-oxo-propionate |
| 328A | 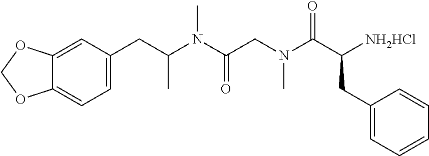 | (2S)-2-amino-N-[2-[[2-(1,3-benzodioxol- 5-y1)-1-methyl-ethyl]-methyl-amino]-2- oxo-ethyl]-N-methyl-3-phenyl- propanamide |
| 329 | 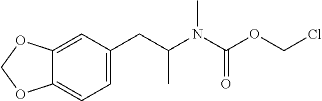 [SMILES: CC(Cc1ccc2c(c1)OCO2)N(C)C(=O)OCCl] | Chloromethyl N-[2-(1,3-benzodioxol-5- y1)-1-methyl-ethyl]-N-methyl-carbamate |
| 330 | 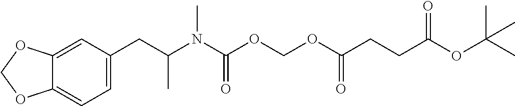 [SMILES: CC(Cc1ccc2c(c1)OCO2)N(C)C(=O)OCOC(=O)CCC(=O)OC(C)(C)C] | {[2-(2H-1,3-Benzodioxol-5-y1)-1- methylethyl]-N- methylaminocarbonyloxy }methyl tert- butyl succinate |
| 331 | 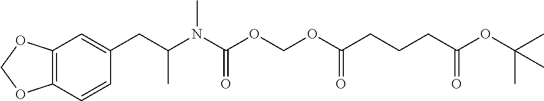 [SMILES: CC(Cc1ccc2c(c1)OCO2)N(C)C(=O)OCOC(=O)CCCC(=O)OC(C)(C)C] | {[2-(2H-1,3-Benzodioxol-5-yl)-1- methylethyl]-N- methylaminocarbonyloxy }methyl tert- butyl glutarate |
| 332 | 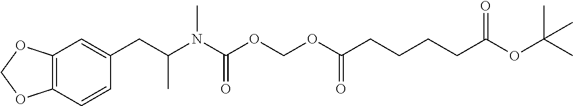 [SMILES: CC(Cc1ccc2c(c1)OCO2)N(C)C(=O)OCOC(=O)CCCCC(=O)OC(C)(C)C] | {[2-(2H-1,3-Benzodioxol-5-y1)-1- methylethyl]-N- methylaminocarbonyloxy }methyl tert- butyl adipate |
| 333 | 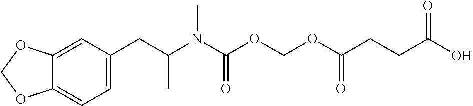 [SMILES: CC(Cc1ccc2c(c1)OCO2)N(C)C(=O)OCOC(=O)CCC(=O)O] | 4-[[[2-(1,3-Benzodioxol-5-y1)-1-methyl- ethyl]-methyl-carbamoyl]oxymethoxy]-4- oxo-butanoic acid |
| 334 | 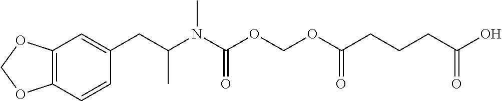 [SMILES: CC(Cc1ccc2c(c1)OCO2)N(C)C(=O)OCOC(=O)CCCC(=O)O] | 5-[[[2-(1,3-Benzodioxol-5-y1)-1-methyl- ethyl]-methyl-carbamoyl]oxymethoxy]-5- oxo-pentanoic acid |
| 335 | 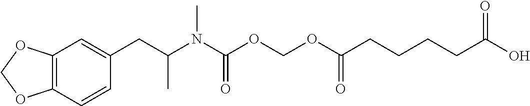 [SMILES: CC(Cc1ccc2c(c1)OCO2)N(C)C(=O)OCOC(=O)CCCCC(=O)O] | 6-[[[2-(1,3-Benzodioxol-5-yl)-1-methyl- ethyl]-methyl-carbamoyl]oxymethoxy]-6- oxo-hexanoic acid |
| 336 | 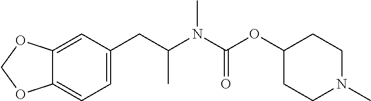 [SMILES: CC(Cc1ccc2c(c1)OCO2)N(C)C(=O)OC1CCN(C)CC1] | (1-Methyl-4-piperidyl) N-[2-(1,3- benzodioxol-5-y1)-1-methyl-ethyl]-N- methyl-carbamate |
| 337 | 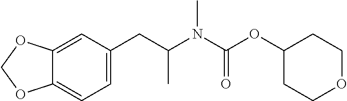 [SMILES: CC(Cc1ccc2c(c1)OCO2)N(C)C(=O)OC1CCOCC1] | Tetrahydropyran-4-yl N-[2-(1,3- benzodioxol-5-y1)-1-methyl-ethyl]-N- methyl-carbamate |
| 338 | 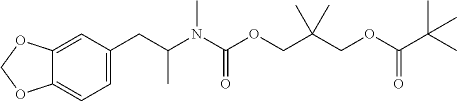 [SMILES: CC(Cc1ccc2c(c1)OCO2)N(C)C(=O)OCC(C)(C)COC(=O)C(C)(C)C] | [3-[[2-(1,3-Benzodioxol-5-y1)-1-methyl- ethyl]-methyl-carbamoyl]oxy-2,2- dimethyl-propyl] 2,2-dimethylpropanoate |
| 339 | 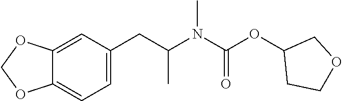 [SMILES: CC(Cc1ccc2c(c1)OCO2)N(C)C(=O)OC1CCOC1] | Tetrahydrofuran-3-y1 N-[2-(1,3- benzodioxol-5-y1)-1-methyl-ethyl]-N- methyl-carbamate |
| 340 | 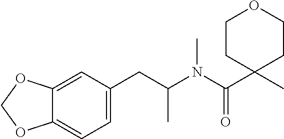 [SMILES: CC(Cc1ccc2c(c1)OCO2)N(C)C(=O)C1(C)CCOCC1] | N-[2-(2H-1,3-Benzodioxol-5-y1)-1- methylethyl]-N-methyl-4- methyltetrahydro-2H-pyran-4- carboxamide |
| 341 | 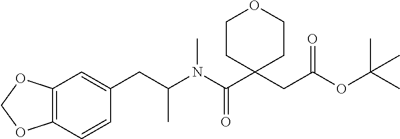 [SMILES: CC(Cc1ccc2c(c1)OCO2)N(C)C(=O)C1(CC(=O)OC(C)(C)C)CCOCC1] | tert-Butyl (4-{[2-(2H-1,3-benzodioxol-5- yl)-1-methyl-ethyl]-N- methylcarbamoyl } tetrahydro-2H-pyran-4- yl)acetate |
| 342 | 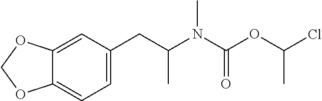 [SMILES: CC(Cl)OC(=O)N(C)C(C)Cc1ccc2c(c1)OCO2] | 1-Chloroethyl N-[2-(1,3-benzodioxol-5- y1)-1-methyl-ethyl]-N-methyl-carbamate |
| 343 | 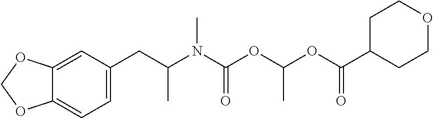 [SMILES: CC(OC(=O)C1CCOCC1)OC(=O)N(C)C(C)Cc1ccc2c(c1)OCO2] | 1-{[2-(2H-1,3-Benzodioxol-5-y1)-1- methyl-ethyl]-N- methylaminocarbonyloxy }ethyl tetrahydro-2H-pyran-4-carboxylate |
| 344 | 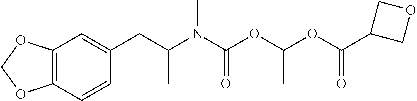 [SMILES: CC(OC(=O)C1COC1)OC(=O)N(C)C(C)Cc1ccc2c(c1)OCO2] | 1-{[2-(2H-1,3-Benzodioxol-5-y1)-1- methyl-ethyl]-N- methylaminocarbonyloxy }ethyl 3- oxetanecarboxylate |
| 345 | 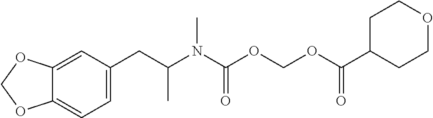 [SMILES: CC(Cc1ccc2c(c1)OCO2)N(C)C(=O)OCOC(=O)C1CCOCC1] | {[2-(2H-1,3-Benzodioxol-5-yl)-1-methyl- ethyl]-N- methylaminocarbonyloxy }methyl tetrahydro-2H-pyran-4-carboxylate |
| 346 | 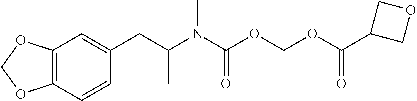 [SMILES: CC(Cc1ccc2c(c1)OCO2)N(C)C(=O)OCOC(=O)C1COC1] | {[2-(2H-1,3-Benzodioxol-5-y1)-1-methyl- ethyl]-N- methylaminocarbonyloxy }methyl 3- oxetanecarboxylate |
| 347 | 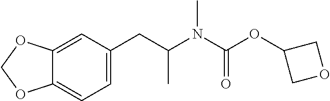 [SMILES: CC(Cc1ccc2c(c1)OCO2)N(C)C(=O)OC1COC1] | Oxetan-3-yl N-[2-(1,3-benzodioxol-5-yl)- 1-methyl-ethyl]-N-methyl-carbamate |
| 348 | 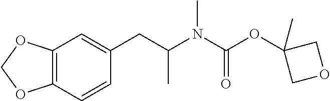 [SMILES: CC(Cc1ccc2c(c1)OCO2)N(C)C(=O)OC1(C)COC1] | (3-Methyloxetan-3-y1) N-[2-(1,3- benzodioxol-5-yl)-1-methyl-ethyl]-N- methyl-carbamate |
| 349 | 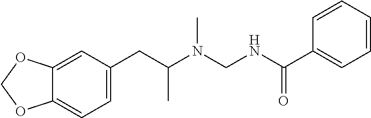 [SMILES: CC(Cc1ccc2c(c1)OCO2)N(C)CNC(=O)c1ccccc1] | N-({[2-(2H-1,3-Benzodioxol-5-y1)-1- methyl-ethyl]-N- methylamino}methyl)benzamide |
| 350 | 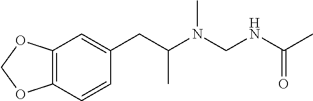 [SMILES: CC(=O)NCN(C)C(C)Cc1ccc2c(c1)OCO2] | N-({[2-(2H-1,3-Benzodioxol-5-yl)-1- methyl-ethyl]-N- methylamino} methyl)acetamide |

In another aspect, the present disclosure provides a pharmaceutically acceptable composition comprising a compound according to any of Formula (I), (I-1), (I-1-1), (Ia), (Ib), (VI-1b), (VI-2), (VI-2a), (VI-2b), (VI-2.1), (VI-2.1a), (VI-2.1b), (VI-2.2), (VI-2.2a), (VI-2.2b) (VI-3), (VI-3a), (VI-3b), (VII), (VIIa), (VIIb), (VII-1), (VII-1a), (VII-1b), (VII-2), (VII-2a), (VII-2b), (VII-2.1), (VII-2.1a), (VII-2.1b), (VII-2.2), (VII-2.2a), (VII-2.2b) (VII-3), (VII-3a), (VII-3b), (VIII), (VIIIa), (VIIIb), (IX), (IXa), or (IXb), or a pharmaceutically acceptable salt thereof, and a pharmaceutically acceptable excipient, carrier, adjuvant, or vehicle.

Pharmaceutical compositions of the present disclosure can comprise racemic, scalemic, or diastereomerically enriched mixtures of any compound described herein.

In some embodiments, the present disclosure provides a pharmaceutical composition comprising a mixture of diastereomers of a compound of Formula (I), (I-1), (I-1-1), (II), (III), 2.2), (VII-3), (VIII), or (IX), or a pharmaceutically acceptable salt thereof, wherein at least about 60%, at least about 70%, at least about 80%, at least about 90%, at least about 95%, at least about 98%, at least about 99%, at least about 99.5%, or at least about 99.9% of molecules in the mixture comprise a ((S)-1-(benzo[d][1,3]dioxol-5-yl)propan-2-yl)(methyl)amino moiety.

In some embodiments, the present disclosure provides a pharmaceutical composition comprising a mixture of diastereomers of a compound of Formula (I), (I-1), (I-1-1), (II), (III), (IV), (V), (VI), (VI-1), (VI-2), (VI-2.1), (VI-2.2), (VI-3), (VII), (VII-1), (VII-2), (VII-2.1), (VII-2.2), (VII-3), (VIII), or (IX), or a pharmaceutically acceptable salt thereof, wherein at least about 60%, at least about 70%, at least about 80%, at least about 90%, at least about 95%, at least about 98%, at least about 99%, at least about 99.5%, or at least about 99.9% of molecules in the mixture comprise a ((R)-1-(benzo[d][1,3]dioxol-5-yl)propan-2-yl)(methyl)amino moiety.

In some embodiments, the present disclosure provides a pharmaceutical composition comprising a mixture of diastereomers of a compound of Formula (I), (I-1), (I-1-1), (II), (III), (IV), (V), (VI), (VI-1), (VI-2), (VI-2.1), (VI-2.2), (VI-3), (VII), (VII-1), (VII-2), (VII-2.1), (VII-2.2), (VII-3), (VIII), or (IX), or a pharmaceutically acceptable salt thereof, wherein about 50% of molecules in the mixture comprise a ((R)-1-(benzo[d][1,3]dioxol-5-yl)propan-2-yl)(methyl)amino moiety.

In some embodiments, the present disclosure provides a pharmaceutical composition comprising a mixture of diastereomers of a compound of Formula (I), (I-1), (I-1-1), (II), (III), (IV), (V), (VI), (VI-1), (VI-2), (VI-2.1), (VI-2.2), (VI-3), (VII), (VII-1), (VII-2), (VII-2.1), (VII-2.2), (VII-3), (VIII), or (IX), or a pharmaceutically acceptable salt thereof, wherein from about 48% to about 52% of molecules in the mixture comprise a ((R)-1-(benzo[d][1,3]dioxol-5-yl)propan-2-yl)(methyl)amino moiety.

In some embodiments, the present disclosure provides a pharmaceutical composition comprising a mixture of diastereomers of a compound of Formula (I), (I-1), (I-1-1), (II), (III), (IV), (V), (VI), (VI-1), (VI-2), (VI-2.1), (VI-2.2), (VI-3), (VII), (VII-1), (VII-2), (VII-2.1), (VII-2.2), (VII-3), (VIII), or (IX), or a pharmaceutically acceptable salt thereof, wherein from about 55% to about 99.99%, from about 60% to about 99.99%, from about 70% to about 99.99%, from about 80% to about 99.99%, from about 90% to about 99.99%, from about 95% to about 99.99%, from about 98% to about 99.99%, from about 99% to about 99.99%, from about 99.5% to about 99.99%, or from about 99.9% to about 99.99% of molecules in the mixture comprise a ((R)-1-(benzo[d][1,3]dioxol-5-yl)propan-2-yl)(methyl)amino moiety.

In some embodiments, the present disclosure provides a pharmaceutical composition comprising a mixture of diastereomers of a compound of Formula (I), (I-1), (I-1-1), (II), (III), (IV), (V), (VI), (VI-1), (VI-2), (VI-2.1), (VI-2.2), (VI-3), (VII), (VII-1), (VII-2), (VII-2.1), (VII-2.2), (VII-3), (VIII), or (IX), or a pharmaceutically acceptable salt thereof, wherein from about 55% to about 99.99%, from about 60% to about 99.99%, from about 70% to about 99.99%, from about 80% to about 99.99%, from about 90% to about 99.99%, from about 95% to about 99.99%, from about 98% to about 99.99%, from about 99% to about 99.99%, from about 99.5% to about 99.99%, or from about 99.9% to about 99.99% of molecules in the mixture comprise a ((S)-1-(benzo[d][1,3]dioxol-5-yl)propan-2-yl)(methyl)amino moiety.

### Methods of Treatment.

In yet another aspect, the present disclosure provides a method of treating or preventing a disease, disorder, or condition in which an increased level of a phenethylamine psychedelic such as MDMA is beneficial, comprising administering to a subject in need thereof an effective amount of a compound of Formula (I), (I-1), (I-1-1), (Ia), (Ib), (II), (IIb), (III), (IIIa), (IIIb), (IV), (IVa), (IVb), (V), (Va), (VI), (VIa), (VIb), (VI-1), (VI-1a), (VI-1b), (VI-2), (VI-2a), (VI-2b), (VI-2.1), (VI-2.1a), (VI-2.1b), (VI-2.2), (VI-2.2a), (VI-2.2b) (VI-3), (VI-3a), (VI-3b), (VII), (VIIa), (VIIb), (VII-1), (VII-1a), (VII-1b), (VII-2), (VII-2a), (VII-2b), (VII-2.1), (VII-2.1a), (VII-2.1b), (VII-2.2), (VII-2.2a), (VII-2.2b) (VII-3), (VII-3a), (VII-3b), (VIII), (VIIIa), (VIIIb), (IX), (IXa), or (IXb), or a pharmaceutically acceptable salt thereof. In some embodiments, the condition comprises post-traumatic stress disorder, major depression, schizophrenia, Alzheimer's disease, frontotemporal dementia, Parkinson's disease, Parkinson's dementia, dementia, Lewy body dementia, multiple system atrophy, or substance abuse. In some embodiments, the condition comprises musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. The compounds of the present invention can also be used to treat any brain disease.

In some embodiments, a compound disclosed herein has activity as a 5-HT_{2A} modulator. In some embodiments a compound disclosed herein elicits a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity. 5-HT_{2A} antagonists abrogate the neuritogenesis and spinogenesis effects of hallucinogenic compounds with 5-HT_{2A} agonist activity, for example, DMT, LSD, and DOI. In some embodiments, a compound disclosed herein is a 5-HT_{2A} modulator and promotes neural plasticity (e.g., cortical structural plasticity). In some embodiments, a compound disclosed herein is a selective 5-HT_{2A} modulator and promotes neural plasticity (e.g., cortical structural plasticity). Promotion of neural plasticity can include, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. In some embodiments, a compound of the present disclosure functions as a 5-HT_{2A} agonist alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, flibanserin, nelotanserin and lorcaserin. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior administration of a compound disclosed herein, such as about three or about hours prior administration of the compound. In some embodiments, the serotonin receptor modulator is administered at most about one hour prior to the compound. In some embodiments, the second therapeutic agent is a serotonin receptor modulator. In some embodiments, the serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, a compound of the present disclosure is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments, a compound herein is given to patients in a low dose that is lower than would produce noticeable psychedelic effects but high enough to provide a therapeutic benefit. This dose range is predicted to be between 200 μg (micrograms) and 2 mg.

In some embodiments, a compound described herein is used to treat a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present disclosure is used for increasing neuronal plasticity. In some embodiments, a compound described herein is used for treating a brain disorder. In some embodiments, a compound described herein is used for increasing translation, transcription, or secretion of neurotrophic factors.

A compound disclosed herein can also be useful for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound the present disclosure can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides a method for increasing neuronal plasticity, comprising contacting a neuronal cell with a compound of the present disclosure. In some embodiments, increasing neuronal plasticity improves a brain disorder described herein.

In some embodiments, a compound disclosed herein is used to increase neuronal plasticity and has, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). Brain disorders can include, for example, migraines, addiction (e.g., substance use disorder), depression, and anxiety.

In some embodiments, the experiment or assay to determine increased neuronal plasticity derived from the administration of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the condition is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, a compound of the present disclosure is used to treat brain disorders. In some embodiments, the compound has, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present disclosure provides a method of treating a brain disorder, comprising administering to a subject in need thereof a therapeutically effective amount of a compound disclosed herein. In some embodiments, the brain disorder is a neurodegenerative disorder, Alzheimer's disease, Parkinson's disease, a psychological disorder, depression, addiction, anxiety, post-traumatic stress disorder, treatment resistant depression, suicidal ideation, major depressive disorder, bipolar disorder, schizophrenia, stroke, traumatic brain injury, or a substance use disorder.

In some embodiments, the brain disorder is a neurodegenerative disorder, Alzheimer's disease or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent. Non-limiting examples of additional therapeutics suitable for administration with a compound of the present disclosure can include lithium, olanzapine (Zyprexa), quetiapine (Seroquel), risperidone (Risperdal), aripiprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), GHB or gamma hydroxybutyrate or sodium oxybate, or clonazepam (Klonopin).

In some embodiments, a compound of the present disclosure is used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors.

As used herein, the term “neurotrophic factor” can refer to a family of soluble peptides or proteins which support the survival, growth, and differentiation of developing and mature neurons. Increasing at least one of translation, transcription, or secretion of neurotrophic factors can be useful for, for example, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors increases neuronal plasticity. In some embodiments, increasing at least one of translation, transcription, or secretion of neurotrophic factors promotes neuronal growth, promotes neuritogenesis, promotes synaptogenesis, promotes dendritogenesis, increases dendritic arbor complexity, and/or increases dendritic spine density.

In some embodiments, a 5-HT_{2A} modulators (e.g., 5-HT_{2A} agonists) is used to increase at least one of translation, transcription, or secretion of neurotrophic factors. In some embodiments, a compound of the present disclosure is used to increase translation, transcription, or secretion of neurotrophic factors. In some embodiments, increasing translation, transcription or secretion of neurotrophic factors is sufficient for the treatment of migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, or addiction (e.g., substance use disorder).

An experiment or assay can be used to detect increased translation of neurotrophic factors, which can include, for example, ELISA, western blot, an immunofluorescence assay, a proteomic experiment, and mass spectrometry. In some embodiments, the experiment or assay used to detect increased transcription of neurotrophic factors is a gene expression assay, PCR, or microarray. In some embodiments, the experiment or assay used to detect increased secretion of neurotrophic factors is ELISA, western blot, an immunofluorescence assay, a proteomic experiment, or a mass spectrometry assay.

In some embodiments, the present disclosure provides a method for increasing translation, transcription, or secretion of neurotrophic factors, wherein the method comprises contacting a neuronal cell with a compound disclosed herein.

### EXAMPLES

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMR. Abbreviations used are those conventional in the art. If not defined, the terms have their generally accepted meanings.

### General Conditions for Characterization:

Mass spectra were run on LC-MS systems using electrospray ionization. These were run using a Waters Acquity Classic UPLC with PDA and SQ mass detection or a Waters Acquity H-Class UPLC with PDA and QDA mass detection. [M+H]+ refers to mono-isotopic molecular weights.

NMR spectra were run on Bruker Ultrashield 400 MHz or 500 MHz NMR spectrometer. Spectra were recorded at 298 K, unless otherwise stated, and were referenced using the solvent peak.

### Abbreviation

app apparent
Boc tert-butyl carbamate
Boc-Sar-OH Boc-sarcosine
br broad
CDCl₃ d₃-chloroform
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EtOAc ethyl acetate
HATU 1-[Bis(dimethylamino)methylene]-1H-1,2,3-triazolo[4,5-b]pyridinium 3-oxid hexafluorophosphate
HCl hydrochloric acid
h hextet; sextet
hr or hrs hour or hours
HPLC high pressure liquid chromatography
LC-MS liquid chromatography and mass spectrometry
MeOH MeOH
MeCN acetonitrile
MS mass spectrometry
m multiplet
min(s) minute(s)
mL milliliter(s)
L microliter(s)
m z mass to charge ratio
p pentet
q quartet
NaHCO₃ sodium hydrogen carbonate
Na₂SO₄ sodium sulfate
NMP N-methyl-2-pyrrolidone
NMR nuclear magnetic resonance
Rt retention time
s singlet
sar sarcosine
t triplet
tert tertiary
THE tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification. More specific compounds required for the syntheses are listed below:
5-(tert-Butoxy)-5-oxopentanoic acid (CAS No: 63128-51-8) purchased from Sigma Aldrich (catalogue number SY3H3D678586)
6-(tert-Butoxy)-6-oxohexanoic acid (CAS No: 52221-07-5) purchased from BLDpharm (catalogue number BD00759729)
3-(2-Acetoxy-4,6-dimethylphenyl)-3-methylbutyric acid (CAS No: 134098-68-3) purchased from Sigma Aldrich (catalogue number 756377)
2-Methoxyethyl chloroformate (CAS No: 628-12-6) purchased from Enamine (catalogue number EN300-222696)
[(chlorocarbonyl)oxy]methyl 2,2-dimethylpropanoate (CAS No: 133217-74-0) purchased from Enamine (catalogue number EN300-371)

### HPLC Conditions

If not indicated otherwise, the analytical HPLC conditions are as follows:

### Instrument: Lc-Ms-1:

Method 2A

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, 0.1% formic acid, B: MeCN |
| Gradient: | 0.0-1.8 min 2-98% B, 1.8-2.1 min 98% B, |
| | 2.1-2.5 98% A. |

Method 2B

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, 0.1% ammonia B: MeCN |
| Gradient: | 0.0-1.8 min 2-98% B, 1.8-2.1 min 98% B, |
| | 2.1-2.5 98% A. |

### Instrument: LC-MS-2:

Method 2A

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: | A: H₂O, B: MeCN, C: 50% H₂O/50% MeCN + 2.0% |
| | formic acid |
| Gradient: | 0.0-1.7 mins 0-95% B, 5% C; 1.7-2.1 mins 95% B, 5% C |
| | 2.1-2.5 mins 95% A, 5% C. |

Method 2B

| | |
|---|---|
| Column: | Acquity UPLC BEH C18 2.1 × 50 mm 1.7 μm |
| Column Temp: | 50° C. |
| Flow rate: | 0.8 mL/min. |
| Eluents: A: | H₂O, B: MeCN, C: 50% H₂O/50% MeCN + 2.0% |
| | ammonia (aq.) |
| Gradient: | 0.0-1.7 mins 0-95% B, 5% D; 1.7-2.1 mins 95% B, |
| | 5% D 2.1-2.5 mins 95% A, 5% D. |

### General Synthesis Methods

As shown in Scheme 1, 3,4-methylenedioxymethamphetamine derivatives described here can be synthesized by acylating 1-(1,3-benzodioxol-5-yl)-N-methyl-propan-2-amine or salt thereof with an appropriate acid chlorid or chloroformate under basic conditions.

Alternatively, compound disclosed herein can be synthesized by reacting an acid (carboxylic acid RCOO₂H) with 1-(1,3-benzodioxol-5-yl)-N-methyl-propan-2-amine or salt thereof under standard amide coupling conditions, employing well-known coupling (activating) reagents such as DCC, EDCI, HATU, COMU, T3P, BOP, BOP-Cl, etc. The solvent for such reactions can be DMF, DCM, 1,2-DCE, ACN, THF, etc.

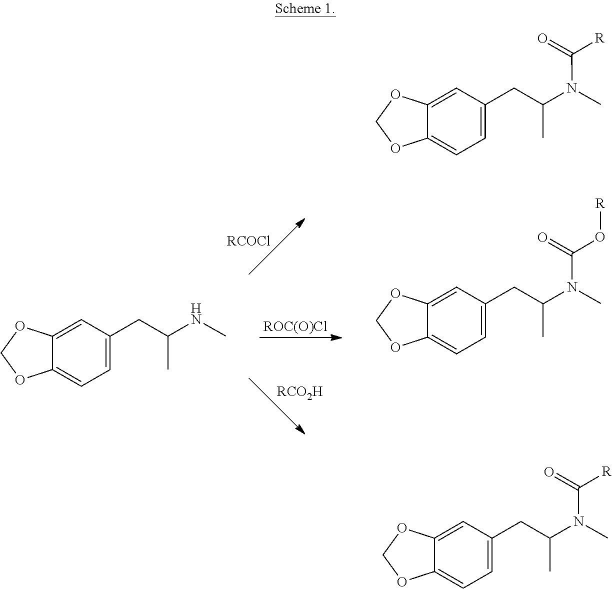

### Example 1: N-[2-(2H-1,3-Benzodioxol-5-yl)-1-methyl-ethyl]-N-methylacetamide (Compound 49)

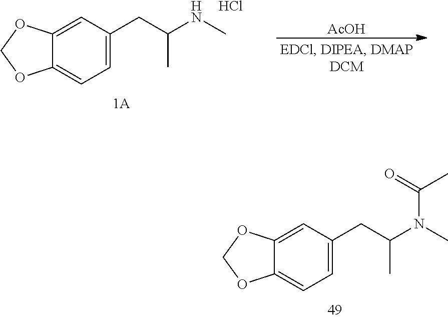 [SMILES: "AcOH" ; Cl ; CNC(C)Cc1ccc2c(c1)OCO2 ; CC(=O)N(C)C(C)Cc1ccc2c(c1)OCO2]

DIPEA (180 mg, 1.39 mmol, 243 μL) was added dropwise over 2 minutes to a stirred mixture of 1-(1,3-benzodioxol-5-yl)-N-methyl-propan-2-amine hydrochloride (1A, 97 mg, 0.42 mmol), AcOH (51 mg, 0.84 mmol, 48 μL), 3-(ethyliminomethyleneamino)-N,N-dimethyl-propan-1-amine hydrochloride (EDCl, 121 mg, 0.63 mmol) and DMAP (5 mg, 0.04 mmol) in DCM (5 mL) at rt under an atmosphere of N₂. The mixture was heated to 40° C. and stirred for 2 hrs. The mixture was diluted with DCM (45 mL) and the organic phase was then washed with saturated aqueous NaHCO₃ (2×50 mL) and brine (2×50 mL). The organic phase was dried over Na₂SO₄ and concentrated in vacuo. The residue was purified by column chromatography on silica gel (0-60% EtOAc in hexanes) to give N-[2-(2H-1,3-benzodioxol-5-yl)-1-methyl-ethyl]-N-methylacetamide (Compound 49, 80 mg, 79%) of as an oil. Spectroscopic data of the title compound was obtained as a mixture of two rotational isomers. LC-MS (LC-MS-2: Method 2A): rt=1.31 mins; MS m/z 236.0=[M+H]⁺; ¹H NMR (400 MHz, CDCl₃) δ 6.75-6.67 (m, 1.5H), 6.65-6.53 (m, 1.5H), 5.95-5.92 (m, 1H), 5.91 (s, 1H), 4.94 (app. h, J=6.9 Hz, 0.5H), 4.04-3.93 (m, 0.5H), 2.82 (s, 1.5H), 2.79 (s, 1.5H), 2.76-2.58 (m, 2H), 2.01 (s, 1.5H), 1.84 (s, 1.5H), 1.23 (d, J=6.9 Hz, 1.5H), 1.09 (d, J=6.9 Hz, 1.5H).

The following compounds listed in Table 2 were prepared using a similar procedure to the procedure for preparing Compound 49 using 1-(1,3-benzodioxol-5-yl)-N-methyl-propan-2-amine hydrochloride and using an appropriate carboxylic acid in lieu of acetic acid.

TABLE 2

| Compound | Structure and Name | Retention Time, [M + H]+, ¹H NMR |
|---|---|---|
| 50 | 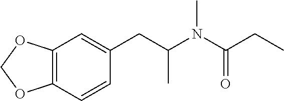 [SMILES: CCC(=O)N(C)C(C)Cc1ccc2c(c1)OCO2] N-[2-(2H-1,3-Benzodioxol-5-yl)-1- methyl-ethyl]-N-methylpropionamide | LC-MS (LC-MS-2: Method 2A): rt = 1.41 mins; MS m/z 249.9 = [M + H]⁺ ¹H NMR (400 MHz, CDCl₃) δ 6.75 − 6.67 (m, 1.5H), 6.66 − 6.51 (m, 1.5H), 5.96 − 5.87 (m, 2H), 4.94 (app. h, J = 7.0 Hz, 0.5H), 4.03 (app. h, J = 7.0 Hz, 0.5H), 2.83 (s, 1.5H), 2.78 (s, 1.5H), 2.75 − 2.57 (m, 2H), 2.29 − 2.12 (m, 1.5H), 2.07 − 1.94 (m, 0.5H), 1.21 (d, J = 7.0 Hz, 1.5H), 1.11 − 1.05 (m, 3H), 1.01 (t, J = 7.4 Hz, 1.5H). |
| 302 | 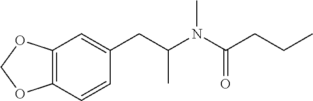 [SMILES: CCCC(=O)N(C)C(C)Cc1ccc2c(c1)OCO2] N-[2-(2H-1,3-Benzodioxol-5-yl)-1- methyl-ethyl]-N-methylbutyramide | LC-MS (LC-MS-2: Method 2A): rt = 1.51 mins; MS m/z 264.0 = [M + H]⁺ ¹H NMR (400 MHz, CDCl₃) δ 6.74 − 6.66 (m, 1.5H), 6.66 − 6.50 (m, 1.5H), 5.92 (s, 1H), 5.90 (s, 1H), 4.96 (app. h, J = 7.0 Hz, 0.5H), 4.04 (app. h, J = 7.0 Hz, 0.5H), 2.83 (s, 1.5H), 2.78 (s, 1.5H), 2.73 − 2.56 (m, 2H), 2.22 − 2.08 (m, 1.5H), 1.98 − 1.88 (m, 0.5H), 1.61 − 1.45 (m, 2H), 1.21 (d, J = 7.0 Hz, 1.5H), 1.08 (d, J = 7.0 Hz, |
| | | 1.5H), 0.92 − 0.81 (m, 3H). |
| 303 | 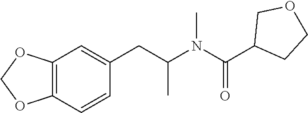 [SMILES: CC(Cc1ccc2c(c1)OCO2)N(C)C(=O)C1CCOC1] N-[2-(2H-1,3-Benzodioxol-5-yl)-1- methyl-ethyl]-N-methyl-tetrahydro- 3-furamide | LC-MS (LC-MS-2: Method 2A): rt = 1.29 mins; MS m/z 292.0 = [M + H]+ Mixture of diastereoisomers: ¹H NMR (400 MHz, CDCl₃) δ 6.75 − 6.65 (m, 1.5H), 6.65 − 6.50 (m, 1.5H), 5.96 − 5.87 (m, 2H), 5.02 − 4.92 (m, 0.5H), 4.14 − 4.03 (m, 0.5H), 3.98 − 3.74 (m, 3H), 3.69 − 3.63 (m, 0.25H) 3.61 − 3.51 (m, 0.5H), 3.48 − 3.42 (m, 0.25H), 3.17 − 3.08 (m, 0.5H), 2.96 − 2.89 (m, 0.5H), 2.86 (br. s, 1.5H), 2.81 (br. s, 1.5H), 2.77 − 2.60 (m, 2H), 2.27 − 2.19 (m, 0.25H), 2.11 − 1.91 |
| | | (m, 1.25H), 1.88 − 1.76 (m, 0.25H), 1.69 − |
| | | 1.59 (m, 0.25H), 1.32 − 1.19 (m, 1.5H), |
| | | 1.12 (br. d, J = 6.8 Hz, 1.5H). |
| 304 | 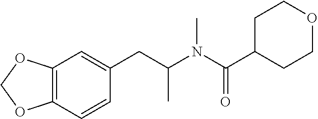 [SMILES: CC(Cc1ccc2c(c1)OCO2)N(C)C(=O)C1CCOCC1] N-[2-(2H-1,3-Benzodioxol-5-yl)-1- methyl-ethyl]-N-methyl-tetrahydro- 2H-pyran-4-carboxamide | LC-MS (LC-MS-2: Method 2A): rt = 1.31 mins; MS m/z 306.0 = [M + H]⁺ ¹H NMR (400 MHz, CDCl₃) δ 6.81 − 6.65 (m, 1.5H), 6.64 − 6.46 (m, 1.5H), 6.01 − 5.80 (m, 2H), 4.98 (app. h, J = 7.0 Hz, 0.5H), 4.14 − 3.85 (m, 2.5H), 3.45 − 3.17 (m, 2H), 2.84 (s, 1.5H), 2.82 (s, 1.5H), 2.77 − 2.57 (m, 2.5H), 2.39 − 2.29 (m, 0.5H), 1.94 − 1.63 (m, 2H), 1.51 − 1.31 (m, 1.5H), 1.27 (d, J = 7.0 Hz, 1.5H), 1.11 (d, J = 7.0 Hz, 1.5H), 0.95 − 0.89 (m, 0.5H). |
| 305 | 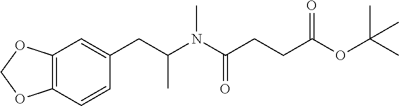 [SMILES: CC(Cc1ccc2c(c1)OCO2)N(C)C(=O)CCC(=O)OC(C)(C)C] tert-Butyl 3-{[2-(2H-1,3- benzodioxol-5-yl)-1-methyl-ethyl]- N-methylcarbamoyl}propionate | LC-MS (LC-MS-2: Method 2A): rt = 1.67 mins; MS m/z 350.0 = [M + H]⁺ ¹H NMR (400 MHz, CDCl₃) δ 6.73 − 6.67 (m, 1.5H), 6.64 − 6.53 (m, 1.5H), 5.93 (s, 1H), 5.91 (s, 1H), 4.90 (app. h, J = 6.9 Hz, 0.5H), 4.15 − 4.03 (m, 0.5H), 2.83 (s, 1.5H), 2.81 (s, 1.5H), 2.75 − 2.57 (m, 2H), 2.55 − 2.32 (m, 3.5H), 2.24 − 2.11 (m, 0.5H), 1.44 (s, 4.5H), 1.43 (s, 4.5H), 1.22 (d, J = 6.9 Hz, 1.5H), 1.08 (d, J = 6.9 Hz, 1.5H). |
| 306 | 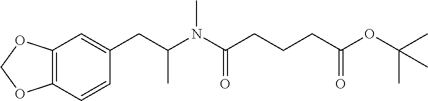 [SMILES: CC(Cc1ccc2c(c1)OCO2)N(C)C(=O)CCCC(=O)OC(C)(C)C] tert-Butyl 4-{[2-(2H-1,3- benzodioxol-5-yl)-1-methyl-ethyl]- N-methylcarbamoyl}butyrate | LC-MS (LC-MS-2: Method 2A): rt = 1.60 mins; MS m/z 364.1 = [M + H]⁺ ¹H NMR (400 MHz, CDCl₃) δ 6.73 − 6.68 (m, 1.5H), 6.64 − 6.54 (m, 1.5H), 5.94 − 5.89 (m, 2H), 4.95 (app. h, J = 6.8 Hz, 0.5H), 4.10 − 4.00 (m, 0.5H), 2.83 (s, 1.5H), 2.78 (s, 1.5H), 2.74 − 2.58 (m, 2H), 2.28 − 2.08 (m, 3.5H), 2.01 − 1.92 (m, 0.5H), 1.87 − 1.71 (m, 2H), 1.43 (s, 9H), 1.22 (d, J = 6.8 Hz, 1.5H), 1.09 (d, J = 6.8 Hz, 1.5H). |
| 307 | 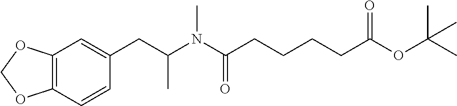 [SMILES: CC(Cc1ccc2c(c1)OCO2)N(C)C(=O)CCCCC(=O)OC(C)(C)C] tert-Butyl 5-{[2-(2H-1,3- benzodioxol-5-yl)-1-methyl-ethyl]- N-methylcarbamoyl}valerate | LC-MS (LC-MS-2: Method 2A): rt = 1.70 mins; MS m/z 378.1 = [M + H]⁺ ¹H NMR (400 MHz, CDCl₃) δ 6.74 − 6.67 (m, 1.5H), 6.64 − 6.53 (m, 1.5H), 5.95 − 5.89 (m, 2H), 4.95 (app. h, J = 6.8 Hz, 0.5H), 4.08 − 3.96 (m, 0.5H), 2.82 (s, 1.5H), 2.77 (s, 1.5H), 2.74 − 2.57 (m, 2H), 2.25 − 2.11 (m, 3.5H), 1.99 − 1.87 (m, 0.5H), 1.59 − 1.46 (m, 4H), 1.43 (s, 9H), 1.21 (d, J = 6.8 Hz, 1.5H), 1.08 (d, J = 6.8 Hz, 1.5H). |
| 259 | 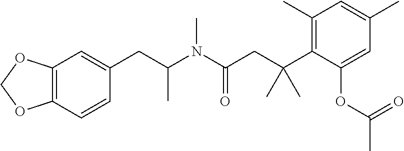 [SMILES: CC(=O)Oc1cc(C)cc(C)c1C(C)(C)CC(=O)N(C)C(C)Cc1ccc2c(c1)OCO2] 2-(2-{[2-(2H-1,3-Benzodioxol-5-yl)- 1-methylethyl]-N-methylcarbamoyl}-1,1- | LC-MS (LC-MS-2: Method 2A): rt = 1.87 mins; MS m/z 440.1 = [M + H]⁺ ¹H NMR (400 MHz, CDCl₃) δ 6.80 − 6.76 (m, 1H), 6.73 − 6.65 (m, 1.6H), 6.61 − 6.51 (m, 2.4H), 5.94 − 5.88 (m, 2H), 4.97 (app. h, J = 7.0 Hz, 0.6H), 4.00 − 3.85 (m, 0.4H), 2.86 − 2.51 (m, 7H), 2.48 (s, 1.8H), 2.45 (s, 1.2H), 2.28 − 2.24 (m, 3H), 2.23 − 2.18 (m, 3H), 1.53 − 1.42 (m, 6H), 1.02 (d, J = 7.0 Hz, 1.8H), 0.99 (d, J = 7.0 Hz, 1.2H). |
| | dimethylethyl)-3,5-xylyl acetate | |
| 94 | 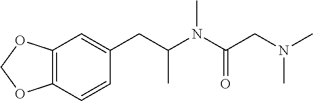 [SMILES: CC(Cc1ccc2c(c1)OCO2)N(C)C(=O)CN(C)C] N-[2-(2H-1,3-Benzodioxol-5-yl)-1- | LC-MS (LC-MS-2: Method 2A): rt = 1.03 mins; MS m/z 279.0 = [M + H]⁺ ¹H NMR (400 MHz, CDCl₃) δ 6.75 − 6.56 (m, 3H), 5.96 − 5.87 (m, 2H), 4.95 (app. h, J = 7.0 Hz, 0.5H), 4.39 − 4.26 (m, 0.5H), 3.14 − 2.82 (m, 5H), 2.76 − 2.57 (m, 2H), 2.37 − 2.18 (m, 6H), 1.19 (d, J = 7.0 Hz, 1.5H), 1.12 (d, J = 7.0 Hz, 1.5H). |
| | methyl-ethyl]-N- | |
| | methyl(dimethylamino)acetamide | |
| 308 | 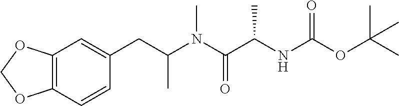 [SMILES: CC(Cc1ccc2c(c1)OCO2)N(C)C(=O)[C@H](C)NC(=O)OC(C)(C)C] (S)-1-{[2-(2H-1,3-Benzodioxol-5- yl)-1-methyl-ethyl]-N-methylcarbamoyl}ethylamino-tert- butylformylate | LC-MS (LC-MS-2: Method 2A): rt = 1.65 mins; MS m/z 365.1 = [M + H]⁺ Mixture of diastereoisomers: ¹H NMR (400 MHz, CDCl₃) δ 6.74 − 6.66 (m, 1.6H), 6.63 − 6.56 (m, 1.4H), 5.92 − 5.89 (m, 2H), 5.50 − 5.42 (m, 0.6H), 5.28 (br. s, 0.1H), 5.07 − 4.95 (m, 0.4H), 4.77 (app. h, J = 6.8 Hz, 0.4H), 4.55 − 4.41 (m, 0.9H), 4.37 − 4.29 (m, 0.2H), 4.24 − 4.16 (m, 0.2H), 4.07 − 3.97 (m, 0.2H), 2.87 − 2.83 (m, 2H), 2.80 (s, 1H), 2.78 − 2.57 (m, 2H), |
| | | 1.44 − 1.40 (m, 9H), 1.27 − 1.20 (m, |
| | | 2.5H), 1.15 (d, J = 6.8 Hz, 1H), 1.10 (d, J = |
| | | 6.8 Hz, 1H), 0.96 (d, J = 6.8 Hz, 1.5H). |
| 309 | 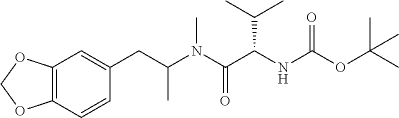 [SMILES: CC(C)[C@H](NC(=O)OC(C)(C)C)C(=O)N(C)C(C)Cc1ccc2c(c1)OCO2] (S)-1-{[2-(2H-1,3-Benzodioxol-5- yl)-1-methyl-ethyl]-N- methylcarbamoyl}-2- methylpropylamino-tert- butylformylate | LC-MS (LC-MS-2: Method 2A): rt = 1.80 mins; MS m/z 393.1 = [M + H]⁺ Mixture of diastereoisomers: ¹H NMR (400 MHz, CDCl₃) δ 6.76 − 6.54 (m, 3H), 5.97 − 5.82 (m, 2H), 5.28 − 5.17 (m, 0.7H), 5.14 − 4.98 (m, 0.5H), 4.86 (app. h, J = 6.9 Hz, 0.3H), 4.40 − 4.07 (m, 1.5H), 2.91 (s, 0.8H), 2.87 − 2.56 (m, 4.2H), 1.95 − 1.84 (m, 0.5H), 1.43 (s, 4H), 1.41 (s, 5H), 1.25 − 1.06 (m, 3.5H), 0.95 − 0.84 (m, 3H), 0.76 (d, J = 6.9 Hz, 1.6H), 0.69 (d, J = 6.9 Hz, 0.6H), 0.61 (d, J = 6.9 Hz, 0.8H). |
| 310 | 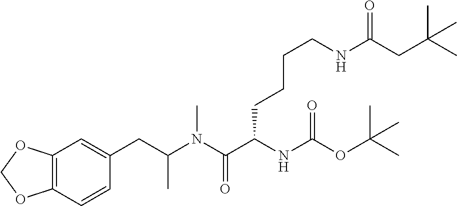 [SMILES: CC(Cc1ccc2c(c1)OCO2)N(C)C(=O)[C@H](CCCCNC(=O)CC(C)(C)C)NC(=O)OC(C)(C)C] | LC-MS (LC-MS-2: Method 2A): rt = 1.85 mins; MS m/z 522.2 = [M + H]⁺ Mixture of diastereoisomers: ¹H NMR (400 MHz, CDCl₃) δ 6.76 − 6.52 (m, 3H), 5.97 − 5.85 (m, 2H), 5.43 − 5.30 (m, 0.7H), 5.14 − 4.97 (m, 0.5H), 4.79 (app. h, J= 6.8 Hz, 0.3H), 4.62 (br. s, 1H), 4.52 − 4.31 (m, 1.1H), 4.29 − 4.20 (m, 0.3H), 4.09 − 4.00 (m, 0.1H), 3.15 − 2.97 (m, 2H), 2.89 − 2.77 (m, 3H), 2.77 − 2.55 (m, 2H), 1.54 − 1.22 (m, 24H), 1.15 (d, J = 6.8 Hz, 1.5H), 1.09 (d, J = 6.8 Hz, 1.5H). |
| | (S)-1-{[2-(2H-1,3-Benzodioxol-5- | |
| | yl)-1-methyl-ethyl]-N- | |
| | methylcarbamoyl}-5-(tert- | |
| | butoxycarbonylamino)pentylamino- | |
| | tert-butylformylate | |
| 68 | 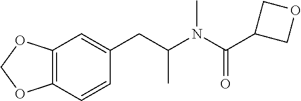 [SMILES: CC(Cc1ccc2c(c1)OCO2)N(C)C(=O)C1COC1] N-[2-(2H-1,3-Benzodioxol-5-yl)-1- methyl-ethyl]-N-methyl-3-oxetanecarboxamide | LC-MS (LCMS2: Method 2A): Rt 1.23 mins; MS m/z 278.0 = [M + H]⁺ ¹H NMR (400 MHz, CDCl₃) δ 6.77 − 6.67 (m, 1.5H), 6.62 (dd, J = 7.9, 1.7 Hz, 0.5H), 6.58 − 6.46 (m, 1H), 5.97 − 5.88 (m, 2H), 4.97 − 4.87 (m, 1H), 4.85 − 4.78 (m, 0.5H), 4.75 − 4.59 (m, 2H), 4.56 − 4.41 (m, 1H), 3.96 − 3.86 (m, 0.5H), 3.72 − 3.61 (m, 0.5H), 3.50 − 3.41 (m, 0.5H), 2.88 (s, 1.5H), 2.73 − 2.58 (m, 2H), 2.57 |
| | | (s, 1.5H), 1.21 (d, J = 6.8 Hz, 1.5H), 1.13 |
| | | (d, J = 6.8 Hz, 1.5H). |
| 320 | 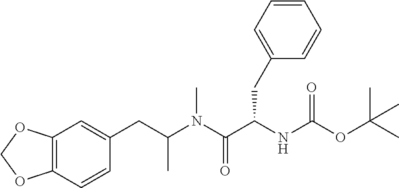 [SMILES: CC(Cc1ccc2c(c1)OCO2)N(C)C(=O)[C@H](Cc1ccccc1)NC(=O)OC(C)(C)C] (S)-1-{[2-(2H-1,3-Benzodioxol-5- | LC-MS (LCMS2: Method 2A): Rt 1.86 mins; MS m/z 441.0 = [M + H]⁺ Mixture of diastereoisomers: ¹H NMR (400 MHz, CDCl₃) δ 7.26 − 7.02 (m, 5H), 6.74 − 6.46 (m, 3H), 5.96 − 5.76 (m, 2H), 5.32 (br. d, J = 7.5 Hz, 0.6H), 5.25 (br. d, J = 8.4 Hz, 0.2H), 5.09 (br. d, J = 9.4 Hz, 0.2H), 4.90 − 4.62 (m, 1.6H), 4.27 − 4.16 (m, 0.2H), 3.89 − 3.79 (m, 0.2H), 2.98 − 2.83 (m, 1.6H), 2.76 − 2.39 (m, 5.4H), 1.48 − 1.29 (m, 9H), 1.18 − 1.04 (m, 1.4H), 0.89 (d, J = 6.8 Hz, 1.2H), 0.60 (d, J = 6.8 Hz, 0.4H). |
| | yl)-1-methyl-ethyl]-N- | |
| | methylcarbamoyl}-2- | |
| | phenylethylamino-tert- | |
| | butylformylate | |
| 106 | 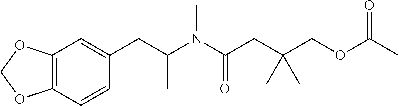 [SMILES: CC(=O)OCC(C)(C)CC(=O)N(C)C(C)Cc1ccc2c(c1)OCO2] (3-{[2-(2H-1,3-Benzodioxol-5-yl)-1- methylethyl]-N-methylcarbamoyl}- 2,2-dimethylpropyl acetate | LC-MS (LCMS2: Method 2A): Rt 1.56 mins; MS m/z 350.0 = [M + H]⁺ ¹H NMR (400 MHz, CDCl₃) δ 6.76 − 6.47 (m, 3H), 5.96 − 5.86 (m, 2H), 5.05 (app. h, J = 6.9 Hz, 0.6H), 4.13 − 4.02 (m, 0.4H), 3.96 − 3.81 (m, 2H), 2.87 − 2.76 (m, 3H), 2.75 − 2.58 (m, 2H), 2.28 − 2.14 (m, 1.6H), 2.05 (s, 3H), 1.88 − 1.78 (m, 0.4H), 1.22 (d, J = 6.9 Hz, 1.2H), 1.10 (d, J = 6.9 Hz, 1.8H), 0.95 (s, 6H). |

### Example 2: N-[2-(1,3-Benzodioxol-5-yl)-1-methyl-ethyl]-N,2-dimethyl-propanamide (Compound 51)

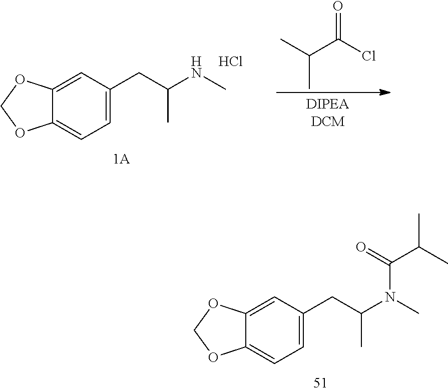

2-Methylpropanoyl chloride (57 mg, 0.54 mmol, 56 μL) was added dropwise over 2 mins to a stirred mixture of 1-(1,3-benzodioxol-5-yl)-N-methyl-propan-2-amine hydrochloride (1A, 103 mg, 0.45 mmol) and DIPEA (127 mg, 0.99 mmol, 172 μL) in DCM (5 mL) at 0° C. under nan atmosphere of N₂. The mixture was stirred at 0° C. for 30 min, warmed to rt and then stirred for 15 min. The mixture was poured into 2N aqueous HCl (20 mL) and the layers were separated. The aqueous layer was extracted with DCM (2×20 mL) and the combined organic layers were dried over Na₂SO₄ and concentrated in vacuo. The residue was purified by column chromatography on silica gel (0-60% EtOAc in hexanes) to give N-[2-(1,3-benzodioxol-5-yl)-1-methyl-ethyl]-N,2-dimethyl-propanamide (Compound 51, 110 mg, 91%) as an oil. Spectroscopic data of the title compound was obtained as a mixture of two rotational isomers. LC-MS (LC-MS-2: Method 2B): rt=1.45 mins; MS m/z 264.0=[M+H]⁺; ¹H NMR (400 MHz, CDCl₃) δ 6.79-6.67 (m, 1.5H), 6.67-6.49 (m, 1.5H), 5.90 (s, 2H), 4.98 (app. h, J=6.9 Hz, 0.5H), 4.22-3.96 (m, 0.5H), 2.85-2.78 (m, 3H), 2.75-2.60 (m, 2.5H), 2.60-2.43 (m, 0.5H), 1.24 (d, J=6.9 Hz, 1.5H), 1.10 (d, J=6.9 Hz, 1.5H), 1.08 (d, J=6.9 Hz, 1.5H), 1.02 (d, J=6.9 Hz, 1.5H), 0.94 (d, J=6.9 Hz, 1.5H), 0.85 (d, J=6.9 Hz, 1.5H).

The following compounds listed in Table 3 were prepared using a similar procedure to the procedure for preparing Compound 51 using 1-(1,3-benzodioxol-5-yl)-N-methyl-propan-2-amine hydrochloride and using an appropriate acid chloride in lieu of 2-methylpropanoyl chloride.

TABLE 3

| Compound | Structure and Name | Retention Time, [M + H]+, ¹H NMR |
|---|---|---|
| 311 | 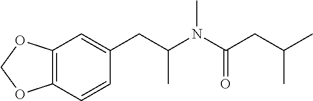 [SMILES: CC(C)CC(=O)N(C)C(C)Cc1ccc2c(c1)OCO2] N-[2-(2H-1,3-Benzodioxol-5-yl)-1- methyl-ethyl]-N-methyl-3-methylbutyramide | LC-MS (LC-MS-2: Method 2B): rt = 1.53 mins; MS m/z 278.0 = [M + H]⁺ ¹H NMR (400 MHz, CDCl₃) δ 6.75 − 6.67 (m, 1.5H), 6.66 − 6.51 (m, 1.5H), 5.95 − 5.90 (m, 1H), 5.90 − 5.87 (m, 1H), 5.01 (app. h, J = 6.9 Hz, 0.5H), 4.12 − 3.98 (m, 0.5H), 2.83 (s, 1.5H), 2.78 (s, 1.5H), 2.75 − 2.58 (m, 2H), 2.11 − 1.96 (m, 2.5H), 1.86 − 1.78 (m, 0.5H), 1.21 (d, J = 6.9 Hz, 1.5H), 1.10 (d, J = 6.9 Hz, 1.5H), 0.90 − 0.83 (m, |
| | | 4.5H), 0.81 (d, J = 6.9 Hz, 1.5H). |
| 52 | 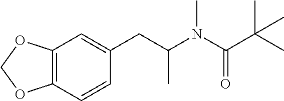 [SMILES: CC(Cc1ccc2c(c1)OCO2)N(C)C(=O)C(C)(C)C] N-[2-(2H-1,3-Benzodioxol-5-yl)-1- methyl-ethyl]-N-methyl-2,2- dimethylpropionamide | LC-MS (LC-MS-2: Method 2B): rt = 1.56 mins; MS m/z 278.0 = [M + H]⁺ ¹H NMR (400 MHz, CDCl₃) δ 6.72 (d, J = 7.9 Hz, 1H), 6.68 (d, J = 1.7 Hz, 1H), 6.62 (dd, J = 7.9, 1.7 Hz, 1H), 5.92 (s, 2H), 4.67 (br. s, 1H), 2.87 (s, 3H), 2.76 (dd, J = 13.7, 7.0 Hz, 1H), 2.63 (dd, J = 13.7, 8.1 Hz, 1H), 1.21 (s, 9H), 1.12 (d, J = 6.7 Hz, 3H). |

### Example 3: Ethyl N-[2-(1,3-benzodioxol-5-yl)-1-methyl-ethyl]-N-methyl-carbamate (Compound 16)

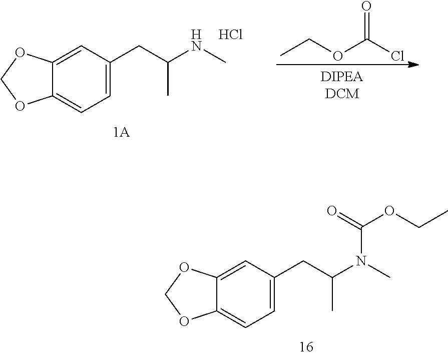

Ethyl chloroformate (68 mg, 0.63 mmol, 60 μL) was added dropwise over 2 min to a stirred mixture of 1-(1,3-benzodioxol-5-yl)-N-methyl-propan-2-amine hydrochloride (120 mg, 0.52 mmol) and DIPEA (149 mg, 1.15 mmol, 200 μL) in DCM (10 mL) at 0° C. under an atmosphere of N₂. The mixture was stirred at 0° C. for 30 min, warmed to rt and then stirred for 15 min. The mixture was poured into 2N aqueous HCl (20 mL) and the layers were separated. The aqueous layer was extracted with DCM (2×20 mL) and the combined organic layers were then dried over Na₂SO₄ and concentrated in vacuo. The residue was purified by column chromatography on silica gel (0-20% EtOAc in hexanes) to give ethyl N-[2-(1,3-benzodioxol-5-yl)-1-methyl-ethyl]-N-methyl-carbamate (Compound 16, 110 mg, 77%) as an oil. LC-MS (LC-MS-2: Method 2A): rt=1.62 mins; MS m/z 266.0=[M+H]⁺; ¹H NMR (400 MHz, DMSO-d₆) δ 6.79 (d, J=7.9 Hz, 1H), 6.74 (d, J=1.6 Hz, 1H), 6.61 (br. d, J=7.9 Hz, 1H), 5.95 (s, 2H), 4.35-4.21 (m, 1H), 3.99-3.80 (m, 2H), 2.69-2.58 (m, 2H), 2.66 (s, 3H), 1.14-0.99 (m, 6H).

The following compounds listed in Table 4 were prepared using a similar procedure to the procedure for preparing Compound 16 using 1-(1,3-benzodioxol-5-yl)-N-methyl-propan-2-amine hydrochloride and using an appropriate chloroformate in lieu of ethyl chloroformate.

TABLE 4

| Compound | Structure and Name | Retention Time, [M + H]+, ¹H NMR |
|---|---|---|
| 300 | 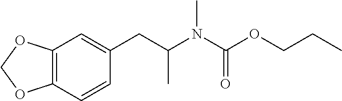 [SMILES: CCCOC(=O)N(C)C(C)Cc1ccc2c(c1)OCO2] propyl N-[2-(1,3-benzodioxol-5-yl)- 1-methyl-ethyl]-N-methyl-carbamate | LC-MS (LC-MS-2: Method 2A): rt = 1.72 mins; MS m/z 280.0 = [M + H]⁺ ¹H NMR (400 MHz, DMSO-d₆) δ 6.78 (d, J = 7.9 Hz, 1H), 6.73 (d, J = 1.6 Hz, 1H), 6.61 (br. d, J = 7.9 Hz, 1H), 5.94 (s, 2H), 4.37 − 4.20 (m, 1H), 3.91 − 3.73 (m, 2H), 2.68 − 2.60 (m, 2H), 2.66 (s, 3H), 1.52 − 1.40 (m, 2H), 1.13 − 1.03 (m, 3H), 0.81 (t, J = 7.4 Hz, 3H). |
| 17 | 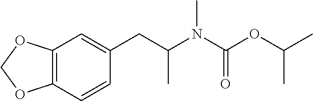 [SMILES: CC(C)OC(=O)N(C)C(C)Cc1ccc2c(c1)OCO2] | LC-MS (LC-MS-2: Method 2A): rt = 1.70 mins; MS m/z 280.0 = [M + H]⁺ ¹H NMR (400 MHz, DMSO-d₆) δ 6.78 (d, J = 7.9 Hz, 1H), 6.72 (d, J = 1.6 Hz, 1H), 6.60 (br. d, J = 7.9 Hz, 1H), 5.94 (s, 2H), 4.70 − 4.55 (m, 1H), 4.35 − 4.21 (m, 1H), 2.69 − 2.56 (m, 2H), 2.64 (s, 3H), 1.20 − 0.90 (m, 9H). |
| | isopropyl N-[2-(1,3-benzodioxol-5- | |
| | yl)-1-methyl-ethyl]-N-methyl- | |
| | carbamate | |
| 301 | 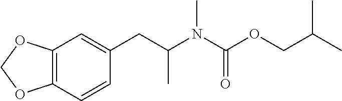 [SMILES: CC(C)COC(=O)N(C)C(C)Cc1ccc2c(c1)OCO2] isobutyl N-[2-(1,3-benzodioxol-5- yl)-1-methyl-ethyl]-N-methyl-carbamate | LC-MS (LC-MS-2: Method 2A): rt = 1.79 mins; MS m/z 294.0 = [M + H]⁺ ¹H NMR (400 MHz, DMSO-d₆, T = 298 K) δ 6.78 (d, J = 7.9 Hz, 1H), 6.73 (d, J = 1.6 Hz, 1H), 6.61 (br. d, J = 7.9 Hz, 1H), 5.94 (s, 2H), 4.39 − 4.21 (m, 1H), 3.82 − 3.57 (m, 2H), 2.70 − 2.58 (m, 2H), 2.67 (s, 3H), 1.82 − 1.68 (m, 1H), 1.13 − 1.03 (m, 3H), 0.81 (d, J = 6.7 Hz, 6H). ¹H NMR (400 MHz, DMSO-d₆, T = 343 K) δ 6.77 (d, J = 7.9 Hz, 1H), 6.72 (d, J = 1.7 |
| | | Hz, 1H), 6.62 (dd, J = 7.9, 1.7 Hz, 1H), 5.93 |
| | | (s, 2H), 4.35 − 4.22 (m, 1H), 3.78 − 3.64 (m, |
| | | 2H), 2.73 − 2.60 (m, 2H), 2.69 (s, 3H), 1.86 − |
| | | 1.74 (m, 1H), 1.11 (d, J = 6.5 Hz, 3H), |
| | | 0.85 (d, J = 6.7 Hz, 6H). |
| 2 | 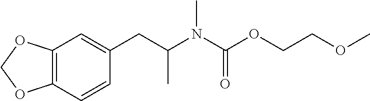 [SMILES: COCCOC(=O)N(C)C(C)Cc1ccc2c(c1)OCO2] 2-methoxyethyl N-[2-(1,3- benzodioxol-5-yl)-1-methyl-ethyl]- N-methyl-carbamate | LC-MS (LC-MS-2: Method 2A): rt = 1.51 mins; MS m/z 295.9 = [M + H]⁺ ¹H NMR (400 MHz, DMSO-d₆) δ 6.79 (d, J = 7.9 Hz, 1H), 6.74 (d, J = 1.7 Hz, 1H), 6.62 (dd, J = 7.9, 1.7 Hz, 1H), 5.95 (s, 2H), 4.36 − 4.18 (m, 1H), 4.04 − 3.88 (m, 2H), 3.45 − 3.38 (m, 2H), 3.25 − 3.21 (m, 3H), 2.69 − 2.59 (m, 2H), 2.66 (s, 3H), 1.12 − 1.04 (m, 3H). |
| 113 | 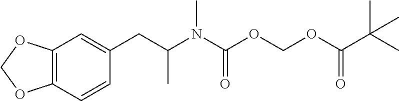 [SMILES: CC(Cc1ccc2c(c1)OCO2)N(C)C(=O)OCOC(=O)C(C)(C)C] [[2-(1,3-benzodioxol-5-yl)-1-methyl- ethyl]-methyl-carbamoyl]oxymethyl- 2,2-dimethylpropanoate | LC-MS (LC-MS-2: Method 2A): rt = 1.80 mins; MS m/z 352.0 = [M + H]⁺ Mixture of two rotational isomers: ¹H NMR (400 MHz, DMSO-d₆) δ 6.80 − 6.71 (m, 2H), 6.64 − 6.58 (m, 1H), 5.95 (s, 1H), 5.94 (s, 1H), 5.63 − 5.56 (m, 2H), 4.33 (app. h, J = 6.9 Hz, 0.5H), 4.16 (app. h, J = 6.9 Hz, 0.5H), 2.70 (s, 1.5H), 2.67 − 2.62 (m, 2H), 2.65 (s, 1.5H), 1.13 − 1.05 (m, 12H). |

### Example 4: tert-Butyl N-[2-(1,3-benzodioxol-5-yl)-1-methyl-ethyl]-N-methyl-carbamate (Compound 18)

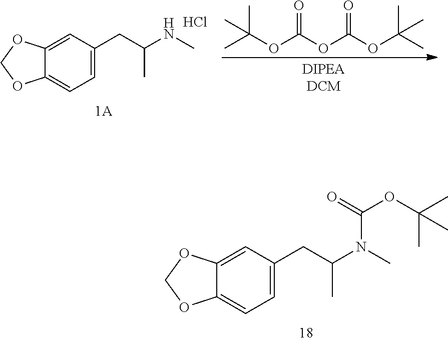

Di-tert-butyl dicarbonate (101 mg, 0.46 mmol) was added in one portion to a stirred mixture of 1-(1,3-benzodioxol-5-yl)-N-methyl-propan-2-amine hydrochloride (1A, 106 mg, 0.46 μmol) and DIPEA (60 mg, 0.46 mmol, 80 μL) in DCM (5 mL) at 0° C. under an atmosphere of N₂. The mixture was stirred at 0° C. for 30 min, warmed to rt and then stirred for 15 min. The mixture was poured into 2N aqueous HCl (20 mL) and the layers were separated. The aqueous layer was extracted with DCM (2×20 mL) and the combined organic layers were then dried over Na₂SO₄ and concentrated in vacuo. The residue was purified by column chromatography on silica gel (0-20% EtOAc in hexanes) to give tert-butyl N-[2-(1,3-benzodioxol-5-yl)-1-methyl-ethyl]-N-methyl-carbamate (Compound 18, 92 mg, 66%) as an oil. Spectroscopic data of Compound 18 was obtained as a mixture of two rotational isomers at 298 K, which coalesced at 343 K. LC-MS (LC-MS-2: Method 2A): rt=1.80 mins; MS m/z 238.0=[M-^{t}Bu+H]⁺; ¹H NMR (400 MHz, DMSO-d₆, T=298 K) δ 6.79 (br. d, J=7.8 Hz, 1H), 6.72 (d, J=1.6 Hz, 1H), 6.60 (br. d, J=7.8 Hz, 1H), 5.93 (s, 2H), 4.33-4.18 (m, 1H), 2.62-2.56 (m, 2H), 2.61 (s, 3H), 1.30 (br. s, 3.5H), 1.23 (br. s, 5.5H), 1.12-1.01 (m, 3H); ¹H NMR (400 MHz, DMSO-d₆, T=343 K) δ 6.77 (d, J=8.0 Hz, 1H), 6.71 (d, J=1.8 Hz, 1H), 6.62 (dd, J=8.0, 1.8 Hz, 1H), 5.92 (s, 2H), 4.24 (app. h, J=6.8 Hz, 1H), 2.68-2.56 (m, 2H), 2.62 (s, 3H), 1.31 (s, 9H), 1.09 (d, J=6.8 Hz, 3H).

### Example 5: 1-[2-(2H-1,3-Benzodioxol-5-yl)-1-methyl-ethyl]-1,3,3-trimethylurea (Compound 312)

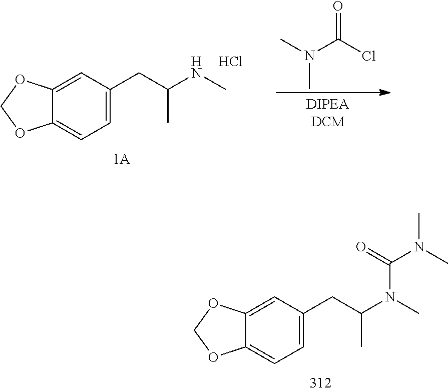

N,N-Dimethylcarbamoyl chloride (59 mg, 0.55 mmol, 51 μL) was added dropwise over 2 min to a stirred mixture of 1-(1,3-benzodioxol-5-yl)-N-methyl-propan-2-amine hydrochloride (1A, 105 mg, 0.46 mmol), DMAP (6 mg, 0.05 mmol) and DIPEA (130 mg, 1.01 mmol, 175 μL) in DCM (5 mL) at 0° C. under an atmosphere of N₂. The mixture was stirred at 0° C. for 30 min, warmed to rt and then stirred for 15 min. The mixture was concentrated in vacuo and the residue was purified by column chromatography on silica gel (0-100% EtOAc in petroleum ether) to give 1-[2-(2H-1,3-benzodioxol-5-yl)-1-methyl-ethyl]-1,3,3-trimethylurea (Compound 312, 101 mg, 83) as an oil. LC-MS (LC-MS-2: Method 2A): rt=1.44 mins; MS m/z 265.0=[M+H]⁺; ¹H NMR (400 MHz, CDCl₃) δ 6.71 (d, J=7.9 Hz, 1H), 6.69 (d, J=1.7 Hz, 1H), 6.63 (dd, J=7.9, 1.7 Hz, 1H), 5.91 (s, 2H), 4.08 (app. h, J=7.0 Hz, 1H), 2.78 (dd, J=13.5, 7.2 Hz, 1H), 2.69 (s, 3H), 2.66 (s, 6H), 2.61 (dd, J=13.5, 7.2 Hz, 1H), 1.15 (d, J=6.8 Hz, 3H).

Compound 25 as listed in Table 5 was prepared using a similar procedure to the procedure for preparing Compound 24 using 1-(1,3-benzodioxol-5-yl)-N-methyl-propan-2-amine hydrochloride and using an appropriate carbamoyl chloride in lieu of N,N-dimethylcarbamoyl chloride.

TABLE 5

| Compound | Structure and Name | Retention Time, [M + H]+, ¹H NMR |
|---|---|---|
| 313 | 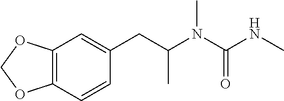 [SMILES: CNC(=O)N(C)C(C)Cc1ccc2c(c1)OCO2] 1-[2-(2H-1,3-Benzodioxol-5-yl)-1- methyl-ethyl]-1,3-dimethylurea | LC-MS (LC-MS-2: Method 2A): rt = 1.23 mins; MS m/z 251.0 = [M + H]⁺ ¹H NMR (400 MHz, CDCl₃) δ 6.72 (d, J = 7.9 Hz, 1H), 6.68 (d, J = 1.7 Hz, 1H), 6.63 (dd, J = 7.9, 1.7 Hz, 1H), 5.91 (s, 2H), 4.52 (app. h, J = 7.1 Hz, 1H), 4.15 (br. s, 1H), 2.75 (s, 3H), 2.75 − 2.67 (m, 1H), 2.69 (s, 3H), 2.58 (dd, J = 13.7, 7.4 Hz, 1H), 1.10 (d, J= 6.8 Hz, 3H). |
| 321 | 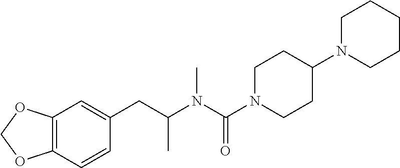 [SMILES: CC(Cc1ccc2c(c1)OCO2)N(C)C(=O)N1CCC(N2CCCCC2)CC1] | LC-MS (LCMS2: Method 2A): Rt 1.14 mins; MS m/z 388.3 = [M + H]⁺ ¹H NMR (400 MHz, CDCl₃) δ 6.74 − 6.65 (m, 2H), 6.62 (dd, J = 7.9, 1.7 Hz, 1H), 5.91 (s, 2H), 4.13 (app. h, J = 6.9 Hz, 1H), 3.53 − 3.39 (m, 2H), 2.77 − 2.32 (m, 12H), 1.80 − 1.71 (m, 2H), 1.70 − 1.56 (m, 4H), 1.55 − 1.41 (m, 3H), 1.37 − 1.26 (m, 1H), 1.16 (d, J = 6.9 Hz, 3H). |
| | N-[2-(1,3-Benzodioxol-5-yl)-1- | |
| | methyl-ethyl]-N-methyl-4-(1- | |
| | piperidyl)piperidine-1-carboxamide | |

### Example 6: 3-{[2-(2H-1,3-Benzodioxol-5-yl)-1-methyl-ethyl]-N-methylcarbamoyl}propionic acid (Compound 314)

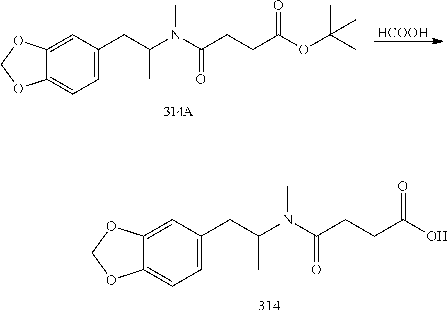 [SMILES: CC(Cc1ccc2c(c1)OCO2)N(C)C(=O)CCC(=O)OC(C)(C)C ; "HCOOH" ; CC(Cc1ccc2c(c1)OCO2)N(C)C(=O)CCC(=O)O]

tert-Butyl 3-{[2-(2H-1,3-benzodioxol-5-yl)-1-methyl-ethyl]-N-methylcarbamoyl}propionate (314A, 187 mg, 0.54 mmol) was dissolved in formic acid (3.05 g, 66.3 mmol, 2.50 mL) and the resulting mixture was stirred at rt under at atmosphere of N₂ for 4 h. The mixture was concentrated in vacuo at 45° C. The residue was dissolved in DCM (5 mL) and the mixture concentrated in vacuo at 45° C. This process was repeated a further two times, to give 3-{[2-(2H-1,3-benzodioxol-5-yl)-1-methyl-ethyl]-N-methylcarbamoyl}propionic acid (Compound 314, 150 mg, 93%) as a gum. Spectroscopic data of Compound 314 was obtained as a mixture of two rotational isomers. LC-MS (LC-MS-2: Method 2A): rt=1.30 mins; MS m/z 294.0=[M+H]+; ¹H NMR (400 MHz, CDCl₃) δ 6.74-6.65 (m, 1.5H), 6.62-6.52 (m, 1.5H), 5.93 (s, 1H), 5.91 (s, 1H), 4.91 (app. h, J=7.0 Hz, 0.5H), 4.11-3.96 (m, 0.5H), 2.87 (s, 1.5H), 2.81 (s, 1.5H), 2.73-2.62 (m, 3H), 2.61-2.46 (m, 2.5H), 2.22-2.10 (m, 0.5H), 1.26 (d, J=7.0 Hz, 1.5H), 1.12 (d, J=7.0 Hz, 1.5H). CO₂H proton not observed.

The following compounds listed in Table 6 were prepared using a similar procedure to the procedure for preparing Compound 314 using an appropriate tert-butyl ester in lieu of tert-butyl 3-{[2-(2H-1,3-benzodioxol-5-yl)-1-methyl-ethyl]-N-methylcarbamoyl}propionate (314A).

TABLE 6

| Compound | Structure and Name | Retention Time, [M + H]+, ¹H NMR |
|---|---|---|
| 315 | 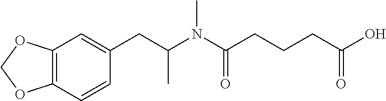 [SMILES: CC(Cc1ccc2c(c1)OCO2)N(C)C(=O)CCCC(=O)O] 4-{[2-(2H-1,3-Benzodioxol-5-yl)-1- methyl-ethyl]-N- methylcarbamoyl}butyric acid | LC-MS (LC-MS-2: Method 2A): rt = 1.23 mins; MS m/z 308.0 = [M + H]⁺ ¹H NMR (400 MHz, CDCl₃) δ 6.74 − 6.65 (m, 1.5H), 6.65 − 6.52 (m, 1.5H), 5.95 − 5.87 (m, 2H), 4.99 (app. h, J = 7.0 Hz, 0.5H), 4.07 − 3.97 (m, 0.5H), 2.86 (s, 1.5H), 2.80 (s, 1.5H), 2.75 − 2.60 (m, 2H), 2.37 − 2.21 (m, 3.5H), 2.05 − 1.97 (m, 0.5H), 1.92 − 1.70 (m, 2H), 1.25 (d, J = 7.0 Hz, 1.5H), 1.13 (d, J = 7.0 Hz, 1.5H). CO₂H not observed. |
| 316 | 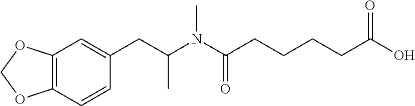 [SMILES: CC(Cc1ccc2c(c1)OCO2)N(C)C(=O)CCCCC(=O)O] 5-{[2-(2H-1,3-Benzodioxol-5-yl)-1- methyl-ethyl]-N- methylcarbamoyl}valeric acid | LC-MS (LC-MS-2: Method 2A): rt = 1.27 mins; MS m/z 322.0 = [M + H]⁺ ¹H NMR (400 MHz, CDCl₃) δ 6.76 − 6.66 (m, 1.5H), 6.64 − 6.51 (m, 1.5H), 5.95 − 5.87 (m, 2H), 4.97 (app. h, J = 6.9 Hz, 0.5H), 4.08 − 3.96 (m, 0.5H), 2.84 (s, 1.5H), 2.78 (s, 1.5H), 2.74 − 2.59 (m, 2H), 2.36 − 2.22 (m, 3H), 2.21 − 2.12 (m, 0.5H), 2.00 − 1.88 (m, 0.5H), 1.65 − 1.45 (m, 4H), 1.23 (d, J = 6.9 Hz, 1.5H), 1.10 (d, J = 6.9 Hz, 1.5H). CO₂H not observed. |

### Example 7: (2S)-2-Amino-N-[2-(1,3-benzodioxol-5-yl)-1-methyl-ethyl]-N-methyl-propanamide hydrochloride (Compound 317)

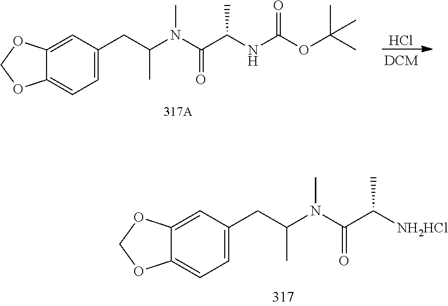

4N HCl in dioxane (1.96 mL) was added to a stirred mixture of tert-butyl ((2S)-1-((1-(benzo[d][1,3]dioxol-5-yl)propan-2-yl)(methyl)amino)-1-oxopropan-2-yl)carbamate (317A, 166 mg, 0.46 mmol) in DCM (5 mL) at rt under an atmosphere of N₂. The mixture was heated to 40° C. and stirred for 2 h. The mixture was concentrated in vacuo to afford (2S)-2-amino-N-[2-(1,3-benzodioxol-5-yl)-1-methyl-ethyl]-N-methyl-propanamide hydrochloride (Compound 317, 96 mg, 69%) as a solid. Spectroscopic data of Compound 317 was obtained as a mixture of rotational isomers and diastereoisomers. LC-MS (LC-MS-2: Method 2A): rt=1.02 mins; MS m/z 265.0=[M+H]⁺; ¹H NMR (400 MHz, DMSO-d₆) δ 8.03 (br. s, 3H), 6.97-6.58 (m, 3H), 6.02-5.87 (m, 2H), 4.92-4.82 (m, 0.3H), 4.55 (app. h, J=7.0 Hz, 0.2H), 4.31-3.96 (m, 1.5H), 2.89-2.56 (m, 5H), 1.30-0.85 (m, 6H).

The following compounds listed in Table 7 were prepared using a similar procedure to the procedure for preparing Compound 317 using an appropriate Boc-protected amine in lieu of tert-butyl ((2S)-1-((1-(benzo[d][1,3]dioxol-5-yl)propan-2-yl)(methyl)amino)-1-oxopropan-2-yl)carbamate (317A).

TABLE 7

| Compound | Structure and Name | Retention Time, [M + H]+, ¹H NMR |
|---|---|---|
| 318 | 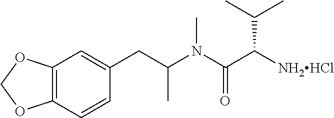 (2S)-2-amino-N-[2-(1,3-benzodioxol- 5-yl)-1-methyl-ethyl]-N,3-dimethyl- butanamide hydrochloride | LC-MS (LC-MS-2: Method 2A): rt = 1.08 mins; MS m/z 293.0 = [M + H]⁺ Mixture of diastereoisomers: ¹H NMR (400 MHz, DMSO-d₆) δ 7.98 (br. s, 3H), 7.05 − 6.50 (m, 3H), 6.13 − 5.73 (m, 2H), 4.97 − 4.87 (m, 0.3H), 4.62 (app. h, J = 7.0 Hz, 0.2H), 4.21 − 3.96 (m, 1.5H), 2.93 − 2.80 (m, 3H), 2.79 − 2.64 (m, 2H), 2.11 − 2.01 (m, 0.3H), 1.71 − 1.61 (m, 0.3H), 1.40 − 0.43 (m, 9.4H). |
| 319 | 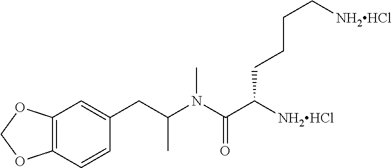 | LC-MS (LC-MS-2: Method 2A): rt = 0.87 mins; MS m/z 322.1 = [M + H]⁺ Mixture of diastereoisomers: ¹H NMR (400 MHz, DMSO-d₆) δ 8.41 − 7.59 (m, 6H), 7.02 − 6.54 (m, 3H), 6.14 − 5.83 (m, 2H), 4.96 − 4.86 (m, 0.3H), 4.57 (app. h, J = 7.1 Hz, 0.3H), 4.29 − 3.98 (m, 1.4H), 2.94 − 2.55 (m, 7H), 1.73 − 1.49 (m, 1.8H), 1.48 − 1.22 (m, 3.2H), 1.22 − 0.97 (m, 3.6H), 0.94 − 0.82 (m, 0.4H). |
| | (2S)-2,6-diamino-N-[2-(1,3-benzodioxol-5-yl)-1-methyl-ethyl]- | |
| | N-methyl-hexanamide dihydrochloride | |
| 322 | 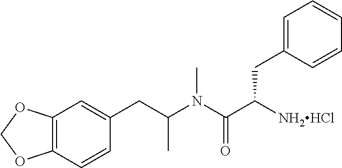 | LC-MS (LCMS2: Method 2A): Rt 1.11 mins; MS m/z 341.1 = [M + H]⁺ Mixture of diastereoisomers: ¹H NMR (400 MHz, CDCl₃) δ 8.81 − 8.35 (br. m, 3H), 7.26 − 7.02 (m, 5H), 6.81 − 6.45 (m, 3H), 5.97 − 5.75 (m, 2H), 4.89 − 4.58 (m, 1.75H), 3.98 (br. s, 0.25H), 3.61 − 3.45 (m, 0.75H), 3.36 − 3.30 (m, 0.25H), 3.18 − 2.71 (m, 2.5H), 2.57 − 2.26 (m, 3.5H), 1.27 (d, J = 6.7 Hz, 0.5H), 1.03 (d, J = 6.7 Hz, 1H), 0.80 (d, J = 6.7 Hz, 1H), 0.46 (d, J = 6.7 Hz, 0.5H). |
| | (2S)-2-Amino-N-[2-(1,3- | |
| | benzodioxol-5-yl)-1-methyl-ethyl]- | |
| | N-methyl-3-phenyl-propanamide hydrochloride | |

### Example 8: tert-Butyl N-[2-[[2-(1,3-benzodioxol-5-yl)-1-methyl-ethyl]-methyl-amino]-2-oxo-ethyl]-N-methyl-carbamate (Compound 323)

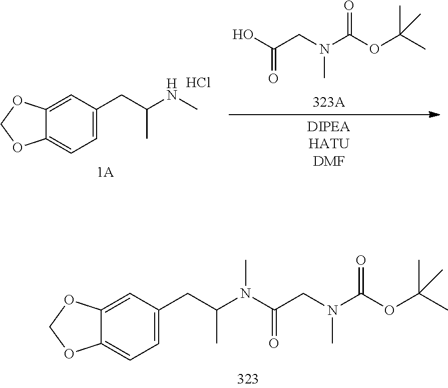 [SMILES: CN(CC(=O)O)C(=O)OC(C)(C)C ; Cl ; CNC(C)Cc1ccc2c(c1)OCO2 ; CC(Cc1ccc2c(c1)OCO2)N(C)C(=O)CN(C)C(=O)OC(C)(C)C]

HATU (1.89 g, 4.96 mmol) and then Boc-Sar-OH (323A, 1.25 g, 6.62 mmol) were added in one portion for each reagent to a stirred mixture of 1-(1,3-benzodioxol-5-yl)-N-methyl-propan-2-amine HCl (1A, 760 mg, 3.31 mmol) and DIPEA (2.14 g, 16.5 mmol, 2.88 mL) in DMF (25 mL) at rt under an atmosphere of N₂. The mixture was stirred at room temperature for 2 h, then diluted with EtOAc (125 mL). The organic phase was washed with a 90% aqueous brine solution (125 mL) and then a 50% aqueous brine solution (3×125 mL) before being dried over Na₂SO₄ and concentrated in vacuo. The residue was purified by column chromatography on silica gel (EtOAc in iso-hexane, 0:1 to 1:0) to afford a gum. The crude material was further purified by column chromatography on silica gel (MeOH in DCM, 0:1 to 5:95) to afford tert-butyl N-[2-[[2-(1,3-benzodioxol-5-yl)-1-methyl-ethyl]-methyl-amino]-2-oxo-ethyl]-N-methyl-carbamate (Compound 323, 797 mg, 63) as a gum. Spectroscopic data of Compound 323 was obtained as a mixture of two rotational isomers. LC-MS (LCMS2: Method 2A): Rt 1.56 mins; MS m/z 365.1=[M+H]⁺. ¹H NMR (400 MHz, CDCl₃) δ 6.75-6.67 (m, 1.5H), 6.66-6.54 (m, 1.5H), 5.95-5.86 (m, 2H), 4.96-4.83 (m, 0.5H), 4.28-3.79 (m, 2H), 3.34-3.13 (m, 0.5H), 2.87-2.57 (m, 8H), 1.48-1.38 (m, 9H), 1.27-1.19 (m, 1.5H), 1.14-1.07 (m, 1.5H).

### Example 9: N-[2-(1,3-Benzodioxol-5-yl)-1-methyl-ethyl]-N-methyl-2-(methylamino)acetamide hydrochloride (Compound 324)

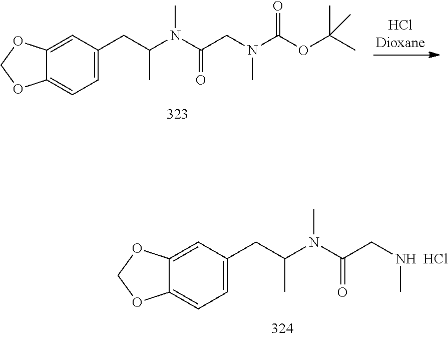 [SMILES: CC(Cc1ccc2c(c1)OCO2)N(C)C(=O)CN(C)C(=O)OC(C)(C)C ; CNCC(=O)N(C)C(C)Cc1ccc2c(c1)OCO2 ; Cl]

A mixture of tert-butyl N-[2-[[2-(1,3-benzodioxol-5-yl)-1-methyl-ethyl]-methyl-amino]-2-oxo-ethyl]-N-methyl-carbamate (323, 795 mg, 2.09 mmol) in 4M HCl in 1,4-dioxane (5.24 mL) was stirred at 0° C. under an atmosphere of N₂ for 1.5 hours. The mixture was concentrated in vacuo and the residue was then co-evaporated with chloroform (3×10 mL) and Et₂O (3×10 mL). The solid was dried under high vacuum to afford N-[2-(1,3-benzodioxol-5-yl)-1-methyl-ethyl]-N-methyl-2-(methylamino)acetamide HCl (Compound 324, 644 mg, 97%) as a solid. Spectroscopic data of the title compound was obtained as a mixture of two rotational isomers. LC-MS (LCMS2: Method 2A): Rt 0.97 mins; MS m/z 265.0=[M+H]+. ¹H NMR (400 MHz, DMSO-d₆) δ 8.76 (br. s, 2H), 6.91-6.75 (m, 2H), 6.73-6.61 (m, 1H), 5.99-5.93 (m, 2H), 4.72 (app. h, J=6.9 Hz, 0.5H), 4.05-3.81 (m, 2H), 3.44-3.38 (m, 0.5H), 2.78 (s, 1.5H), 2.77 (s, 1.5H), 2.73-2.65 (m, 2H), 2.47 (s, 1.5H), 2.43 (s, 1.5H), 1.16 (d, J=6.9 Hz, 1.5H), 1.08 (d, J=6.9 Hz, 1.5H).

### Example 10: tert-Butyl [({[2-(2H-1,3-benzodioxol-5-yl)-1-methyl-ethyl]-N-methylcarbamoyl}methyl)-N-methylcarbamoyl]acetate (Compound 325)

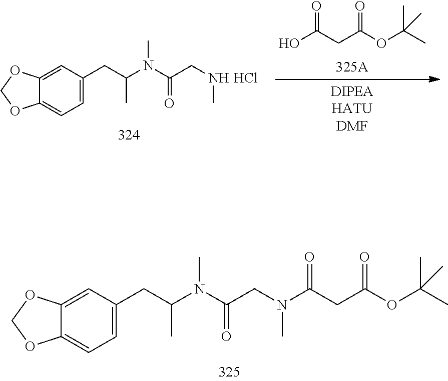 [SMILES: CNCC(=O)N(C)C(C)Cc1ccc2c(c1)OCO2 ; Cl ; CC(C)(C)OC(=O)CC(=O)O ; CC(Cc1ccc2c(c1)OCO2)N(C)C(=O)CN(C)C(=O)CC(=O)OC(C)(C)C]

HATU (362 mg, 0.95 mmol) was added in one portion to a stirred solution of N-[2-(1,3-benzodioxol-5-yl)-1-methyl-ethyl]-N-methyl-2-(methylamino)acetamide hydrochloride (324, 201 mg, 0.63 mmol), 3-tert-butoxy-3-oxo-propanoic acid (325A, 203 mg, 1.27 mmol) and DIPEA (492 mg, 3.81 mmol, 663 μL) in DMF (7 mL) at room temperature under an atmosphere of N₂. The mixture was stirred at room temperature overnight, then diluted with EtOAc (50 mL). The organic phase was washed with a 90% aqueous brine solution (50 mL) and then a 50% aqueous brine solution (3×50 mL) before being dried over Na₂SO₄ and concentrated in vacuo. The residue was purified by column chromatography on silica gel (eluting with a gradient of 0-2% MeOH in DCM) to give tert-butyl [({[2-(2H-1,3-benzodioxol-5-yl)-1-methyl-ethyl]-N-methylcarbamoyl}methyl)-N-methylcarbamoyl]acetate (Compound 325, 220 mg, 84%). Spectroscopic data of Compound 325 was obtained as a mixture of rotational isomers. LC-MS (LCMS2: Method 2A): Rt 1.47 mins; MS m/z 429.0=[M+Na]⁺. ¹H NMR (400 MHz, CDCl₃) δ 6.78-6.66 (m, 1.5H), 6.66-6.50 (m, 1.5H), 5.98-5.83 (m, 2H), 5.08-4.78 (m, 0.6H), 4.51 (d, J=15.7 Hz, 0.4H), 4.27-3.74 (m, 2H), 3.44-3.30 (m, 1.5H), 3.18-3.06 (m, 0.5H), 2.96-2.59 (m, 8H), 1.47 (s, 4.5H), 1.46 (s, 4.5H), 1.32 (d, J=6.8 Hz, 0.4H), 1.26 (d, J=6.8 Hz, 1.1H), 1.17 (d, J=6.8 Hz, 0.4H), 1.11 (d, J=6.8 Hz, 1.1H).

The following compounds listed in Table 8 were prepared using a similar procedure to the procedure for preparing Compound 325 using Compound 324 and an appropriate carboxylic acid in lieu of 3-tert-butoxy-3-oxo-propanoic acid (325A).

TABLE 8

| Compound | Structure and Name | Retention Time, [M + H]+, ¹H NMR |
|---|---|---|
| 326 | 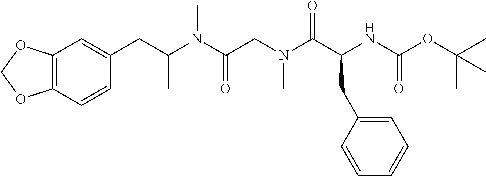 [SMILES: CC(Cc1ccc2c(c1)OCO2)N(C)C(=O)CN(C)C(=O)[C@H](Cc1ccccc1)NC(=O)OC(C)(C)C] | LC-MS (LCMS2: Method 2A): Rt 1.70 mins; MS m/z 512.1 = [M + H]⁺ Mixture of diastereoisomers: ¹H NMR (400 MHz, CDCl₃) δ 7.33 − 7.10 (m, 5H), 6.79 − 6.51 (m, 3H), 5.99 − 5.80 (m, 2H), 5.42 − 5.12 (m, 1.25H), 4.92 − 4.79 (m, 1.25H), 4.52 − 4.37 (m, 0.5H), 4.24 − 4.07 (m, 0.5H), 3.97 − 3.51 (m, 1.5H), 3.12 − 2.56 (m, 10H), 1.42 − 1.21 (m, 10.5H), 1.15 − 1.08 (m, 1.5H). |
| | (S)-1-[{{[2-(2H-1,3-Benzodioxol-5- | |
| | yl)-1-methylethyl]-N- | |
| | methylcarba}methyl)-N- | |
| | methylcarbamoyl]-2- | |
| | phenylethylamino-tert- | |
| | butylformylate | |
| 327 | 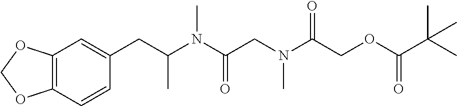 [SMILES: CC(Cc1ccc2c(c1)OCO2)N(C)C(=O)CN(C)C(=O)COC(=O)C(C)(C)C] | LC-MS (LCMS2: Method 2B): Rt 1.48 mins; MS m/z 407.3 = [M + H]⁺ ¹H NMR (400 MHz, CDCl₃) δ 6.83 − 6.50 (m, 3H), 5.98 − 5.84 (m, 2H), 5.02 − 3.15 (m, 5H), 3.13 − 2.54 (m, 8H), 1.36 − 1.09 (m, 12H). |
| | [{{[2-(2H-1,3-Benzodioxol-5-yl)-1- | |
| | methylethyl]-N- | |
| | methylcarbamoyl}methyl)-N- | |
| | methylcarbamoyl]methyl 2,2- | |
| | dimethylpropionate | |

### Example 11: Ammonium 3-[[2-[[2-(1,3-benzodioxol-5-yl)-1-methyl-ethyl]-methyl-amino]-2-oxo-ethyl]-methyl-amino]-3-oxo-propionate (Compound 328)

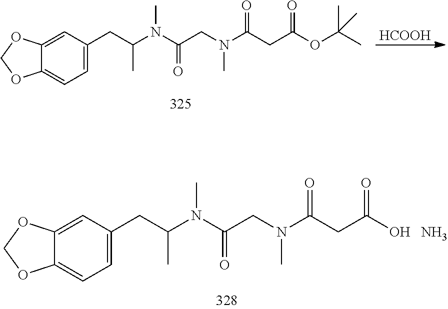

A mixture of tert-butyl [({[2-(2H-1,3-benzodioxol-5-yl)-1-methyl-ethyl]-N-methylcarbamoyl}methyl)-N-methylcarbamoyl]acetate (325, 166 mg, 0.40 mmol) in formic acid (3.66 g, 79.5 mmol, 3.00 mL) was stirred at room temperature under an atmosphere of N₂ for 18 h. The mixture was concentrated in vacuo and the residue was then azeotroped with chloroform (3×5 mL) and DCM (3×5 mL) before being dried under vacuum at 45° C. overnight. The residue was dissolved in 7M NH₃ in MeOH (2 mL) and then purified by reverse phase chromatography (eluting with a gradient of 10-50% MeCN in water with 0.1% aqueous ammonia). The combined product fractions were freeze dried to afford ammonium 3-[[2-[[2-(1,3-benzodioxol-5-yl)-1-methyl-ethyl]-methyl-amino]-2-oxo-ethyl]-methyl-amino]-3-oxo-propionate (Compound 328, 93 mg, 63%) as a glassy solid. Spectroscopic data of Compound 328 was obtained as a mixture of rotational isomers and as keto-enol tautomers. LC-MS (LCMS2: Method 2A): Rt 1.16 mins; MS m/z 351.2=[M+H]⁺. ¹H NMR (400 MHz, DMSO-d₆) δ 6.88-6.57 (m, 3H), 5.99-5.87 (m, 2H), 4.83-4.58 (m, 1H), 4.36-3.68 (m, 2H), 3.59 (br. s, 4H), 3.30-2.95 (m, 2H), 2.87-2.58 (m, 8H), 1.22-1.12 (m, 1.5H), 1.10-0.98 (m, 1.5H).

### Example 12: (2S)-2-amino-N-[2-[[2-(1,3-benzodioxol-5-yl)-1-methyl-ethyl]-methyl-amino]-2-oxo-ethyl]-N-methyl-3-phenyl-propanamide hydrochloride (328A)

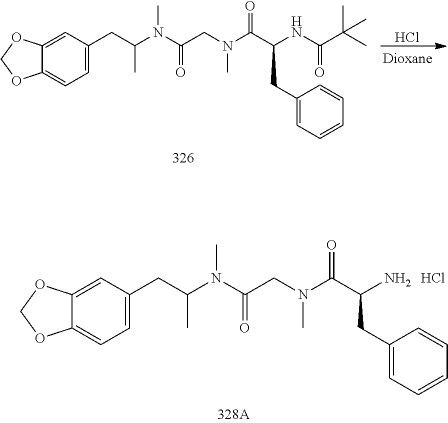 [SMILES: CC(Cc1ccc2c(c1)OCO2)N(C)C(=O)CN(C)C(=O)[C@H](Cc1ccccc1)NC(=O)C(C)(C)C ; Cl ; CC(Cc1ccc2c(c1)OCO2)N(C)C(=O)CN(C)C(=O)[C@@H](N)Cc1ccccc1 ; Cl]

A mixture of (S)-1-[({[2-(2H-1,3-Benzodioxol-5-yl)-1-methylethyl]-N-methylcarbamoyl}methyl)-N-methylcarbamoyl]-2-phenylethylamino-tert-butylformylate (326, 164 mg, 0.31 mmol) in 4M HCl in 1,4-dioxane (2 mL) was stirred at 0° C. under an atmosphere of N₂ for 4 h. The mixture was concentrated in vacuo and the residue was then co-evaporated with chloroform (3×5 mL) and DCM (3×5 mL) before being dried under vacuum at 45° C. overnight to afford (2S)-2-amino-N-[2-[[2-(1,3-benzodioxol-5-yl)-1-methyl-ethyl]-methyl-amino]-2-oxo-ethyl]-N-methyl-3-phenyl-propanamide HCl (Compound 328A, 143 mg, 99%) as a solid. Spectroscopic data of Compound 328 was obtained as a mixture of rotational isomers and diastereoisomers. LC-MS (LCMS2: Method 2A): Rt 1.16 mins; MS m/z 412.1=[M+H]⁺. ¹H NMR (400 MHz, DMSO-d₆) δ 8.18 (br. s, 3H), 7.36-7.17 (m, 5H), 6.91-6.54 (m, 3H), 6.03-5.67 (m, 2H), 4.82-3.38 (m, 4H), 3.23-2.52 (m, 10H), 1.24-1.02 (m, 3H).

### Example 13: Chloromethyl N-[2-(1,3-benzodioxol-5-yl)-1-methyl-ethyl]-N-methyl-carbamate (Compound 329)

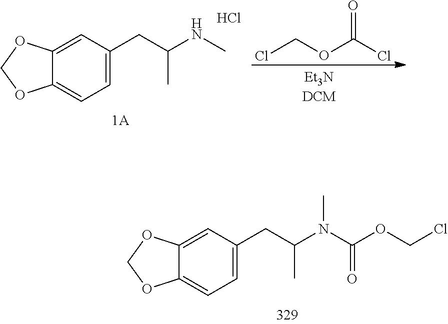

Chloromethyl chloroformate (569 mg, 4.41 mmol, 393 μL) was added dropwise over 2 min to a stirred mixture of 1-(1,3-benzodioxol-5-yl)-N-methyl-propan-2-amine HCl (1A, 507 mg, 2.21 mmol) and Et₃N (670 mg, 6.62 mmol, 728 μL) in DCM (4.5 mL) at −10° C. under an atmosphere of N₂. The mixture was stirred at −10° C. for 30 min, then warmed to rt and stirred for 2 h. The mixture was concentrated in vacuo and the residue was purified by chromatography on silica gel (EtOAc/PE, 0:1 to 1:3) to afford chloromethyl N-[2-(1,3-benzodioxol-5-yl)-1-methyl-ethyl]-N-methyl-carbamate (Compound 329, 520 mg, 82%) as an oil. Spectroscopic data of Compound 329 was obtained as a mixture of two rotational isomers. LC-MS (LCMS2: Method 2A): Rt 1.75 mins; MS m/z 286.0 and 288.0=[M+H]⁺. ¹H NMR (400 MHz, CDCl₃) δ 6.72 (d, J=7.9 Hz, 1H), 6.69-6.56 (m, 2H), 5.92 (s, 2H), 5.76-5.69 (m, 2H), 4.48-4.28 (m, 1H), 2.82 (s, 1.5H), 2.78-2.59 (m, 3.5H), 1.19-1.14 (m, 3H).

### Example 14: {[2-(2H-1,3-Benzodioxol-5-yl)-1-methylethyl]-N-methylaminocarbonyloxy}methyl tert-butyl succinate (Compound 330)

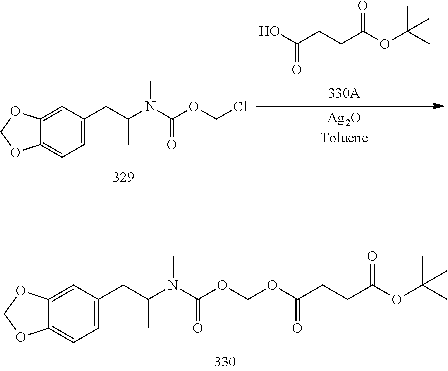 [SMILES: CC(C)(C)OC(=O)CCC(=O)O ; CC(Cc1ccc2c(c1)OCO2)N(C)C(=O)OCCl ; CC(Cc1ccc2c(c1)OCO2)N(C)C(=O)OCOC(=O)CCC(=O)OC(C)(C)C]

Silver (I) oxide (253 mg, 1.09 mmol) and 4-tert-butoxy-4-oxo-butanoic acid (330A, 190 mg, 1.09 mmol) were added in one portion for each reagent to a stirred mixture of chloromethyl N-[2-(1,3-benzodioxol-5-yl)-1-methyl-ethyl]-N-methyl-carbamate (329, 260 mg, 910 μmol) in toluene (20 mL) at rt under an atmosphere of N₂. The mixture was heated to 65° C. and stirred overnight. The mixture was cooled to room temperature before being filtered through a plug of celite. The filtrate was concentrated in vacuo and the residue was purified by column chromatography on silica gel (EtOAc/PE, 0:1 to 1:0) to afford {[2-(2H-1,3-benzodioxol-5-yl)-1-methylethyl]-N-methylaminocarbonyloxy}methyl tert-butyl succinate (Compound 330, 235 mg, 60) as an oil. Spectroscopic data of Compound 330 was obtained as a mixture of two rotational isomers. LC-MS (LCMS2: Method 2A): Rt 1.92 mins; MS m/z 446.2=[M+Na]⁺. ¹H NMR (400 MHz, CDCl₃) δ 6.75-6.66 (m, 1.5H), 6.66-6.52 (m, 1.5H), 5.92 (br. s, 2H), 5.76-5.69 (m, 2H), 4.48-4.28 (m, 1H), 2.80-2.52 (m, 9H), 1.44 (br. s, 9H), 1.14 (d, J=6.8 Hz, 3H).

The following compounds listed in Table 9 were prepared using a similar procedure to the procedure for preparing Compound 330 using Compound 329 and an appropriate carboxylic acid in lieu of 4-tert-butoxy-4-oxo-butanoic acid (330A).

TABLE 9

| | | Retention Time, [M + H]⁺, |
|---|---|---|
| Compound | Structure and Name | ¹H NMR |
| 331 | 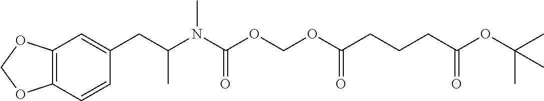 [SMILES: CC(Cc1ccc2c(c1)OCO2)N(C)C(=O)OCOC(=O)CCCC(=O)OC(C)(C)C] {[2-(2H-1,3-Benzodioxol-5-yl)-1- methylethyl]-N- methylaminocarbonyloxy}methyl tert-butyl glutarate | LC-MS (LCMS2: Method 2A): Rt 1.94 mins; MS m/z 460.2 = [M + Na]⁺ ¹H NMR (400 MHz, CDCl₃) δ 6.75 − 6.65 (m, 1.5H), 6.65 − 6.52 (m, 1.5H), 5.92 (br. s, 2H), 5.77 − 5.65 (m, 2H), 4.47 − 4.27 (m, 1H), 2.82 − 2.57 (m, 5H), 2.43 − 2.36 (m, 2H), 2.30 − 2.22 (m, 2H), 1.91 (app. p, J = 7.4 Hz, 2H), 1.44 (s, 4.5H), 1.43 (s, 4.5H), 1.14 (d, J = 6.8 Hz, 3H). |
| 332 | 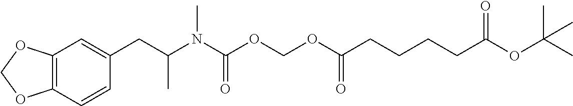 [SMILES: CC(Cc1ccc2c(c1)OCO2)N(C)C(=O)OCOC(=O)CCCCC(=O)OC(C)(C)C] {[2-(2H-1,3-Benzodioxol-5-yl)-1-methylethyl]-N- methylaminocarbonyloxy}methyl tert-butyl adipate | LC-MS (LCMS2: Method 2A): Rt 2.00 mins; MS m/z 474.2 = [M + Na]⁺ ¹H NMR (400 MHz, CDCl₃) δ 6.74 − 6.65 (m, 1.5H), 6.65 − 6.51 (m, 1.5H), 5.92 (br. s, 2H), 5.76 − 5.66 (m, 2H), 4.42 (app. h, J = 6.9 Hz, 0.5H), 4.32 (app. h, J = 6.9 Hz, 0.5H), 2.83 − 2.56 (m, 5H), 2.39 − 2.31 (m, 2H), 2.25 − 2.17 (m, 2H), |
| | | 1.70 − 1.56 (m, 4H), 1.43 (br. s, |
| | | 9H), 1.14 (d, J = 6.9 Hz, 3H). |

### Example 15: 4-[[[2-(1,3-Benzodioxol-5-yl)-1-methyl-ethyl]-methyl-carbamoyl]oxymethoxy]-4-oxo-butanoic acid (Compound 333)

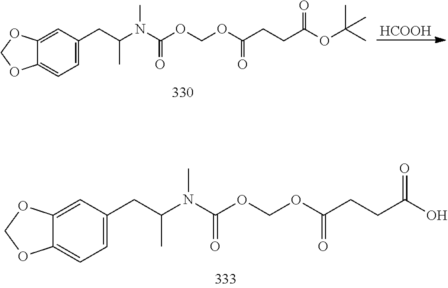 [SMILES: CC(Cc1ccc2c(c1)OCO2)N(C)C(=O)OCOC(=O)CCC(=O)OC(C)(C)C ; "HCOOH" ; CC(Cc1ccc2c(c1)OCO2)N(C)C(=O)OCOC(=O)CCC(=O)O]

A mixture of {[2-(2H-1,3-benzodioxol-5-yl)-1-methylethyl]-N-methylaminocarbonyloxy}methyl tert-butyl succinate (330, 179 mg, 423 μmol) in formic acid (10.88 g, 236.4 mmol, 8.92 mL) was stirred at rt under an atmosphere of N₂ overnight. The mixture was concentrated in vacuo and then chloroform (5 mL) was added to the residue. The mixture was concentrated in vacuo to give 4-[[[2-(1,3-benzodioxol-5-yl)-1-methyl-ethyl]-methyl-carbamoyl]oxymethoxy]-4-oxo-butanoic acid (Compound 333, 153 mg, 98%) as an oil. Spectroscopic data of Compound 333 was obtained as a mixture of two rotational isomers. LC-MS (LCMS2: Method 2A): Rt 1.58 mins; MS m/z 368.1=[M+H]⁺. ¹H NMR (400 MHz, CDCl₃) δ 6.84-6.66 (m, 1.6H), 6.66-6.53 (m, 1.4H), 5.97-5.88 (m, 2H), 5.78-5.68 (m, 2H), 4.47-4.29 (m, 1H), 2.82-2.57 (m, 9H), 1.21-1.12 (m, 3H).

The following compounds listed in Table 10 were prepared using a similar procedure to the procedure for preparing Compound 333 using Compound 331 or Compound 332.

TABLE 10

| Compound | Structure and Name | Retention Time, [M + H]+, ¹H NMR |
|---|---|---|
| 334 | 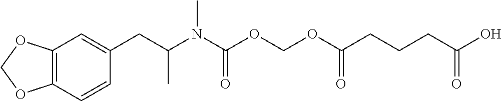 [SMILES: CC(Cc1ccc2c(c1)OCO2)N(C)C(=O)OCOC(=O)CCCC(=O)O] 5-[[[2-(1,3-Benzodioxol-5-yl)-1-methyl-ethyl]-methyl- carbamoyl]oxymethoxy]-5-oxo-pentanoic acid | LC-MS (LCMS2: Method 2A): Rt 1.61 mins; MS m/z 382.1 = [M + H]+ ¹H NMR (400 MHz, CDCl₃) δ 6.84 − 6.66 (m, 1.6H), 6.64 − 6.54 (m, 1.4H), 5.95 − 5.90 (m, 2H), 5.75 − 5.67 (m, 2H), 4.50 − 4.26 (m, 1H), 2.83 − 2.57 (m, 5H), 2.47 − 2.40 (m, 4H), 1.96 (app. p, J = 7.3 Hz, 2H), 1.20 − 1.12 (m, 3H). CO₂H not observed. |
| 335 | 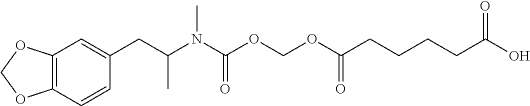 [SMILES: CC(Cc1ccc2c(c1)OCO2)N(C)C(=O)OCOC(=O)CCCCC(=O)O] 6-[[[2-(1,3-Benzodioxol-5-yl)-1-methyl-ethyl]-methyl- carbamoyl]oxymethoxy]-6-oxo-hexanoic acid | LC-MS (LCMS2: Method 2A): Rt 1.65 mins; MS m/z 396.2 = [M + H]+ ¹H NMR (400 MHz, CDCl₃) δ 6.88 − 6.51 (m,3H), 5.97 − 5.89 (m, 2H), 5.76 − 5.65 (m, 2H), 4.48 − 4.28 (m, 1H), 2.86 − 2.57 (m, 5H), 2.42 − 2.34 (m, 4H), 1.73 − 1.66 (m, 4H), 1.21 − 1.13 (m, 3H). CO₂H not observed. |

### Example 16: (1-Methyl-4-piperidyl)N-[2-(1,3-benzodioxol-5-yl)-1-methyl-ethyl]-N-methyl-carbamate (Compound 336)

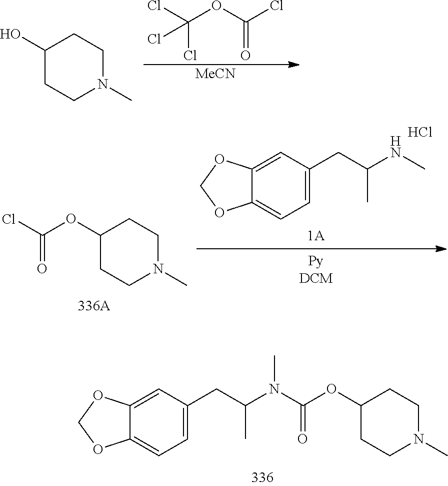

Trichloromethyl chloroformate (893 mg, 4.51 mmol, 545 μL) was added dropwise over 2 min to a stirred mixture of 1-methylpiperidin-4-ol (0.40 g, 3.47 mmol) in MeCN (5 mL) at 0° C. under an atmosphere of N₂. The mixture was stirred at 0° C. for 30 min, then warmed to rt and stirred overnight. The mixture was concentrated in vacuo to give 1-methyl-4-piperidinyl chloroformate HCl (intermediate 336A, 744 mg, assumed 100%) as an oil, which was used directly in the next step without further purification.

1-(1,3-Benzodioxol-5-yl)-N-methyl-propan-2-amine HCl (1A, 250 mg, 1.09 mmol) was added in several portions over 10 min to a stirred mixture of 1-methyl-4-piperidinyl chloroformate HCl (336A, 292 mg, 1.36 mmol) in DCM (5 mL) at 0° C. under an atmosphere of N₂. The mixture was stirred at 0° C. for 10 min, then pyridine (237 mg, 3.00 mmol, 242 μL) was added dropwise over 10 min. The mixture was stirred at 0° C. 10 min, then warmed to room temperature and stirred for 1 h. The mixture was diluted with DCM (50 mL) and washed with saturated aqueous NaHCO₃ (2×50 mL). The organic phase was dried over Na₂SO₄ and then concentrated in vacuo. The residue was purified by reverse phase chromatograph (eluting with a gradient of 0-50% MeCN in water with 0.1% w/w ammonia) to give (1-methyl-4-piperidyl)N-[2-(1,3-benzodioxol-5-yl)-1-methyl-ethyl]-N-methyl-carbamate (Compound 336, 125 mg, 34) as an oil. Spectroscopic data of Compound 336 was obtained as a mixture of two rotational isomers at 298 K, which coalesced at 343 K. LC-MS (LCMS2: Method 2A): Rt 1.11 mins; MS m/z 335.2=[M+H]⁺. ¹H NMR (400 MHz, DMSO-d₆, T=298 K) δ 6.85-6.75 (m, 1H), 6.72 (br. s, 1H), 6.61 (d, J=7.9 Hz, 1H), 5.94 (br. s, 2H), 4.43 (br. s, 1H), 4.35-4.20 (m, 1H), 2.66 (s, 3H), 2.65-2.56 (m, 2H), 2.45-2.28 (m, 2H), 2.24-2.01 (m, 5H), 1.78-1.47 (m, 3H), 1.43-1.28 (m, 1H), 1.14-1.01 (m, 3H). ¹H NMR (400 MHz, DMSO-d₆, T=343 K) δ 6.77 (d, J=7.9 Hz, 1H), 6.71 (d, J=1.7 Hz, 1H), 6.62 (dd, J=7.9, 1.7 Hz, 1H), 5.93 (s, 2H), 4.48 (tt, J=7.6, 3.9 Hz, 1H), 4.34-4.23 (m, 1H), 2.72-2.59 (m, 5H), 2.46-2.37 (m, 2H), 2.21-2.12 (m, 5H), 1.79-1.64 (m, 2H), 1.58-1.41 (m, 2H), 1.11 (d, J=6.8 Hz, 3H).

### Example 17: Tetrahydropyran-4-yl N-[2-(1,3-benzodioxol-5-yl)-1-methyl-ethyl]-N-methyl-carbamate (Compound 337)

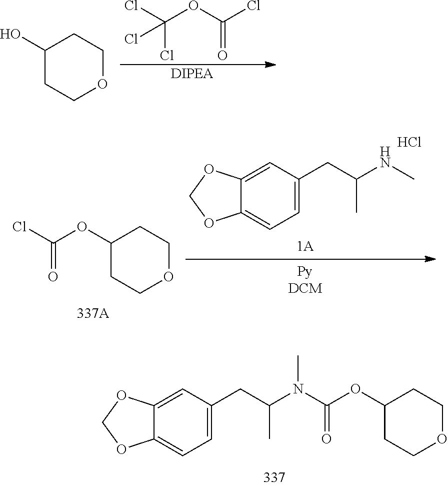

Trichloromethyl chloroformate (581 mg, 2.94 mmol, 354 μL) was added dropwise over 2 min to a stirred mixture of tetrahydropyran-4-ol (0.20 g, 1.96 mmol, 187 μL) and DIPEA (557 mg, 4.31 mmol, 750 μL) in MeCN (5 mL) at 0° C. under an atmosphere of N₂. The mixture was stirred at 0° C. for 30 min, then warmed to room temperature and stirred overnight. The mixture was concentrated in vacuo to afford 322 mg (assumed 100% yield) of tetrahydro-2H-pyranyl-4-chloroformate (intermediate 337A) as an oil, which was used directly in the next step without further purification.

1-(1,3-Benzodioxol-5-yl)-N-methyl-propan-2-amine HCl (1A, 250 mg, 1.09 mmol) was added in several portions over 10 min to a stirred mixture of tetrahydro-2H-pyranyl-4-chloroformate (337A, 211 mg, 1.28 mmol) in DCM (5 mL) at 0° C. under an atmosphere of N₂. The mixture was stirred at 0° C. for 10 min, then pyridine (237 mg, 3.00 mmol, 242 μL) was added dropwise over 10 min. The mixture was stirred at 0° C. for 10 min, warmed to room temperature and then stirred for 1 h. The mixture was diluted with DCM (50 mL) and then washed with saturated aqueous NaHCO₃ (2×50 mL). The organic layer was dried over Na₂SO₄ and then concentrated in vacuo. The residue was purified by reverse phase chromatography (eluting with a gradient of 0-50% MeCN in water with 0.1% w/w ammonia) to give tetrahydropyran-4-yl N-[2-(1,3-benzodioxol-5-yl)-1-methyl-ethyl]-N-methyl-carbamate (Compound 337, 305 mg, 68%) of as an oil. Spectroscopic data of Compound 337 was obtained as a mixture of two rotational isomers at 298 K, which coalesced at 343 K. LC-MS (LCMS2: Method 2A): Rt 1.61 mins; MS m/z 322.2=[M+H]⁺. ¹H NMR (400 MHz, DMSO-d₆, T=298 K) δ 6.83-6.69 (m, 2H), 6.61 (dd, J=7.9, 1.7 Hz, 1H), 5.93 (br. s, 2H), 4.67-4.54 (m, 1H), 4.37-4.24 (m, 1H), 3.75-3.56 (m, 2H), 3.48-3.38 (m, 2H), 2.67 (s, 3H), 2.66-2.59 (m, 2H), 1.84-1.60 (m, 2H), 1.50-1.41 (m, 1H), 1.37-1.21 (m, 1H), 1.16-1.03 (m, 3H). ¹H NMR (400 MHz, DMSO-d₆, T=343 K) δ 6.77 (d, J=7.8 Hz, 1H), 6.72 (s, 1H), 6.62 (d, J=7.8 Hz, 1H), 5.93 (s, 2H), 4.65 (tt, J=8.0, 4.0 Hz, 1H), 4.35-4.25 (m, 1H), 3.75-3.65 (m, 2H), 3.49-3.40 (m, 2H), 2.72-2.59 (m, 5H), 1.82-1.69 (m, 2H), 1.51-1.34 (m, 2H), 1.12 (d, J=6.8 Hz, 3H).

### Example 18: [3-[[2-(1,3-Benzodioxol-5-yl)-1-methyl-ethyl]-methyl-carbamoyl]oxy-2,2-dimethyl-propyl] 2,2-dimethylpropanoate (Compound 338)

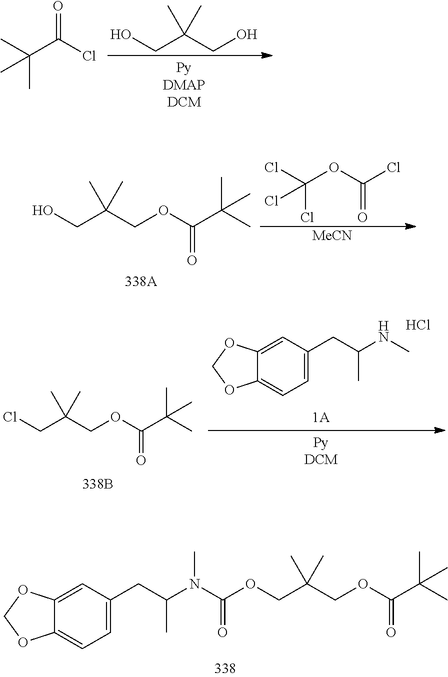

### (3-Hydroxy-2,2-dimethyl-propyl) 2,2-dimethylpropanoate (338A)

Pivaloyl chloride (2.00 g, 16.6 mmol, 2.03 mL) was added dropwise over 15 min to a stirred mixture of 2,2-dimethylpropane-1,3-diol (5.18 g, 49.8 mmol), pyridine (2.62 g, 33.2 mmol, 2.68 mL) and DMAP (405 mg, 3.32 mmol) in DCM (50 mL) at 0° C. under an atmosphere of N₂. The mixture was stirred at 0° C. for 10 min, then warmed to room temperature and stirred overnight. The mixture was cooled to 0° C., then 1M HCl (50 mL) was added. The layers were separated, and the aqueous phase was extracted with DCM (2×50 mL). The combined organic layers were washed with saturated aqueous NaHCO₃ (100 mL) and brine (50 mL), dried over Na₂SO₄ and then concentrated in vacuo. The residue was purified by column chromatography on silica gel (EtOAc/PE, 0:1 to 2:3) to give (3-hydroxy-2,2-dimethyl-propyl) 2,2-dimethylpropanoate (intermediate 338A, 2.57 g, 82%) as an oil. ¹H NMR (400 MHz, CDCl₃) δ 3.92 (s, 2H), 3.27 (s, 2H), 2.32 (br s, 1H), 1.22 (s, 9H), 0.92 (s, 6H).

Trichloromethyl chloroformate (775 mg, 3.92 mmol, 473 μL) was added dropwise to a stirred mixture of (3-hydroxy-2,2-dimethyl-propyl) 2,2-dimethylpropanoate (338A, 177 mg, 0.94 mmol) in MeCN (2 mL) at 0° C. under an atmosphere of N₂. The mixture was stirred at 0° C. for 30 min and then warmed to rt and stirred overnight. The mixture was concentrated in vacuo to afford 236 mg (assumed 100% yield) of 3-chloroformate-2,2-dimethylpropyl 2,2-dimethylpropionate (intermediate 338B) as an oil, that was used directly in the next step without further purification.

1-(1,3-Benzodioxol-5-yl)-N-methyl-propan-2-amine HCl (1A, 180 mg, 0.78 mmol) was added in several portions over 10 min to a stirred mixture of 3-chloroformate-2,2-dimethylpropyl 2,2-dimethylpropionate (338B, 236 mg, 0.94 mmol) in MeCN (3 mL) at 0° C. under an atmosphere of N₂. The mixture was stirred at 0° C. for 10 min, then pyridine (310 mg, 3.92 mmol, 317 μL) was added dropwise over 10 min. The mixture was stirred at 0° C. for 1 h, then warmed to room temperature and stirred overnight. The mixture was diluted with EtOAc (25 mL) and washed with 1M HCl (2×25 mL), saturated aqueous NaHCO₃ (25 mL) and brine (25 mL). The organic phase was dried over Na₂SO₄ and then concentrated in vacuo. The residue was purified by column chromatography on silica gel (EtOAc/isohexane, 0:1 to 1:3) to give [3-[[2-(1,3-benzodioxol-5-yl)-1-methyl-ethyl]-methyl-carbamoyl]oxy-2,2-dimethyl-propyl] 2,2-dimethylpropanoate (Compound 338, 224 mg, 69%) as an oil. Spectroscopic data of Compound 338 was obtained as a mixture of two rotational isomers. LC-MS (LCMS2: Method 2B): Rt 1.91 mins; MS m/z 408.3=[M+H]⁺. ¹H NMR (400 MHz, CDCl₃) δ 6.72-6.55 (m, 3H), 5.90 (s, 2H), 4.50-4.38 (m, 0.5H), 4.37-4.23 (m, 0.5H), 3.89-3.77 (m, 4H), 2.80-2.66 (m, 4H), 2.59 (dd, J=13.8, 7.2 Hz, 1H), 1.20 (s, 9H), 1.16-1.09 (m, 3H), 0.95-0.89 (m, 6H).

### Example 19: Tetrahydrofuran-3-yl N-[2-(1,3-benzodioxol-5-yl)-1-methyl-ethyl]-N-methyl-carbamate (Compound 339)

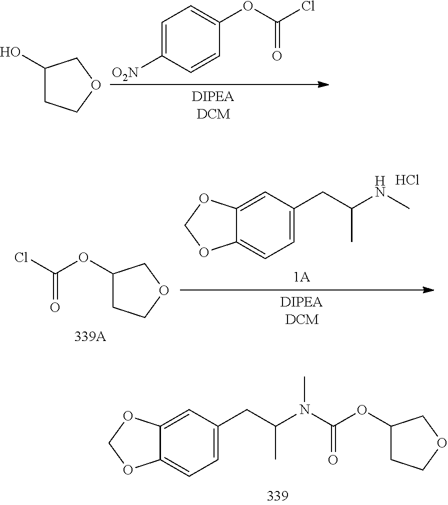

Tetrahydrofuran-3-ol (91 mg, 1.04 mmol, 83 μL) and DIPEA (441 mg, 3.42 mmol, 595 L) were added sequentially, dropwise over 2 min for each reagent, to a stirred solution of 4-nitrophenylchloroformate (intermediate 339A, 209 mg, 1.04 mmol) in DCM (5 mL) at 0° C. under an atmosphere of N₂. The mixture was stirred at 0° C. for 10 min, warmed to rt and then stirred for 1 h. 1-(1,3-Benzodioxol-5-yl)-N-methyl-propan-2-amine HCl salt (1A, 300 mg, 1.31 mmol) was added in one portion, followed by DIPEA (441 mg, 3.42 mmol, 595 μL) which was added dropwise over 2 min to the mixture. The mixture was warmed to 40° C. and stirred overnight. The mixture was concentrated in vacuo and the residue was purified by column chromatography on silica gel (EtOAc/PE, 0:1 to 1:0) to give tetrahydrofuran-3-yl N-[2-(1,3-benzodioxol-5-yl)-1-methyl-ethyl]-N-methyl-carbamate (Compound 339, 63 mg, 19%) as an oil. Spectroscopic data of Compound 339 was obtained as a mixture of two rotational isomers and diastereoisomers. LC-MS (LCMS2: Method 1B): Rt 1.29 mins; MS m/z 330.1=[M+Na]⁺. ¹H NMR (400 MHz, CDCl₃) δ 6.77-6.50 (m, 3H), 5.91 (s, 2H), 5.24-5.07 (m, 1H), 4.51-4.25 (m, 1H), 3.93-3.78 (m, 3.5H), 3.70 (d, J=10.6 Hz, 0.25H), 3.47 (d, J=10.6 Hz, 0.25H), 2.80-2.55 (m, 5H), 2.15-1.87 (m, 1.75H), 1.75-1.68 (m, 0.25H), 1.19-1.05 (m, 3H).

### Example 20: N-[2-(2H-1,3-Benzodioxol-5-yl)-1-methylethyl]-N-methyl-4-methyltetrahydro-2H-pyran-4-carboxamide (Compound 340)

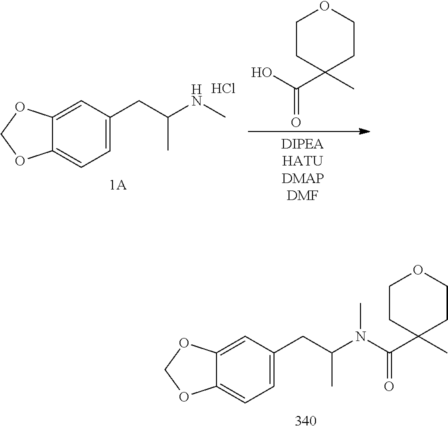

HATU (745 mg, 1.96 mmol) was added in one portion, followed by DIPEA (557 mg, 4.31 mmol, 751 μL) which was added dropwise over 5 min to a stirred solution of 1-(1,3-benzodioxol-5-yl)-N-methyl-propan-2-amine HCl (1A, 300 mg, 1.31 mmol), 4-methyltetrahydropyran-4-carboxylic acid (207 mg, 1.44 mmol) and DMAP (16 mg, 0.13 mmol) in DMF (5 mL) at room temperature under N₂. The mixture was heated to 40° C. and stirred for 3 h, then cooled to rt, filtered through a plug of celite and concentrated in vacuo. The residue was purified by column chromatography on silica gel, eluting with a gradient of 0-10% MeOH in DCM to afford N-[2-(2H-1,3-benzodioxol-5-yl)-1-methyl-ethyl]-N-methyl-4-methyltetrahydro-2H-pyran-4-carboxamide (Compound 340, 86 mg, 20% yield) as an oil. LC-MS (LCMS2: Method 2A): Rt 1.90 mins; MS m/z 320.2=[M+H]^{+ 1}H NMR (400 MHz, CDCl₃) δ 6.72 (d, J=7.9 Hz, 1H), 6.67 (br. s, 1H), 6.62 (dd, J=7.9, 1.7 Hz, 1H), 5.94-5.89 (m, 2H), 4.75 (br. s, 1H), 3.70-3.60 (m, 2H), 3.52-3.28 (m, 2H), 2.87 (s, 3H), 2.78-2.61 (m, 2H), 2.16-2.02 (m, 2H), 1.51-1.42 (m, 2H), 1.21 (s, 3H), 1.14 (d, J=6.8 Hz, 3H).

The following compound listed in Table 11 was prepared using a similar procedure to the procedure for preparing Compound 340 using 1-(1,3-benzodioxol-5-yl)-N-methyl-propan-2-amine hydrochloride and the appropriate carboxylic acid.

TABLE 11

| Compound | Structure and Name | Retention Time, [M + H]+, 1H NMR |
|---|---|---|
| 341 | 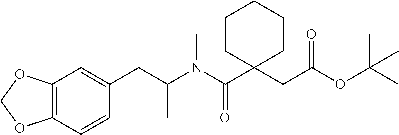 [SMILES: CC(Cc1ccc2c(c1)OCO2)N(C)C(=O)C1(CC(=O)OC(C)(C)C)CCCCC1] | LC-MS (LCMS2: Method 2A): Rt 1.65 mins; MS m/z 420.3 = [M + H]⁺ |
| | tert-Butyl (4-{[2-(2H-1,3- | |
| | benzodioxol-5-yl)-1-methyl-ethyl]- | |
| | N-methylcarbamoyl}tetrahydro-2H- | |
| | pyran-4-yl)acetate | |

### Example 21: 1-Chloroethyl N-[2-(1,3-benzodioxol-5-yl)-1-methyl-ethyl]-N-methyl-carbamate (Compound 342)

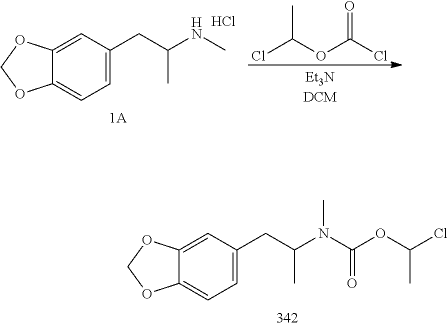

1-Chloroethyl chloroformate (188 mg, 1.32 mmol, 142 μL) was added dropwise over 2 min to a stirred solution of 1-(1,3-benzodioxol-5-yl)-N-methyl-propan-2-amine HCl (252 mg, 1.10 mmol) and Et₃N (333 mg, 3.29 mmol, 459 μL) in DCM (10 mL) at 0° C. under an atmosphere of N₂. The mixture was stirred at 0° C. for 1.5 h, then directly purified by column chromatography on silica gel, eluting with a gradient of 0-100% EtOAc in petroleum ether, to afford 1-chloroethyl N-[2-(1,3-benzodioxol-5-yl)-1-methyl-ethyl]-N-methyl-carbamate (Compound 342, 151 mg, 46% yield) as a gum. Spectroscopic data of Compound 342 was obtained as a mixture of rotational isomers and diastereoisomers. LC-MS (LCMS2: Method 2A): Rt 1.64 mins; MS m/z 300.1 and 302.2=[M+H]⁺. ¹H NMR (400 MHz, CDCl₃) δ 6.74-6.45 (m, 4H), 5.95-5.88 (m, 2H), 4.48-4.29 (m, 1H), 2.83-2.57 (m, 5H), 1.82-1.77 (m, 1.9H), 1.69 (d, J=5.7 Hz, 0.7H), 1.53-1.49 (m, 0.4H), 1.22-1.12 (m, 3H).

### Example 22: 1-{[2-(2H-1,3-Benzodioxol-5-yl)-1-methyl-ethyl]-N-methylaminocarbonyloxy}ethyl tetrahydro-2H-pyran-4-carboxylate (Compound 343)

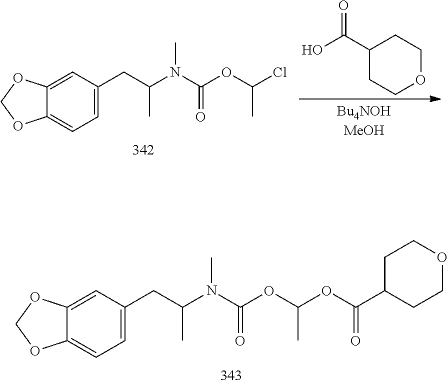 [SMILES: CC(Cl)OC(=O)N(C)C(C)Cc1ccc2c(c1)OCO2 ; O=C(O)C1CCOCC1 ; CC(OC(=O)C1CCOCC1)OC(=O)N(C)C(C)Cc1ccc2c(c1)OCO2]

A solution of 1M Bu₄NOH in MeOH (0.75 mmol, 751 μL) was added dropwise over 2 min to a stirred solution of tetrahydropyran-4-carboxylic acid (98 mg, 0.75 mmol) in MeOH (2 mL) at rt under an atmosphere of N₂. The mixture was stirred at rt for 1 h and then concentrated in vacuo. A solution of 1-chloroethyl N-[2-(1,3-benzodioxol-5-yl)-1-methyl-ethyl]-N-methyl-carbamate (342, 150 mg, 0.50 mmol) in THE (3 mL) was added to the residue and the mixture was then stirred at rt overnight, then concentrated in vacuo and the residue was dissolved in EtOAc (25 mL). The organic phase was washed with H₂O (2×25 mL) and brine (25 mL), dried over Na₂SO₄, filtered and concentrated in vacuo. The residue was purified by column chromatography on silica gel, eluting with a gradient of 0-50% EtOAc in iso-hexane, to afford 1-{[2-(2H-1,3-benzodioxol-5-yl)-1-methyl-ethyl]-N-methylaminocarbonyloxy}ethyl tetrahydro-2H-pyran-4-carboxylate (Compound 343, 136 mg, 68% yield) as a gum. Spectroscopic data of Compound 343 was obtained as a mixture of rotational isomers and diastereoisomers. LC-MS (LCMS2: Method 2A): Rt 1.61 mins; MS m/z 416.2=[M+Na]⁺. ¹H NMR (400 MHz, CDCl₃) δ 6.85-6.51 (m, 4H), 5.99-5.85 (m, 2H), 4.47-4.22 (m, 1H), 4.00-3.88 (m, 2H), 3.49-3.36 (m, 2H), 2.81-2.45 (m, 6H), 1.87-1.69 (m, 4H), 1.51-1.41 (m, 2.3H), 1.36 (d, J=5.5 Hz, 0.7H), 1.20-1.10 (m, 3H).

The following compound listed in Table 12 was prepared using a similar procedure to the procedure for preparing Compound 343 using 1-chloroethyl N-[2-(1,3-benzodioxol-5-yl)-1-methyl-ethyl]-N-methyl-carbamate and the appropriate carboxylic acid.

TABLE 12

| Compound | Structure and Name | Retention Time, [M + H]⁺, ¹H NMR |
|---|---|---|
| 344 | 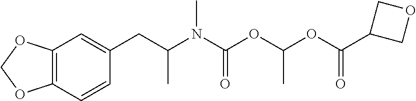 [SMILES: CC(OC(=O)C1COC1)OC(=O)N(C)C(C)Cc1ccc2c(c1)OCO2] | LC-MS (LCMS2: Method 2B): Rt 1.49 mins; MS m/z 388.2 = [M + Na]⁺ ¹H NMR (400 MHz, CDCl₃) δ 6.87 − 6.54 (m, 4H), 5.97 − 5.89 (m, 2H), 4.91 − 4.70 (m, 4H), 4.46 − 4.26 (m, 1H), 3.92 − 3.71 (m, 1H), 2.79 − 2.54 (m, 5H), 1.53 − 1.34 (m, 3H), 1.20 − 1.09 (m, 3H). |
| | 1-{[2-(2H-1,3-Benzodioxol-5-yl)-1- | |
| | methyl-ethyl]-N- | |
| | methylaminocarbonyloxy}ethyl 3- | |
| | oxetanecarboxylate | |

### Example 23: {[2-(2H-1,3-Benzodioxol-5-yl)-1-methyl-ethyl]-N-methylaminocarbonyloxy}methyl tetrahydro-2H-pyran-4-carboxylate (Compound 345)

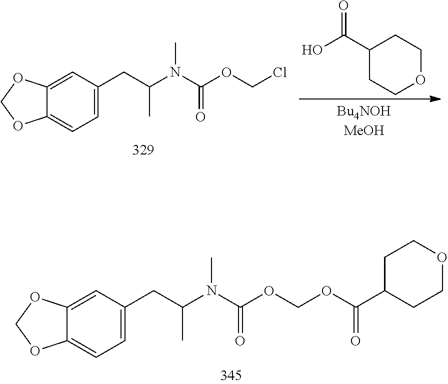 [SMILES: CC(Cc1ccc2c(c1)OCO2)N(C)C(=O)OCCl ; O=C(O)C1CCOCC1 ; CC(Cc1ccc2c(c1)OCO2)N(C)C(=O)OCOC(=O)C1CCOCC1]

A solution of 1 M Bu₄NOH in MeOH (1.32 mmol, 1.32 mL) was added dropwise over 2 min to a stirred solution of tetrahydropyran-4-carboxylic acid (172 mg, 1.32 mmol) in MeOH (3.5 mL) at rt under N₂. The mixture was stirred at rt for 1 h and then concentrated in vacuo. A solution of chloromethyl N-[2-(1,3-benzodioxol-5-yl)-1-methyl-ethyl]-N-methyl-carbamate (329, 252 mg, 0.88 mmol) in THE (5 mL) was added to the residue and the mixture was then stirred at rt overnight. The mixture was concentrated in vacuo and the residue was then dissolved in EtOAc (25 mL). The organic phase was washed with water (2×25 mL) and brine (25 mL), dried over Na₂SO₄ and concentrated in vacuo. The residue was purified by chromatography on silica, eluting with a gradient of 0-50% EtOAc in iso-hexane, to afford {[2-(2H-1,3-benzodioxol-5-yl)-1-methyl-ethyl]-N-methylaminocarbonyloxy}methyl tetrahydro-2H-pyran-4-carboxylate (Compound 345, 242 mg, 70% yield) as a gum. Spectroscopic data of Compound 345 was obtained as a mixture of two rotational isomers. LC-MS (LCMS2: Method 2B): Rt 1.44 mins; MS m/z 380.0=[M+H]⁺. ¹H NMR (400 MHz, CDCl₃) δ 6.73-6.65 (m, 1.5H), 6.65-6.54 (m, 1.5H), 5.95-5.89 (m, 2H), 5.74 (s, 1H), 5.72 (s, 1H), 4.43 (app. h, J=7.0 Hz, 0.5H), 4.31 (app. h, J=7.0 Hz, 0.5H), 3.99-3.90 (m, 2H), 3.46-3.36 (m, 2H), 2.79 (s, 1.5H), 2.76-2.53 (m, 4.5H), 1.87-1.69 (m, 4H), 1.18-1.11 (m, 3H).

The following compound listed in Table 13 was prepared using a similar procedure to the procedure for preparing Compound 345 using chloromethyl N-[2-(1,3-benzodioxol-5-yl)-1-methyl-ethyl]-N-methyl-carbamate and the appropriate carboxylic acid.

TABLE 13

| Compound | Structure and Name | Retention Time, [M + H]+, ¹H NMR |
|---|---|---|
| 346 | 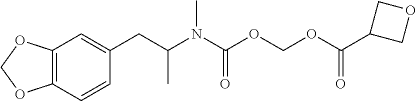 [SMILES: CC(Cc1ccc2c(c1)OCO2)N(C)C(=O)OCOC(=O)C1COC1] {[2-(2H-1,3-Benzodioxol-5-yl)-1-methyl-ethyl]-N- | LC-MS (LCMS2: Method 2A): Rt 1.18 mins; MS m/z 352.0 = [M + H]⁺ ¹H NMR (400 MHz, CDCl₃) δ 6.73 − 6.66 (m, 1.5H), 6.64 − 6.55 (m, 1.5H), 5.94 − 5.90 (m, 2H), 5.78 (s, 1H), 5.75 (s, 1H), 4.87 − 4.76 (m, 4H), 4.48 − 4.29 (m, 1H), 3.92 − 3.82 (m, 1H), 2.80 (s, 1.5H), 2.77 − 2.58 (m, 3.5H), 1.18 − 1.12 (m, 3H). |
| | methylaminocarbonyloxy}methyl 3- | |
| | oxetanecarboxylate | |

### Example 24: Oxetan-3-yl N-[2-(1,3-benzodioxol-5-yl)-1-methyl-ethyl]-N-methyl-carbamate (Compound 347)

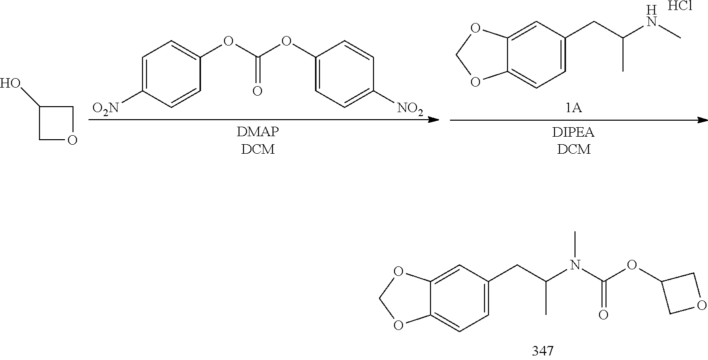

Oxetan-3-ol (99 mg, 1.33 mmol, 85 μL) was added dropwise over 2 min to a stirred solution of bis(4-nitrophenyl) carbonate (407 mg, 1.33 mmol) and DMAP (15 mg, 0.12 mmol) in DCM (4 mL) at rt under an atmosphere of N₂. The mixture was stirred at rt for 1 h. 1-(1,3-Benzodioxol-5-yl)-N-methyl-propan-2-amine HCl (1A, 323 mg, 1.21 mmol) was added in one portion to the mixture at room temperature, followed by DIPEA (157 mg, 1.21 mmol, 211 μL) which was added dropwise over 5 min. The mixture was stirred at room temperature for 1 h, then H₂O (5 mL) and DCM (5 mL) were added to the mixture. The separated aqueous phase was extracted with DCM (5 mL) and the combined organic fractions were dried over Na₂SO₄, filtered and concentrated in vacuo. The residue was purified by column chromatography on silica gel, eluting with a gradient of 40-50% EtOAc in petroleum ether, to afford oxetan-3-yl N-[2-(1,3-benzodioxol-5-yl)-1-methyl-ethyl]-N-methyl-carbamate (Compound 347, 62 mg, 17% yield) as an oil. Spectroscopic data of Compound 347 was obtained as a mixture of two rotational isomers. LC-MS (LCMS2: Method 2A): Rt 1.30 mins; MS m/z 293.9=[M+H]⁺. ¹H NMR (400 MHz, CDCl₃) δ 6.76-6.69 (m, 1H), 6.69-6.64 (m, 1H), 6.63-6.55 (m, 1H), 5.92 (br. s, 2H), 5.31 (app. p, J=6.0 Hz, 0.5H), 5.20 (app. p, J=6.0 Hz, 0.5H), 4.89-4.73 (m, 2H), 4.67-4.60 (m, 0.5H), 4.59-4.50 (m, 1H), 4.45-4.35 (m, 1H), 4.34-4.26 (m, 0.5H), 2.78 (s, 3H), 2.74-2.59 (m, 2H), 1.20 (d, J=6.8 Hz, 1.5H), 1.14 (d, J=6.8 Hz, 1.5H).

The following compound listed in Table 14 was prepared using a similar procedure to the procedure for preparing Compound 345 using 1-(1,3-benzodioxol-5-yl)-N-methyl-propan-2-amine hydrochloride and the appropriate alcohol.

TABLE 14

| Compound | Structure and Name | Retention Time, [M + H]⁺, ¹H NMR |
|---|---|---|
| 348 | 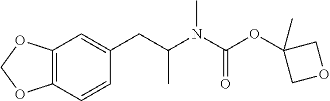 [SMILES: CC(Cc1ccc2c(c1)OCO2)N(C)C(=O)OC1(C)COC1] (3-Methyloxetan-3-yl) N-[2-(1,3-benzodioxol-5-yl)-1- methyl-ethyl]-N-methyl-carbamate | LC-MS (LCMS2: Method 2A): Rt 1.24 mins; MS m/z 308.1 = [M + H]⁺ ¹H NMR (400 MHz, CDCl₃) δ 6.77 − 6.69 (m, 1H), 6.68 − 6.54 (m, 2H), 5.95 − 5.88 (m, 2H), 4.75 (d, J = 6.9 Hz, 0.5H), 4.67 − 4.59 (m, 1H), 4.48 − 4.27 (m, 3.5H), 2.75 (s, 1.5H), 2.72 (s, 1.5H), 2.69 − 2.56 (m, 2H), 1.63 (s, 1.5H), 1.58 (s, 1.5H), 1.20 (d, J = 6.8 Hz, 1.5H), 1.14 (d, J = 6.8 Hz, 1.5H). |

### Example 25: N-({[2-(2H-1,3-Benzodioxol-5-yl)-1-methyl-ethyl]-N-methylamino}methyl)benzamide (Compound 349)

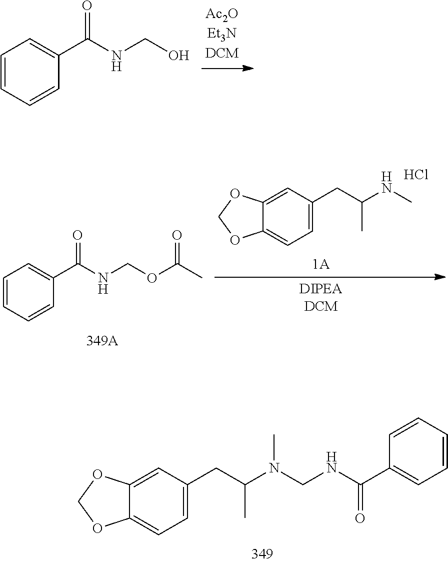

### Benzylaminomethyl acetate (349A)

Ac₂O (2.70 g, 26.5 mmol, 2.50 mL) was added dropwise over 10 min to a stirred suspension of N-(hydroxymethyl)benzamide (2.00 g, 13.2 mmol) and Et₃N (4.02 g, 39.7 mmol, 5.53 mL) in DCM (50 mL) at rt under an atmosphere of N₂. The mixture was stirred at rt for 24 h, then diluted with DCM (100 mL) and the organic phase was washed with H₂O (2×100 mL), dried over Na₂SO₄, filtered and concentrated in vacuo. The residue was purified by column chromatography on silica gel, eluting with a gradient of 0-50% EtOAc in petroleum ether, to afford benzamidomethyl acetate (intermediate 349A, 1.27 g, 49% yield) as an oil. LC-MS (LCMS2: Method 2A): Rt 1.04 mins; MS m/z 216.1=[M+Na]⁺. ¹H NMR (400 MHz, CDCl₃) δ 7.83-7.77 (m, 2H), 7.57-7.51 (m, 1H), 7.49-7.42 (m, 2H), 7.32 (br. s, 1H), 5.46 (d, J=7.2 Hz, 2H), 2.09 (s, 3H).

### Acetylaminomethyl acetate (349B)

Acetylaminomethyl acetate (349B) was prepared analogously to Benzylaminomethyl acetate (349A) using N-(hydroxymethyl)acetamide in lieu of N-(hydroxymethyl)benzamide. ¹H NMR (400 MHz, CDCl₃) δ 6.80 (br. s, 1H), 5.21 (d, J=7.3 Hz, 2H), 2.06 (s, 3H), 2.01 (s, 3H).

A suspension of 1-(1,3-benzodioxol-5-yl)-N-methyl-propan-2-amine HCl (1A, 253 mg, 1.10 mmol), benzamidomethyl acetate (349A, 255 mg, 1.32 mmol) and potassium carbonate (381 mg, 2.75 mmol) in MeCN (7.5 mL) was stirred at 40° C. under an atmosphere of N₂, in a sealed tube overnight. The mixture was cooled to rt and then filtered through celite, eluting with MeCN (20 mL). The filtrate was concentrated in vacuo and the residue was purified by column chromatography on silica gel, eluting with a gradient of 0-2% MeOH with ammonia in DCM, to afford a solid contained within an oil. The mixture was dissolved in MeOH (2 mL) and re-purified using an SCX-2 cartridge, to afford N-({[2-(2H-1,3-benzodioxol-5-yl)-1-methyl-ethyl]-N-methylamino}methyl)benzamide (Compound 349, 85 mg, 22% yield) as a gum. LC-MS (LCMS2: Method 2A): Rt 0.99 mins; MS m/z 327.0=[M+H]⁺. ¹H NMR (400 MHz, CDCl₃) δ 7.75-7.69 (m, 2H), 7.52-7.40 (m, 3H), 6.72 (d, J=7.9 Hz, 1H), 6.69 (d, J=1.7 Hz, 1H), 6.62 (dd, J=7.9, 1.7 Hz, 1H), 6.22 (br. s, 1H), 5.91 (s, 2H), 4.41-4.31 (m, 2H), 3.07-2.98 (m, 1H), 2.84 (dd, J=13.4, 6.3 Hz, 1H), 2.48-2.40 (m, 4H), 1.05 (d, J=6.8 Hz, 3H).

The following compound listed in Table 15 was prepared using a similar procedure to the procedure for preparing Compound 349 using acetylaminomethyl acetate (349B) in lieu of benzamidomethyl acetate (349A).

TABLE 15

| Compound | Structure and Name | Retention Time, [M + H]⁺, ¹H NMR |
|---|---|---|
| 350 | 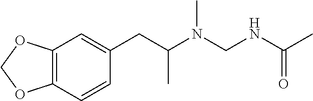 [SMILES: CC(=O)NCN(C)C(C)Cc1ccc2c(c1)OCO2] N-({[2-(2H-1,3-Benzodioxol-5-yl)-1- methyl-ethyl]-N-methylamino}methyl)acetamide | LC-MS (LCMS2: Method 2B): Rt 0.94 mins; MS m/z 265.1 = [M + H]⁺ ¹H NMR (400 MHz, CDCl₃) δ 6.73 (d, J = 7.9 Hz, 1H), 6.67 (d, J = 1.7 Hz, 1H), 6.61 (dd, J = 7.9, 1.7 Hz, 1H), 5.92 (s, 2H), 5.49 (br. s, 1H), 4.16 − 4.06 (m, 2H), 2.97 − 2.88 (m, 1H), 2.79 (dd, J = 13.4, 5.9 Hz, 1H), 2.39 (dd, J = 13.4, 8.4 Hz, 1H), 2.33 (s, 3H), 1.96 (s, 3H), 0.99 (d, J = 6.8 Hz, 3H). |

### Example A: Pharmacokinetics of Selected Compounds Following a Single Intravenous or Oral Administration in Rats

A pharmacokinetic (PK) study was performed in three male Sprague-Dawley (SD) rats following intravenous (IV) or oral (PO) administration of 3,4-Methylenedioxy methamphetamine (MDMA) and its derivatives described herein at 1 mg/kg (IV) or 10 mg/kg (PO).

### In Vivo Methods.

### Rat Strain.

Sprague-Dawley rats were supplied by Charles River (Margate UK) and were specific pathogen free. Male rats weighed between 175-225 g on receipt and were allowed to acclimate for 5-7 days prior to administration.

### Animal Housing.

Rats were group housed in sterilised individual ventilated cages that exposed the animals at all times to HEPA filtered sterile air. Animals had free access to food and water (sterile) and sterile aspen chip bedding (changed at least once weekly). The room temperature was maintained at 22° C. +/−1° C., with a relative humidity of 60% and maximum background noise of 56 dB. Rats were exposed to 12-hour light/dark cycles.

### Treatment.

Each test compound and control (MDMA) were diluted with 10% v/v DMSO, 40% v/v PEG-400, 50% v/v water. The test compound or the control (MDMA) were administered in a dose volume of 2 mL/kg for intravenous administration (IV) and 5 mL/kg for oral administration (PO).

### Single IV/PO Dose Pharmacokinetics Study in Rats.

Each test compound was administered as a single IV bolus (via a lateral tail-vein) or a single oral gavage in cohorts of 3 rats per administration route. Following dose administrations, a 100 μL whole blood sample (EDTA) was collected via the tail-vein at timepoints described in Table 16. The blood sample was centrifuged to separate plasma. Approximately 40 μL of the separated plasma was dispensed per time-point, per rat, in a 96 well plate and frozen until analysis. Bioanalysis was carried out on the separated plasma samples.

TABLE 16
Sample collection points for single IV
and oral dose pharmacokinetics study.

| | | | | Blood sample | |
|---|---|---|---|---|---|
| | | | Dose | collection | No. of |
| Group | Drug | Route | (mg/kg) | (post dose) | rats |
| 1 | MDMA | IV | 1 | 15 min, 30 min, | 3 |
| | | | | 45 min, 1 h, 2 h, | |
| | | | | 4 h, 7 h, 24 h | |
| 2 | MDMA | PO | 10 | 15 min, 30 min, | 3 |
| | | | | 45 min, 1 h, 2 h, | |
| | | | | 4 h, 7 h, 24 h | |

### Bioanalysis Methods.

### MDMA—Stock Preparation.

2.4 mL of DMSO was pipetted into an amber vial containing 2.4 mg salt-free MDMA. The contents were mixed by vortex to provide a ˜1000 μg/mL standard solution in DMDO.

### Preparation of Calibration and Quality Control Standards.

Separate calibration curve and QC standards were prepared from individual standard to minimise the chance of multiple-reaction monitoring (MRM) crosstalk during analysis. The dilutions were performed as detailed in Table 17.

TABLE 17
Preparation of 1 to 5000 ng/mL Cal and QC working solution.

| | | | | |
|---|---|---|---|---|
| | 50/50 | Working | | |
| Working | MeOH/H2O | Solution | Calibrant | |
| Solution | Volume | Conc. | Conc. | Calibrant ID |
| ID | (μL) | (μg/mL) | (ng/mL) | (for sample list) |

Preparation of Calibrator Working Solutions

| | | | | |
|---|---|---|---|---|
| DMSO | — | 1000 | — | — |
| WS1 | 950 | 50 | 5000 | Cal 12 5000 ng/mL |
| WS2 | 975 | 25 | 2508 | Cal 11 2500 ng/mL |
| WS3 | 990 | 10 | 1000 | Cal 10 1000 ng/mL |
| WS4 | 900 | 5 | 500 | Cal 9 500 ng/mL |
| WS5 | 900 | 2.5 | 250 | Cal 8 250 ng/mL |
| WS6 | 900 | 1 | 100 | Cal 7 100 ng/mL |
| WS7 | 900 | 0.5 | 50 | Cal 6 50 ng/mL |
| WS8 | 900 | 0.25 | 25 | Cal 5 25 ng/mL |
| WS9 | 900 | 0.1 | 10 | Cal 4 10 ng/mL |
| WS10 | 900 | 0.05 | 5 | Cal 3 5 ng/mL |
| WS11 | 900 | 0.025 | 2.5 | Cal 2 2.5 ng/mL |
| WS12 | 900 | 0.01 | 1 | Cal 1 1 ng/mL |

Preparation of QC Working Solutions

| | | | | |
|---|---|---|---|---|
| DMSO | — | 1000 | — | — |
| QC-WS1 | 960 | 40 | 4000 | QC 4 4000 ng/mL |
| QC-WS2 | 900 | 4 | 400 | QC 3 400 ng/mL |
| QC-WS3 | 900 | 0.4 | 40 | QC 2 40 ng/mL |
| QC-WS4 | 900 | 0.04 | 4 | QC 1 4 ng/mL |

All samples were diluted to volume with 50:50 methanol/water (v/v) in individual 1.5 mL Eppendorf tubes and mixed by vortexing.

The control matrix was rat plasma (male Sprague Dawley, EDTA). Calibration and quality control (QC) standards were prepared by spiking control matrix with working solutions containing MDMA.

### Dose Formulation Samples.

Dose formulation samples were diluted in two steps with 50:50 (v/v) methanol/water to an appropriate concentration, then diluted 10:90 (v/v) with control matrix to match to the calibration standard in plasma.

### Sample Extraction Procedure.

Calibration and QC standards, incurred samples, blank matrix, and dose formulation samples were extracted by protein precipitation, via the addition of a bespoke acetonitrile (CH₃CN)-based Internal Standard (IS) solution, containing compounds including Metoprolol and Rosuvastatin, both of which were monitored for during analysis. Following centrifugation, a 40 μL aliquot of supernatant was diluted by the addition of 80 μL water. The prepared sample extracts were analysed by LC-MS/MS.

### Example Bioanalytical Method and Assay Procedure.

1 According to the plate layout, aliquot to wells in 0.8 mL 96-well plate (Abgene). 30 μL for Calibration, QC standards, blanks and dose formulation check.
2 Prepare Calibration and QC standards according to the assay information. Dilute dose formulation according to the assay information. Aliquot incurred samples according to the plate layout & assay information.
3 Add 90 μL of CH₃CN internal standard and vortex mix for 5 minutes at 850 rpm.
4 Centrifuge at nominally 4000 rpm for 10 minutes.
6 Transfer 40 μL of supernatant into a new 0.8 mL Abgene plate.
6 Add 80 μL of water to all transferred supernatant.
7 Vortex mix for 30 seconds at 1400 rpm.
8 Analyse immediately by LC-MS/MS or store at +4° C. until analysis.

The analysis was performed using the following solvent system and gradient described in Table 18.

TABLE 18

| Instrument Name | Agilent ™ 1290 Infinity Binary HPLC Pump Column Oven | | | |
|---|---|---|---|---|
| | Agilent ™ 1290 Infinity HPLC dual needle injection autosampler | | | |
| Column | Kinetex ™ XB-C18, 2.6 μm, 50 × 2.1 mm | | | |
| Column Temperature | 50° C. | | | |
| Autosampler | 10° C. | | | |
| Temperature | | | | |
| Mobile Phase | Eluent A: 2.5 mmol/L ammonium formate (aq) + 0.1% formic acid | | | |
| | (v/v) | | | |
| | Eluent B: Methanol | | | |
| | | Flow Rate | % Mobile | % Mobile |
| | Time (min) | (μL/min) | Phase A | Phase B |
| Gradient Profile | 0 | 800 | 98 | 2 |
| | 0.1 | 800 | 98 | 2 |
| | 1 | 800 | 5 | 95 |
| | 1.5 | 800 | 5 | 95 |
| | 1.55 | 800 | 98 | 2 |
| | 1.8 | 800 | 98 | 2 |

| | |
|---|---|
| Flow | 0.8 mL/min |
| Stop time | 1.8 minutes |
| Injection Volume | 2 μL |

Measurement of Concentration of MDMA after IV or Oral Administration of Compounds

The pharmacokinetic properties of the synthesized MDMA derivative compounds after IV or oral administration in a rat model were assessed. The concentration of MDMA was measured in each rat at various sampling timepoints after IV or oral administration of the synthesized MDMA derivative compounds to rats.

Dose formulations were made at equivalent concentrations of active compound (MDMA) adjusted for molecular weight of the compounds. The synthesized MDMA derivative compounds were dosed at 1 mg/kg IV and 10 mg/kg PO nominal dose respectively. The IV dose formulation was a clear solution and the PO dose formulation was a white homogeneous suspension. Nominal doses are used in PK parameter determinations.

A comparison of the results from Example 2 through Example 58 reveals that various derivative forms of MDMA described herein have vastly different pharmacokinetic properties. Oral administration of the compounds tested in Examples 2 through Example 58 resulted in total measured bodily plasma exposure to MDMA spanning a range of several orders of magnitude when comparing different MDMA derivative compounds. These results were unexpected and not predictable based solely on structural knowledge of the compounds.

### Example 2-L. MDMA Parent Compound

| | |
|---|---|
| Dosed Test Article: | MDMA |
| Dose Route: | IV & Oral |
| Nominal Dose Concentration: | 1 & 10 mg/Kg |
| Analyte: | MDMA |

Chemical name: MDMA 3,4-Methylenedioxymethamphetamine [1-(1,3-benzodioxol-5-yl)-N-methyl-propan-2-amine]
Structural class: parent drug
Mechanistic class: n/a

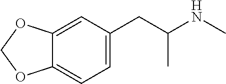

TABLE 2-1
MDMA PK Parameters
Mean* PK Parameters

| PK Parameter | IV | PO |
|---|---|---|
| Dose (mg/Kg) | 1 | 10 |
| C0/Cmax (ng/mL) | 150 | 512 |
| Tmax (h) | — | 0.50 |
| MRT (h) | 0.423 | 3.32 |
| Tlast (h) | 2.00 | 7.00 |
| AUC0-last (h*ng/mL) | 61.6 | 1710 |
| AUC0-2 (h*ng/mL) | 61.6 | 638 |
| AUC0-24 (h*ng/mL) | — | 1810 |
| AUC0-inf (h*ng/mL) | 63.3 | 1800 |
| T½ (h) | 0.420 | 2.97 |
| Cl (mL/min/kg) | 274 | — |
| Vdss (L/kg | 8.18 | — |
| F (%)^{A} | — | 104 |

*Median calculated for Tmax and Tlast.
^{A}Bioavailability (F %) calculated using last common timepoint (AUC0-2)

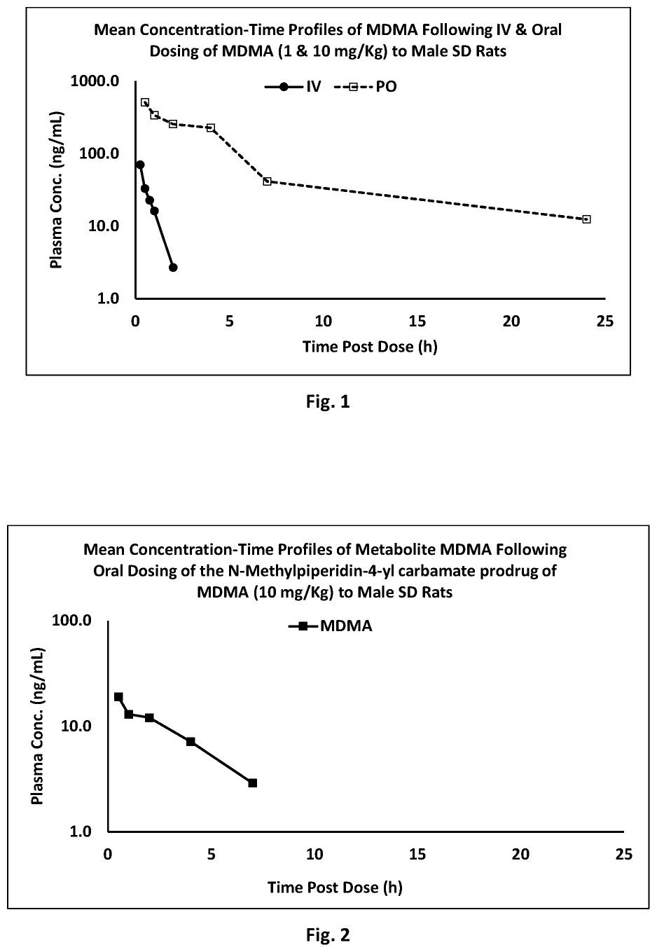

FIG. 1. Mean Concentration-Time Profiles of MDMA Following IV & Oral Dosing of MDMA (1 & 10 mg/Kg) to Male SD Rats

### Example 2-2. N-Methylpiperidin-4-yl carbamate Prodrug of MDMA

| | |
|---|---|
| Dosed Test Article: | N-Methylpiperidin-4-yl carbamate |
| | prodrug of MDMA |
| Dose Route: | Oral |
| Nominal Dose Concentration: | 10 mg/Kg |
| Analyte: | MDMA |

Chemical name: (1-Methyl-4-piperidyl)N-[2-(1,3-benzodioxol-5-yl)-1-methyl-ethyl]-N-methyl-carbamate
Structural class: carbamate
Mechanistic class: presumed carboxyesterases

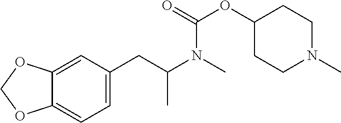 [SMILES: CC(Cc1ccc2c(c1)OCO2)N(C)C(=O)OC1CCN(C)CC1]

TABLE 2-2
MDMA PK Parameters
Mean* Pharmacokinetic Parameters

| PK Parameter | MDMA |
|---|---|
| Cmax (ng/mL) | 20.4 |
| Tmax (h) | 0.50 |
| MRT (h) | 2.57 |
| Tlast (h) | 7.00 |
| AUC0-last (h*ng/mL) | 59.7 |
| AUC0-24 (h*ng/mL) | — |
| AUC0-inf (h*ng/mL) | 76.4 |
| T½ (h) | 3.13 |

*Median calculated for Tmax and Tlast.

FIG. 2. Mean Concentration-Time Profiles of Metabolite MDMA Following Oral Dosing of the N-Methylpiperidin-4-yl carbamate prodrug of MDMA (10 mg/Kg) to Male SD Rats

### Example 2-3. Pyran-4-yl carbamate Prodrug of MDMA

| | |
|---|---|
| Dosed Test Article: | Pyran-4-yl carbamate prodrug of MDMA |
| Dose Route: | Oral |
| Nominal Dose Concentration: | 10 mg/Kg |
| Analyte: | MDMA |

Chemical name: Tetrahydropyran-4-yl N-[2-(1,3-benzodioxol-5-yl)-1-methyl-ethyl]-N-methyl-carbamate
Structural class: carbamate
Mechanistic class: presumed carboxyesterases

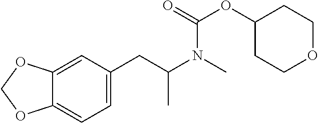 [SMILES: CC(Cc1ccc2c(c1)OCO2)N(C)C(=O)OC1CCOCC1]

TABLE 2-3
MDMA PK Parameters
Mean* Pharmacokinetic Parameters

| PK Parameter | MDMA |
|---|---|
| Cmax (ng/mL) | 0.557 |
| Tmax (h) | 0.50 |
| MRT (h) | 0.50 |
| Tlast (h) | 0.50 |
| AUC0-last (h*ng/mL) | 0.139 |
| AUC0-24 (h*ng/mL) | — |
| AUC0-inf (h*ng/mL) | NC |
| T½ (h) | NC |

*Median calculated for Tmax and Tlast.
NC: Not Calculated

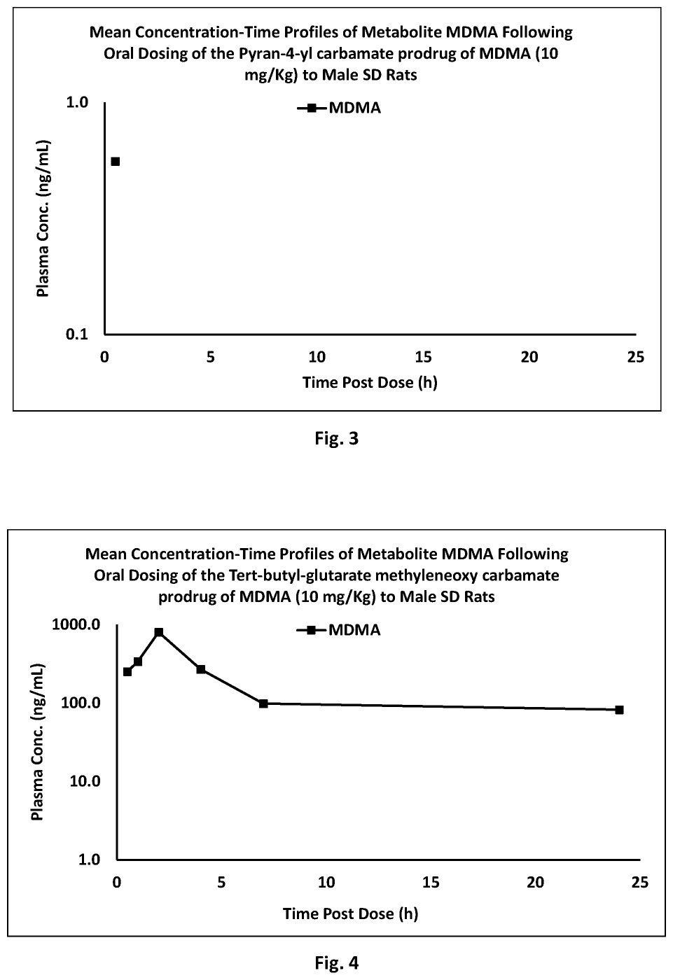

FIG. 3. Mean Concentration-Time Profiles of Metabolite MDMA Following Oral Dosing of the Pyran-4-yl carbamate prodrug of MDMA (10 mg/Kg) to Male SD Rats

### Example 2-4. Tert-butyl-glutarate methyleneoxy carbamate Prodrug of MDMA

| | |
|---|---|
| Dosed Test Article: | Tert-butyl-glutarate methyleneoxy |
| | carbamate prodrug of MDMA |
| Dose Route: | Oral |
| Nominal Dose Concentration: | 10 mg/Kg |
| Analyte: | MDMA |

Chemical name: {[2-(2H-1,3-Benzodioxol-5-yl)-1-methylethyl]-N-methylaminocarbonyloxy}methyl tert-butyl glutarate
Structural class: carbamate
Mechanistic class: presumed carboxyesterases+intramolecular cyclization+chemical breakdown

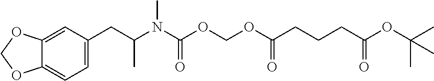 [SMILES: CC(Cc1ccc2c(c1)OCO2)N(C)C(=O)OCOC(=O)CCCC(=O)OC(C)(C)C]

TABLE 2-4
MDMA PK Parameters
Mean* Pharmacokinetic Parameters

| PK Parameter | MDMA |
|---|---|
| Cmax (ng/mL) | 646 |
| Tmax (h) | 4.00 |
| MRT (h) | 4.96 |
| Tlast (h) | 7.00 |
| AUC0-last (h*ng/mL) | 1830 |
| AUC0-24 (h*ng/mL) | 2570 |
| AUC0-inf (h*ng/mL) | NC |
| T½ (h) | NC |

*Median calculated for Tmax and Tlast.
NC: Not Calculated

FIG. 4. Mean Concentration-Time Profiles of Metabolite MDMA Following Oral Dosing of the Tert-butyl-glutarate methyleneoxy carbamate prodrug of MDMA (10 mg/Kg) to Male SD Rats

### Example 2-5. Tetrahydrofuran-3-yl amide Prodrug of MDMA

| | |
|---|---|
| Dosed Test Article: | Tetrahydrofuran-3-yl amide |
| | prodrug of MDMA |
| Dose Route: | Oral |
| Nominal Dose Concentration: | 10 mg/Kg |
| Analyte: | MDMA |

Chemical name: N-[2-(2H-1,3-Benzodioxol-5-yl)-1-methyl-ethyl]-N-methyl-tetrahydro-3-furamide
Structural class: amide
Mechanistic class: presumed carboxyesterases

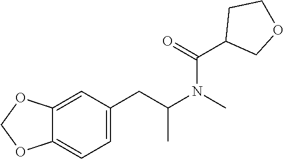 [SMILES: CC(Cc1ccc2c(c1)OCO2)N(C)C(=O)C1CCOC1]

TABLE 2-5
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-6. Glutarate Prodrug of MDMA

| | |
|---|---|
| Dosed Test Article: | Glutarate prodrug of MDMA |
| Dose Route: | Oral |
| Nominal Dose Concentration: | 10 mg/Kg |
| Analyte: | MDMA |

Chemical name: 4-{[2-(2H-1,3-Benzodioxol-5-yl)-1-methyl-ethyl]-N-methylcarbamoyl}butyric acid
Structural class: amide
Mechanistic class: presumed amidases

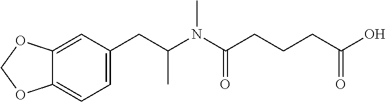 [SMILES: CC(Cc1ccc2c(c1)OCO2)N(C)C(=O)CCCC(=O)O]

TABLE 2-6
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-7. Pyran-Acyloxy-Substituted-Methylene Prodrug of MDMA

| | |
|---|---|
| Dosed Test Article: | Pyran-acyloxy- substituted-methylene prodrug of |
| | MDMA ((Tetrahydropyran-4-carboxy)-1- |
| | ethyleneoxy carbamate) |
| Dose Route: | Oral |
| Nominal Dose | 10 mg/Kg |
| Concentration: | |
| Analyte: | MDMA |

Chemical name: 1-{[2-(2H-1,3-Benzodioxol-5-yl)-1-methyl-ethyl]-N-methylaminocarbonyloxy}ethyl tetrahydro-2H-pyran-4-carboxylate
Structural class: carbamate
Mechanistic class: presumed carboxyesterases+chemical breakdown

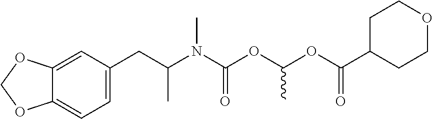 [SMILES: CC(OC(=O)C1CCOCC1)OC(=O)N(C)C(C)Cc1ccc2c(c1)OCO2]

TABLE 2-7
MDMA PK Parameters
Mean* Pharmacokinetic Parameters

| PK Parameter | MDMA |
|---|---|
| Cmax (ng/mL) | 189 |
| Tmax (h) | 1.00 |
| MRT (h) | 4.50 |
| Tlast (h) | 24.0 |
| AUC0-last (h*ng/mL) | 1000 |
| AUC0-24 (h*ng/mL) | 1055 |
| AUC0-inf (h*ng/mL) | 1190 |
| T½ (h) | 7.14 |

*Median calculated for Tmax and Tlast.

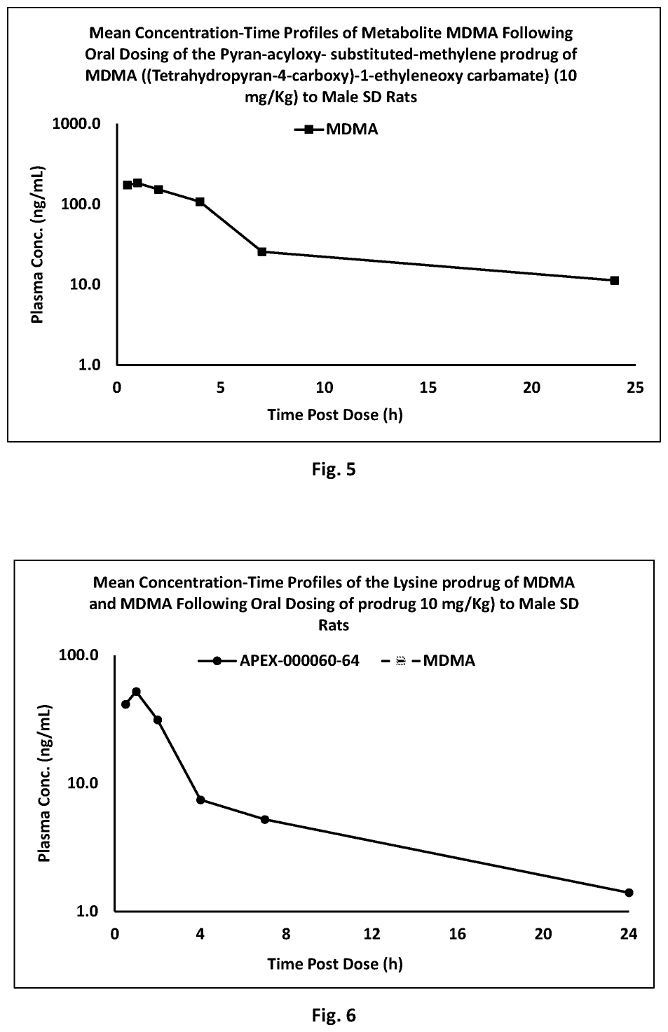

FIG. 5. Mean Concentration-Time Profiles of Metabolite MDMA Following Oral Dosing of the Pyran-acyloxy-substituted-methylene prodrug of MDMA ((Tetrahydropyran-4-carboxy)-1-ethyleneoxy carbamate) (10 mg/Kg) to Male SD Rats

### Example 2-8. Ethyl Carbamate Prodrug of MDMA

| | |
|---|---|
| Dosed Test Article: | Ethyl carbamate prodrug of MDMA |
| Dose Route: | Oral |
| Nominal Dose Concentration: | 10 mg/Kg |
| Analyte: | MDMA |

Chemical name: Ethyl N-[2-(1,3-benzodioxol-5-yl)-1-methyl-ethyl]-N-methyl-carbamate Structural class: carbamate
Mechanistic class: presumed carboxyesterases

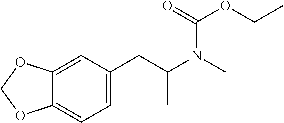 [SMILES: CCOC(=O)N(C)C(C)Cc1ccc2c(c1)OCO2]

TABLE 2-8
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-9. Isobutyl Carbamate Prodrug of MDMA

| | |
|---|---|
| Dosed Test Article: | Isobutyl carbamate prodrug of MDMA |
| Dose Route: | Oral |
| Nominal Dose Concentration: | 10 mg/Kg |
| Analyte: | MDMA |

Chemical name: isobutyl N-[2-(1,3-benzodioxol-5-yl)-1-methyl-ethyl]-N-methyl-carbamate
Structural class: carbamate
Mechanistic class: presumed carboxyesterases

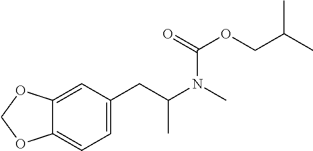 [SMILES: CC(C)COC(=O)N(C)C(C)Cc1ccc2c(c1)OCO2]

TABLE 2-9
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-10. Lysine Prodrug of MDMA

| | |
|---|---|
| Dosed Test Article: | Lysine prodrug of MDMA |
| Dose Route: | Oral |
| Nominal Dose Concentration: | 10 mg/Kg |
| Analyte: | MDMA |

Chemical name: (2S)-2,6-diamino-N-[2-(1,3-benzodioxol-5-yl)-1-methyl-ethyl]-N-methyl-hexanamide
Structural class: amide
Mechanistic class: presumed amidases or aminopeptidases

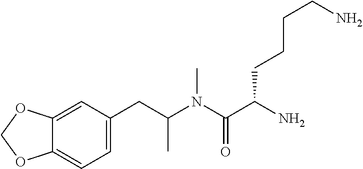 [SMILES: CC(Cc1ccc2c(c1)OCO2)N(C)C(=O)[C@@H](N)CCCCN]

TABLE 2-10
Mean Concentration-Time Profile of the Lysine prodrug of
MDMA and Metabolite MDMA in Rat Plasma (ng/mL) Following
Oral Dosing of MDMA Prodrug
Mean Plasma Concentration (ng/mL) of:

| Time (h) | MDMA Prodrug | MDMA |
|---|---|---|
| 0.50 | 41.5 | BLQ |
| 1.00 | 52.2 | BLQ |
| 2.00 | 31.3 | BLQ |
| 4.00 | 7.42 | BLQ |
| 7.00 | 5.23 | BLQ |
| 24.0 | 1.40 | BLQ |

BLQ: Below Lower Limit of Quantification (MDMA = 0.5 ng/m)

TABLE 2-11
PK Parameters of the Lysine prodrug of MDMA and Metabolite MDMA
Mean PK Parameters of:

| PK Parameter | MDMA prodrug | MDMA |
|---|---|---|
| Cmax (ng/mL) | 56.1 | NC |
| Tmax (h) | 1.00 | NC |
| MRT (h) | 4.50 | NC |
| Tlast (h) | 24.0 | NC |
| AUC0-last (h*ng/mL) | 190 | NC |
| AUC0-24 (h*ng/mL) | 190 | NC |
| AUC0-inf (h*ng/mL) | 208 | NC |
| T½ (h) | 9.37 | NC |

NC: Not Calculated. Insufficient data to permit PK parameter determination.

FIG. 6. Mean Concentration-Time Profiles of the Lysine prodrug of MDMA and MDMA Following Oral Dosing of the Lysine prodrug of MDMA (10 mg/Kg) to Male SD Rats

### Example 2-11. Methyl Amide (Acetyl) Prodrug of MDMA

| | |
|---|---|
| Dosed Test Article: | Methyl amide (acetyl) prodrug of MDMA |
| Dose Route: | Oral |
| Nominal Dose Concentration: | 10 mg/Kg |
| Analyte: | MDMA |

Chemical name: N-[2-(2H-1,3-Benzodioxol-5-yl)-1-methyl-ethyl]-N-methylacetamide Structural class: amide
Mechanistic class: presumed amidases

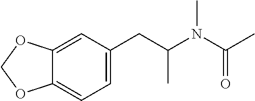 [SMILES: CC(=O)N(C)C(C)Cc1ccc2c(c1)OCO2]

TABLE 2-12
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-12. (Carbamoyloxy)Methyl Pivalate Prodrug of MDMA

| | |
|---|---|
| Dosed Test Article: | (carbamoyloxy)methyl pivalate |
| | prodrug of MDMA |
| Dose Route: | Oral |
| Nominal Dose Concentration: | 10 mg/Kg |
| Analyte: | MDMA |

Chemical name: [[2-(1,3-benzodioxol-5-yl)-1-methyl-ethyl]-methyl-carbamoyl]oxymethyl-2,2-dimethylpropanoate
Structural class: carbamate
Mechanistic class: presumed carboxyesterases+chemical breakdown

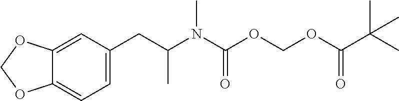 [SMILES: CC(Cc1ccc2c(c1)OCO2)N(C)C(=O)OCOC(=O)C(C)(C)C]

TABLE 2-13
MDMA PK Parameters
Mean* Pharmacokinetic Parameters

| PK Parameter | MDMA |
|---|---|
| Cmax (ng/mL) | 154 |
| Tmax (h) | 1.00 |
| MRT (h) | 2.76 |
| Tlast (h) | 7.00 |
| AUC0-last (h*ng/mL) | 519 |
| AUC0-24 (h*ng/mL) | — |
| AUC0-inf (h*ng/mL) | 717 |
| T½ (h) | 3.88 |

*Median calculated for Tmax and Tlast.

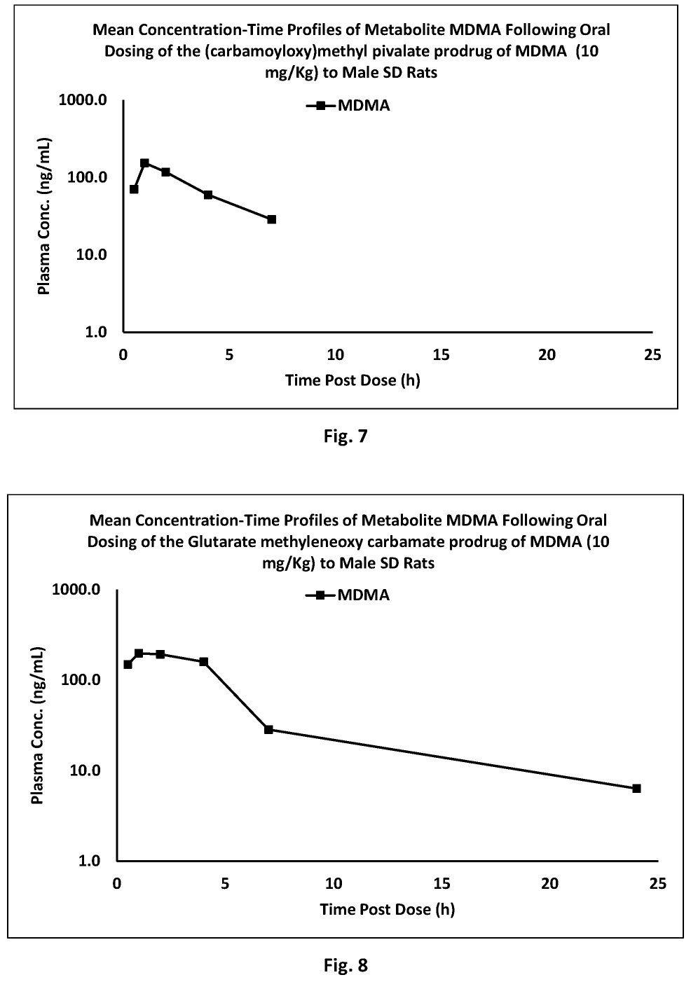

FIG. 7. Mean Concentration-Time Profiles of Metabolite MDMA Following Oral Dosing of the (carbamoyloxy)methyl pivalate prodrug of MDMA (10 mg/Kg) to Male SD Rats

### Example 2-13. Glutarate Methyleneoxy Carbamate Prodrug of MDMA

| | |
|---|---|
| Dosed Test Article: | Glutarate methyleneoxy carbamate |
| | prodrug of MDMA |
| Dose Route: | Oral |
| Nominal Dose Concentration: | 10 mg/Kg |
| Analyte: | MDMA |

Chemical name: 5-[[[2-(1,3-Benzodioxol-5-yl)-1-methyl-ethyl]-methyl-carbamoyl]oxymethoxy]-5-oxo-pentanoic acid
Structural class: carbamate
Mechanistic class: presumed pH-dependent cyclization+chemical breakdown

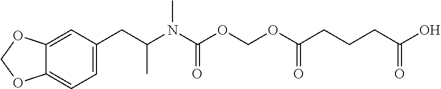 [SMILES: CC(Cc1ccc2c(c1)OCO2)N(C)C(=O)OCOC(=O)CCCC(=O)O]

TABLE 2-14
MDMA PK Parameters
Mean* Pharmacokinetic Parameters

| PK Parameter | MDMA |
|---|---|
| Cmax (ng/mL) | 211 |
| Tmax (h) | 1.00 |
| MRT (h) | 3.49 |
| Tlast (h) | 7.00 |
| AUC0-last (h*ng/mL) | 987 |
| AUC0-24 (h*ng/mL) | 569 |
| AUC0-inf (h*ng/mL) | 843 |
| T½ (h | 5.30 |

*Median calculated for Tmax and Tlast.

FIG. 8. Mean Concentration-Time Profiles of Metabolite MDMA Following Oral Dosing of the Glutarate methyleneoxy carbamate prodrug of MDMA (10 mg/Kg) to Male SD Rats

### Example 2-14. Trimethyllock Prodrug of MDMA

| | |
|---|---|
| Dosed Test Article: | Trimethyllock prodrug of MDMA |
| Dose Route: | Oral |
| Nominal Dose Concentration: | 10 mg/Kg |
| Analyte: | MDMA |

Chemical name: 2-(2-{[2-(2H-1,3-Benzodioxol-5-yl)-1-methylethyl]-N-methylcarbamoyl}-1,1-dimethylethyl)-3,5-xylyl acetate
Structural class: amide
Mechanistic class: presumed carboxyesterases+intramolecular cyclization

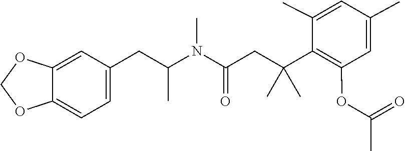 [SMILES: CC(=O)Oc1cc(C)cc(C)c1C(C)(C)CC(=O)N(C)C(C)Cc1ccc2c(c1)OCO2]

TABLE 2-15
MDMA PK Parameters
Mean* Pharmacokinetic Parameters

| PK Parameter | MDMA |
|---|---|
| Cmax (ng/mL) | 67.2 |
| Tmax (h) | 2.00 |
| MRT (h) | 3.48 |
| Tlast (h) | 7.00 |
| AUC0-last (h*ng/mL) | 286 |
| AUC0-24 (h*ng/mL) | — |
| AUC0-inf (h*ng/mL) | NC |
| T½ (h) | NC |

*Median calculated for Tmax and Tlast.
NC: Not Calculated

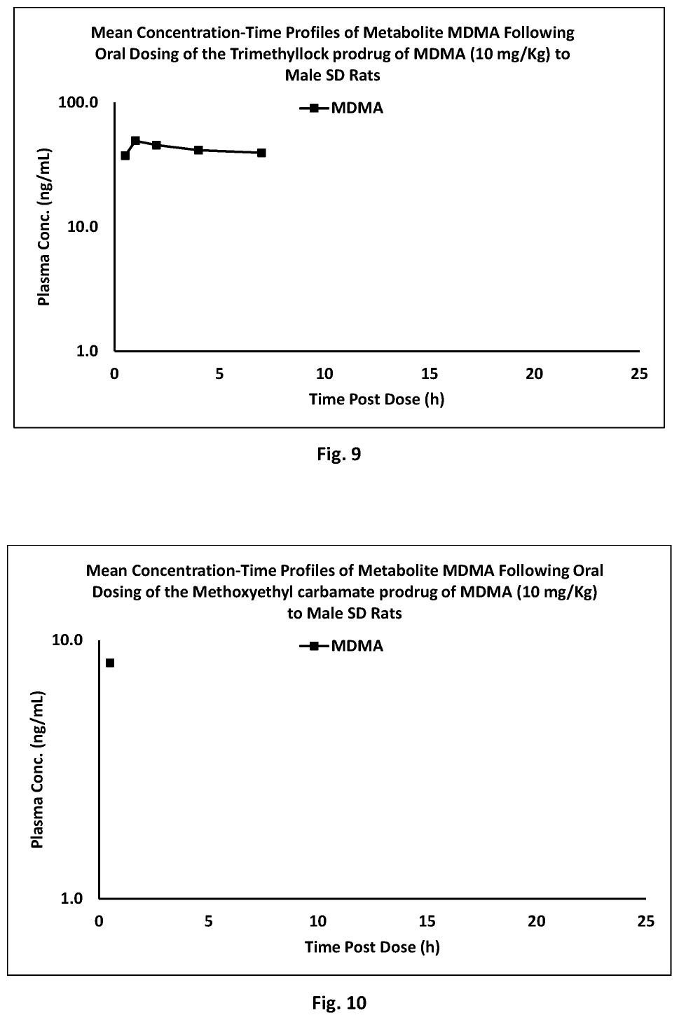

FIG. 9. Mean Concentration-Time Profiles of Metabolite MDMA Following Oral Dosing of the Trimethyllock prodrug of MDMA (10 mg/Kg) to Male SD Rats

### Example 2-15. 2,2-Dimethylpropyl Pivalate Carbamate Prodrug of MDMA

| | |
|---|---|
| Dosed Test Article: | 2,2-Dimethylpropyl pivalate |
| | carbamate prodrug of MDMA |
| Dose Route: | Oral |
| Nominal Dose Concentration: | 10 mg/Kg |
| Analyte: | MDMA |

Chemical name: [3-[[2-(1,3-Benzodioxol-5-yl)-1-methyl-ethyl]-methyl-carbamoyl]oxy-2,2-dimethyl-propyl] 2,2-dimethylpropanoate
Structural class: carbamate
Mechanistic class: presumed carboxyesterases+cyclization

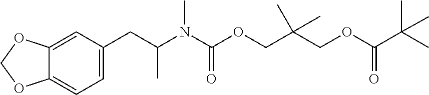 [SMILES: CC(Cc1ccc2c(c1)OCO2)N(C)C(=O)OCC(C)(C)COC(=O)C(C)(C)C]

TABLE 2-16
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-16. SarcPhe Prodrug of MDMA

| | |
|---|---|
| Dosed Test Article: | SarcPhe prodrug of MDMA |
| Dose Route: | Oral |
| Nominal Dose Concentration: | 10 mg/Kg |
| Analyte: | MDMA |

Chemical name: (2S)-2-amino-N-[2-[[2-(1,3-benzodioxol-5-yl)-1-methyl-ethyl]-methyl-amino]-2-oxo-ethyl]-N-methyl-3-phenyl-propanamide
Structural class: amide
Mechanistic class: pH-dependent cyclization

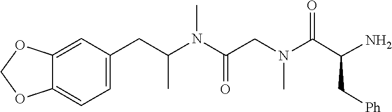 [SMILES: CC(Cc1ccc2c(c1)OCO2)N(C)C(=O)CN(C)C(=O)[C@@H](N)Cc1ccccc1]

TABLE 2-17
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-17. Dimethylurea prodrug of MDMA

| | |
|---|---|
| Dosed Test Article: | Dimethylurea prodrug of MDMA |
| Dose Route: | Oral |
| Nominal Dose Concentration: | 10 mg/Kg |
| Analyte: | MDMA |

Chemical name: 1-[2-(2H-1,3-Benzodioxol-5-yl)-1-methyl-ethyl]-1,3,3-trimethylurea
Structural class: urea
Mechanistic class: presumed amidases

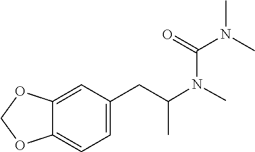 [SMILES: CC(Cc1ccc2c(c1)OCO2)N(C)C(=O)N(C)C]

TABLE 2-18
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-18. Valine Prodrug of MDMA

| | |
|---|---|
| Dosed Test Article: | Valine prodrug of MDMA |
| Dose Route: | Oral |
| Nominal Dose Concentration: | 10 mg/Kg |
| Analyte: | MDMA |

Chemical name: (2S)-2-amino-N-[2-(1,3-benzodioxol-5-yl)-1-methyl-ethyl]-N,3-dimethyl-butanamide
Structural class: amide
Mechanistic class: presumed amidases

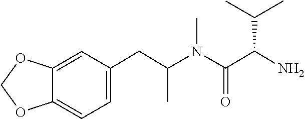 [SMILES: CC(C)[C@H](N)C(=O)N(C)C(C)Cc1ccc2c(c1)OCO2]

TABLE 2-19
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-19. Isopropyl Carbamate Prodrug of MDMA

| | |
|---|---|
| Dosed Test Article: | Isopropyl carbamate prodrug of MDMA |
| Dose Route: | Oral |
| Nominal Dose Concentration: | 10 mg/Kg |
| Analyte: | MDMA |

Chemical name: isopropyl N-[2-(1,3-benzodioxol-5-yl)-1-methyl-ethyl]-N-methyl-carbamate
Structural class: carbamate
Mechanistic class: presumed carboxyesterases

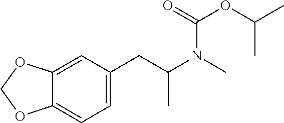 [SMILES: CC(C)OC(=O)N(C)C(C)Cc1ccc2c(c1)OCO2]

TABLE 2-20
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-20. Propyl Carbamate Prodrug of MDMA

| | |
|---|---|
| Dosed Test Article: | Propyl carbamate prodrug of MDMA |
| Dose Route: | Oral |
| Nominal Dose Concentration: | 10 mg/Kg |
| Analyte: | MDMA |

Chemical name: propyl N-[2-(1,3-benzodioxol-5-yl)-1-methyl-ethyl]-N-methyl-carbamate
Structural class: carbamate
Mechanistic class: presumed carboxyesterases

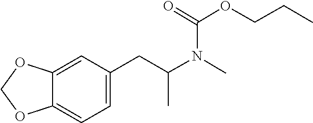 [SMILES: CCCOC(=O)N(C)C(C)Cc1ccc2c(c1)OCO2]

TABLE 2-21
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-21. Methoxyethyl Carbamate Prodrug of MDMA

| | |
|---|---|
| Dosed Test Article: | Methoxyethyl carbamate |
| | prodrug of MDMA |
| Dose Route: | Oral |
| Nominal Dose Concentration: | 10 mg/Kg |
| Analyte: | MDMA |

Chemical name: 2-methoxyethyl N-[2-(1,3-benzodioxol-5-yl)-1-methyl-ethyl]-N-methyl-carbamate
Structural class: carbamate
Mechanistic class: presumed carboxyesterases

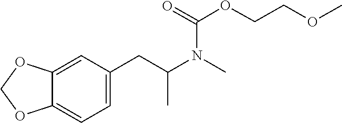 [SMILES: COCCOC(=O)N(C)C(C)Cc1ccc2c(c1)OCO2]

TABLE 2-22
MDMA PK Parameters
Mean* Pharmacokinetic Parameters

| PK Parameter | MDMA |
|---|---|
| Cmax (ng/mL) | 8.18 |
| Tmax (h) | 0.50 |
| MRT (h) | 0.50 |
| Tlast (h) | 0.50 |
| AUC0-last (h*ng/mL) | 2.05 |
| AUC0-24 (h*ng/mL) | — |
| AUC0-inf (h*ng/mL) | NC |
| T½ (h) | NC |

*Median calculated for Tmax and Tlast.
NC: Not Calculated

FIG. 10. Mean Concentration-Time Profiles of Metabolite MDMA Following Oral Dosing of the Methoxyethyl carbamate prodrug of MDMA (10 mg/Kg) to Male SD Rats

### Example 2-22. Methyleneoxyadipate Carbamate Prodrug of MDMA

| | |
|---|---|
| Dosed Test Article: | Methyleneoxyadipate carbamate |
| | prodrug of MDMA |
| Dose Route: | Oral |
| Nominal Dose Concentration: | 10 mg/Kg |
| Analyte: | MDMA |

Chemical name: 6-[[[2-(1,3-Benzodioxol-5-yl)-1-methyl-ethyl]-methyl-carbamoyl]oxymethoxy]-6-oxo-hexanoic acid
Structural class: carbamate
Mechanistic class: presumed pH-dependent cyclization+chemical breakdown

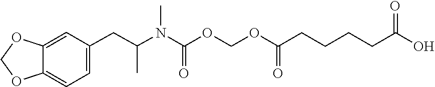 [SMILES: CC(Cc1ccc2c(c1)OCO2)N(C)C(=O)OCOC(=O)CCCCC(=O)O]

TABLE 2-23
MDMA PK Parameters
Mean* Pharmacokinetic Parameters

| PK Parameter | MDMA |
|---|---|
| Cmax (ng/mL) | 257 |
| Tmax (h) | 0.50 |
| MRT (h) | 2.91 |
| Tlast (h) | 7.00 |
| AUC0-last (h*ng/mL) | 1290 |
| AUC0-24 (h*ng/mL) | 1550 |
| AUC0-inf (h*ng/mL) | 1600 |
| T½ (h) | 4.61 |

*Median calculated for Tmax and Tlast.

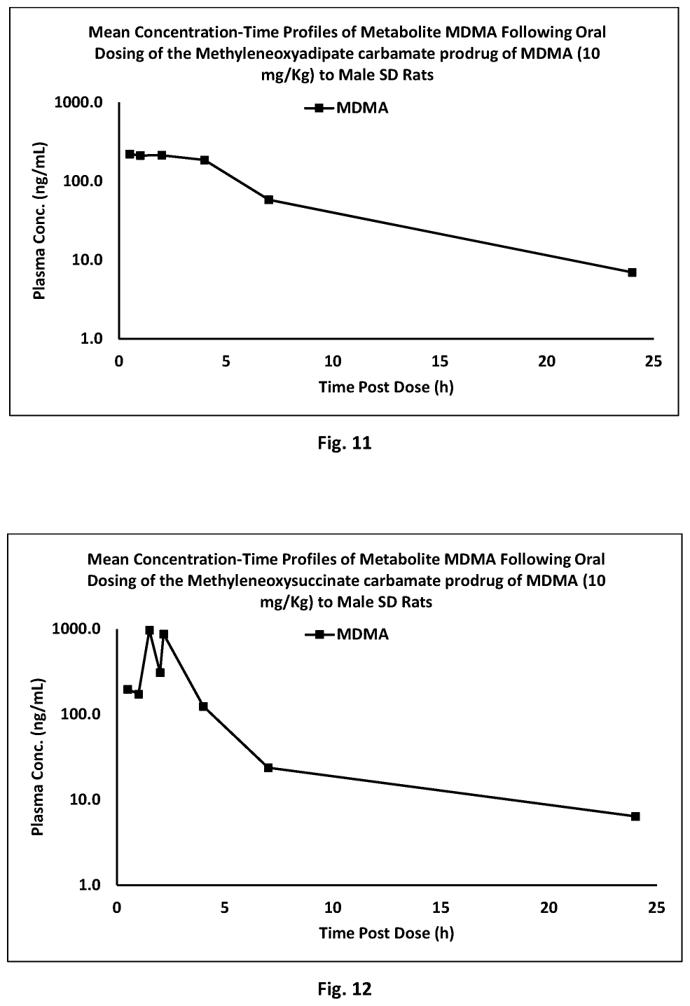

FIG. 11. Mean Concentration-Time Profiles of Metabolite MDMA Following Oral Dosing of the Methyleneoxyadipate carbamate prodrug of MDMA (10 mg/Kg) to Male SD Rats

### Example 2-23. Methyleneoxysuccinate Carbamate Prodrug of MDMA

| | |
|---|---|
| Dosed Test Article: | Methyleneoxysuccinate carbamate |
| | prodrug of MDMA |
| Dose Route: | Oral |
| Nominal Dose Concentration: | 10 mg/Kg |
| Analyte: | MDMA |

Chemical name: 4-[[[2-(1,3-Benzodioxol-5-yl)-1-methyl-ethyl]-methyl-carbamoyl]oxymethoxy]-4-oxo-butanoic acid
Structural class: carbamate
Mechanistic class: presumed pH-dependent cyclization+chemical breakdown

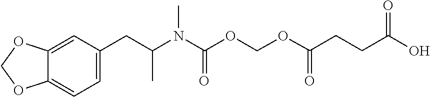 [SMILES: CC(Cc1ccc2c(c1)OCO2)N(C)C(=O)OCOC(=O)CCC(=O)O]

TABLE 2-24
MDMA PK Parameters
Mean* Pharmacokinetic Parameters

| PK Parameter | MDMA |
|---|---|
| Cmax (ng/mL) | 672 |
| Tmax (h) | 1.50 |
| MRT (h) | 2.46 |
| Tlast (h) | 2.16 |
| AUC0-last (h*ng/mL) | 756 |
| AUC0-24 (h*ng/mL) | 1050 |
| AUC0-inf (h*ng/mL) | 1100 |
| T½ (h) | 5.59 |

*Median calculated for Tmax and Tlast.

FIG. 12. Mean Concentration-Time Profiles of Metabolite MDMA Following Oral Dosing of the Methyleneoxysuccinate carbamate prodrug of MDMA (10 mg/Kg) to Male SD Rats

### Example 2-24. Dimethylglycine Prodrug of MDMA

| | |
|---|---|
| Dosed Test Article: | Dimethylglycine prodrug of MDMA |
| Dose Route: | Oral |
| Nominal Dose Concentration: | 10 mg/Kg |
| Analyte: | MDMA |

Chemical name: N-[2-(2H-1,3-Benzodioxol-5-yl)-1-methyl-ethyl]-N-methyl(dimethylamino)acetamide
Structural class: amide
Mechanistic class: presumed amidases

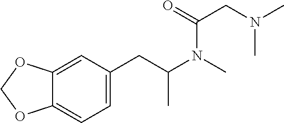 [SMILES: CC(Cc1ccc2c(c1)OCO2)N(C)C(=O)CN(C)C]

TABLE 2-25
MDMA PK Parameters
Mean* Pharmacokinetic Parameters

| PK Parameter | MDMA |
|---|---|
| Cmax (ng/mL) | 1.79 |
| Tmax (h) | 0.50 |
| MRT (h) | 1.04 |
| Tlast (h) | 2.00 |
| AUC0-last (h*ng/mL) | 2.74 |
| AUC0-24 (h*ng/mL) | — |
| AUC0-inf (h*ng/mL) | NC |
| T½ (h) | NC |

*Median calculated for Tmax and Tlast.
NC: Not Calculated

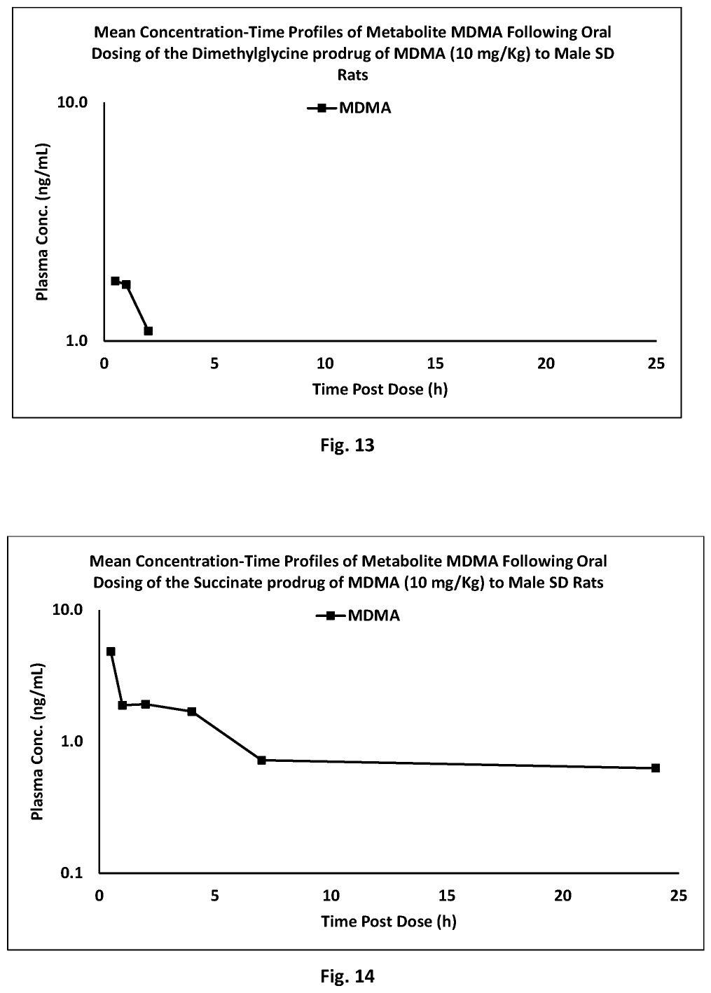

FIG. 13. Mean Concentration-Time Profiles of Metabolite MDMA Following Oral Dosing of the Dimethylglycine prodrug of MDMA (10 mg/Kg) to Male SD Rats

### Example 2-25. Tert-butyl amide prodrug of MDMA

| | |
|---|---|
| Dosed Test Article: | Tert-butyl amide prodrug of MDMA |
| Dose Route: | Oral |
| Nominal Dose Concentration: | 10 mg/Kg |
| Analyte: | MDMA |

Chemical name: N-[2-(2H-1,3-Benzodioxol-5-yl)-1-methyl-ethyl]-N-methyl-2,2-dimethylpropionamide
Structural class: amide
Mechanistic class: presumed amidases

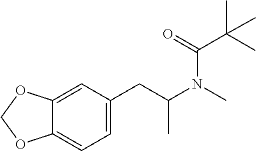 [SMILES: CC(Cc1ccc2c(c1)OCO2)N(C)C(=O)C(C)(C)C]

TABLE 2-26
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-26. Pyran Amide Prodrug of MDMA

| | |
|---|---|
| Dosed Test Article: | Pyran amide prodrug of MDMA |
| Dose Route: | Oral |
| Nominal Dose Concentration: | 10 mg/Kg |
| Analyte: | MDMA |

Chemical name: N-[2-(2H-1,3-Benzodioxol-5-yl)-1-methyl-ethyl]-N-methyl-tetrahydro-2H-pyran-4-carboxamide
Structural class: amide
Mechanistic class: presumes amidases

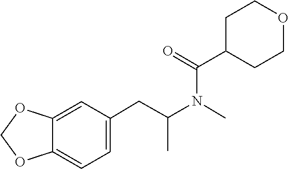 [SMILES: CC(Cc1ccc2c(c1)OCO2)N(C)C(=O)C1CCOCC1]

TABLE 2-27
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-27. Succinate Prodrug of MDMA

| | |
|---|---|
| Dosed Test Article: | Succinate prodrug of MDMA |
| Dose Route: | Oral |
| Nominal Dose Concentration: | 10 mg/Kg |
| Analyte: | MDMA |

Chemical name: 3-{[2-(2H-1,3-Benzodioxol-5-yl)-1-methyl-ethyl]-N-methylcarbamoyl}propionic acid
Structural class: amide
Mechanistic class: presumed pH-dependent cyclization

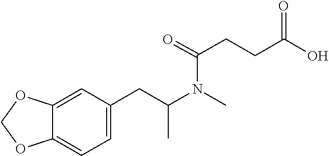 [SMILES: CC(Cc1ccc2c(c1)OCO2)N(C)C(=O)CCC(=O)O]

TABLE 2-28
MDMA PK Parameters
Mean* Pharmacokinetic Parameters

| PK Parameter | MDMA |
|---|---|
| Cmax (ng/mL) | 4.83 |
| Tmax (h) | 0.50 |
| MRT (h) | 5.16 |
| Tlast (h) | 7.00 |
| AUC0-last (h*ng/mL) | 12.8 |
| AUC0-24 (h*ng/mL) | 16.4 |
| AUC0-inf (h*ng/mL) | 15.4 |
| T½ (h) | 3.13 |

*Median calculated for Tmax and Tlast.

FIG. 14. Mean Concentration-Time Profiles of Metabolite MDMA Following Oral Dosing of the Succinate prodrug of MDMA (10 mg/Kg) to Male SD Rats

### Example 2-28. Phenylalanine Prodrug of MDMA

| | |
|---|---|
| Dosed Test Article: | Phenylalanine prodrug of MDMA |
| Dose Route: | Oral |
| Nominal Dose Concentration: | 10 mg/Kg |
| Analyte: | MDMA |

Chemical name: (2S)-2-Amino-N-[2-(1,3-benzodioxol-5-yl)-1-methyl-ethyl]-N-methyl-3-phenyl-propanamide
Structural class: amide
Mechanistic class: presumed amidases

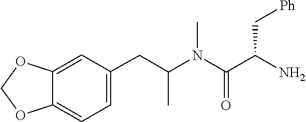 [SMILES: CC(Cc1ccc2c(c1)OCO2)N(C)C(=O)[C@@H](N)Cc1ccccc1]

TABLE 2-29
MDMA PK Parameters
Mean* Pharmacokinetic Parameters

| PK Parameter | MDMA |
|---|---|
| Cmax (ng/mL) | 3.50 |
| Tmax (h) | 1.00 |
| MRT (h) | 1.73 |
| Tlast (h) | 4.00 |
| AUC0-last (h*ng/mL) | 8.49 |
| AUC0-24 (h*ng/mL) | — |
| AUC0-inf (h*ng/mL) | 11.3 |
| T½ (h) | 2.21 |

*Median calculated for Tmax and Tlast.

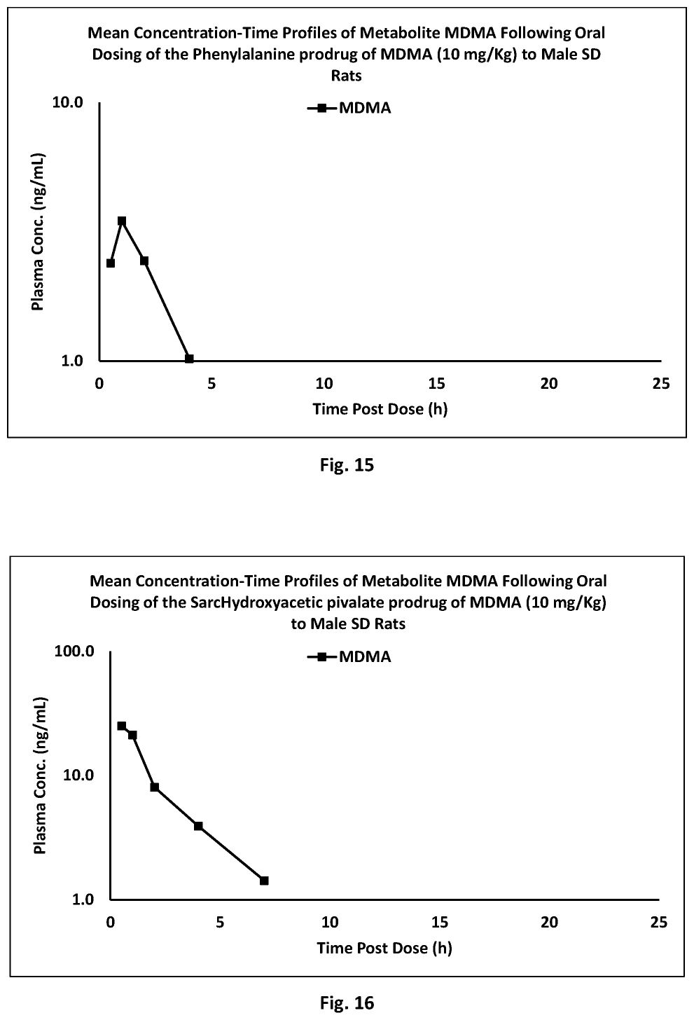

FIG. 15. Mean Concentration-Time Profiles of Metabolite MDMA Following Oral Dosing of the Phenylalanine prodrug of MDMA (10 mg/Kg) to Male SD Rats Example 2-29. THF carbamate prodrug of MDMA

Dosed Test Article: THE Carbamate Prodrug of MDMA

| | |
|---|---|
| Dosed Test Article: | THF carbamate prodrug of MDMA |
| Dose Route: | Oral |
| Nominal Dose Concentration: | 10 mg/Kg |
| Analyte: | MDMA |

Chemical name: tetrahydrofuran-3-yl N-[2-(1,3-benzodioxol-5-yl)-1-methyl-ethyl]-N-methyl-carbamate
Structural class: carbamate
Mechanistic class: presumed carboxyesterases

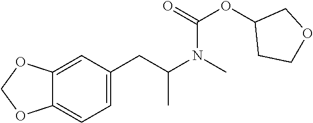 [SMILES: CC(Cc1ccc2c(c1)OCO2)N(C)C(=O)OC1CCOC1]

TABLE 2-30
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-30. 4-Acetoxy-3,3-dimethylbutanoic amide prodrug of MDMA

| | |
|---|---|
| Dosed Test Article: | 4-Acetoxy-3,3-dimethylbutanoic |
| | amide prodrug of MDMA |
| Dose Route: | Oral |
| Nominal Dose Concentration: | 10 mg/Kg |
| Analyte: | MDMA |

Chemical name: (3-{[2-(2H-1,3-Benzodioxol-5-yl)-1-methylethyl]-N-methylcarbamoyl}-2,2-dimethylpropyl acetate
Structural class: amide
Mechanistic class: presumed carboxyesterases+cyclization

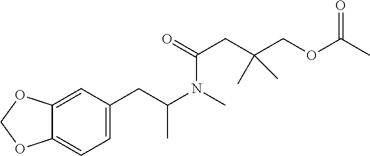 [SMILES: CC(=O)OCC(C)(C)CC(=O)N(C)C(C)Cc1ccc2c(c1)OCO2]

TABLE 2-31
Mean Concentration-Time Profile of Metabolite MDMA
Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-31. SarcHydroxyacetic Pivalate Prodrug of MDMA

| | |
|---|---|
| Dosed Test Article: | SarcHydroxyacetic pivalate |
| | prodrug of MDMA |
| Dose Route: | Oral |
| Nominal Dose Concentration: | 10 mg/Kg |
| Analyte: | MDMA |

Chemical name: [({[2-(2H-1,3-Benzodioxol-5-yl)-1-methylethyl]-N-methylcarbamoyl}methyl)-N-methylcarbamoyl]methyl 2,2-dimethylpropionate
Structural class: amide.
Mechanistic class: presumed carboxyesterases+cyclization

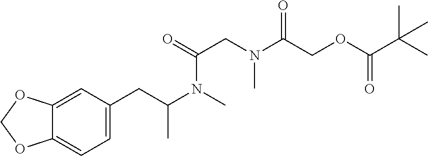 [SMILES: CC(Cc1ccc2c(c1)OCO2)N(C)C(=O)CN(C)C(=O)COC(=O)C(C)(C)C]

TABLE 2-32
MDMA PK Parameters
Mean* Pharmacokinetic Parameters

| PK Parameter | MDMA |
|---|---|
| Cmax (ng/mL) | 25.1 |
| Tmax (h) | 0.50 |
| MRT (h) | 1.95 |
| Tlast (h) | 7.00 |
| AUC0-last (h*ng/mL) | 52.6 |
| AUC0-24 (h*ng/mL) | — |
| AUC0-inf (h*ng/mL) | 57.1 |
| T½ (h) | 2.08 |

*Median calculated for Tmax and Tlast.

FIG. 16. Mean Concentration-Time Profiles of Metabolite MDMA Following Oral Dosing of the SarcHydroxyacetic pivalate prodrug of MDMA (10 mg/Kg) to Male SD Rats

### Example 2-32. Tert-Butyl Carbamate Prodrug of MDMA

| | |
|---|---|
| Dosed Test Article: | Tert-butyl carbamate prodrug of MDMA |
| Dose Route: | Oral |
| Nominal Dose Concentration: | 10 mg/Kg |
| Analyte: | MDMA |

Chemical name: tert-butyl N-[2-(1,3-benzodioxol-5-yl)-1-methyl-ethyl]-N-methyl-carbamate
Structural class: carbamate
Mechanistic class: presumed carboxyesterases

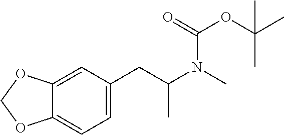 [SMILES: CC(Cc1ccc2c(c1)OCO2)N(C)C(=O)OC(C)(C)C]

TABLE 2-33
Mean Concentration-Time Profile of Metabolite
MDMA Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-33. Pyran Amide Prodrug of MDMA ((4-methyl tetrahydropyran)-4-yl-amide)

| | |
|---|---|
| Dosed Test Article: | Pyran amide prodrug |
| | of MDMA ((4-Methyl |
| | tetrahydropyran)-4-yl-amide) |
| Dose Route: | Oral |
| Nominal Dose Concentration: | 10 mg/Kg |
| Analyte: | MDMA |

Chemical name: N-[2-(2H-1,3-Benzodioxol-5-yl)-1-methylethyl]-N-methyl-4-methyltetrahydro-2H-pyran-4-carboxamide
Structural class: amide
Mechanistic class: presumed amidase

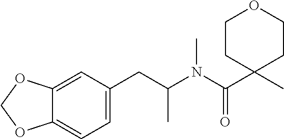 [SMILES: CC(Cc1ccc2c(c1)OCO2)N(C)C(=O)C1(C)CCOCC1]

TABLE 2-34
Mean Concentration-Time Profile of Metabolite
MDMA Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-34. Benzamide Aminal Prodrug of MDMA

| | |
|---|---|
| Dosed Test Article: | Benzamide aminal prodrug of MDMA |
| Dose Route: | Oral |
| Nominal Dose Concentration: | 10 mg/Kg |
| Analyte: | MDMA |

Chemical name: N-({[2-(2H-1,3-Benzodioxol-5-yl)-1-methyl-ethyl]-N-methylamino}methyl)benzamide
Structural class: Mannich base
Mechanistic class: presumed amidase+chemical breakdown

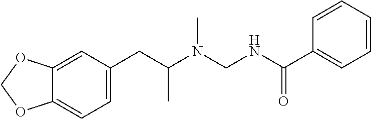 [SMILES: CC(Cc1ccc2c(c1)OCO2)N(C)CNC(=O)c1ccccc1]

TABLE 2-35
MDMA PK Parameters
Mean* Pharmacokinetic Parameters

| PK Parameter | MDMA |
|---|---|
| Cmax (ng/mL) | 263 |
| Tmax (h) | 1.00 |
| MRT (h) | 3.08 |
| Tlast (h) | 24.0 |
| AUC0-last (h*ng/mL) | 719 |
| AUC0-24 (h*ng/mL) | 982 |
| AUC0-inf (h*ng/mL) | 1140 |
| T½ (h) | 10.2 |

*Median calculated for Tmax and Tlast.

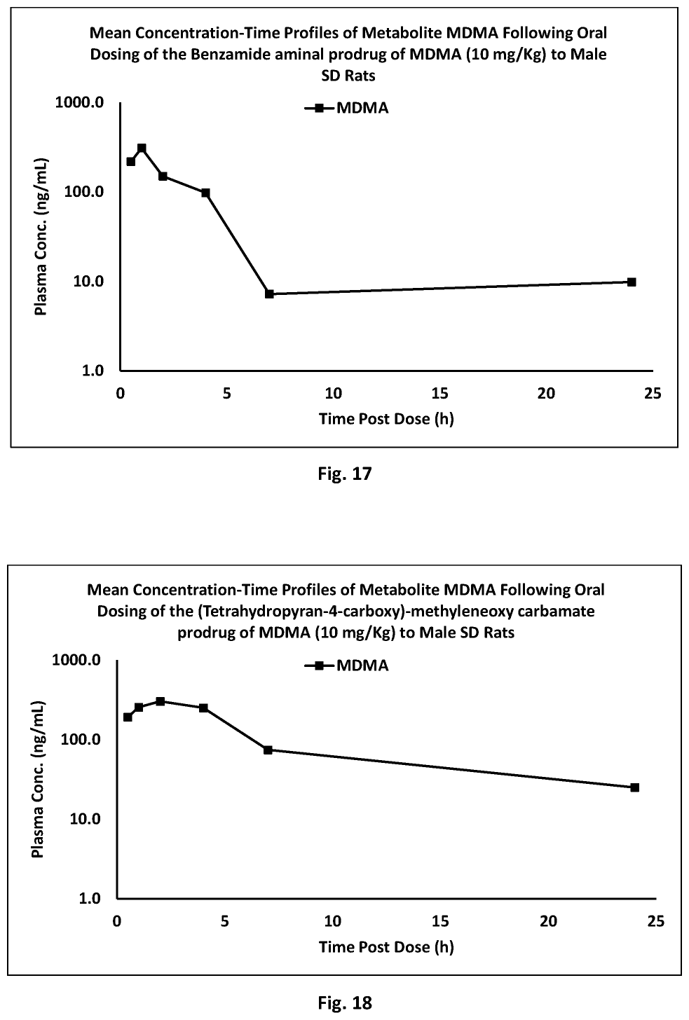

FIG. 17. Mean Concentration-Time Profiles of Metabolite MDMA Following Oral Dosing of the Benzamide aminal prodrug of MDMA (10 mg/Kg) to Male SD Rats

### Example 2-35. oxetane-3-yl carbamate Prodrug of MDMA

| | |
|---|---|
| Dosed Test Article: | Oxetane-3-yl carbamate |
| | prodrug of MDMA |
| Dose Route: | Oral |
| Nominal Dose Concentration: | 10 mg/Kg |
| Analyte: | MDMA |

Chemical name: Oxetan-3-yl N-[2-(1,3-benzodioxol-5-yl)-1-methyl-ethyl]-N-methyl-carbamate
Structural class: carbamate
Mechanistic class: presumed carboxyesterase

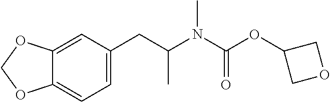 [SMILES: CC(Cc1ccc2c(c1)OCO2)N(C)C(=O)OC1COC1]

TABLE 2-36
Mean Concentration-Time Profile of Metabolite
MDMA Following Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-36. (Tetrahydropyran-4-carboxy)-methyleneoxy carbamate Prodrug of MDMA

| | |
|---|---|
| Dosed Test Article: | (Tetrahydropyran-4-carboxy)-methyleneoxy carbamate prodrug |
| | of MDMA |
| Dose Route: | Oral |
| Nominal Dose Concentration: | 10 mg/Kg |
| Analyte: | MDMA |

Chemical name: {[2-(2H-1,3-Benzodioxol-5-yl)-1-methyl-ethyl]-N-methylaminocarbonyloxy}methyl tetrahydro-2H-pyran-4-carboxylate
Structural class: carbamate
Mechanistic class: presumed carboxyesterase+chemical breakdown

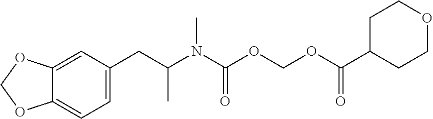 [SMILES: CC(Cc1ccc2c(c1)OCO2)N(C)C(=O)OCOC(=O)C1CCOCC1]

TABLE 2-37
MDMA PK Parameters
Mean* Pharmacokinetic Parameters

| PK Parameter | MDMA |
|---|---|
| Cmax (ng/mL) | 310 |
| Tmax (h) | 2.00 |
| MRT (h) | 4.28 |
| Tlast (h) | 7.00 |
| AUC0-last (h*ng/mL) | 1640 |
| AUC0-24 (h*ng/mL) | 1440 |
| AUC0-inf (h*ng/mL) | 1820 |
| T½ (h) | 10.7 |

*Median calculated for Tmax and Tlast.

FIG. 18. Mean Concentration-Time Profiles of Metabolite MDMA Following Oral Dosing of the (Tetrahydropyran-4-carboxy)-methyleneoxy carbamate prodrug of MDMA (10 mg/Kg) to Male SD Rats

### Example 2-37. Tert-Butyl-Adipate Methyleneoxy Carbamate Prodrug of MDMA

| | |
|---|---|
| Dosed Test Article: | Tert-butyl-adipate methyleneoxy carbamate prodrug of MDMA |
| Dose Route: | Oral |
| Nominal Dose Concentration: | 10 mg/Kg |
| Analyte: | MDMA |

Chemical name: {[2-(2H-1,3-Benzodioxol-5-yl)-1-methylethyl]-N-methylaminocarbonyloxy}methyl tert-butyl adipate
Structural class: carbamate
Mechanistic class: presumed carboxyesterase+pH-dependent cyclization+chemical breakdown

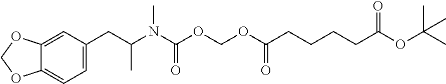 [SMILES: CC(Cc1ccc2c(c1)OCO2)N(C)C(=O)OCOC(=O)CCCCC(=O)OC(C)(C)C]

TABLE 2-38
MDMA PK Parameters
Mean* Pharmacokinetic Parameters

| PK Parameter | MDMA |
|---|---|
| Cmax (ng/mL) | 295 |
| Tmax (h) | 2.00 |
| MRT (h) | 3.48 |
| Tlast (h) | 7.00 |
| AUC0-last (h*ng/mL) | 1500 |
| AUC0-24 (h*ng/mL) | 1760 |
| AUC0-inf (h*ng/mL) | 1790 |
| T½ (h) | 4.25 |

*Median calculated for Tmax and Tlast.

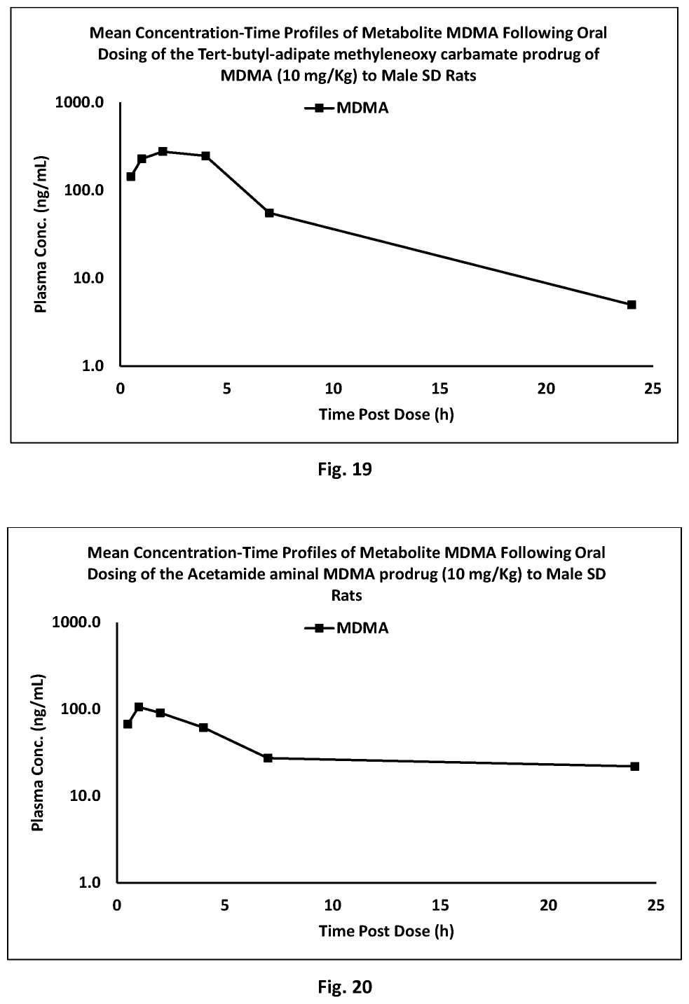

FIG. 19. Mean Concentration-Time Profiles of Metabolite MDMA Following Oral Dosing of the Tert-butyl-adipate methyleneoxy carbamate prodrug of MDMA (10 mg/Kg) to Male SD Rats

### Example 2-38. Acetamide Aminal Prodrug of MDMA

| | |
|---|---|
| Dosed Test Article: | Acetamide aminal prodrug of MDMA |
| Dose Route: | Oral |
| Nominal Dose Concentration: | 10 mg/Kg |
| Analyte: | MDMA |

Chemical name: N-({[2-(2H-1,3-Benzodioxol-5-yl)-1-methyl-ethyl]-N-methylamino}methyl)acetamide
Structural class: Mannich base
Mechanistic class: presumed amidase+chemical breakdown

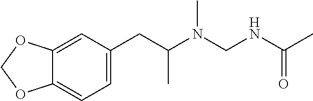 [SMILES: CC(=O)NCN(C)C(C)Cc1ccc2c(c1)OCO2]

TABLE 2-39
MDMA PK Parameters
Mean* Pharmacokinetic Parameters

| PK Parameter | MDMA |
|---|---|
| Cmax (ng/mL) | 106 |
| Tmax (h) | 1.00 |
| MRT (h) | 4.70 |
| Tlast (h) | 7.00 |
| AUC0-last (h*ng/mL) | 571 |
| AUC0-24 (h*ng/mL) | 858 |
| AUC0-inf (h*ng/mL) | 1080 |
| T½ (h) | 10.2 |

*Median calculated for Tmax and Tlast.

FIG. 20. Mean Concentration-Time Profiles of Metabolite MDMA Following Oral Dosing of the Acetamide aminal MDMA prodrug (10 mg/Kg) to Male SD Rats

### Example 2-39. Methyleneoxysuccinate (Protected) Carbamate Prodrug of MDMA

| | |
|---|---|
| Dosed Test Article: | Methyleneoxysuccinate (protected) |
| | carbamate prodrug of MDMA |
| Dose Route: | Oral |
| Nominal Dose Concentration: | 10 mg/Kg |
| Analyte: | MDMA |

Chemical name: {[2-(2H-1,3-Benzodioxol-5-yl)-1-methylethyl]-N-methylaminocarbonyloxy}methyl tert-butyl succinate
Structural class: carbamate
Mechanistic class: presumed carboxyesterase+pH-dependent cyclization+chemical breakdown

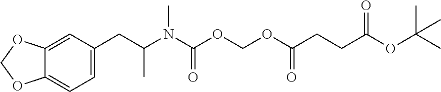 [SMILES: CC(Cc1ccc2c(c1)OCO2)N(C)C(=O)OCOC(=O)CCC(=O)OC(C)(C)C]

TABLE 2-40
MDMA PK Parameters
Mean* Pharmacokinetic Parameters

| PK Parameter | MDMA |
|---|---|
| Cmax (ng/mL) | 144 |
| Tmax (h) | 4.00 |
| MRT (h) | 4.26 |
| Tlast (h) | 7.00 |
| AUC0-last (h*ng/mL) | 892 |
| AUC0-24 (h*ng/mL) | 1360 |
| AUC0-inf (h*ng/mL) | 1250 |
| T½ (h) | 5.37 |

*Median calculated for Tmax and Tlast.

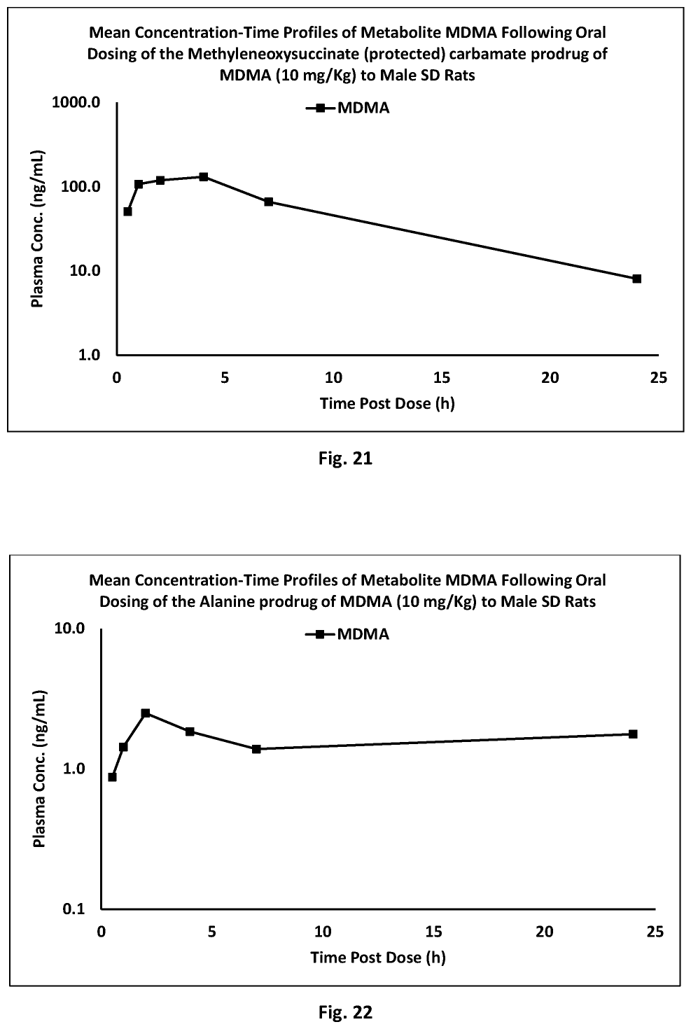

FIG. 21. Mean Concentration-Time Profiles of Metabolite MDMA Following Oral Dosing of the Methyleneoxysuccinate (protected) carbamate prodrug of MDMA (10 mg/Kg) to Male SD Rats

### Example 2-40. Adipate Prodrug of MDMA

| | |
|---|---|
| Dosed Test Article: | Adipate prodrug of MDMA |
| Dose Route: | Oral |
| Nominal Dose Concentration: | 10 mg/Kg |
| Analyte: | MDMA |

Chemical name: 5-{[2-(2H-1,3-Benzodioxol-5-yl)-1-methyl-ethyl]-N-methylcarbamoyl}valeric acid
Structural class: amide
Mechanistic class: presumed pH-dependent cyclization

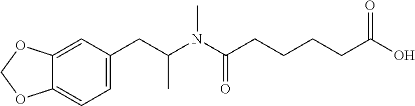 [SMILES: CC(Cc1ccc2c(c1)OCO2)N(C)C(=O)CCCCC(=O)O]

TABLE 2-41
Mean Concentration-Time Profile of Metabolite MDMA Following
Oral Dosing of MDMA Prodrug
Mean Plasma Concentrations (ng/mL)

| Time (h) | MDMA |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-41. Alanine Prodrug of MDMA

| | |
|---|---|
| Dosed Test Article: | Alanine prodrug of MDMA |
| Dose Route: | Oral |
| Nominal Dose Concentration: | 10 mg/Kg |
| Analyte: | MDMA |

Chemical name: (2S)-2-Amino-N-[2-(1,3-benzodioxol-5-yl)-1-methyl-ethyl]-N-methyl-propanamide hydrochloride
Structural class: amino acid
Mechanistic class: presumed amidase or peptidase

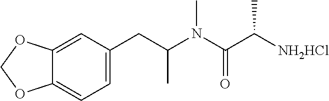

TABLE 2-42
MDMA PK Parameters
Mean* Pharmacokinetic Parameters

| PK Parameter | MDMA |
|---|---|
| Cmax (ng/mL) | 2.56 |
| Tmax (h) | 2.00 |
| MRT (h) | 12.5 |
| Tlast (h) | 24.0 |
| AUC0-last (h*ng/mL) | 38.7 |
| AUC0-24 (h*ng/mL) | 38.7 |
| AUC0-inf (h*ng/mL) | 274 |
| T½ (h) | 93.6 |

*Median calculated for Tmax and Tlast.

FIG. 22. Mean Concentration-Time Profiles of Metabolite MDMA Following Oral Dosing of the Alanine prodrug of MDMA (10 mg/Kg) to Male SD Rats

### Example 2-42. 3-Methyl-Oxetan-3-Yl Carbamate Prodrug of MDMA

| | |
|---|---|
| Dosed Test Article: | 3-Methyl-oxetan-3-yl carbamate |
| | prodrug of MDMA |
| Dose Route: | Oral |
| Nominal Dose Concentration: | 10 mg/Kg |
| Analyte: | MDMA |

Chemical name: (3-Methyloxetan-3-yl)N-[2-(1,3-benzodioxol-5-yl)-1-methyl-ethyl]-N-methyl-carbamate
Structural class: carbamate
Mechanistic class: presumed carboxyesterase

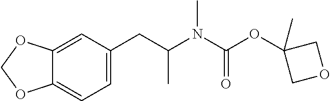 [SMILES: CC(Cc1ccc2c(c1)OCO2)N(C)C(=O)OC1(C)COC1]

TABLE 2-43
MDMA PK Parameters
Mean* Pharmacokinetic Parameters

| PK Parameter | MDMA |
|---|---|
| Cmax (ng/mL) | 24.6 |
| Tmax (h) | 2.00 |
| MRT (h) | 2.61 |
| Tlast (h) | 2.00 |
| AUC0-last (h*ng/mL) | 80.30 |
| AUC0-24 (h*ng/mL) | NC |
| AUC0-inf (h*ng/mL) | NC |
| T½ (h) | NC |

*Median calculated for Tmax and Tlast.
NC: Not Calculated

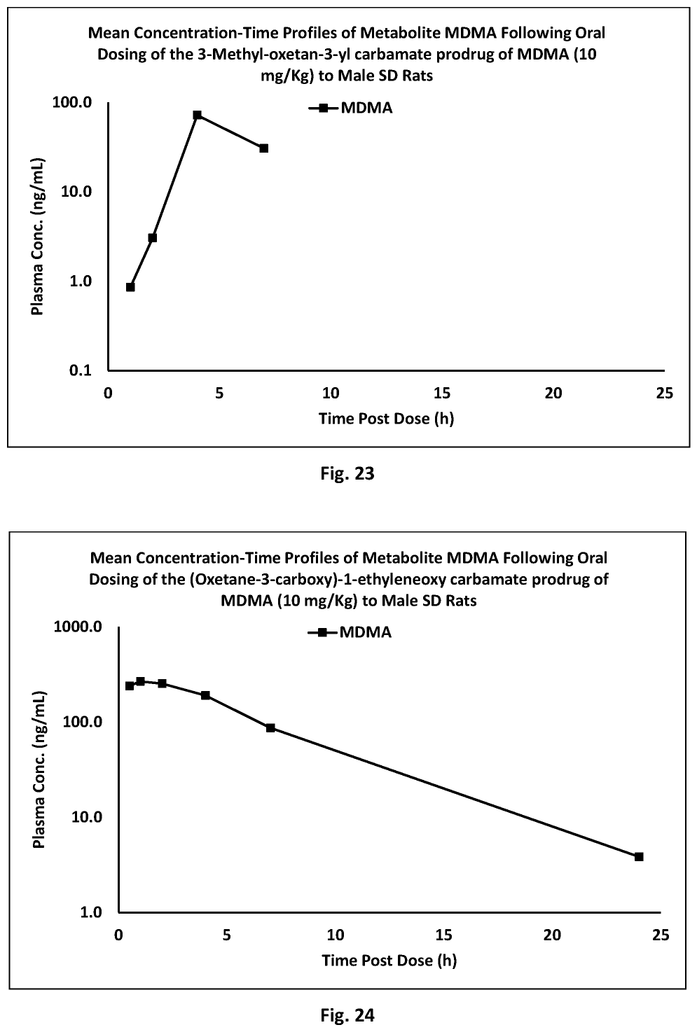

FIG. 23. Mean Concentration-Time Profiles of Metabolite MDMA Following Oral Dosing of the 3-Methyl-oxetan-3-yl carbamate prodrug of MDMA (10 mg/Kg) to Male SD Rats

### Example 2-43. (Oxetane-3-carboxy)-1-ethyleneoxy carbamate Prodrug of MDMA

| | |
|---|---|
| Dosed Test Article: | (Oxetane-3-carboxy)-1-ethyleneoxy |
| | carbamate prodrug of MDMA |
| Dose Route: | Oral |
| Nominal Dose Concentration: | 10 mg/Kg |
| Analyte: | MDMA |

Chemical name: 1-{[2-(2H-1,3-Benzodioxol-5-yl)-1-methyl-ethyl]-N-methylaminocarbonyloxy}ethyl 3-oxetanecarboxylate
Structural class: carbamate
Mechanistic class: presumed carboxyesterase+chemical breakdown

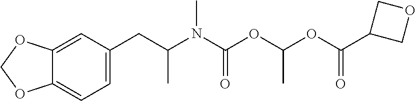 [SMILES: CC(OC(=O)C1COC1)OC(=O)N(C)C(C)Cc1ccc2c(c1)OCO2]

TABLE 2-44
MDMA PK Parameters
Mean* Pharmacokinetic Parameters

| PK Parameter | MDMA |
|---|---|
| Cmax (ng/mL) | 269 |
| Tmax (h) | 1.00 |
| MRT (h) | 4.09 |
| Tlast (h) | 24.0 |
| AUC0-last (h*ng/mL) | 1760 |
| AUC0-24 (h*ng/mL) | — |
| AUC0-inf (h*ng/mL) | 1970 |
| T½ (h) | 3.72 |

*Median calculated for Tmax and Tlast.

FIG. 24. Mean Concentration-Time Profiles of Metabolite MDMA Following Oral Dosing of the (Oxetane-3-carboxy)-1-ethyleneoxy carbamate prodrug of MDMA (10 mg/Kg) to Male SD Rats

### Example 2-44. (Oxetane-3-carboxy)-methyleneoxy carbamate Prodrug of MDMA

| | |
|---|---|
| Dosed Test Article: | (Oxetane-3-carboxy)-methyleneoxy |
| | carbamate prodrug of MDMA |
| Dose Route: | Oral |
| Nominal Dose Concentration: | 10 mg/Kg |
| Analyte: | MDMA |

Chemical name: {[2-(2H-1,3-Benzodioxol-5-yl)-1-methyl-ethyl]-N-methylaminocarbonyloxy}methyl 3-oxetanecarboxylate
Structural class: carbamate
Mechanistic class: presumed carboxyesterase+chemical breakdown

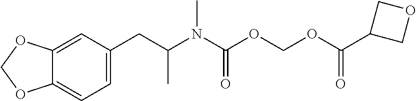 [SMILES: CC(Cc1ccc2c(c1)OCO2)N(C)C(=O)OCOC(=O)C1COC1]

TABLE 2-45
MDMA PK Parameters
Mean* Pharmacokinetic Parameters

| PK Parameter | MDMA |
|---|---|
| Cmax (ng/mL) | 505 |
| Tmax (h) | 2.00 |
| MRT (h) | 4.37 |
| Tlast (h) | 24.0 |
| AUC0-last (h*ng/mL) | 3220 |
| AUC0-24 (h*ng/mL) | 3220 |
| AUC0-inf (h*ng/mL) | 3600 |
| T½ (h) | 3.20 |

*Median calculated for Tmax and Tlast.

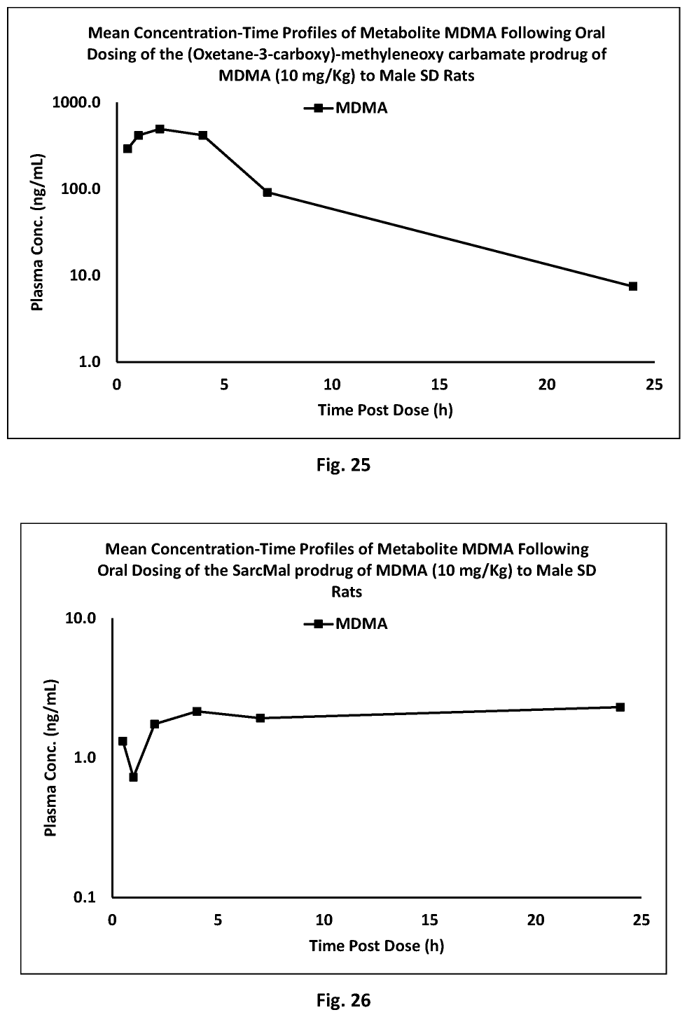

FIG. 25. Mean Concentration-Time Profiles of Metabolite MDMA Following Oral Dosing of the (Oxetane-3-carboxy)-methyleneoxy carbamate prodrug of MDMA (10 mg/Kg) to Male SD Rats

### Example 2-45. SarcMal Prodrug of MDMA

| | |
|---|---|
| Dosed Test Article: | SarcMal prodrug of MDMA |
| Dose Route: | Oral |
| Nominal Dose Concentration: | 10 mg/Kg |
| Analyte: | MDMA |

Chemical name: Ammonium 3-[[2-[[2-1,3-benzodioxol-5-yl)-1-methyl-ethyl]-methyl-amino]-2-oxo-ethyl]-methyl-amino]-3-oxo-propionate
Structural class: amide
Mechanistic class: presumed pH-dependent cyclization and/or presumed amidase

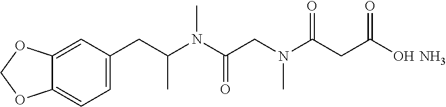

TABLE 2-46
MDMA PK Parameters
Mean* Pharmacokinetic Parameters

| PK Parameter | MDMA |
|---|---|
| Cmax (ng/mL) | 2.36 |
| Tmax (h) | 4.00 |
| MRT (h) | 10.3 |
| Tlast (h) | 24.0 |
| AUC0-last (h*ng/mL) | 31.5 |
| AUC0-24 (h*ng/mL) | 46.1 |
| AUC0-inf (h*ng/mL) | NC |
| T½ (h) | NC |

*Median calculated for Tmax and Tlast.

FIG. 26. Mean Concentration-Time Profiles of Metabolite MDMA Following Oral Dosing of the SarcMal prodrug of MDMA (10 mg/Kg) to Male SD Rats

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A compound of Formula (I′), or a stereoisomer, a hydrate, or a pharmaceutically acceptable salt thereof: 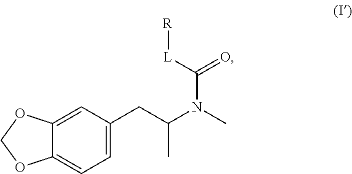
wherein:
L is bond, —O—, or NR′;
R and R are each alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein the compound of Formula (I′) is a compound of Formula (I), or a stereoisomer, a hydrate, or a pharmaceutically acceptable salt thereof: 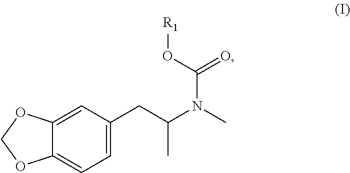
wherein:
R¹ is alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₅ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

3. The compound of claim 2, wherein the compound of Formula (I) has a structure of Formula (Ia), or a stereoisomer, a hydrate, or a pharmaceutically acceptable salt thereof:

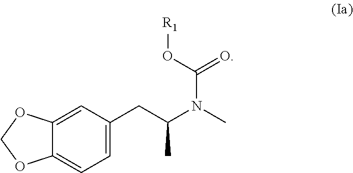

4. The compound of claim 2, wherein the compound of Formula (I) has a structure of Formula (Ib), or a stereoisomer, a hydrate, or a pharmaceutically acceptable salt thereof:

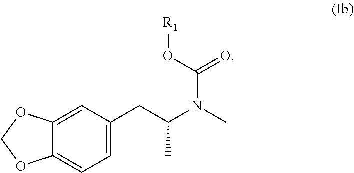

5. The compound of any one of claims 2-4, wherein R¹ is substituted alkyl.

6. The compound of any one of claims 2-5, wherein R¹ is alkyl substituted with heteroalkyl, heterocyclylalkyl, or heteroaryl, wherein each of heteroalkyl, heterocyclylalkyl, and heteroaryl is unsubstituted or substituted.

7. The compound of any one of claims 2-6, wherein R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

8. The compound of claim 7, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

9. The compound of claim 7, wherein R¹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

10. The compound of claim 2, wherein the compound of Formula (I) has a structure of Formula (I-1), or a stereoisomer, a hydrate, or a pharmaceutically acceptable salt thereof: 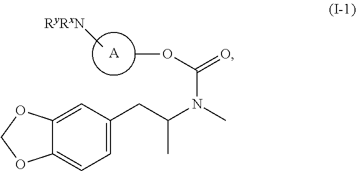
wherein 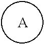
is cycloalkyl or heterocyclylalkyl, and each of R^{x} and R^{y} is alkyl or hydrogen, or R^{x} and R^{y} together with the atom to which they are attached form a heterocyclylalkyl ring.

11. The compound of claim 10, wherein the compound of Formula (I-1) has a structure of Formula (I-1a), or a stereoisomer, a hydrate, or a pharmaceutically acceptable salt thereof: 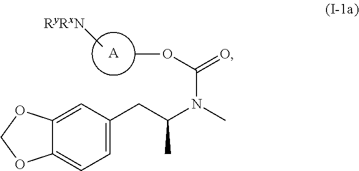
wherein 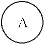
is cycloalkyl or heterocyclylalkyl, and each of R^{x} and R^{y} is alkyl or hydrogen, or R^{x} and R^{y} together with the atom to which they are attached form a heterocyclylalkyl ring.

12. The compound of claim 10, wherein the compound of Formula (I-1) has a structure of Formula (I-1b), or a stereoisomer, a hydrate, or a pharmaceutically acceptable salt thereof: 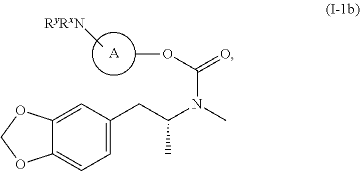
wherein 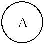
is cycloalkyl or heterocyclylalkyl, and each of R^{x} and R^{y} is alkyl or hydrogen, or R^{x} and R^{y} together with the atom to which they are attached form a heterocyclylalkyl ring.

13. The compound of claim 1, wherein the compound of Formula (I′) has a structure of Formula (II), or a stereoisomer, a hydrate, or a pharmaceutically acceptable salt thereof: 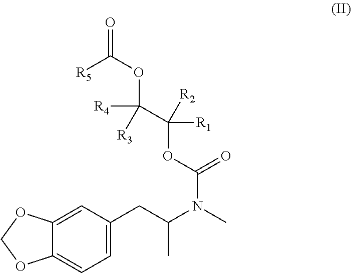
wherein:
each of R¹ and R² is independently hydrogen or alkyl optionally substituted with one or more Q, or R¹ and R² together with the atom to which they are attached form a cycloalkyl ring;
each of R³ and R⁴ is independently hydrogen or alkyl optionally substituted with one or more Q, or R³ and R⁴ together with the atom to which they are attached form a cycloalkyl ring; and
R⁵ is hydrogen, alkyl, cycloalkyl, heteroalkyl, heterocyclylalkyl, aryl, or heteroaryl, each of which is optionally substituted with one or more Q, or R⁵ together with the carbonyl to which R⁵ is attached form an amino acid residue;
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

14. The compound of claim 13, wherein the compound of Formula (II) has a structure of Formula (IIa), or a stereoisomer, a hydrate, or a pharmaceutically acceptable salt thereof:

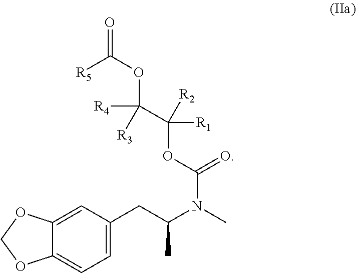

15. The compound of claim 13, wherein the compound of Formula (II) has a structure of Formula (IIb), or a stereoisomer, a hydrate, or a pharmaceutically acceptable salt thereof:

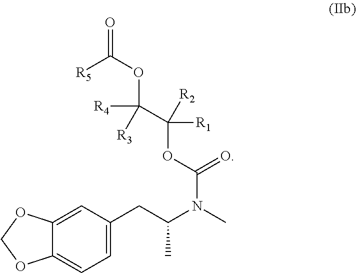

16. The compound of any one of claims 13-15, wherein R³ and R⁴ are each hydrogen.

17. The compound of any one of claims 13-15, wherein R³ and R⁴ are each independently alkyl.

18. The compound of claim 17, wherein R³ and R⁴ are each independently methyl.

19. The compound of any one of claims 13-15, wherein R³ and R⁴ together with the atom to which they are attached form a cycloalkyl or heterocyclyl ring.

20. The compound of any one of claims 13-19, wherein R¹ and R² are each hydrogen.

21. The compound of any one of claims 13-15, wherein R³ and R⁴ are each independently alkyl, and R¹ and R² are each hydrogen.

22. The compound of any one of claims 13-15, wherein each of R¹, R², R³, and R⁴ is hydrogen.

23. The compound of any one of claims 13-22, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl, each of which is optionally substituted with one or more Q.

24. The compound of any one of claims 13-22, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl.

25. The compound of claim 1, wherein the compound of Formula (I′) is a compound of Formula (III), or a stereoisomer, a hydrate, or a pharmaceutically acceptable salt thereof: 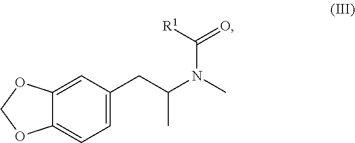
wherein:
R¹ is alkyl, alkenyl, heteroalkyl, cycloalkyl, heterocyclylalkyl, aryl, or heteroaryl, each of which is optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

26. The compound of claim 25, wherein the compound of Formula (III) has a structure of Formula (IIIa), or a stereoisomer, a hydrate, or a pharmaceutically acceptable salt thereof:

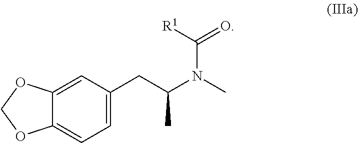

27. The compound of claim 25, wherein the compound of Formula (III) has a structure of Formula (IIIb), or a stereoisomer, a hydrate, or a pharmaceutically acceptable salt thereof:

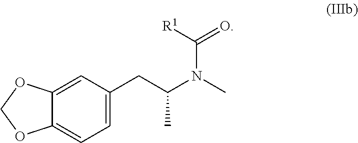

28. The compound of any one of claims 25-27, wherein R¹ is unsubstituted alkyl.

29. The compound of any one of claims 25-27, wherein R¹ is substituted alkyl.

30. The compound of any one of claims 25-27 and 29, wherein R¹ is alkyl substituted with heteroalkyl, heterocyclylalkyl, or heteroaryl, wherein each of heteroalkyl, heterocyclylalkyl, and heteroaryl is unsubstituted or substituted.

31. The compound of any one of claims 25-27, wherein R¹ is methyl, ethyl, n-propyl, isopropyl, tert-butyl, n-pentyl, iso-amyl, n-hexyl, n-heptyl, n-octyl, n-nonyl, —CH₂CH₂OCH₃, —CH₂C(O)C(CH₃)₃, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

32. The compound of claim 31, wherein R¹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

33. The compound of claim 31, wherein R¹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

34. The compound of claim 1, wherein the compound of Formula (I) has a structure of Formula (IV), or a stereoisomer, a hydrate, or a pharmaceutically acceptable salt thereof: 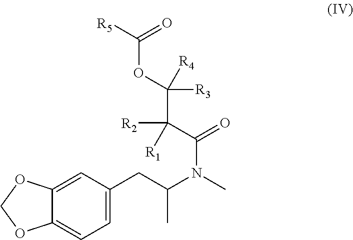
wherein:
each of R¹ and R² is independently hydrogen or alkyl optionally substituted with one or more Q, or R¹ and R² together with the atom to which they are attached form a cycloalkyl or heterocyclyl ring;
each of R³ and R⁴ is independently hydrogen or alkyl optionally substituted with one or more Q, or R³ and R⁴ together with the atom to which they are attached form a cycloalkyl or heterocyclyl ring;
and R⁵ is alkyl, cycloalkyl, heteroalkyl, heterocyclylalkyl, aryl, or heteroaryl, each of which is optionally substituted with one or more Q, or R⁵ together with the carbonyl to which R⁵ is attached form an amino acid residue;
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

35. The compound of claim 34, wherein the compound of Formula (IV) has a structure of Formula (IVa), or a stereoisomer, a hydrate, or a pharmaceutically acceptable salt thereof:

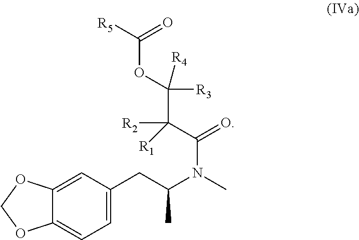

36. The compound of claim 34, wherein the compound of Formula (IV) has a structure of Formula (IVb), or a stereoisomer, a hydrate, or a pharmaceutically acceptable salt thereof:

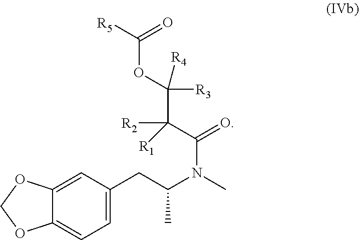

37. The compound of claim 1, wherein the compound of Formula (I′) has a structure of Formula (V), or a stereoisomer, a hydrate, or a pharmaceutically acceptable salt thereof: 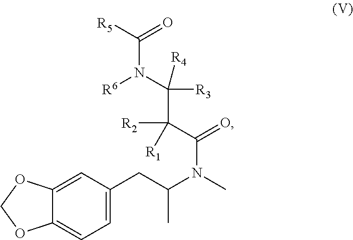
wherein:
each of R¹ and R² is independently hydrogen or alkyl optionally substituted with one or more Q, or R¹ and R² together with the atom to which they are attached form a cycloalkyl or heterocyclyl ring;
each of R³ and R⁴ is independently hydrogen or alkyl optionally substituted with one or more Q, or R³ and R⁴ together with the atom to which they are attached form a cycloalkyl or heterocyclyl ring; and
each of R⁵ and R⁶ is independently hydrogen, alkyl, cycloalkyl, heteroalkyl, heterocyclylalkyl, aryl, or heteroaryl, each of which is optionally substituted with one or more Q;
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

38. The compound of claim 37, wherein the compound of Formula (V) has a structure of Formula (Va), or a stereoisomer, a hydrate, or a pharmaceutically acceptable salt thereof:

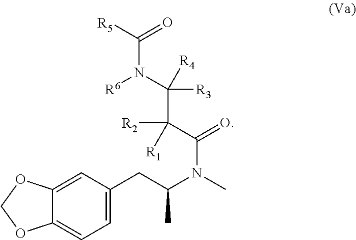

39. The compound of claim 37, wherein the compound of Formula (V) has a structure of Formula (Vb), or a stereoisomer, a hydrate, or a pharmaceutically acceptable salt thereof:

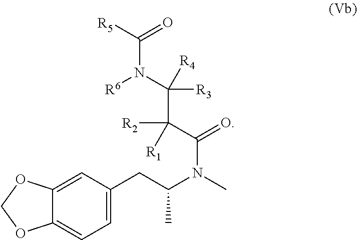

40. The compound of any one of claims 37-39, wherein R³ and R⁴ are each hydrogen.

41. The compound of any one of claims 37-39, wherein R³ and R⁴ are each independently alkyl.

42. The compound of claim 41, wherein R³ and R⁴ are each independently methyl.

43. The compound of any one of claims 37-39, wherein R³ and R⁴ together with the atom to which they are attached form a cycloalkyl or heterocyclyl ring.

44. The compound of any one of claims 37-43, wherein R¹ and R² are each hydrogen.

45. The compound of any one of claims 37-39, wherein R³ and R⁴ are each independently alkyl, and R¹ and R² are each hydrogen.

46. The compound of any one of claims 37-39, wherein each of R¹, R², R³, and R⁴ is hydrogen.

47. The compound of any one of claims 37-46, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl, each of which is optionally substituted with one or more Q.

48. The compound of any one of claims 37-46, wherein R⁵ is methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, or 6-pyrimidyl.

49. The compound of any one of claims 37-48, wherein R⁶ is hydrogen or alkyl.

50. The compound of claim 49, wherein R⁶ is hydrogen.

51. The compound of claim 49, wherein R⁶ is alkyl.

52. The compound of claim 51, wherein R⁶ is methyl.

53. The compound of claim 1, wherein the compound of Formula (I′) has a structure of Formula (VI), or a stereoisomer, a hydrate, or a pharmaceutically acceptable salt thereof: 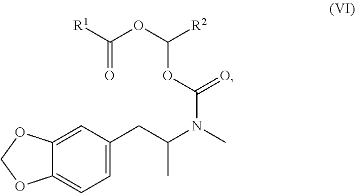
wherein:
R¹ is hydrogen, or R¹ is alkyl, cycloalkyl, heteroalkyl, heterocyclylalkyl, aryl, or heteroaryl, each of which is optionally substituted with one or more Q; or R¹ and the carbonyl to which R¹ is attached form an amino acid residue;
R² is hydrogen or alkyl optionally substituted with one or more Q; and
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —N^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

54. The compound of claim 53, wherein the compound of Formula (VI) has a structure of Formula (VI-1), or a stereoisomer, a hydrate, or a pharmaceutically acceptable salt thereof: 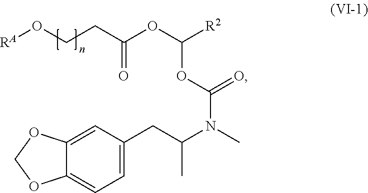
wherein R^{A} is alkyl, heteroalkyl, cycloalkyl, or heterocyclylalkyl, each of which is substituted or unsubstituted; R² is alkyl that is substituted or unsubstituted, or hydrogen; and n is 1, 2, 3, 4, 5, or 6.

55. The compound of claim 54, wherein R^{A} is methyl, ethyl, isopropyl, n-propyl, tert-butyl, n-butyl, n-pentyl, iso-amyl, cyclopropyl, cyclobutyl, cyclopentyl, or cyclohexyl.

56. The compound of claim 55, wherein R^{A} is methyl.

57. The compound of claim 53, wherein the compound of Formula (VI) has a structure of Formula (VI-2), or a stereoisomer, a hydrate, or a pharmaceutically acceptable salt thereof: 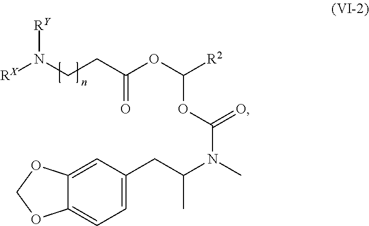
wherein each of R^{X} and R^{Y} is independently hydrogen, alkyl, heteroalkyl, cycloalkyl, or heterocyclylalkyl, wherein alkyl, heteroalkyl, cycloalkyl, or heterocyclylalkyl are substituted or unsubstituted; or R^{X} and R^{Y} together with the atom to which they are attached form a heterocyclylalkyl ring that is substituted or unsubstituted; R² is alkyl that is substituted or unsubstituted, or hydrogen; and n is 1, 2, 3, 4, 5, or 6.

58. The compound of claim 57, wherein each of R^{X} and R^{Y} is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

59. The compound of claim 53, wherein the compound of Formula (VI) has a structure of Formula (VI-3), or a stereoisomer, a hydrate, or a pharmaceutically acceptable salt thereof: 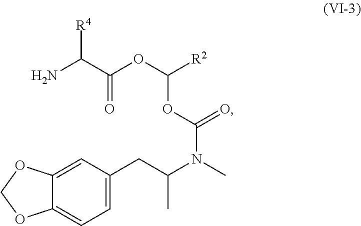
wherein R⁴ is hydrogen, substituted or unsubstituted alkyl, substituted or unsubstituted heteroalkyl, or an amino acid side chain; and R² is alkyl that is substituted or unsubstituted, or hydrogen.

60. The compound of claim 1, wherein the compound of Formula (I′) has a structure of Formula (VIII), or a stereoisomer, a hydrate, or a pharmaceutically acceptable salt thereof: 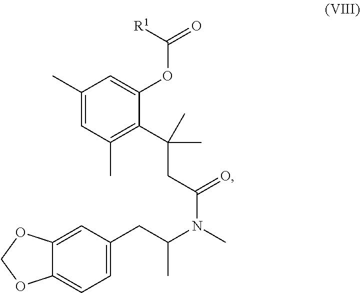
wherein R¹ is hydrogen, or R¹ is alkyl, alkenyl, heteroalkyl, cycloalkyl, heterocyclylalkyl, aryl, or heteroaryl, each of which is optionally substituted with one or more Q, or R¹ and the carbonyl to which R¹ is attached form an amino acid residue.
In certain embodiments, R¹ is hydrogen.

61. The compound of claim 60, wherein R¹ is optionally substituted alkyl or heteroalkyl.

62. The compound of claim 60, wherein R¹ is optionally substituted alkyl.

63. The compound of claim 60, wherein R¹ is unsubstituted alkyl.

64. The compound of claim 63, wherein R¹ is methyl, ethyl, n-propyl, isopropyl, tert-butyl, or n-pentyl.

65. The compound of claim 1, wherein L is bond.

66. The compound of claim 1, wherein L is —O—.

67. The compound of claim 1, wherein L is —NR′—.

68. The compound of any one of claims 65-67, wherein R is alkyl, cycloalkyl, heteroalkyl, heterocyclylalkyl, aryl, or heteroaryl, each of which is optionally substituted with one or more Q;
wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroalkyl, heteroaryl, and heterocyclylalkyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —N^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclylalkyl, each of which is further optionally substituted with one, two, three, or four, substituents Q^{a}; or (iii) R^{b} and R^{c} together with the N atom to which they are attached form heterocyclylalkyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

69. The compound of any one of claims 65-67, wherein R is alkyl or heterocyclylalkyl optionally substituted with one or more Q.

70. The compound of claim 69, wherein R is alkyl substituted with one or more Q.

71. The compound of claim 69, wherein R is heterocyclylalkyl.

72. The compound of any one of claims 65-67, wherein R is optionally substituted methyl, ethyl, n-propyl, n-butyl, i-butyl, sec-butyl, t-butyl, tetrahydrofuranyl, or tetrahydropyranyl.

73. The compound of any one of claims 65-67, wherein R is optionally substituted ethyl, n-propyl, n-butyl, i-butyl, sec-butyl, t-butyl, tetrahydrofuranyl, or tetrahydropyranyl, or R is substituted methyl.

74. The compound of any one of claims 65-67, wherein R is ethyl, n-propyl, i-propyl, i-butyl, sec-butyl, t-butyl, tetrahydrofuranyl, tetrahydropyranyl, —CH₂CH₂—OCH₃, —CH₂CH₂—COOH, —CH₂CH₂CH₂—COOH, —CH₂CH₂CH₂CH₂—COOH, —CH₂—OC(O)C(CH₃)₃, —CH₂CH₂—C(O)OC(CH₃)₃, —CH₂CH₂CH₂—C(O)OC(CH₃)₃, —CH₂CH₂CH₂CH₂—C(O)OC(CH₃)₃, —CH₂N(CH₃)₂, —C(CH₃)NHC(O)OC(CH₃)₃, —C[CH(CH₃)₂]NHC(O)OC(CH₃)₃,

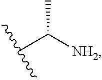 [SMILES: *[C@H](C)N]

75. The compound of any one of claims 65-67, wherein R is optionally substituted alkyl, R′ is hydrogen or unsubstituted alkyl.

76. The compound of claim 75, wherein R′ is hydrogen or methyl.

77. The compound of claim 1, wherein the compound is a compound in Table 1.

78. The compound of claim 1, wherein the compound is a compound in Table 1A.

79. The compound of claim 1, wherein the compound is a compound in Tables 2-15.

80. The compound of claim 1, wherein the compound is selected from the group consisting of: 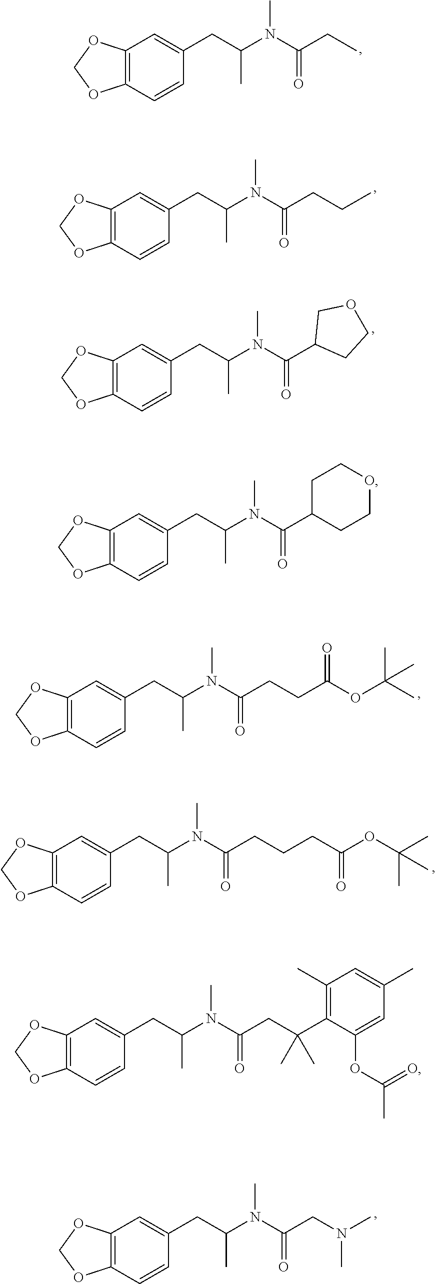 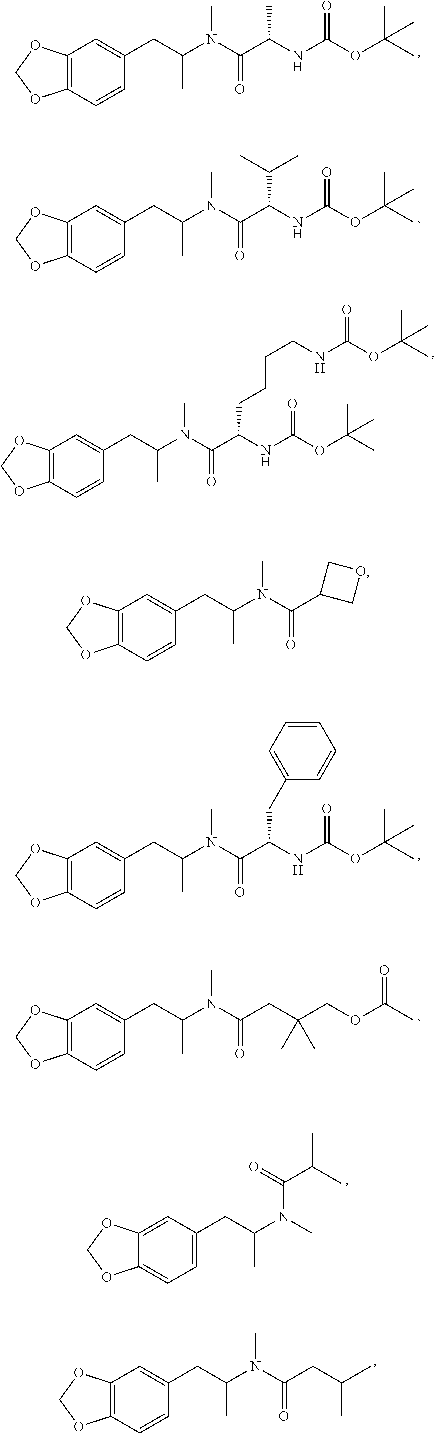 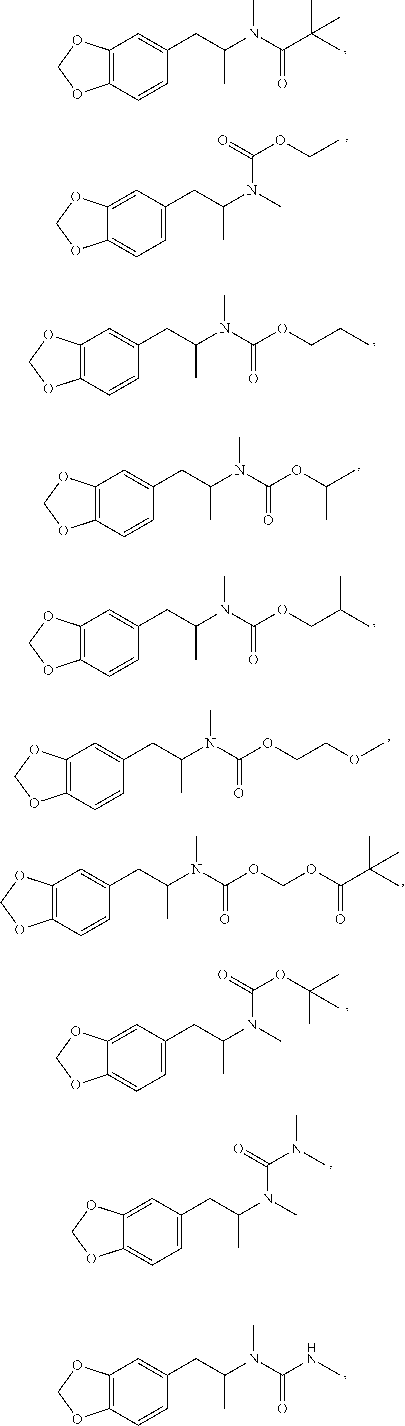 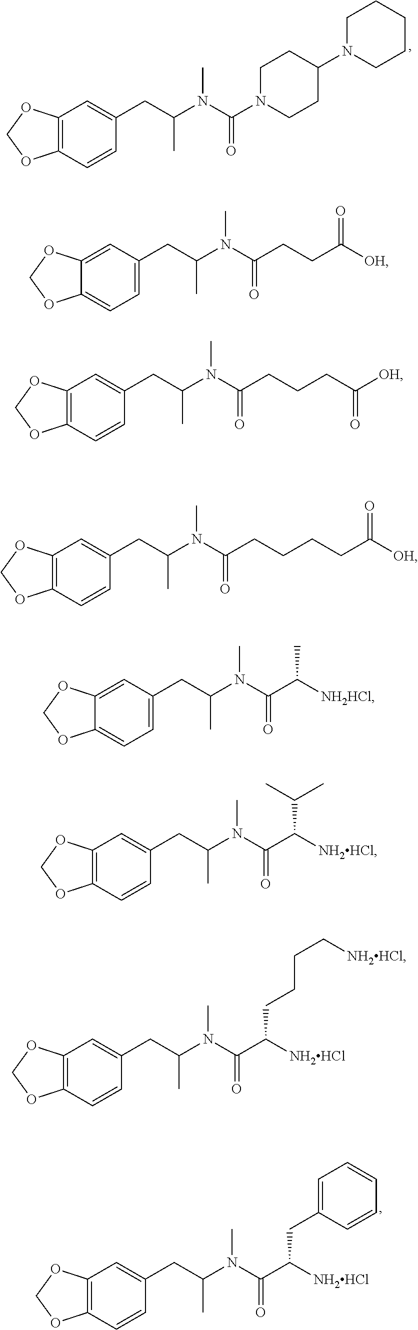 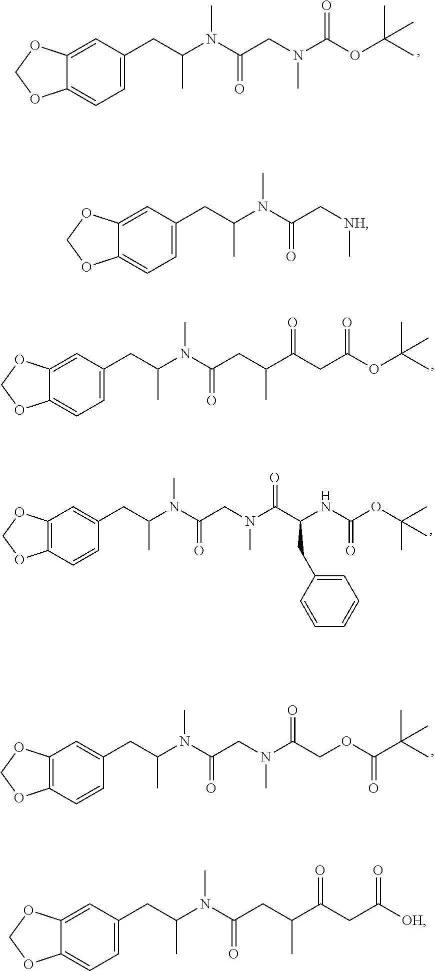 [SMILES: CC(Cc1ccc2c(c1)OCO2)N(C)C(=O)CN(C)C(=O)OC(C)(C)C ; CNCC(=O)N(C)C(C)Cc1ccc2c(c1)OCO2 ; CC(CC(=O)N(C)C(C)Cc1ccc2c(c1)OCO2)C(=O)CC(=O)OC(C)(C)C ; CC(Cc1ccc2c(c1)OCO2)N(C)C(=O)CN(C)C(=O)[C@H](Cc1ccccc1)NC(=O)OC(C)(C)C ; CC(Cc1ccc2c(c1)OCO2)N(C)C(=O)CN(C)C(=O)COC(=O)C(C)(C)C ; CC(CC(=O)N(C)C(C)Cc1ccc2c(c1)OCO2)C(=O)CC(=O)O] 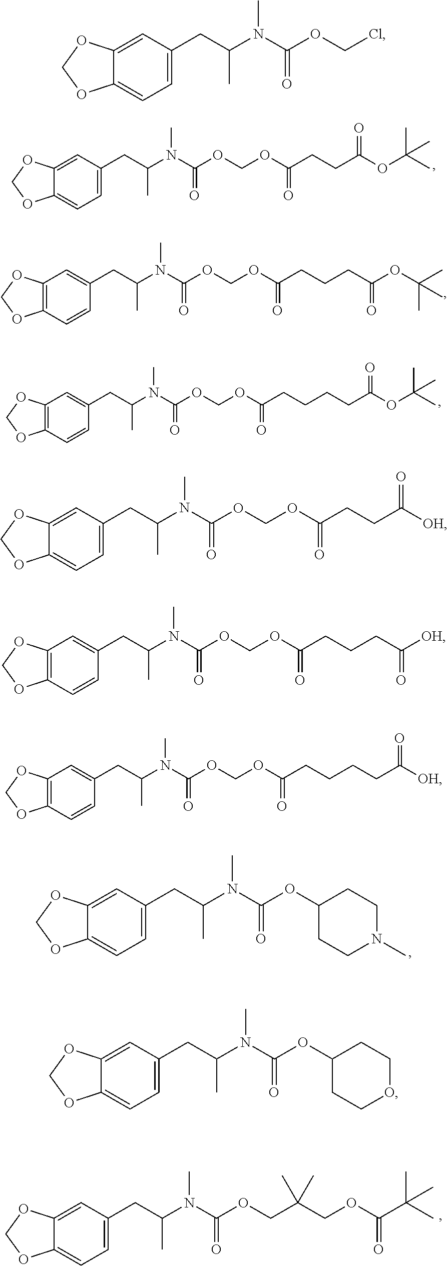 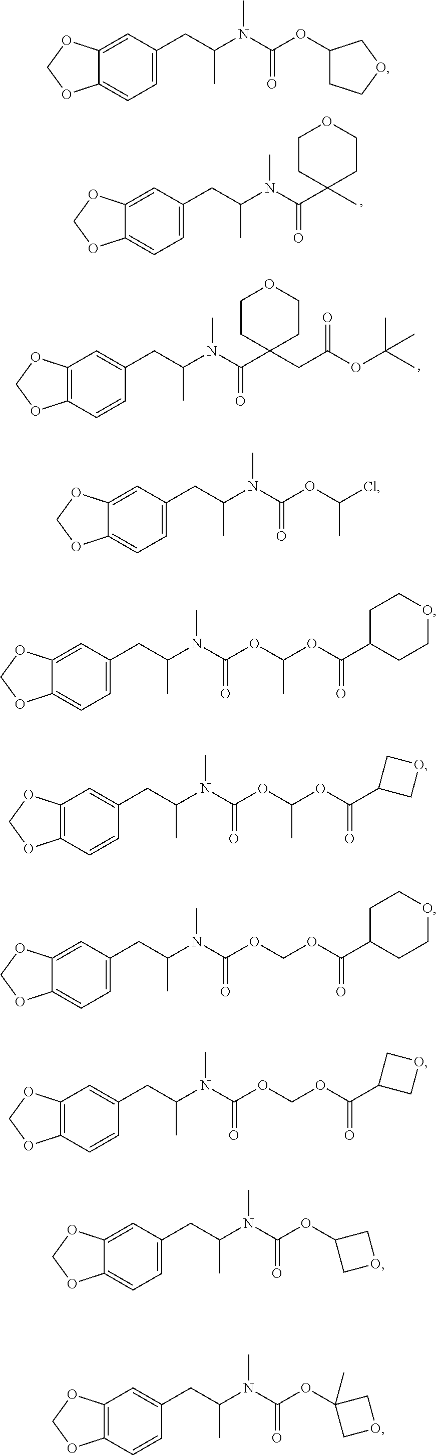 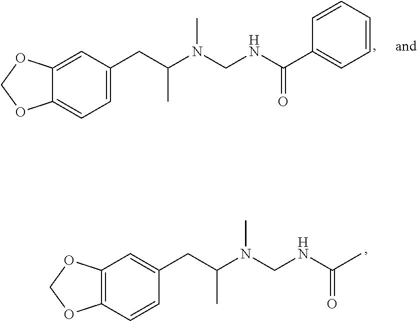 [SMILES: CC(Cc1ccc2c(c1)OCO2)N(C)CNC(=O)c1ccccc1 ; CC(=O)NCN(C)C(C)Cc1ccc2c(c1)OCO2]
or a stereoisomer, a hydrate, or a pharmaceutically acceptable salt thereof.

81. A pharmaceutically composition comprising a compound according to any one of claims 1-80, or a stereoisomer, hydrate, pharmaceutically acceptable salt thereof, and a pharmaceutically acceptable excipient or carrier.

82. A method of treating or preventing a disease, disorder, or condition in which an increased level of 3,4-methylenedioxymethamphetamine (MDMA) is beneficial, comprising administering to a subject in need thereof an effective amount of a compound or stereoisomer, hydrate, pharmaceutically acceptable salt thereof according to any one of claims 1-80, or a pharmaceutically composition of claim 81.

83. The method of claim 82, wherein the disease, disorder, or condition comprises post-traumatic stress disorder, major depression, schizophrenia, alzheimer's disease, frontotemporal dementia, Parkinson's disease, Parkinson's dementia, dementia, lewy body dementia, multiple system atrophy, or substance abuse.

84. The method of claim 82, wherein the disease, disorder, or condition comprises musculoskeletal pain disorder including fibromyalgia, muscle pain, joint stiffness, osteoarthritis, rheumatoid arthritis, muscle cramps.
